SEQUENCE LISTING (126 sequences)

SEQ ID NO: 1 - DNA, 402 bp, Homo sapiens
atggcaatggtctctgcgatgtcctgggtcctgtatttgtggataagtgcttgtgcaatgctactctgccatggatcccttcagcacactttccagcagcatcacctgcacagaccagaaggagggacgtgtgaagtgatagcagcacaccgatgttgcaacaagaatcgcattgaggagcggtcacaaacagtaaagtgttcctgtctacctggaaaagtggctggaacaacaagaaaccggccttcttgcgtcgatgcctccatagtgatttggaaatggtggtgtgagatggagccttgcctagaaggagaagaatgtaagacactccctgacaattctggatggatgtgcgcaacaggcaacaaaattaagaccacgagaattcacccaagaacctaa

SEQ ID NO: 2 - protein, 133 aa, Homo sapiens
MAMVSAMSWVLYLWISACAMLLCHGSLQHTFQQHHLHRPEGGTCEVIAAHRCCNKNRIEERSQTVKCSCLPGKVAGTTRNRPSCVDASIVIWKWWCEMEPCLEGEECKTLPDNSGWMCATGNKIKTTRIHPRT

SEQ ID NO: 3 - DNA, 327 bp, Homo sapiens
tcccttcagcacactttccagcagcatcacctgcacagaccagaaggagggacgtgtgaagtgatagcagcacaccgatgttgcaacaagaatcgcattgaggagcggtcacaaacagtaaagtgttcctgtctacctggaaaagtggctggaacaacaagaaaccggccttcttgcgtcgatgcctccatagtgatttggaaatggtggtgtgagatggagccttgcctagaaggagaagaatgtaagacactccctgacaattctggatggatgtgcgcaacaggcaacaaaattaagaccacgagaattcacccaagaacctaa

SEQ ID NO: 4 - protein, 108 aa, Homo sapiens
SLQHTFQQHHLHRPEGGTCEVIAAHRCCNKNRIEERSQTVKCSCLPGKVAGTTRNRPSCVDASIVIWKWWCEMEPCLEGEECKTLPDNSGWMCATGNKIKTTRIHPRT

SEQ ID NO: 5 - DNA, 396 bp, Homo sapiens
atgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggtaacccattaa

SEQ ID NO: 6 - protein, 130 aa, Homo sapiens
MSKRYLQKATKGKLLIIIFIVTLWGKVVSSANHHKAHHVKTGTCEVVALHCCNKNKIEERSQTVKCSCFPGQVAGTTRAAPSCVDASIVEQKWWCHMQPCLEGEECKVLPDRKGWSCSSGNKVKTTRVTH

SEQ ID NO: 7 - DNA, 306 bp, Homo sapiens
gcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggtaacccattaa

SEQ ID NO: 8 - protein, 101 aa, Homo sapiens
ANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRVTH

SEQ ID NO: 9 - DNA, 399 bp, Homo sapiens
atggcgccatcgcccaggaccggcagccggcaagatgcgaccgccctgcccagcatgtcctcaactttctgggcgttcatgatcctggccagcctgctcatcgcctactgcagtcagctggccgccggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctga

SEQ ID NO: 10 - protein, 132 aa, Homo sapiens
MAPSPRTGSRQDATALPSMSSTFWAFMILASLLIAYCSQLAAGTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 11 - DNA, 273 bp, Homo sapiens
ggcacctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctga

SEQ ID NO: 12 - protein, 90 aa, Homo sapiens
GTCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 13 - DNA, 423 bp, Homo sapiens
atgaggtccccaaggatgagagtctgtgctaagtcagtgttgctgtcgcactggctctttctagcctacgtgttaatggtgtgctgtaagctgatgtccgcctcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtag

SEQ ID NO: 14 - protein, 140 aa, Homo sapiens
MRSPRMRVCAKSVLLSHWLFLAYVLMVCCKLMSASSQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 15 - DNA, 321 bp, Homo sapiens
tcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggtag

SEQ ID NO: 16 - protein, 106 aa, Homo sapiens
SSQHLRGHAGHHQIKQGTCEVVAVHRCCNKNRIEERSQTVKCSCFPGQVAGTTRAQPSCVEASIVIQKWWCHMNPCLEGEDCKVLPDYSGWSCSSGNKVKTTKVTR

SEQ ID NO: 17 - DNA, 115 bp, Homo sapiens
atgagtgagagggtcgagcggaactggagcacgggcggctggctgctggcactgtgcctggcctggctgtggacccacctgaccttggctgccttgcagcctcccactgccacag

SEQ ID NO: 18 - protein, 39 aa, Homo sapiens
MSERVERNWSTGGWLLALCLAWLWTHLTLAALQPPTATV

SEQ ID NO: 19 - DNA, 150 bp, Homo sapiens
tgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacg

SEQ ID NO: 20 - protein, 50 aa, Homo sapiens
LVQQGTCEVIAAHRCCNRNRIEERSQTVKCSCFSGQVAGTTRAKPSCVDA

SEQ ID NO: 21 - DNA, 125 bp, Homo sapiens
cctccatcgtcctgcagagatggtggtgtcagatggagccctgcctgccgggggaggagtgtaaggtgctcccggacctgtcgggatggagctgcagcagtggacacaaagtcaaaaccaccaag

SEQ ID NO: 22 - protein, 41 aa, Homo sapiens
SIVLQRWWCQMEPCLPGEECKVLPDLSGWSCSSGHKVKTTK

SEQ ID NO: 23 - DNA, 12 bp, Homo sapiens
gtcacacgatag

SEQ ID NO: 24 - protein, 3 aa, Homo sapiens
VTR

SEQ ID NO: 25 - DNA, 402 bp, Homo sapiens
atgagtgagagggtcgagcggaactggagcacgggcggctggctgctggcactgtgcctggcctggctgtggacccacctgaccttggctgccttgcagcctcccactgccacagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacgcctccatcgtcctgcagagatggtggtgtcagatggagccctgcctgccgggggaggagtgtaaggtgctcccggacctgtcgggatggagctgcagcagtggacacaaagtcaaaaccaccaaggtcacacgatag

SEQ ID NO: 26 - protein, 133 aa, Homo sapiens
MSERVERNWSTGGWLLALCLAWLWTHLTLAALQPPTATVLVQQGTCEVIAAHRCCNRNRIEERSQTVKCSCFSGQVAGTTRAKPSCVDASIVLQRWWCQMEPCLPGEECKVLPDLSGWSCSSGHKVKTTKVTR

SEQ ID NO: 27 - DNA, 25 bp, Homo sapiens
gccttgcagcctcccactgccacag

SEQ ID NO: 28 - protein, 9 aa, Homo sapiens
ALQPPTATV

SEQ ID NO: 29 - DNA, 312 bp, Homo sapiens
gccttgcagcctcccactgccacagtgcttgtgcagcagggcacctgcgaggtgattgcggctcaccgctgctgcaaccggaaccgcatcgaggagcgctcccagacggtgaaatgctcctgtttttctggccaggtggccggcaccacgcgggcaaagccctcctgcgtggacgcctccatcgtcctgcagagatggtggtgtcagatggagccctgcctgccgggggaggagtgtaaggtgctcccggacctgtcgggatggagctgcagcagtggacacaaagtcaaaaccaccaaggtcacacgatag

SEQ ID NO: 30 - protein, 103 aa, Homo sapiens
ALQPPTATVLVQQGTCEVIAAHRCCNRNRIEERSQTVKCSCFSGQVAGTTRAKPSCVDASIVLQRWWCQMEPCLPGEECKVLPDLSGWSCSSGHKVKTTKVTR

SEQ ID NO: 31 - protein, 134 aa, Mus. musculus
MRVCAKWVLLSRWLVLTYVLMVCCKLMSASSQHLRGHAGHHLIKPGTCEVVAVHRCCNKNRIEERSQTVKCSCFPGQVAGTTRAQPSCVEAAIVIEKWWCHMNPCLEGEDCKVLPDSSGWSCSSGNKVKTTKAS

SEQ ID NO: 32 - protein, 131 aa, Mus. musculus
MNKRYLQKATQGKLLIIIFIVTLWGKAVSSANHHKAHHVRTGTCEVVALHRCCNKNKIEERSQTVKCSCFPGQVAGTTRAAPSCVDASIVEQKWWCHMQPCLEGEECKVLPDRKGWSCSSGNKVKTTRVTH

SEQ ID NO: 33 - protein, 133 aa, Rattus rattus
MAERSTSNWSPGSWVLALCLAWLWTRLASASLQPPTSTVKQGTCEVIAAHRCCNRNRIEERSQTVKCSCLSGQVAGTTRAKPSCVDASIVLQKWWCQMEPCLLGEECKVLPDLSGWSCSRGHKVKTTKVLRWT

SEQ ID NO: 34 - protein, 131 aa, Rattus rattus
MTMVSAMSWVLYLWISACAMLLCHGSLQHTFQQHHLHRPEGGTCEVIAAHRCCNKNRIEERSQTVKCSCLPGKVAGTTRNRPSCVDASIVIGKWWCEMEPCLEGEECKTLPDNSGWMCATGNKIKTTRVSP

SEQ ID NO: 35 - protein, 153 aa, Pufferfish
MNVIRSVRPSHWGLLLLCTAAFCSQLVATGNQSSRGQRGSEQERTGTCEVVAAHRCCNKNKIEERSQTVKCSCFPGQVAGTTRALPSCVDASIVRQKWWCNMEPCVVGEECRVLPDLTGWSCISGNKVKTTKVSRGAALVVKKPNRTSLQCRI

SEQ ID NO: 36 - DNA, 216 bp, Homo sapiens
atggcaatggtctctgcgatgtcctgggtcctgtatttgtggataagtgcttgtgcaatgctactctgccatggatcccttcagcacactttccagcagcatcacctgcacagaccaggtgcagagcaaaaccagtgtggctggaaaggagaaagaaggaaagggggcttcaagcaagatcatgtgcattgtcagacttcagatcatccaaggcct

SEQ ID NO: 37 - protein, 72 aa, Homo sapiens
MAMVSAMSWVLYLWISACAMLLCHGSLQHTFQQHHLHRPGAEQNQCGWKGERRKGGFKQDHVHCQTSDHPRP

SEQ ID NO: 38 - DNA, 141 bp, Homo sapiens
tcccttcagcacactttccagcagcatcacctgcacagaccaggtgcagagcaaaaccagtgtggctggaaaggagaaagaaggaaagggggcttcaagcaagatcatgtgcattgtcagacttcagatcatccaaggcct

SEQ ID NO: 39 - protein, 47 aa, Homo sapiens
SLQHTFQQHHLHRPGAEQNQCGWKGERRKGGFKQDHVHCQTSDHPRP

SEQ ID NO: 40 - DNA, 390 bp, Homo sapiens
atgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactaggtggtga

SEQ ID NO: 41 - protein, 129 aa, Homo sapiens
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRW

SEQ ID NO: 42 - DNA, 300 bp, Homo sapiens
gcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactaggtggtga

SEQ ID NO: 43 - protein, 99 aa, Homo sapiens
ANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRW

SEQ ID NO: 44 - DNA, 399 bp, Homo sapiens; identical to SEQ ID NO: 9

SEQ ID NO: 45 - protein, 132 aa, Homo sapiens; identical to SEQ ID NO: 10

SEQ ID NO: 46 - DNA, 270 bp, Homo sapiens
acctgtgagattgtgaccttggaccgggacagcagccagcctcggaggacgatcgcccggcagaccgcccgctgtgcgtgtagaaaggggcagatcgccggcaccacgagagcccggcccgcctgtgtggacgcaagaatcatcaagaccaagcagtggtgtgacatgcttccgtgtctggagggggaaggctgcgacttgttaatcaaccggtcaggctggacgtgcacgcagcccggcgggaggataaagaccaccacggtctcctga

SEQ ID NO: 47 - protein, 89 aa, Homo sapiens
TCEIVTLDRDSSQPRRTIARQTARCACRKGQIAGTTRARPACVDARIIKTKQWCDMLPCLEGEGCDLLINRSGWTCTQPGGRIKTTTVS

SEQ ID NO: 48 - DNA, 423 bp, Homo sapiens; identical to SEQ ID NO: 13

SEQ ID NO: 49 - protein, 140 aa, Homo sapiens; identical to SEQ ID NO: 14

SEQ ID NO: 50 - DNA, 320 bp, Homo sapiens
tcaagccagcacctccggggacatgcaggtcaccaccaaatcaagcaagggacctgtgaggtggtcgccgtgcacaggtgctgcaataagaaccgcatagaagagcggtcacaaacggtcaagtgctcttgcttcccgggacaggtggcgggcacaactcgggctcaaccttcttgtgttgaagcttccattgtgattcagaaatggtggtgtcacatgaatccgtgtttggaaggagaggattgtaaagtgctgccagattactcaggttggtcctgtagcagtggcaataaagtcaaaactacgaaggtaacgcggta

SEQ ID NO: 51 - protein, 106 aa, Homo sapiens; identical to SEQ ID NO: 16

SEQ ID NO: 52 - DNA, 393 bp, Homo sapiens
atgagtaagagatacttacagaaagcaacaaaaggaaaactgctaataataatatttattgtaaccttgtgggggaaagttgtatccagtgcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggcaaacgtg

SEQ ID NO: 53 - protein, 131 aa, Homo sapiens
MSKRYLQKATKGKLLIIIFIVTLWGKVVSSANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRANV

SEQ ID NO: 54 - DNA, 303 bp, Homo sapiens
gcaaaccatcataaagctcaccatgttaaaacgggaacttgtgaggtggtggcactccacagatgctgtaataagaacaagatagaagaacggtcacaaacagtcaagtgctcctgcttccctgggcaggtggcaggcaccacgcgagctgctccatcatgtgtggatgcttcaatagtggaacagaaatggtggtgccatatgcagccatgtctagagggagaagaatgtaaagttcttccggatcggaaaggatggagctgttcctctgggaataaagtcaaaacaactagggcaaacgtg

SEQ ID NO: 55 - protein, 101 aa, Homo sapiens
ANHHKAHHVKTGTCEVVALHRCCNKNKIEERSQTVKCSCFPGQVAGTTRAAPSCVDASIVEQKWWCHMQPCLEGEECKVLPDRKGWSCSSGNKVKTTRANV

SEQ ID NO: 56 - protein, 87 aa, Homo sapiens
  5 or 6: Xaa is Val or Ile
  7: Xaa is Ala or Thr
  8: Xaa is Ala, Val or Leu
  9: Xaa is His or Asp
  11: Xaa is Cys or Asp
  12: Xaa is Cys or Ser
  13: Xaa is Asn or Ser
  14: Xaa is Lys, Arg or Gln
  15: Xaa is Asn or Pro
  16 or 25: Xaa is Arg or Lys
  17: Xaa is Ile or Arg
  18: Xaa is Glu or Thr
  19: Xaa is Glu or Ile
  20: Xaa is Arg or Ala
  21: Xaa is Ser or Arg
  24: Xaa is Val or Ala
  27 or 42: Xaa is Ser or Ala
  29: Xaa is Leu, Phe or Arg
  30: Xaa is Pro, Ser or Lys
  32: Xaa is Lys or Gln
  33: Xaa is Val or Ile
  39: Xaa is Asn or Ala
  40: Xaa is Arg, Gln, Leu, Ala or Lys
  45: Xaa is Asp or Glu
  47: Xaa is Ser, Ala or Arg
  49: Xaa is Val or Ile
  50: Xaa is Ile, Glu, Leu, Lys or Arg
  51: Xaa is Trp, Gly, Gln, Glu or Thr
  52: Xaa is Lys or Arg
  53: Xaa is Trp or Gln
  56: Xaa is Glu, His, Asn, Gln or Asp
  58: Xaa is Glu, Asn, Gln or Leu
  61: Xaa is Leu or Val
  62: Xaa is Glu, Val, Leu or Pro
  65: Xaa is Asp, Glu or Gly
  67: Xaa is Lys, Arg or Asp
  68: Xaa is Thr, Val or Leu
  70: Xaa is Pro or Ile
  71: Xaa is Asp or Asn
  72: Xaa is Asn, Tyr, Ser, Leu or Arg
  73: Xaa is Ser, Thr or Lys
  76: Xaa is Met, Ser or Thr
  78: Xaa is Ala, Ser, Ile or Thr
  79: Xaa is Thr, Ser, Arg or Gln
  82: Xaa is Asn, His or Gly
  83: Xaa is Lys or Arg
  84: Xaa is Ile or Val
GTCEXXXXXRXXXXXXXXXXXQTXXCXCXXGXXAGTTRXXPXCVXAXIXXXXXWCXMXPCXXGEXCXXLXXXXGWXCXXPGXXXKTT

SEQ ID NO: 57 - protein, 86 aa, Homo sapiens
  5 or 6: Xaa is Val or Ile
  7: Xaa is Ala or Thr
  8: Xaa is Ala, Val or Leu
  9: Xaa is His or Asp
  11: Xaa is Cys or Asp
  12: Xaa is Cys or Ser
  13: Xaa is Asn or Ser
  14: Xaa is Lys, Arg or Gln
  15: Xaa is Asn or Pro
  16 or 25: Xaa is Arg or Lys
  17: Xaa is Ile or Arg
  18: Xaa is Glu or Thr
  19: Xaa is Glu or Ile
  20: Xaa is Arg or Ala
  21: Xaa is Ser or Arg
  24: Xaa is Val or Ala
  27 or 42: Xaa is Ser or Ala
  29: Xaa is Leu, Phe or Arg
  30: Xaa is Pro, Ser or Lys
  32: Xaa is Lys or Gln
  33: Xaa is Val or Ile
  39: Xaa is Asn or Ala
  40: Xaa is Arg, Gln, Leu, Ala or Lys
  45: Xaa is Asp or Glu
  47: Xaa is Ser, Ala or Arg
  49: Xaa is Val or Ile
  50: Xaa is Ile, Glu, Leu, Lys or Arg
  51: Xaa is Trp, Gly, Gln, Glu or Thr
  52: Xaa is Lys or Arg
  53: Xaa is Trp or Gln
  56: Xaa is Glu, His, Asn, Gln or Asp
  58: Xaa is Glu, Asn, Gln or Leu
  61: Xaa is Leu or Val
  62: Xaa is Glu, Val, Leu or Pro
  65: Xaa is Asp, Glu or Gly
  67: Xaa is Lys, Arg or Asp
  68: Xaa is Thr, Val or Leu
  70: Xaa is Pro or Ile
  71: Xaa is Asp or Asn
  72: Xaa is Asn, Tyr, Ser, Leu or Arg
  73: Xaa is Ser, Thr or Lys
  76: Xaa is Met, Ser or Thr
  78: Xaa is Ala, Ser, Ile or Thr
  79: Xaa is Thr, Ser, Arg or Gln
  81: Xaa is Asn, His or Gly
  82: Xaa is Lys or Arg
  83: Xaa is Ile or Val
GTCEXXXXXRXXXXXXXXXXXQTXXCXCXXGXXAGTTRXXPXCVXAXIXXXXXWCXMXPCXXGEXCXXLXXXXGWXCXXGXXXKTT

SEQ ID NO: 58 - protein, 19 aa, Artificial Sequence
  oligonucleotide primer
AGAATGGCAATGGTCTCTG

SEQ ID NO: 59 - DNA, 20 bp, Artificial Sequence
  oligonucleotide primer
ccacaaatgcttctgttagg

SEQ ID NO: 60 - DNA, 37 bp, Artificial Sequence
  oligonucleotide primer
aagcaggcttcgccaccatggcaatggtctctgcgat

SEQ ID NO: 61 - DNA, 35 bp, Artificial Sequence
  oligonucleotide primer
gtgatggtgatggtgggttcttgggtgaattctcg

SEQ ID NO: 62 - DNA, 37 bp, Artificial Sequence; identical to SEQ ID NO: 60
  oligonucleotide primer

SEQ ID NO: 63 - DNA, 35 bp, Artificial Sequence
  oligonucleotide primer
gtgatggtgatggtgaggccttggatgatctgaag

SEQ ID NO: 64 - DNA, 37 bp, Artificial Sequence
  oligonucleotide primer
ggggacaagtttgtacaaaaaagcaggcttcgccacc

SEQ ID NO: 65 - DNA, 51 bp, Artificial Sequence
  oligonucleotide primer
ggggaccactttgtacaagaaagctgggtttcaatggtgatggtgatggtg

SEQ ID NO: 66 - DNA, 20 bp, Artificial Sequence
  oligonucleotide primer
gccagcttggcacttgatgt

SEQ ID NO: 67 - DNA, 20 bp, Artificial Sequence
  oligonucleotide primer
gatggaggtggacgtgtcag

SEQ ID NO: 68 - DNA, 18 bp, Artificial Sequence
  oligonucleotide primer
tgtaaaacgacggccagt

SEQ ID NO: 69 - DNA, 18 bp, Artificial Sequence
  oligonucleotide primer
caggaaacagctatgacc

SEQ ID NO: 70 - DNA, 19 bp, Artificial Sequence
  oligonucleotide primer
taatacgactcactatagg

SEQ ID NO: 71 - DNA, 18 bp, Artificial Sequence
  oligonucleotide primer
attaaccctcactaaagg

SEQ ID NO: 72 - DNA, 18 bp, Artificial Sequence
  oligonucleotide primer
atttaggtgacactatag

SEQ ID NO: 73 - DNA, 20 bp, Artificial Sequence
  oligonucleotide primer
gctgcaggatgagtaagaga

SEQ ID NO: 74 - DNA, 20 bp, Artificial Sequence
  oligonucleotide primer
tcatcagccttgaggatcac

SEQ ID NO: 75 - DNA, 26 bp, Artificial Sequence
  oligonucleotide primer
atgagtaagagatacttacagaaagc

SEQ ID NO: 76 - DNA, 28 bp, Artificial Sequence
  oligonucleotide primer
tcaccacctagttgttttgactttattc

SEQ ID NO: 77 - DNA, 23 bp, Artificial Sequence
  oligonucleotide primer
acgcgagctgctccatcatgtgt

SEQ ID NO: 78 - DNA, 23 bp, Artificial Sequence
  oligonucleotide primer
tggtgccatatgcagccatgtct

SEQ ID NO: 79 - DNA, 25 bp, Artificial Sequence
  oligonucleotide primer
gctgtcaacgatacgctacgtaacg

SEQ ID NO: 80 - DNA, 23 bp, Artificial Sequence
  oligonucleotide primer
cgctacgtaacggcatgacagtg

SEQ ID NO: 81 - DNA, 54 bp, Artificial Sequence
  oligonucleotide primer
gctgtcaacgatacgctacgtaacggcatgacagtgtttttttttttttttttt

SEQ ID NO: 82 - DNA, 37 bp, Artificial Sequence
  oligonucleotide primer
aagcaggcttcgccaccatgagtaagagatacttaca

SEQ ID NO: 83 - DNA, 35 bp, Artificial Sequence
  oligonucleotide primer
gtgatggtgatggtgatgggttaccctagttgttt

SEQ ID NO: 84 - DNA, 35 bp, Artificial Sequence
  oligonucleotide primer
gtgatggtgatggtgcacgtttgccctagttgttt

SEQ ID NO: 85 - DNA, 35 bp, Artificial Sequence
  oligonucleotide primer
gtgatggtgatggtgccacctagttgttttgactt

SEQ ID NO: 86 - DNA, 37 bp, Artificial Sequence; identical to SEQ ID NO: 64
  oligonucleotide primer

SEQ ID NO: 87 - DNA, 51 bp, Artificial Sequence; identical to SEQ ID NO: 65
  oligonucleotide primer

SEQ ID NO: 88 - DNA, 20 bp, Artificial Sequence
  oligonucleotide primer
gccagcttggcacttgatgt

SEQ ID NO: 89 - DNA, 20 bp, Artificial Sequence
  oligonucleotide primer
gatggaggtggacgtgtcag

SEQ ID NO: 90 - DNA, 18 bp, Artificial Sequence
  oligonucleotide primer
tgtaaaacgacggccagt

SEQ ID NO: 91 - DNA, 18 bp, Artificial Sequence
  oligonucleotide primer
caggaaacagctatgacc

SEQ ID NO: 92 - DNA, 19 bp, Artificial Sequence
  oligonucleotide primer
taatacgactcactatagg

SEQ ID NO: 93 - DNA, 18 bp, Artificial Sequence
  oligonucleotide primer
attaaccctcactaaagg

SEQ ID NO: 94 - DNA, 18 bp, Artificial Sequence
  oligonucleotide primer
atttaggtgacactatag

SEQ ID NO: 95 - DNA, 19 bp, Artificial Sequence
  oligonucleotide primer
taatacgactcactatagg

SEQ ID NO: 96 - DNA, 18 bp, Artificial Sequence
  oligonucleotide primer
attaaccctcactaaagg

SEQ ID NO: 97 - DNA, 37 bp, Artificial Sequence
  oligonucleotide primer
aagcaggcttcgccaccatggcgccatcgcccaggac

SEQ ID NO: 98 - DNA, 35 bp, Artificial Sequence
  oligonucleotide primer
gtgatggtgatggtgggagaccgtggtggtcttta

SEQ ID NO: 99 - DNA, 37 bp, Artificial Sequence; identical to SEQ ID NO: 64
  oligonucleotide primer

SEQ ID NO: 100 - DNA, 51 bp, Artificial Sequence; identical to SEQ ID NO: 65
  oligonucleotide primer

SEQ ID NO: 101 - DNA, 20 bp, Artificial Sequence
  oligonucleotide primer
gccagcttggcacttgatgt

SEQ ID NO: 102 - DNA, 20 bp, Artificial Sequence
  oligonucleotide primer
gatggaggtggacgtgtcag

SEQ ID NO: 103 - DNA, 18 bp, Artificial Sequence
  oligonucleotide primer
tgtaaaacgacggccagt

SEQ ID NO: 104 - DNA, 18 bp, Artificial Sequence
  oligonucleotide primer
caggaaacagctatgacc

SEQ ID NO: 105 - DNA, 19 bp, Artificial Sequence
  oligonucleotide primer
taatacgactcactatagg

SEQ ID NO: 106 - DNA, 18 bp, Artificial Sequence
  oligonucleotide primer
attaaccctcactaaagg

SEQ ID NO: 107 - DNA, 37 bp, Artificial Sequence
  oligonucleotide primer
aagcaggcttcgccaccatgaggtccccaaggatgag

SEQ ID NO: 108 - DNA, 35 bp, Artificial Sequence
  oligonucleotide primer
gtgatggtgatggtgccgcgttaccttcgtagttt

SEQ ID NO: 109 - DNA, 37 bp, Artificial Sequence; identical to SEQ ID NO: 64
  oligonucleotide primer

SEQ ID NO: 110 - DNA, 51 bp, Artificial Sequence; identical to SEQ ID NO: 65
  oligonucleotide primer

SEQ ID NO: 111 - DNA, 20 bp, Artificial Sequence
  oligonucleotide primer
gccagcttggcacttgatgt

SEQ ID NO: 112 - DNA, 20 bp, Artificial Sequence
  oligonucleotide primer
gatggaggtggacgtgtcag

SEQ ID NO: 113 - DNA, 18 bp, Artificial Sequence
  oligonucleotide primer
tgtaaaacgacggccagt

SEQ ID NO: 114 - DNA, 18 bp, Artificial Sequence
  oligonucleotide primer
caggaaacagctatgacc

SEQ ID NO: 115 - DNA, 21 bp, Artificial Sequence
  oligonucleotide primer
ttctctccgcaggatgagtga

SEQ ID NO: 116 - DNA, 20 bp, Artificial Sequence
  oligonucleotide primer
tcgtgtgaccttggtggttt

SEQ ID NO: 117 - DNA, 37 bp, Artificial Sequence
  oligonucleotide primer
aagcaggcttcgccaccatgagtgagagggtcgagcg

SEQ ID NO: 118 - DNA, 35 bp, Artificial Sequence
  oligonucleotide primer
gtgatggtgatggtgtcgtgtgaccttggtggttt

SEQ ID NO: 119 - DNA, 37 bp, Artificial Sequence; identical to SEQ ID NO: 64
  oligonucleotide primer

SEQ ID NO: 120 - DNA, 51 bp, Artificial Sequence; identical to SEQ ID NO: 65
  oligonucleotide primer

SEQ ID NO: 121 - DNA, 20 bp, Artificial Sequence
  oligonucleotide primer
gccagcttggcacttgatgt

SEQ ID NO: 122 - DNA, 20 bp, Artificial Sequence
  oligonucleotide primer
gatggaggtggacgtgtcag

SEQ ID NO: 123 - DNA, 18 bp, Artificial Sequence
  oligonucleotide primer
tgtaaaacgacggccagt

SEQ ID NO: 124 - DNA, 18 bp, Artificial Sequence
  oligonucleotide primer
caggaaacagctatgacc

SEQ ID NO: 125 - DNA, 19 bp, Artificial Sequence
  oligonucleotide primer
taatacgactcactatagg

SEQ ID NO: 126 - DNA, 18 bp, Artificial Sequence
  oligonucleotide primer
atttaggtgacactatag

(19) United States
(12) Patent Application Publication
(10) Pub. No.: US 2007/0021590 A1
(43) Pub. Date: Jan. 25, 2007

(54) Secreted protein family

(76) Inventors: Christine Power, Thoiry (FR); Melanie Yorke, Confignon (CH); Richard Joseph Fagan, London (GB); David Miachalovich, London (GB); Iain McKendrick, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/547,529

(22) PCT Filed: Mar. 24, 2004
(86) PCT No.: PCT/GB04/01248

§ 371 Date: Jun. 5, 2006

(30) Foreign Application Priority Data
Mar. 24, 2003 (GB) 0306771.7

(51) Int. Cl. C07K 14/71; C12Q 1/68; G06F 19/00

(52) U.S. Cl. 530/350; 435/6; 702/20

(57) ABSTRACT

This invention relates to a new family of secreted proteins, termed the SECFAMI family, its family members including the novel proteins INSP113, INSP114, INSP115, INSP116 and INSP117, herein identified as secreted proteins containing epidermal growth factor (EGF) fold-containing domains, ranging from 125-153 amino acids in length and containing eight conserved cysteine residues and to the use of these proteins and nucleic acid sequences from the encoding genes in the diagnosis, prevention and treatment of disease.

This invention relates to a new family of proteins, termed the SECFAM1 family, its family members including the novel proteins INSP113, INSP114, INSP115, INSP116 and INSP117, herein identified as secreted proteins containing epidermal growth factor (EGF) fold-containing domains, ranging from 125-153 amino acids in length and containing eight conserved cysteine residues and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### INTRODUCTION

Secreted Proteins

The ability of cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

EGF Domain-Containing Proteins

The epidermal growth factor (EGF)-like proteins stimulate cells to divide by activating members of the EGF receptor (EGF-R) family (Yarden et al., Eur J Cancer. 2001 September; 37 Suppl 4:S3-81). EGF is a short peptide with a distinctive motif of six conserved cysteines that are all involved in the formation of disulphide bonds. Indeed, this motif is found in many different proteins of diverse function (Davis et al., New Biol. 1990 May; 2(5): 410-9). While such domains can be found across too functionally diverse a range of protein types as to draw any definitive functional conclusions, it may be noted that all EGF domains (with the exception of prostaglandin G/H synthase) are found either within the extracellular domain of membrane-bound proteins, or within proteins known to be secreted into the extracellular matrix. It is known that EGF domains are involved in the interaction between proteins and, as such, have a large proportion of hydrophobic residues exposed on the surface of the unbound protein. This domain, with its highly conserved cysteine pattern, plays an important role in signalling and regulation events across a wide spectrum of organisms from nematode worms to humans. EGF domains have been implicated in broad cross-section of processes from tissue repair and regulation of blood factor coagulation, to processes involved in the growth and development of many types of human tumours. In the latter case, EGF-like proteins stimulate cells to divide by activating members of the EGF signalling pathway (Yarden et al., Eur J Cancer, 2001 September; 37 Suppl 4: S3-81). Due to such observations, their role in tumour progression has been of great interest in the pursuit of anti-cancer therapeutic strategies and recent novel therapeutic agents in this field have been developed based on the interference of EGF signalling pathways (Waksal et al., Cancer Metastasis Rev. 1999; 18(4): 427-36).

Increasing knowledge of these domains is therefore of extreme importance in increasing the understanding of the underlying pathways that lead to the disease states and associated disease states mentioned above, and in developing more effective gene and/or drug therapies to treat these disorders.

Detailed herein is the identification of an entirely novel family of secreted protein ligands. The definition of a secreted protein ligand is a protein that is secreted from a particular cell and elicits a phenotypic response in the same/or another cell by modulating (including ligand-antagonism, as demonstrated by the Dan family) the activity of a cognate receptor and downstream signal transduction pathway. An example of an already known secreted protein ligand family is the glycoprotein hormone family.

Follicle-stimulating hormone (FSH) is a member of the glycoprotein hormone family. In males, FSH is secreted by the cells of the anterior lobe of the pituitary gland, enters the bloodstream, and then binds cognate receptors on the Sertoli cells of the testes to regulate the process of spermatogenesis. In females FSH binds receptors on the thecal, stromal and granulosa cells of the ovary to regulate ovulation. FSH deficiencies can lead to infertility problems in both men and women. Restoring the levels of FSH by administering FSH in the form of a protein therapeutic can be used to combat FSH-triggered infertility. FSH is available as GONAL-f™ (Serono).

By analogy to this example, it can be seen that the identification of a novel secreted ligand protein family paves the way to the delineation of novel ligand-receptor pathways, and critically, to elucidation of the phenotypic consequences of ligand binding. If human disorders are identified which are a consequence of dysfunction of any member of the novel secreted ligand protein family, then that member can be administered as a protein therapeutic to combat the disorder.

### THE INVENTION

The invention is based on the discovery that the INSP113, INSP114, INSP115, INSP116 and INSP117 polypeptides are secreted proteins, more specifically, EGF-like domain containing secreted proteins and even more preferably have biological activity similar to Coagulation Factor X. Together, INSP113, INSP114, INSP115, INSP116 and INSP117 form part of a family of proteins herein identified as the SECFAM1 family of proteins.

Annotation of the SECFAM1 family of Proteins

The proteins of the present invention have no associated publicly available annotation, contain a strong secretory protein signature in the form of a signal peptide, and can be clustered with similar proteins, supported by orthologues from other animal species. Further examination has permitted the construction of a hitherto uncharacterised family of proteins consisting of 5 human genes (INSP113, INSP114, INSP115, INSP116 and INSP117) and which, including mammalian and fish orthologues, comprises 15 sequences in total. These sequences all display a strong signal peptide region of a variable composition with the remainder of the sequences displaying a high degree of similarity. Overall, within the human sequences, sequence identity is 49% or above, with a strong profile of conserved residues (FIG. 1). This cluster of related sequences will herein be referred to as the “SECFAM1 family.”

In one embodiment of the first aspect of the invention, there is provided a method of identifying a member of the SECFAM1 family comprising:

searching a database of translated nucleic acid sequences or polypeptide sequences to identify a polypeptide sequence that matches the following sequence profile:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −1 | −1 | −2 | −3 | −1 | 0 | −2 | −3 | −2 | 0 | 1 | −1 | 7 | 0 | −2 | −1 | −1 | −1 | −1 | 0 |
| 2 | N | 1 | 2 | 2 | −1 | −1 | 0 | −1 | −1 | −1 | −1 | −1 | 0 | 3 | −2 | −2 | 1 | 0 | −3 | −2 | −1 |
| 3 | K | 0 | 0 | −1 | 0 | −2 | 1 | 2 | −2 | −1 | 0 | −1 | 1 | −1 | −3 | 2 | −1 | −1 | −3 | −2 | 1 |
| 4 | R | 0 | 3 | −1 | −2 | 4 | 0 | −1 | −2 | −1 | 0 | 0 | 0 | 0 | −2 | −2 | 1 | −1 | −3 | −2 | −1 |
| 5 | Y | 1 | 0 | −2 | −2 | −2 | −1 | −1 | −2 | −1 | 0 | 0 | −1 | 0 | −1 | 3 | 0 | −1 | −2 | 2 | 0 |
| 6 | L | −1 | 2 | −1 | −1 | −2 | 0 | 0 | −2 | −1 | −1 | 0 | 3 | 2 | −2 | −2 | 0 | 1 | −2 | −2 | −1 |
| 7 | Q | 1 | 0 | 0 | −1 | −1 | 1 | 0 | −1 | −1 | −2 | −2 | 0 | −1 | −2 | −1 | 2 | 2 | 3 | −1 | −1 |
| 8 | K | −1 | −1 | 2 | −1 | −2 | −1 | −1 | 0 | −1 | 0 | 0 | 1 | 2 | −1 | −2 | 0 | 0 | −3 | −2 | 2 |
| 9 | A | 0 | 0 | −2 | −2 | −1 | −1 | −2 | −2 | −2 | 0 | 0 | −1 | 0 | −1 | −2 | 0 | −1 | 7 | 0 | 0 |
| 10 | T | 0 | 1 | 0 | −1 | −1 | 0 | −1 | −1 | −1 | −1 | 0 | 0 | 0 | −2 | 1 | 2 | 1 | −3 | −2 | −1 |
| 11 | Q | 1 | 0 | 0 | −1 | −1 | 2 | 0 | −1 | −1 | −1 | 0 | 0 | 0 | −2 | 0 | 2 | 0 | −3 | −2 | −1 |
| 12 | G | 0 | 0 | 0 | 2 | −2 | −1 | 0 | 3 | 4 | −2 | −2 | −1 | 2 | −2 | −2 | 0 | −1 | −2 | −1 | −2 |
| 13 | K | 1 | −1 | −1 | −1 | −2 | −1 | −1 | 2 | −2 | −3 | −2 | 0 | −1 | −1 | −2 | 2 | −1 | 7 | −1 | −2 |
| 14 | L | 0 | −2 | −2 | −3 | −1 | −2 | −2 | 1 | −2 | −1 | 0 | −2 | 0 | 0 | −2 | −1 | 1 | 8 | 0 | −1 |
| 15 | L | 2 | −2 | −3 | −3 | −1 | −2 | −2 | −2 | −3 | 1 | 3 | −2 | 0 | −1 | −2 | −1 | 0 | −3 | −1 | 2 |
| 16 | I | −1 | −2 | −3 | −4 | −1 | −2 | −3 | −4 | −3 | 2 | 4 | −2 | 1 | 1 | −3 | −2 | −1 | −2 | 0 | 1 |
| 17 | I | 1 | −2 | −2 | −2 | −1 | −2 | −2 | −2 | −1 | 1 | 1 | −2 | 0 | 0 | 3 | −1 | −1 | −1 | 2 | 0 |
| 18 | I | 0 | −2 | −1 | −2 | −1 | −1 | −2 | −2 | −2 | 1 | 3 | −1 | 0 | −1 | −2 | 1 | 0 | −2 | −1 | 0 |
| 19 | F | −1 | −2 | −3 | −3 | 6 | −2 | −3 | −3 | −1 | −1 | 0 | −2 | 3 | 2 | −3 | −2 | −1 | 6 | 3 | −1 |
| 20 | I | 0 | −2 | −1 | −2 | −1 | −2 | −2 | −2 | −2 | 3 | 1 | −2 | 0 | −1 | −2 | 1 | 1 | −3 | −1 | 2 |
| 21 | V | 2 | −2 | −1 | −2 | 3 | −1 | −1 | −1 | −2 | 0 | 0 | −1 | 0 | −2 | −2 | 2 | 0 | −3 | −2 | 0 |
| 22 | T | 1 | −2 | −2 | −2 | 3 | −1 | −2 | −2 | −2 | −1 | 0 | −2 | 2 | −1 | −2 | 0 | 2 | 6 | −1 | 0 |
| 23 | L | −1 | −3 | −3 | −3 | 4 | −3 | −3 | −3 | −2 | 0 | 2 | −3 | 0 | 4 | −3 | −2 | −1 | −1 | 0 | 1 |
| 24 | W | 0 | −3 | −3 | −3 | 5 | −2 | −3 | −2 | −2 | −1 | 0 | −3 | −1 | 0 | −3 | −1 | −1 | 9 | 0 | −1 |
| 25 | G | 1 | −2 | −1 | −2 | 4 | −1 | −2 | 1 | −2 | 0 | 0 | −1 | 2 | −2 | −2 | 0 | 1 | −2 | −2 | 0 |
| 26 | K | −1 | 0 | −1 | −2 | 1 | 1 | 0 | −3 | 1 | 0 | 1 | 2 | 0 | 2 | −2 | −1 | −1 | −2 | 0 | −1 |
| 27 | A | 0 | −2 | −3 | −3 | −1 | −2 | −2 | −3 | −3 | 1 | 3 | −2 | 3 | 0 | 0 | −2 | −1 | −2 | −1 | 1 |
| 28 | V | 0 | −2 | −2 | −3 | 4 | −2 | −2 | −3 | −3 | 3 | 0 | −2 | 3 | −1 | −2 | −1 | 0 | −2 | −1 | 2 |
| 29 | S | 0 | −1 | 0 | −1 | −1 | −1 | −1 | −1 | 5 | −1 | 1 | −1 | 0 | −1 | −2 | 2 | 0 | −3 | 0 | −1 |
| 30 | S | 4 | −1 | −1 | −2 | −1 | −1 | −1 | 2 | −2 | −2 | −2 | −1 | −1 | −2 | −1 | 1 | 1 | −3 | −2 | −1 |
| 31 | A | 1 | −1 | 0 | 0 | −1 | 1 | 0 | 1 | −1 | −2 | −2 | 0 | −1 | −2 | −1 | 4 | 0 | −3 | −2 | −2 |
| 32 | N | −1 | −1 | 2 | −1 | −2 | −1 | −2 | −2 | −1 | 0 | 3 | −1 | 0 | 1 | −3 | 1 | −1 | −2 | −1 | 0 |
| 33 | H | −1 | 0 | 0 | −1 | −2 | 5 | 0 | −3 | 3 | −1 | 1 | 0 | 0 | −2 | −2 | −1 | −1 | −2 | −1 | −1 |
| 34 | H | −1 | 0 | 0 | −1 | −2 | 0 | 0 | −2 | 6 | 0 | −1 | 0 | −1 | −2 | 2 | 0 | −1 | −3 | 0 | −1 |
| 35 | K | −1 | 0 | 0 | −1 | −3 | 3 | 0 | −2 | 4 | −3 | −2 | 1 | −1 | −3 | 3 | 0 | −1 | −2 | 0 | −2 |
| 36 | A | 2 | 1 | 0 | −1 | −1 | 0 | −1 | −1 | 4 | −1 | −1 | 0 | −1 | −2 | −1 | 0 | 2 | −2 | 0 | −1 |
| 37 | H | 0 | −1 | 0 | −1 | −2 | −1 | −1 | 3 | 6 | −3 | −3 | −1 | −2 | −2 | −2 | 1 | −1 | −2 | 0 | −2 |
| 38 | H | 0 | −1 | 0 | −2 | 5 | −1 | −1 | −2 | 5 | −1 | 0 | −1 | −1 | −1 | −2 | 0 | 1 | −2 | 0 | −1 |
| 39 | V | −1 | −2 | −2 | −2 | −2 | −1 | 0 | −3 | −2 | 2 | 2 | −2 | 0 | −1 | 2 | −2 | −1 | −3 | −1 | 2 |
| 40 | R | 1 | 2 | −1 | −1 | −2 | 1 | 1 | −1 | −1 | −2 | −2 | 3 | −1 | −3 | −1 | 0 | −1 | −3 | −2 | −2 |
| 41 | T | 2 | −1 | −1 | −1 | −2 | 3 | 0 | 1 | −1 | −2 | −2 | 0 | −1 | −3 | 0 | 0 | 2 | −2 | −2 | −1 |
| 42 | G | 0 | −2 | 0 | −1 | −3 | −2 | −2 | 6 | −2 | −4 | −4 | −2 | −3 | −3 | −2 | 0 | −2 | −2 | −3 | −3 |
| 43 | T | 0 | −1 | 0 | −1 | −1 | −1 | −1 | −2 | −2 | −1 | −1 | −1 | −1 | −2 | −1 | 0 | 6 | −2 | −2 | 0 |
| 44 | C | 0 | −3 | −3 | −3 | 10 | −3 | −4 | −3 | −3 | −1 | −1 | −3 | −1 | −2 | −3 | −1 | −1 | −2 | −2 | −1 |
| 45 | E | −1 | 0 | 0 | 1 | −4 | 1 | 6 | −2 | 0 | −3 | −3 | 0 | −2 | −3 | −1 | 0 | −1 | −3 | −2 | −2 |
| 46 | V | 0 | −3 | −3 | −3 | −1 | −2 | −2 | −3 | −3 | 3 | 0 | −2 | 0 | −1 | −2 | −2 | 0 | −3 | −1 | 4 |
| 47 | V | 0 | −3 | −3 | −3 | −1 | −2 | −2 | −3 | −3 | 4 | 0 | −2 | 0 | −1 | −2 | −2 | 0 | −3 | −1 | 4 |
| 48 | A | 4 | −1 | −1 | −2 | 0 | −1 | −1 | −1 | −2 | −1 | −1 | −1 | −1 | −2 | −1 | 0 | 2 | −3 | −2 | 0 |
| 49 | L | 3 | −2 | −3 | −3 | −1 | −2 | −2 | −2 | −3 | 0 | 2 | −2 | 0 | −1 | −2 | 0 | 0 | −3 | −1 | 1 |
| 50 | H | −2 | −1 | 0 | 3 | −3 | 0 | 0 | −2 | 8 | −3 | −3 | −1 | −2 | −2 | −2 | −1 | −2 | −3 | 0 | −3 |
| 51 | R | −1 | 6 | 0 | −2 | −3 | 0 | 0 | −2 | 0 | −3 | −2 | 1 | −1 | −3 | −2 | −1 | −1 | −3 | −2 | −3 |
| 52 | C | −1 | −3 | −1 | 3 | 8 | −2 | −1 | −2 | −2 | −2 | −2 | −2 | −2 | −2 | −2 | −1 | −1 | −3 | −2 | −2 |
| 53 | C | 0 | −2 | −1 | −2 | 8 | −2 | −2 | −2 | −2 | −1 | −1 | −2 | 1 | −2 | −2 | 2 | 0 | −2 | −2 | −1 |
| 54 | N | −1 | 0 | 6 | 0 | −2 | 0 | 0 | 0 | 0 | −3 | −3 | 0 | −2 | −3 | −2 | 2 | 0 | −4 | −2 | −3 |
| 55 | K | −1 | 2 | 0 | −1 | −3 | 3 | 0 | −2 | −1 | −3 | −2 | 4 | −1 | −3 | −1 | 0 | −1 | −3 | −2 | −2 |
| 56 | N | −2 | −1 | 6 | 0 | −3 | 0 | 0 | −1 | 0 | −3 | −3 | 0 | −2 | −3 | 3 | 0 | 0 | −4 | −2 | −3 |
| 57 | K | −1 | 5 | 0 | −2 | −3 | 0 | 0 | −2 | 0 | −3 | −2 | 3 | −1 | −3 | −2 | −1 | −1 | −3 | −2 | −3 |
| 58 | I | −1 | 2 | −2 | −3 | −2 | −1 | −2 | −3 | −2 | 4 | 0 | −1 | 0 | −1 | −3 | −2 | −1 | −3 | −1 | 1 |
| 59 | E | −1 | 0 | 0 | 0 | −3 | 0 | 5 | −2 | −1 | −2 | −2 | 0 | −2 | −3 | −1 | 0 | 2 | −3 | −2 | −1 |
| 60 | E | −1 | −1 | −1 | 0 | −3 | 0 | 5 | −3 | −1 | 2 | −1 | 0 | −1 | −2 | −2 | −1 | −1 | −3 | −2 | 0 |
| 61 | R | 1 | 5 | −1 | −2 | −2 | 0 | 0 | −1 | −1 | −2 | −2 | 0 | −1 | −3 | −2 | 0 | −1 | −3 | −2 | −2 |
| 62 | S | 0 | 2 | 0 | −1 | −2 | 0 | 0 | −1 | −1 | −2 | −2 | 0 | −1 | −2 | −1 | 4 | 0 | −3 | −2 | −2 |
| 63 | Q | −1 | 0 | 0 | 0 | −3 | 6 | 1 | −2 | 0 | −3 | −2 | 0 | 0 | −3 | −1 | 0 | −1 | −2 | −1 | −2 |
| 64 | T | 0 | −1 | 0 | −1 | −1 | −1 | −1 | −2 | −2 | −1 | −1 | −1 | −1 | −2 | −1 | 0 | 6 | −2 | −2 | 0 |
| 65 | V | 2 | −2 | −3 | −3 | −1 | −2 | −2 | −2 | −3 | 1 | 0 | −2 | 0 | −1 | −2 | −1 | 0 | −3 | −1 | 4 |
| 66 | K | −1 | 3 | 0 | −1 | −3 | 0 | 0 | −2 | −1 | −3 | −2 | 5 | −1 | −3 | −1 | 0 | −1 | −3 | −2 | −2 |
| 67 | C | 0 | −3 | −3 | −3 | 10 | −3 | −4 | −3 | −3 | −1 | −1 | −3 | −1 | −2 | −3 | −1 | −1 | −2 | −2 | −1 |
| 68 | S | 2 | −1 | 0 | −1 | −1 | 0 | 0 | 0 | −1 | −2 | −2 | 0 | −1 | −2 | −1 | 4 | 0 | −3 | −2 | −1 |
| 69 | C | 0 | −3 | −3 | −3 | 10 | −3 | −4 | −3 | −3 | −1 | −1 | −3 | −1 | −2 | −3 | −1 | −1 | −2 | −2 | −1 |
| 70 | F | −2 | 2 | −2 | −3 | −2 | −1 | −2 | −3 | −1 | 0 | 1 | −1 | 0 | 4 | −3 | −2 | −2 | −1 | 0 | −1 |
| 71 | P | −1 | 0 | −1 | −1 | −3 | 0 | 0 | −2 | −2 | −3 | −3 | 2 | −2 | −3 | 5 | 1 | −1 | −4 | −3 | −2 |
| 72 | G | 0 | −2 | 0 | −1 | −3 | −2 | −2 | 6 | −2 | −4 | −4 | −2 | −3 | −3 | −2 | 0 | −2 | −2 | −3 | −3 |
| 73 | Q | −1 | 0 | 0 | 0 | −3 | 6 | 1 | −2 | 0 | −3 | −2 | 2 | 0 | −3 | −1 | 0 | −1 | −2 | −1 | −2 |
| 74 | V | 0 | −3 | −3 | −3 | −1 | −2 | −2 | −3 | −3 | 3 | 0 | −2 | 0 | −1 | −2 | −2 | 0 | −3 | −1 | 4 |
| 75 | A | 5 | −1 | −2 | −2 | 0 | −1 | −1 | 0 | −2 | −1 | −1 | −1 | −1 | −2 | −1 | 0 | 0 | −3 | −2 | 0 |
| 76 | G | 0 | −2 | 0 | −1 | −3 | −2 | −2 | 6 | −2 | −4 | −4 | −2 | −3 | −3 | −2 | 0 | −2 | −2 | −3 | −3 |
| 77 | T | 0 | −1 | 0 | −1 | −1 | −1 | −1 | −2 | −2 | −1 | −1 | −1 | −1 | −2 | −1 | 0 | 6 | −2 | −2 | 0 |
| 78 | T | 0 | −1 | 0 | −1 | −1 | −1 | −1 | −2 | −2 | −1 | −1 | −1 | −1 | −2 | −1 | 0 | 6 | −2 | −2 | 0 |
| 79 | R | −1 | 6 | 0 | −2 | −3 | 0 | 0 | −2 | 0 | −3 | −2 | 1 | −1 | −3 | −2 | −1 | −1 | −3 | −2 | −3 |
| 80 | A | 4 | −1 | 1 | −1 | 0 | −1 | −1 | 0 | −1 | −1 | −1 | −1 | −1 | −2 | −1 | 0 | 0 | −3 | −2 | 0 |
| 81 | A | 0 | 4 | 0 | −2 | −2 | 2 | 0 | −2 | −1 | −2 | 0 | 2 | 0 | −3 | −2 | 0 | −1 | −3 | −2 | −2 |
| 82 | P | −1 | −2 | −2 | −1 | −3 | −1 | −1 | −2 | −2 | −3 | −3 | −1 | −2 | −4 | 7 | −1 | −1 | −4 | −3 | −2 |
| 83 | S | 2 | −1 | 0 | −1 | −1 | 0 | 0 | 0 | −1 | −2 | −2 | 0 | −1 | −2 | −1 | 4 | 0 | −3 | −2 | −1 |
| 84 | C | 0 | −3 | −3 | −3 | 10 | −3 | −4 | −3 | −3 | −1 | −1 | −3 | −1 | −2 | −3 | −1 | −1 | −2 | −2 | −1 |
| 85 | V | 0 | −3 | −3 | −3 | −1 | −2 | −2 | −3 | −3 | 2 | 0 | −2 | 0 | −1 | −2 | −2 | 0 | −3 | −1 | 5 |
| 86 | D | −2 | −2 | 0 | 6 | −3 | 0 | 2 | −1 | −1 | −3 | −4 | −1 | −3 | −3 | −1 | 0 | −1 | −4 | −3 | −3 |
| 87 | A | 5 | −1 | −2 | −2 | 0 | −1 | −1 | 0 | −2 | −1 | −1 | −1 | −1 | −2 | −1 | 0 | 0 | −3 | −2 | 0 |
| 88 | S | 0 | 2 | 0 | −1 | −1 | 0 | 0 | −1 | −1 | −2 | −2 | 0 | −1 | −2 | −1 | 4 | 0 | −3 | −2 | −2 |
| 89 | I | −1 | −3 | −3 | −3 | −1 | −3 | −3 | −4 | −3 | 5 | 1 | −3 | 0 | 0 | −3 | −2 | −1 | −3 | −1 | 2 |
| 90 | V | 0 | −3 | −3 | −3 | −1 | −2 | −2 | −3 | −3 | 3 | 0 | −2 | 0 | −1 | −2 | −2 | 0 | −3 | −1 | 4 |
| 91 | E | −1 | 1 | −1 | −1 | −2 | 0 | 1 | −3 | −2 | 2 | 1 | 2 | 0 | −1 | −2 | −1 | −1 | −3 | −2 | 0 |
| 92 | Q | −1 | 0 | 0 | 0 | −2 | 4 | 1 | 0 | −1 | −2 | −2 | 0 | −1 | −2 | −1 | 0 | 2 | 2 | −1 | −2 |
| 93 | K | −1 | 2 | 0 | −1 | −3 | 0 | 0 | −2 | −1 | −3 | −2 | 6 | −1 | −3 | −1 | 0 | −1 | −3 | −2 | −2 |
| 94 | W | −2 | −1 | −2 | −2 | −2 | 2 | −1 | −2 | −1 | −3 | −2 | −1 | −1 | 0 | −3 | −2 | −2 | 10 | 0 | −3 |
| 95 | W | −3 | −3 | −4 | −4 | −2 | −2 | −3 | −2 | −2 | −3 | −2 | −3 | −1 | 0 | −4 | −3 | −2 | 11 | 1 | −3 |
| 96 | C | 0 | −3 | −3 | −3 | 10 | −3 | −4 | −3 | −3 | −1 | −1 | −3 | −1 | −2 | −3 | −1 | −1 | −2 | −2 | −1 |
| 97 | H | −2 | 0 | 1 | 3 | −3 | 2 | 2 | −2 | 5 | −3 | −3 | 0 | −2 | −2 | −1 | 0 | −1 | −3 | 0 | −3 |
| 98 | M | −1 | −1 | −2 | −3 | −1 | 0 | −2 | −3 | −2 | 0 | 1 | −1 | 7 | 0 | −2 | −1 | −1 | −1 | −1 | 0 |
| 99 | Q | −1 | 0 | 2 | 0 | −3 | 2 | 3 | −2 | 0 | −1 | 1 | 0 | 0 | −2 | −2 | 0 | −1 | −3 | −2 | −1 |
| 100 | P | −1 | −2 | −2 | −1 | −3 | −1 | −1 | −2 | −2 | −3 | −3 | −1 | −2 | −4 | 7 | −1 | −1 | −4 | −3 | −2 |
| 101 | C | 0 | −3 | −3 | −3 | 10 | −3 | −4 | −3 | −3 | −1 | −1 | −3 | −1 | −2 | −3 | −1 | −1 | −2 | −2 | −1 |
| 102 | L | −1 | −2 | −3 | −4 | −1 | −2 | −3 | −4 | −3 | 1 | 4 | −2 | 1 | 0 | −3 | −2 | −1 | −2 | −1 | 1 |
| 103 | E | −1 | −1 | −1 | 0 | −3 | 0 | 5 | −2 | −1 | −1 | 0 | 0 | −1 | −2 | 0 | 0 | −1 | −3 | −2 | 0 |
| 104 | G | 0 | −2 | 0 | −1 | −3 | −2 | −2 | 6 | −2 | −4 | −4 | −2 | −3 | −3 | −2 | 0 | −2 | −2 | −3 | −3 |
| 105 | E | −1 | 0 | 0 | 1 | −4 | 1 | 6 | −2 | 0 | −3 | −3 | 0 | −2 | −3 | −1 | 0 | −1 | −3 | −2 | −2 |
| 106 | E | −1 | −1 | 0 | 2 | −4 | 0 | 4 | 2 | −1 | −3 | −4 | 0 | −3 | −3 | −1 | 0 | −1 | −3 | −3 | −3 |
| 107 | C | 0 | −3 | −3 | −3 | 10 | −3 | −4 | −3 | −3 | −1 | −1 | −3 | −1 | −2 | −3 | −1 | −1 | −2 | −2 | −1 |
| 108 | K | −1 | 1 | 0 | 3 | −3 | 0 | 0 | −2 | −1 | −3 | −3 | 4 | −2 | −3 | −1 | 0 | −1 | −3 | −2 | −2 |
| 109 | V | 0 | −2 | −2 | −3 | −1 | −2 | −2 | −3 | −3 | 1 | 2 | −2 | 0 | −1 | −2 | −1 | 1 | −3 | −1 | 4 |
| 110 | L | −1 | −2 | −3 | −4 | −1 | −2 | −3 | −4 | −3 | 1 | 4 | −2 | 1 | 0 | −3 | −2 | −1 | −2 | −1 | 0 |
| 111 | P | −1 | −2 | −2 | −2 | −2 | −2 | −2 | −3 | −2 | 2 | −1 | −2 | −1 | −2 | 6 | −1 | −1 | −4 | −2 | 0 |
| 112 | D | −2 | −1 | 3 | 6 | −3 | 0 | 1 | −1 | 0 | −3 | −4 | −1 | −3 | −3 | −1 | 0 | −1 | −4 | −3 | −3 |
| 113 | R | −1 | 3 | 2 | −2 | −2 | 0 | −1 | −2 | 0 | −1 | 1 | 0 | 0 | −1 | −2 | 0 | −1 | −2 | 1 | −1 |
| 114 | K | 0 | −1 | 0 | 0 | −1 | 0 | 0 | 0 | −1 | −2 | −2 | 1 | −1 | −2 | −1 | 4 | 1 | −3 | −2 | −2 |
| 115 | G | 0 | −2 | 0 | −1 | −3 | −2 | −2 | 6 | −2 | −4 | −4 | −2 | −3 | −3 | −2 | 0 | −2 | −2 | −3 | −3 |
| 116 | W | −3 | −3 | −4 | −4 | −2 | −2 | −3 | −2 | −2 | −3 | −2 | −3 | −1 | 0 | −4 | −3 | −2 | 11 | 1 | −3 |
| 117 | S | 0 | −1 | 0 | −1 | −1 | 0 | −1 | −1 | −1 | −1 | −1 | 0 | 2 | −2 | −1 | 3 | 3 | −2 | −2 | −1 |
| 118 | C | 0 | −3 | −3 | −3 | 10 | −3 | −4 | −3 | −3 | −1 | −1 | −3 | −1 | −2 | −3 | −1 | −1 | −2 | −2 | −1 |
| 119 | S | 1 | −1 | 0 | −1 | −1 | −1 | −1 | −1 | −2 | 0 | −1 | −1 | −1 | −2 | −1 | 3 | 3 | −3 | −2 | 0 |
| 120 | S | 0 | 0 | 0 | 0 | −2 | 3 | 0 | −1 | −1 | −2 | −2 | 0 | −1 | −2 | −1 | 3 | 1 | −3 | −2 | −2 |
| 121 | G | 0 | −2 | 0 | −1 | −3 | −2 | −2 | 6 | −2 | −4 | −4 | −2 | −3 | −3 | −2 | 0 | −2 | −2 | −3 | −3 |
| 122 | N | −1 | −1 | 5 | 0 | −3 | −1 | −1 | 3 | 4 | −3 | −3 | −1 | −2 | −3 | −2 | 0 | −1 | −3 | −1 | −3 |
| 123 | K | −1 | 3 | 0 | −1 | −3 | 0 | 0 | −2 | −1 | −3 | −2 | 5 | −1 | −3 | −1 | 0 | −1 | −3 | −2 | −2 |
| 124 | V | 0 | −3 | −3 | −3 | −1 | −2 | −2 | −4 | −3 | 4 | 0 | −2 | 0 | −1 | −2 | −2 | 0 | −3 | −1 | 4 |
| 125 | K | −1 | 1 | 0 | −1 | −3 | 0 | 0 | −2 | −1 | −3 | −2 | 6 | −1 | −3 | −1 | 0 | −1 | −3 | −2 | −2 |
| 126 | T | 0 | −1 | 0 | −1 | −1 | −1 | −1 | −2 | −2 | −1 | −1 | −1 | −1 | −2 | −1 | 0 | 6 | −2 | −2 | 0 |
| 127 | T | 0 | −1 | 0 | −1 | −1 | −1 | −1 | −2 | −2 | −1 | −1 | −1 | −1 | −2 | −1 | 0 | 6 | −2 | −2 | 0 |
| 128 | R | −1 | 3 | 0 | −1 | −2 | 0 | 0 | −2 | −1 | −2 | −2 | 4 | −1 | −3 | −1 | 0 | 2 | −3 | −2 | −2 |
| 129 | V | 0 | −3 | −3 | −3 | −1 | −2 | −2 | −3 | −3 | 2 | 0 | −2 | 0 | −1 | −2 | −2 | 0 | −3 | −1 | 5 |
| 130 | T | 0 | −1 | 0 | −1 | −1 | 0 | −1 | −1 | 2 | −1 | 0 | −1 | −1 | −2 | −1 | 3 | 3 | −3 | −1 | −1 |
| 131 | H | −1 | 4 | 0 | −1 | −3 | 0 | 0 | −2 | 4 | −3 | −2 | 0 | −1 | −3 | 3 | −1 | −1 | −3 | −1 | −3 |

wherein, when this profile is input as query sequence into the search program BLAST, using the default parameters specified by the NCBI (the National Center for Biotechnology Information; http://www.ncbi.nlm.nih.gov/) [Blosum 62 matrix; gap open penalty=11 and gap extension penalty=1], members of the SECFAM1 family are those which have an E value of 10⁻² or less.

A “member of the SECFAM1 family” is thus to be interpreted herein as a polypeptide sequence that satisfies the profile described above with a maximum threshold E value of 10⁻² when used as a query sequence in BLAST using the parameters described above. Preferably, the polypeptide sequence has a minimum threshold E value of 10⁻⁵ or less, 10⁻¹⁰ or less, 10⁻⁵⁰ or less, most preferably, 10⁻⁷⁰ or less. For example, when the family member INSP113 is compared to the profile of the first aspect of the invention, the E value generated is 4e⁻⁸⁰. An E value represents the expected number of better or equally good matches found in a database at random, or alternatively may be described as the probability that a match has occurred at random. Accordingly, all hits are ranked according to their E-values, which, in turn, depend on a) the number of candidates available for each sequence position (20 in the case of amino acids), the length of the sequence or matching region, and the size of the database searched. Shorter sequences such as the members of the SECFAM1 family therefore tend to have larger E-values than a comparable match between two longer sequences.

The above profile takes into account the existence of a signal sequence and an EGF-like domain. The profile allows for a higher degree of variability in the amino acid sequence of the signal peptide region (amino acids 1 to 30) compared to the EGF-like domain. “Variability” in this context, relates to the degree of similarity and identity between the amino acid sequences. This reflects the situation found with the fifteen members of the SECFAM1 family that are identified herein. The high degree of similarity shared in the EGF-like domains between the fifteen members also suggests that the EGF-like domain is likely to be involved in an important function of the molecule. If this domain was of less importance, the degree of conservation amongst its members would not be so high.

The database of translated nucleic acid sequences that is searched, may include, but is not limited to, translated nucleic acid sequences derived from cDNAs, ESTs, mRNAs, whole or partial genome databases.

In the second aspect of the invention, there is provided an isolated polypeptide which:

i) comprises or consists of a polypeptide sequence that has an E value of 10⁻² or less when the profile below is input as query sequence into the search program BLAST, using the default parameters specified by the NCBI (the National Center for Biotechnology Information; http://www.ncbi.nlm.nih.gov/) [Blosum 62 matrix; gap open penalty=11 and gap extension penalty=1]

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −1 | −1 | −2 | −3 | −1 | 0 | −2 | −3 | −2 | 0 | 1 | −1 | 7 | 0 | −2 | −1 | −1 | −1 | −1 | 0 |
| 2 | N | 1 | 2 | 2 | −1 | −1 | 0 | −1 | −1 | −1 | −1 | −1 | 0 | 3 | −2 | −2 | 1 | 0 | −3 | −2 | −1 |
| 3 | K | 0 | 0 | −1 | 0 | −2 | 1 | 2 | −2 | −1 | 0 | −1 | 1 | −1 | −3 | 2 | −1 | −1 | −3 | −2 | 1 |
| 4 | R | 0 | 3 | −1 | −2 | 4 | 0 | −1 | −2 | −1 | 0 | 0 | 0 | 0 | −2 | −2 | 1 | −1 | −3 | −2 | −1 |
| 5 | Y | 1 | 0 | −2 | −2 | −2 | −1 | −1 | −2 | −1 | 0 | 0 | −1 | 0 | −1 | 3 | 0 | −1 | −2 | 2 | 0 |
| 6 | L | −1 | 2 | −1 | −1 | −2 | 0 | 0 | −2 | −1 | −1 | 0 | 3 | 2 | −2 | −2 | 0 | 1 | −2 | −2 | −1 |
| 7 | Q | 1 | 0 | 0 | −1 | −1 | 1 | 0 | −1 | −1 | −2 | −2 | 0 | −1 | −2 | −1 | 2 | 2 | 3 | −1 | −1 |
| 8 | K | −1 | −1 | 2 | −1 | −2 | −1 | −1 | 0 | −1 | 0 | 0 | 1 | 2 | −1 | −2 | 0 | 0 | −3 | −2 | 2 |
| 9 | A | 0 | 0 | −2 | −2 | −1 | −1 | −2 | −2 | −2 | 0 | 0 | −1 | 0 | −1 | −2 | 0 | −1 | 7 | 0 | 0 |
| 10 | T | 0 | 1 | 0 | −1 | −1 | 0 | −1 | −1 | −1 | −1 | 0 | 0 | 0 | −2 | 1 | 2 | 1 | −3 | −2 | −1 |
| 11 | Q | 1 | 0 | 0 | −1 | −1 | 2 | 0 | −1 | −1 | −1 | 0 | 0 | 0 | −2 | 0 | 2 | 0 | −3 | −2 | −1 |
| 12 | G | 0 | 0 | 0 | 2 | −2 | −1 | 0 | 3 | 4 | −2 | −2 | −1 | 2 | −2 | −2 | 0 | −1 | −2 | −1 | −2 |
| 13 | K | 1 | −1 | −1 | −1 | −2 | −1 | −1 | 2 | −2 | −3 | −2 | 0 | −1 | −1 | −2 | 2 | −1 | 7 | −1 | −2 |
| 14 | L | 0 | −2 | −2 | −3 | −1 | −2 | −2 | 1 | −2 | −1 | 0 | −2 | 0 | 0 | −2 | −1 | 1 | 8 | 0 | −1 |
| 15 | L | 2 | −2 | −3 | −3 | −1 | −2 | −2 | −2 | −3 | 1 | 3 | −2 | 0 | −1 | −2 | −1 | 0 | −3 | −1 | 2 |
| 16 | I | −1 | −2 | −3 | −4 | −1 | −2 | −3 | −4 | −3 | 2 | 4 | −2 | 1 | 1 | −3 | −2 | −1 | −2 | 0 | 1 |
| 17 | I | 1 | −2 | −2 | −2 | −1 | −2 | −2 | −2 | −1 | 1 | 1 | −2 | 0 | 0 | 3 | −1 | −1 | −1 | 2 | 0 |
| 18 | I | 0 | −2 | −1 | −2 | −1 | −1 | −2 | −2 | −2 | 1 | 3 | −1 | 0 | −1 | −2 | 1 | 0 | −2 | −1 | 0 |
| 19 | F | −1 | −2 | −3 | −3 | 6 | −2 | −3 | −3 | −1 | −1 | 0 | −2 | 3 | 2 | −3 | −2 | −1 | 6 | 3 | −1 |
| 20 | I | 0 | −2 | −1 | −2 | −1 | −2 | −2 | −2 | −2 | 3 | 1 | −2 | 0 | −1 | −2 | 1 | 1 | −3 | −1 | 2 |
| 21 | V | 2 | −2 | −1 | −2 | 3 | −1 | −1 | −1 | −2 | 0 | 0 | −1 | 0 | −2 | −2 | 2 | 0 | −3 | −2 | 0 |
| 22 | T | 1 | −2 | −2 | −2 | 3 | −1 | −2 | −2 | −2 | −1 | 0 | −2 | 2 | −1 | −2 | 0 | 2 | 6 | −1 | 0 |
| 23 | L | −1 | −3 | −3 | −3 | 4 | −3 | −3 | −3 | −2 | 0 | 2 | −3 | 0 | 4 | −3 | −2 | −1 | −1 | 0 | 1 |
| 24 | W | 0 | −3 | −3 | −3 | 5 | −2 | −3 | −2 | −2 | −1 | 0 | −3 | −1 | 0 | −3 | −1 | −1 | 9 | 0 | −1 |
| 25 | G | 1 | −2 | −1 | −2 | 4 | −1 | −2 | 1 | −2 | 0 | 0 | −1 | 2 | −2 | −2 | 0 | 1 | −2 | −2 | 0 |
| 26 | K | −1 | 0 | −1 | −2 | 1 | 1 | 0 | −3 | 1 | 0 | 1 | 2 | 0 | 2 | −2 | −1 | −1 | −2 | 0 | −1 |
| 27 | A | 0 | −2 | −3 | −3 | −1 | −2 | −2 | −3 | −3 | 1 | 3 | −2 | 3 | 0 | 0 | −2 | −1 | −2 | −1 | 1 |
| 28 | V | 0 | −2 | −2 | −3 | 4 | −2 | −2 | −3 | −3 | 3 | 0 | −2 | 3 | −1 | −2 | −1 | 0 | −2 | −1 | 2 |
| 29 | S | 0 | −1 | 0 | −1 | −1 | −1 | −1 | −1 | 5 | −1 | 1 | −1 | 0 | −1 | −2 | 2 | 0 | −3 | 0 | −1 |
| 30 | S | 4 | −1 | −1 | −2 | −1 | −1 | −1 | 2 | −2 | −2 | −2 | −1 | −1 | −2 | −1 | 1 | 1 | −3 | −2 | −1 |
| 31 | A | 1 | −1 | 0 | 0 | −1 | 1 | 0 | 1 | −1 | −2 | −2 | 0 | −1 | −2 | −1 | 4 | 0 | −3 | −2 | −2 |
| 32 | N | −1 | −1 | 2 | −1 | −2 | −1 | −2 | −2 | −1 | 0 | 3 | −1 | 0 | 1 | −3 | 1 | −1 | −2 | −1 | 0 |
| 33 | H | −1 | 0 | 0 | −1 | −2 | 5 | 0 | −3 | 3 | −1 | 1 | 0 | 0 | −2 | −2 | −1 | −1 | −2 | −1 | −1 |
| 34 | H | −1 | 0 | 0 | −1 | −2 | 0 | 0 | −2 | 6 | 0 | −1 | 0 | −1 | −2 | 2 | 0 | −1 | −3 | 0 | −1 |
| 35 | K | −1 | 0 | 0 | −1 | −3 | 3 | 0 | −2 | 4 | −3 | −2 | 1 | −1 | −3 | 3 | 0 | −1 | −2 | 0 | −2 |
| 36 | A | 2 | 1 | 0 | −1 | −1 | 0 | −1 | −1 | 4 | −1 | −1 | 0 | −1 | −2 | −1 | 0 | 2 | −2 | 0 | −1 |
| 37 | H | 0 | −1 | 0 | −1 | −2 | −1 | −1 | 3 | 6 | −3 | −3 | −1 | −2 | −2 | −2 | 1 | −1 | −2 | 0 | −2 |
| 38 | H | 0 | −1 | 0 | −2 | 5 | −1 | −1 | −2 | 5 | −1 | 0 | −1 | −1 | −1 | −2 | 0 | 1 | −2 | 0 | −1 |
| 39 | V | −1 | −2 | −2 | −2 | −2 | −1 | 0 | −3 | −2 | 2 | 2 | −2 | 0 | −1 | 2 | −2 | −1 | −3 | −1 | 2 |
| 40 | R | 1 | 2 | −1 | −1 | −2 | 1 | 1 | −1 | −1 | −2 | −2 | 3 | −1 | −3 | −1 | 0 | −1 | −3 | −2 | −2 |
| 41 | T | 2 | −1 | −1 | −1 | −2 | 3 | 0 | 1 | −1 | −2 | −2 | 0 | −1 | −3 | 0 | 0 | 2 | −2 | −2 | −1 |
| 42 | G | 0 | −2 | 0 | −1 | −3 | −2 | −2 | 6 | −2 | −4 | −4 | −2 | −3 | −3 | −2 | 0 | −2 | −2 | −3 | −3 |
| 43 | T | 0 | −1 | 0 | −1 | −1 | −1 | −1 | −2 | −2 | −1 | −1 | −1 | −1 | −2 | −1 | 0 | 6 | −2 | −2 | 0 |
| 44 | C | 0 | −3 | −3 | −3 | 10 | −3 | −4 | −3 | −3 | −1 | −1 | −3 | −1 | −2 | −3 | −1 | −1 | −2 | −2 | −1 |
| 45 | E | −1 | 0 | 0 | 1 | −4 | 1 | 6 | −2 | 0 | −3 | −3 | 0 | −2 | −3 | −1 | 0 | −1 | −3 | −2 | −2 |
| 46 | V | 0 | −3 | −3 | −3 | −1 | −2 | −2 | −3 | −3 | 3 | 0 | −2 | 0 | −1 | −2 | −2 | 0 | −3 | −1 | 4 |
| 47 | V | 0 | −3 | −3 | −3 | −1 | −2 | −2 | −3 | −3 | 4 | 0 | −2 | 0 | −1 | −2 | −2 | 0 | −3 | −1 | 4 |
| 48 | A | 4 | −1 | −1 | −2 | 0 | −1 | −1 | −1 | −2 | −1 | −1 | −1 | −1 | −2 | −1 | 0 | 2 | −3 | −2 | 0 |
| 49 | L | 3 | −2 | −3 | −3 | −1 | −2 | −2 | −2 | −3 | 0 | 2 | −2 | 0 | −1 | −2 | 0 | 0 | −3 | −1 | 1 |
| 50 | H | −2 | −1 | 0 | 3 | −3 | 0 | 0 | −2 | 8 | −3 | −3 | −1 | −2 | −2 | −2 | −1 | −2 | −3 | 0 | −3 |
| 51 | R | −1 | 6 | 0 | −2 | −3 | 0 | 0 | −2 | 0 | −3 | −2 | 1 | −1 | −3 | −2 | −1 | −1 | −3 | −2 | −3 |
| 52 | C | −1 | −3 | −1 | 3 | 8 | −2 | −1 | −2 | −2 | −2 | −2 | −2 | −2 | −2 | −2 | −1 | −1 | −3 | −2 | −2 |
| 53 | C | 0 | −2 | −1 | −2 | 8 | −2 | −2 | −2 | −2 | −1 | −1 | −2 | −1 | −2 | −2 | 2 | 0 | −2 | −2 | −1 |
| 54 | N | −1 | 0 | 6 | 0 | −2 | 0 | 0 | 0 | 0 | −3 | −3 | 0 | −2 | −3 | −2 | 2 | 0 | −4 | −2 | −3 |
| 55 | K | −1 | 2 | 0 | −1 | −3 | 3 | 0 | −2 | −1 | −3 | −2 | 4 | −1 | −3 | −1 | 0 | −1 | −3 | −2 | −2 |
| 56 | N | −2 | −1 | 6 | 0 | −3 | 0 | 0 | −1 | 0 | −3 | −3 | 0 | −2 | −3 | 3 | 0 | 0 | −4 | −2 | −3 |
| 57 | K | −1 | 5 | 0 | −2 | −3 | 0 | 0 | −2 | 0 | −3 | −2 | 3 | −1 | −3 | −2 | −1 | −1 | −3 | −2 | −3 |
| 58 | I | −1 | 2 | −2 | −3 | −2 | −1 | −2 | −3 | −2 | 4 | 0 | −1 | 0 | −1 | −3 | −2 | −1 | −3 | −1 | 1 |
| 59 | E | −1 | 0 | 0 | 0 | −3 | 0 | 5 | −2 | −1 | −2 | −2 | 0 | −2 | −3 | −1 | 0 | 2 | −3 | −2 | −1 |
| 60 | E | −1 | −1 | −1 | 0 | −3 | 0 | 5 | −3 | −1 | 2 | −1 | 0 | −1 | −2 | −2 | −1 | −1 | −3 | −2 | 0 |
| 61 | R | 1 | 5 | −1 | −2 | −2 | 0 | 0 | −1 | −1 | −2 | −2 | 0 | −1 | −3 | −2 | 0 | −1 | −3 | −2 | −2 |
| 62 | S | 0 | 2 | 0 | −1 | −2 | 0 | 0 | −1 | −1 | −2 | −2 | 0 | −1 | −2 | −1 | 4 | 0 | −3 | −2 | −2 |
| 63 | Q | −1 | 0 | 0 | 0 | −3 | 6 | 1 | −2 | 0 | −3 | −2 | 0 | 0 | −3 | −1 | 0 | −1 | −2 | −1 | −2 |
| 64 | T | 0 | −1 | 0 | −1 | −1 | −1 | −1 | −2 | −2 | −1 | −1 | −1 | −1 | −2 | −1 | 0 | 6 | −2 | −2 | 0 |
| 65 | V | 2 | −2 | −3 | −3 | −1 | −2 | −2 | −2 | −3 | 1 | 0 | −2 | 0 | −1 | −2 | −1 | 0 | −3 | −1 | 4 |
| 66 | K | −1 | 3 | 0 | −1 | −3 | 0 | 0 | −2 | −1 | −3 | −2 | 5 | −1 | −3 | −1 | 0 | −1 | −3 | −2 | −2 |
| 67 | C | 0 | −3 | −3 | −3 | 10 | −3 | −4 | −3 | −3 | −1 | −1 | −3 | −1 | −2 | −3 | −1 | −1 | −2 | −2 | −1 |
| 68 | S | 2 | −1 | 0 | −1 | −1 | 0 | 0 | 0 | −1 | −2 | −2 | 0 | −1 | −2 | −1 | 4 | 0 | −3 | −2 | −1 |
| 69 | C | 0 | −3 | −3 | −3 | 10 | −3 | −4 | −3 | −3 | −1 | −1 | −3 | −1 | −2 | −3 | −1 | −1 | −2 | −2 | −1 |
| 70 | F | −2 | 2 | −2 | −3 | −2 | −1 | −2 | −3 | −1 | 0 | 1 | −1 | 0 | 4 | −3 | −2 | −2 | −1 | 0 | −1 |
| 71 | P | −1 | 0 | −1 | −1 | −3 | 0 | 0 | −2 | −2 | −3 | −3 | 2 | −2 | −3 | 5 | 1 | −1 | −4 | −3 | −2 |
| 72 | G | 0 | −2 | 0 | −1 | −3 | −2 | −2 | 6 | −2 | −4 | −4 | −2 | −3 | −3 | −2 | 0 | −2 | −2 | −3 | −3 |
| 73 | Q | −1 | 0 | 0 | 0 | −3 | 6 | 1 | −2 | 0 | −3 | −2 | 2 | 0 | −3 | −1 | 0 | −1 | −2 | −1 | −2 |
| 74 | V | 0 | −3 | −3 | −3 | −1 | −2 | −2 | −3 | −3 | 3 | 0 | −2 | 0 | −1 | −2 | −2 | 0 | −3 | −1 | 4 |
| 75 | A | 5 | −1 | −2 | −2 | 0 | −1 | −1 | 0 | −2 | −1 | −1 | −1 | −1 | −2 | −1 | 0 | 0 | −3 | −2 | 0 |
| 76 | G | 0 | −2 | 0 | −1 | −3 | −2 | −2 | 6 | −2 | −4 | −4 | −2 | −3 | −3 | −2 | 0 | −2 | −2 | −3 | −3 |
| 77 | T | 0 | −1 | 0 | −1 | −1 | −1 | −1 | −2 | −2 | −1 | −1 | −1 | −1 | −2 | −1 | 0 | 6 | −2 | −2 | 0 |
| 78 | T | 0 | −1 | 0 | −1 | −1 | −1 | −1 | −2 | −2 | −1 | −1 | −1 | −1 | −2 | −1 | 0 | 6 | −2 | −2 | 0 |
| 79 | R | −1 | 6 | 0 | −2 | −3 | 0 | 0 | −2 | 0 | −3 | −2 | 1 | −1 | −3 | −2 | −1 | −1 | −3 | −2 | −3 |
| 80 | A | 4 | −1 | 1 | −1 | 0 | −1 | −1 | 0 | −1 | −1 | −1 | −1 | −1 | −2 | −1 | 0 | 0 | −3 | −2 | 0 |
| 81 | A | 0 | 4 | 0 | −2 | −2 | 2 | 0 | −2 | −1 | −2 | 0 | 2 | 0 | −3 | −2 | 0 | −1 | −3 | −2 | −2 |
| 82 | P | −1 | −2 | −2 | −1 | −3 | −1 | −1 | −2 | −2 | −3 | −3 | −1 | −2 | −4 | 7 | −1 | −1 | −4 | −3 | −2 |
| 83 | S | 2 | −1 | 0 | −1 | −1 | 0 | 0 | 0 | −1 | −2 | −2 | 0 | −1 | −2 | −1 | 4 | 0 | −3 | −2 | −1 |
| 84 | C | 0 | −3 | −3 | −3 | 10 | −3 | −4 | −3 | −3 | −1 | −1 | −3 | −1 | −2 | −3 | −1 | −1 | −2 | −2 | −1 |
| 85 | V | 0 | −3 | −3 | −3 | −1 | −2 | −2 | −3 | −3 | 2 | 0 | −2 | 0 | −1 | −2 | −2 | 0 | −3 | −1 | 5 |
| 86 | D | −2 | −2 | 0 | 6 | −3 | 0 | 2 | −1 | −1 | −3 | −4 | −1 | −3 | −3 | −1 | 0 | −1 | −4 | −3 | −3 |
| 87 | A | 5 | −1 | −2 | −2 | 0 | −1 | −1 | 0 | −2 | −1 | −1 | −1 | −1 | −2 | −1 | 0 | 0 | −3 | −2 | 0 |
| 88 | S | 0 | 2 | 0 | −1 | −1 | 0 | 0 | −1 | −1 | −2 | −2 | 0 | −1 | −2 | −1 | 4 | 0 | −3 | −2 | −2 |
| 89 | I | −1 | −3 | −3 | −3 | −1 | −3 | −3 | −4 | −3 | 5 | 1 | −3 | 0 | 0 | −3 | −2 | −1 | −3 | −1 | 2 |
| 90 | V | 0 | −3 | −3 | −3 | −1 | −2 | −2 | −3 | −3 | 3 | 0 | −2 | 0 | −1 | −2 | −2 | 0 | −3 | −1 | 4 |
| 91 | E | −1 | 1 | −1 | −1 | −2 | 0 | 1 | −3 | −2 | 2 | 1 | 2 | 0 | −1 | −2 | −1 | −1 | −3 | −2 | 0 |
| 92 | Q | −1 | 0 | 0 | 0 | −2 | 4 | 1 | 0 | −1 | −2 | −2 | 0 | −1 | −2 | −1 | 0 | 2 | 2 | −1 | −2 |
| 93 | K | −1 | 2 | 0 | −1 | −3 | 0 | 0 | −2 | −1 | −3 | −2 | 6 | −1 | −3 | −1 | 0 | −1 | −3 | −2 | −2 |
| 94 | W | −2 | −1 | −2 | −2 | −2 | 2 | −1 | −2 | −1 | −3 | −2 | −1 | −1 | 0 | −3 | −2 | −2 | 10 | 0 | −3 |
| 95 | W | −3 | −3 | −4 | −4 | −2 | −2 | −3 | −2 | −2 | −3 | −2 | −3 | −1 | 0 | −4 | −3 | −2 | 11 | 1 | −3 |
| 96 | C | 0 | −3 | −3 | −3 | 10 | −3 | −4 | −3 | −3 | −1 | −1 | −3 | −1 | −2 | −3 | −1 | −1 | −2 | −2 | −1 |
| 97 | H | −2 | 0 | 1 | 3 | −3 | 2 | 2 | −2 | 5 | −3 | −3 | 0 | −2 | −2 | −1 | 0 | −1 | −3 | 0 | −3 |
| 98 | M | −1 | −1 | −2 | −3 | −1 | 0 | −2 | −3 | −2 | 0 | 1 | −1 | 7 | 0 | −2 | −1 | −1 | −1 | −1 | 0 |
| 99 | Q | −1 | 0 | 2 | 0 | −3 | 2 | 3 | −2 | 0 | −1 | 1 | 0 | 0 | −2 | −2 | 0 | −1 | −3 | −2 | −1 |
| 100 | P | −1 | −2 | −2 | −1 | −3 | −1 | −1 | −2 | −2 | −3 | −3 | −1 | −2 | −4 | 7 | −1 | −1 | −4 | −3 | −2 |
| 101 | C | 0 | −3 | −3 | −3 | 10 | −3 | −4 | −3 | −3 | −1 | −1 | −3 | −1 | −2 | −3 | −1 | −1 | −2 | −2 | −1 |
| 102 | L | −1 | −2 | −3 | −4 | −1 | −2 | −3 | −4 | −3 | 1 | 4 | −2 | 1 | 0 | −3 | −2 | −1 | −2 | −1 | 1 |
| 103 | E | −1 | −1 | −1 | 0 | −3 | 0 | 5 | −2 | −1 | −1 | 0 | 0 | −1 | −2 | 0 | 0 | −1 | −3 | −2 | 0 |
| 104 | G | 0 | −2 | 0 | −1 | −3 | −2 | −2 | 6 | −2 | −4 | −4 | −2 | −3 | −3 | −2 | 0 | −2 | −2 | −3 | −3 |
| 105 | E | −1 | 0 | 0 | 1 | −4 | 1 | 6 | −2 | 0 | −3 | −3 | 0 | −2 | −3 | −1 | 0 | −1 | −3 | −2 | −2 |
| 106 | E | −1 | −1 | 0 | 2 | −4 | 0 | 4 | 2 | −1 | −3 | −4 | 0 | −3 | −3 | −1 | 0 | −1 | −3 | −3 | −3 |
| 107 | C | 0 | −3 | −3 | −3 | 10 | −3 | −4 | −3 | −3 | −1 | −1 | −3 | −1 | −2 | −3 | −1 | −1 | −2 | −2 | −1 |
| 108 | K | −1 | 1 | 0 | 3 | −3 | 0 | 0 | −2 | −1 | −3 | −3 | 4 | −2 | −3 | −1 | 0 | −1 | −3 | −2 | −2 |
| 109 | V | 0 | −2 | −2 | −3 | −1 | −2 | −2 | −3 | −3 | 1 | 2 | −2 | 0 | −1 | −2 | −1 | 1 | −3 | −1 | 4 |
| 110 | L | −1 | −2 | −3 | −4 | −1 | −2 | −3 | −4 | −3 | 1 | 4 | −2 | 1 | 0 | −3 | −2 | −1 | −2 | −1 | 0 |
| 111 | P | −1 | −2 | −2 | −2 | −2 | −2 | −2 | −3 | −2 | 2 | −1 | −2 | −1 | −2 | 6 | −1 | −1 | −4 | −2 | 0 |
| 112 | D | −2 | −1 | 3 | 6 | −3 | 0 | 1 | −1 | 0 | −3 | −4 | −1 | −3 | −3 | −1 | 0 | −1 | −4 | −3 | −3 |
| 113 | R | −1 | 3 | 2 | −2 | −2 | 0 | −1 | −2 | 0 | −1 | 1 | 0 | 0 | −1 | −2 | 0 | −1 | −2 | 1 | −1 |
| 114 | K | 0 | −1 | 0 | 0 | −1 | 0 | 0 | 0 | −1 | −2 | −2 | 1 | −1 | −2 | −1 | 4 | 1 | −3 | −2 | −2 |
| 115 | G | 0 | −2 | 0 | −1 | −3 | −2 | −2 | 6 | −2 | −4 | −4 | −2 | −3 | −3 | −2 | 0 | −2 | −2 | −3 | −3 |
| 116 | W | −3 | −3 | −4 | −4 | −2 | −2 | −3 | −2 | −2 | −3 | −2 | −3 | −1 | 0 | −4 | −3 | −2 | 11 | 1 | −3 |
| 117 | S | 0 | −1 | 0 | −1 | −1 | 0 | −1 | −1 | −1 | −1 | −1 | 0 | 2 | −2 | −1 | 3 | 3 | −2 | −2 | −1 |
| 118 | C | 0 | −3 | −3 | −3 | 10 | −3 | −4 | −3 | −3 | −1 | −1 | −3 | −1 | −2 | −3 | −1 | −1 | −2 | −2 | −1 |
| 119 | S | 1 | −1 | 0 | −1 | −1 | −1 | −1 | −1 | −2 | 0 | −1 | −1 | −1 | −2 | −1 | 3 | 3 | −3 | −2 | 0 |
| 120 | S | 0 | 0 | 0 | 0 | −2 | 3 | 0 | −1 | −1 | −2 | −2 | 0 | −1 | −2 | −1 | 3 | 1 | −3 | −2 | −2 |
| 121 | G | 0 | −2 | 0 | −1 | −3 | −2 | −2 | 6 | −2 | −4 | −4 | −2 | −3 | −3 | −2 | 0 | −2 | −2 | −3 | −3 |
| 122 | N | −1 | −1 | 5 | 0 | −3 | −1 | −1 | 3 | 4 | −3 | −3 | −1 | −2 | −3 | −2 | 0 | −1 | −3 | −1 | −3 |
| 123 | K | −1 | 3 | 0 | −1 | −3 | 0 | 0 | −2 | −1 | −3 | −2 | 5 | −1 | −3 | −1 | 0 | −1 | −3 | −2 | −2 |
| 124 | V | 0 | −3 | −3 | −3 | −1 | −2 | −2 | −4 | −3 | 4 | 0 | −2 | 0 | −1 | −2 | −2 | 0 | −3 | −1 | 4 |
| 125 | K | −1 | 1 | 0 | −1 | −3 | 0 | 0 | −2 | −1 | −3 | −2 | 6 | −1 | −3 | −1 | 0 | −1 | −3 | −2 | −2 |
| 126 | T | 0 | −1 | 0 | −1 | −1 | −1 | −1 | −2 | −2 | −1 | −1 | −1 | −1 | −2 | −1 | 0 | 6 | −2 | −2 | 0 |
| 127 | T | 0 | −1 | 0 | −1 | −1 | −1 | −1 | −2 | −2 | −1 | −1 | −1 | −1 | −2 | −1 | 0 | 6 | −2 | −2 | 0 |
| 128 | R | −1 | 3 | 0 | −1 | −2 | 0 | 0 | −2 | −1 | −2 | −2 | 4 | −1 | −3 | −1 | 0 | 2 | −3 | −2 | −2 |
| 129 | V | 0 | −3 | −3 | −3 | −1 | −2 | −2 | −3 | −3 | 2 | 0 | −2 | 0 | −1 | −2 | −2 | 0 | −3 | −1 | 5 |
| 130 | T | 0 | −1 | 0 | −1 | −1 | 0 | −1 | −1 | 2 | −1 | 0 | −1 | −1 | −2 | −1 | 3 | 3 | −3 | −1 | −1 |
| 131 | H | −1 | 4 | 0 | −1 | −3 | 0 | 0 | −2 | 4 | −3 | −2 | 0 | −1 | −3 | 3 | −1 | −1 | −3 | −1 | −3 |

(ii) is a fragment thereof which is a member of the EGF domain containing protein family, preferably having biological activity similar to Coagulation Factor X, or has an antigenic determinant in common with the polypeptides of (i); or
(iii) is a functional equivalent of (i) or (ii).

Preferably, in the above test, the polypeptide gives a maximum threshold E value of 10⁻². More preferably, the polypeptide sequence has a minimum threshold E value of 10⁻⁵ or less, 10⁻¹⁰ or less, 10⁻⁵⁰ or less, most preferably, 10⁻⁷⁰ or less. Lowering the threshold value acts as a more stringent filter to separate polypeptides comprising a signal peptide and EGF-like domain from the general background polypeptide sequences.

In a third embodiment of the second aspect of the invention, there is provided an isolated polypeptide which

(i) comprises a polypeptide satisfying the consensus amino acid sequence:

G-T-C-E-[VI]-[VI]-[AT]-[AVL]-[HD]-R-[CD]-[CS]-
[NS]-[KRQ]-[NP]-[RK]-[IR]-[ET]-[EI]-[RA]-[SR]-
Q-T-[VA]-[KR]-C-[SA]-C-[LFR]-[PSK]-G-[KQ]-[VI]-
A-G-T-T-R-[NA]-[RQLAK]-P-[SA]-C-V-[DE]-A-[SAR]-
I-[VI]-[IELKR]-[WGQET]-[KR]-[WQ]-W-C-[EHNQD]-M-
[ENQL]-P-C-[LV]-[EVLP]-G-E-[DEG]-C-[KRD]-[TVL]-
L-[PI]-[DN]-[NYSLR]-[STK]-G-W-[MST]-C-[ASIT]-
[TSRQ]-P(0,1)-G-[NHG]-[KR]-[IV]-K-T-T;

(ii) is a fragment thereof which is a member of the EGF domain containing protein family, preferably having biological activity similar to Coagulation Factor X, or has an antigenic determinant in common with the polypeptides of (i); or
(iii) is a functional equivalent of (i) or (ii).

In a fourth embodiment of the second aspect of the invention, there is provided an isolated polypeptide which consists of a polypeptide satisfying the consensus amino acid sequence:

G-T-C-E-[VI]-[VI]-[AT]-[AVL]-[HD]-R-[CD]-[CS]-
[NS]-[KRQ]-[NP]-[RK]-[IR]-[ET]-[EI]-[RA]-[SR]-
Q-T-[VA]-[KR]-C-[SA]-C-[LFR]-[PSK]-G-[KQ]-[VI]-
A-G-T-T-R-[NA]-[RQLAK]-P-[SA]-C-V-[DE]-A-[SAR]-
I-[VI]-[IELKR]-[WGQET]-[KR]-[WQ]-W-C-[EHNQD]-M-
[ENQL]-P-C-[LV]-[EVLP]-G-E-[DEG]-C-[KRD]-[TVL]-
L-[PI]-[DN]-[NYSLR]-[STK]-G-W-[MST]-C-[ASIT]-
[TSRQ]-P(0,1)-G-[NHG]-[KR]-[IV]-K-T-T.

In a fifth embodiment of the second aspect of the invention, there is provided an isolated polypeptide of the third embodiment of the second aspect of the invention, wherein the isolated polypeptide comprises one or more, preferably, all of the four cysteine residues at amino acid positions 55, 60, 66, and 77 of the consensus amino acid sequence. The amino acid sequences of the third and fourth embodiments of the invention are written in PROSITE (protein sites and patterns) notation, with the amino acids being represented by their one-letter codes (Bairoch, A., Bucher, P., and Hofmann, K., (1997). The PROSITE Database: Its status in 1997. Nucl. Acids Res. 25, 217-221). Briefly, a peptide comprising the following formula:
A(1)−x(i1,j1)−A2−x(i2,j2)− . . . A{p−1}−x(i{p1},j{p−1})−Ap
is to be interpreted in the following manner.
A(k) is a component, either specifying one amino acid, e.g. C, or a set of possible amino acids, e.g. [ILVF]. A component A(k) is an identity component if it specifies exactly one amino acid (for instance C or L) or an ambiguous component if it specifies more than one (for instance [ILVF] or [FWY]). i(k), j(k) are integers so that i(k)<=j(k) for all k. The part x(ik,jk) specifies a wildcard region of the pattern matching between ik and jk arbitrary amino acids. A wildcard region x(ik,jk) is “flexible” if jk is bigger than ik (for example x(2,3). The flexibility of such a region is jk-ik.br> For example the flexibility of x(2,3) is 1. The wildcard region is fixed if j(k) is equal to i(k), e.g., x(2,2) which can be written as x(2). The product of flexibility for a pattern is the product of the flexibilities of the flexible wildcard regions in the pattern, if any, otherwise it is defined to be one. For example, C-x(2)-H is a pattern with two components (C and H) and one fixed wildcard region. It matches any sequence containing a C followed by any two arbitrary amino acids followed by an H. Amino acid sequences ChgHyw and liChgHlyw would be included in the formula. C-x(2,3)-H is a pattern with two components (C and H) and one flexible wildcard region. It matches any sequence containing a C followed by any two or three arbitrary amino acids followed by an H such as aaChgiywk and liChgaHlyw. C-x(2,3)-[ILV] is a pattern with two components (C and [ILV]) and one flexible wildcard region. It matches any sequence containing a C followed by any two or three arbitrary amino acids followed by an I, L or V.

The sequence recited in this embodiment of the invention covers the high identity region from INSP117 (SEQ ID NO:26) amino acid position 44-129 (amino acids 52-138 of the alignment, see FIG. 1).

Although the Applicant does not wish to be bound by this theory, it is postulated that the polypeptides of the above-described embodiments of the invention all possess signal peptide sequences. Accordingly, mature forms of the described polypeptides which lack the signal peptides form a further aspect of the present invention.

In one embodiment of the third aspect of the invention, there is provided a polypeptide which:
(i) comprises the amino acid sequence as recited in SEQ ID NO:2, SEQ ID NO:4, SEQ ID NO:37 and/or SEQ ID NO:39;
(ii) is a fragment thereof which is a member of the EGF domain containing protein family, preferably having biological activity similar to Coagulation Factor X, or has an antigenic determinant in common with the polypeptides of (i); or
(ii) is a functional equivalent of (i) or (ii).

According to a second embodiment of this third aspect of the invention, there is provided a polypeptide which:
(i) consists of the amino acid sequence as recited in SEQ ID NO:2, SEQ ID NO:4, SEQ ID NO:37 and/or SEQ ID NO:39.

The polypeptide having the sequence recited in SEQ ID NO:2 (accession number CAD28501.1) is referred to hereafter as the “INSP113 polypeptide”.

Although the Applicant does not wish to be bound by this theory, it is postulated that the first 25 amino acids of the INSP113 polypeptide form the signal peptide. The INSP113 full length polypeptide sequences with and without the signal sequence are recited in SEQ ID NO: 2 and SEQ ID NO:4, respectively. The polypeptide having the sequence recited in SEQ ID NO:4 is referred to hereafter as “the INSP113 mature polypeptide”.

The polypeptide having the sequence recited in SEQ ID NO:37 is a splice variant of the INSP113 polypeptide and is referred to hereafter as the “INSP113 sv polypeptide”.

Although the Applicant does not wish to be bound by this theory, it is postulated that the first 25 amino acids of the INSP113sv polypeptide form the signal peptide. The sequence of the INSP113sv polypeptide without the signal sequence is recited in SEQ ID NO:39. The polypeptide having the sequence recited in SEQ ID NO:39 is referred to hereafter as “the INSP113sv mature polypeptide”.

Preferably, the antigenic determinant, fragment or functional equivalent of the second embodiment of the third aspect of the invention comprises one or more of the four cysteine residues at amino acid positions 96, 101, 107 and 118 of SEQ ID NO:2. More preferably, one or more of these cysteine residues participate in disulphide bond formation under physiological conditions. In this aspect of the invention, by “physiological conditions” is meant the natural environment in which the native or wildtype form of the polypeptide would be found. Disulphide bond formation is often integral to the correct conformation of a protein and thus, its function. Disulphide bond formation is often integral to the correct conformation of a protein and thus, its function. It is therefore important that such cysteine residues be conserved.

In a third embodiment of the third aspect of the invention, there is provided a polypeptide which:
(i) comprises the amino acid sequence as recited in SEQ ID NO:6, SEQ ID NO:8; SEQ ID NO:41, SEQ ID NO:43, SEQ ID NO:53 and/or SEQ ID NO:55;
(ii) is a fragment thereof which is a member of the EGF domain containing protein family, preferably having biological activity similar to Coagulation Factor X, or having an antigenic determinant in common with the polypeptides of (i); or
(iii) is a functional equivalent of (i) or (ii).

According to a fourth embodiment of this third aspect of the invention, there is provided a polypeptide which:
(i) consists of the amino acid sequence as recited in SEQ ID NO:6, SEQ ID NO:8, SEQ ID NO:41, SEQ ID NO:43, SEQ ID NO:53 and/or SEQ ID NO:55.

The polypeptide having the sequence recited in SEQ ID NO:6 (accession number CAD38865.1) is referred to hereafter as the “INSP114 polypeptide”.

Although the Applicant does not wish to be bound by this theory, it is postulated that the first 30 amino acids of the INSP114 polypeptide form the signal peptide. The INSP114 full length polypeptide sequences without the signal sequence is recited in SEQ ID NO: 8. The polypeptide having the sequence recited in SEQ ID NO:8 is referred to hereafter as “the INSP114 mature polypeptide”.

The polypeptide having the sequence recited in SEQ ID NO:41 is a splice variant of the INSP14 polypeptide and is referred to hereafter as the “INSP114-SV2 polypeptide”.

Although the Applicant does not wish to be bound by this theory, it is postulated that the first 30 amino acids of the INSP114-SV2 polypeptide form the signal peptide. The sequence of the INSP114-SV2 polypeptide without the signal sequence is recited in SEQ ID NO:43. The polypeptide having the sequence recited in SEQ ID NO:43 is referred to hereafter as “the INSP114-SV2 mature polypeptide”.

The polypeptide having the sequence recited in SEQ ID NO:53 is a splice variant of the INSP114 polypeptide and is referred to hereafter as the “INSP114-SV1 polypeptide”.

Although the Applicant does not wish to be bound by this theory, it is postulated that the first 30 amino acids of the INSP114-SV1 polypeptide form the signal peptide. The sequence of the INSP114-SV1 polypeptide without the signal sequence is recited in SEQ ID NO:55. The polypeptide having the sequence recited in SEQ ID NO:55 is referred to hereafter as “the INSP114-SV1 mature polypeptide”.

Preferably, the antigenic determinant, fragment or functional equivalent of the fourth embodiment of the third aspect of the invention comprises one or more of the four cysteine residues at amino acid positions 96, 101, 107 and 118 of SEQ ID NO:6, SEQ ID NO:41 or SEQ ID NO:53. More preferably, one or more of these cysteine residues participate in disulphide bond formation under physiological conditions. Disulphide bond formation is often integral to the correct conformation of a protein and thus, its function. It is therefore important that such cysteine residues be conserved.

In a fifth embodiment of the third aspect of the invention, there is provided a polypeptide which:
(i) comprises the amino acid sequence as recited in SEQ ID NO:10, SEQ ID NO:12, SEQ ID NO:45 and/or SEQ ID NO:47;
(ii) is a fragment thereof which is a member of the EGF domain containing protein family, preferably having biological activity similar to Coagulation Factor X, or having an antigenic determinant in common with the polypeptides of (i); or
(i) is a functional equivalent of (i) or (ii).

According to a sixth embodiment of this third aspect of the invention, there is provided a polypeptide which:
(i) consists of the amino acid sequence as recited in SEQ ID NO:10, SEQ ID NO:12, SEQ ID NO:45 and/or SEQ ID NO:47.

The polypeptide having the sequence recited in SEQ ID NO:10 (accession number AAY53016) is referred to hereafter as the “NSP115 polypeptide”.

Although the Applicant does not wish to be bound by this theory, it is postulated that the first 42 amino acids of the INSP115 polypeptide form the signal peptide. The INSP115 full length polypeptide sequences without the signal sequence is recited in SEQ ID NO: 12. The polypeptide having the sequence recited in SEQ ID NO:12 is referred to hereafter as “the INSP115 mature polypeptide”.

The polypeptide having the sequence recited in SEQ ID NO:45 is referred to hereafter as the “INSP115 cloned polypeptide”.

Although the Applicant does not wish to be bound by this theory, it is postulated that the first 45 amino acids of the INSP115 cloned polypeptide form the signal peptide. The sequence of the INSP115 cloned polypeptide without the signal sequence is recited in SEQ ID NO: 47. The polypeptide having the sequence recited in SEQ ID NO:47 is referred to hereafter as “the INSP115 cloned mature polypeptide”.

Preferably, the antigenic determinant, fragment or functional equivalent of the sixth embodiment of the third aspect of the invention comprises one or more of the four cysteine residues at amino acid positions 97, 102, 108 and 119 of SEQ ID NO:10 or of SEQ ID NO:45. More preferably, one or more of these cysteine residues participate in disulphide bond formation under physiological conditions. Disulphide bond formation is often integral to the correct conformation of a protein and thus, its function. It is therefore important that such cysteine residues be conserved.

In a seventh embodiment of the third aspect of the invention, there is provided a polypeptide which:
(i) comprises the amino acid sequence as recited in SEQ ID NO:14, SEQ ID NO:16, SEQ ID NO:49 and/or SEQ ID NO:51;
(ii) is a fragment thereof which is a member of the EGF domain containing protein family, preferably having biological activity similar to Coagulation Factor X, or having an antigenic determinant in common with the polypeptides of (i); or
(iii) is a functional equivalent of (i) or (ii).

According to an eighth embodiment of this third aspect of the invention, there is provided a polypeptide which:
(i) consists of the amino acid sequence as recited in SEQ ID NO:14, SEQ ID NO:16, SEQ ID NO:49 and/or SEQ ID NO:51.

The polypeptide having the sequence recited in SEQ ID NO:14 (accession number XP_{—}087261.1) is referred to hereafter as the “INSP116 polypeptide”.

Although the Applicant does not wish to be bound by this theory, it is postulated that the first 34 amino acids of the INSP116 polypeptide form the signal peptide. The INSP116 full length polypeptide sequences without the signal sequence is recited in SEQ ID NO: 16. The polypeptide having the sequence recited in SEQ ID NO:16 is referred to hereafter as “the INSP116 mature polypeptide”.

The polypeptide having the sequence recited in SEQ ID NO:49 is referred to hereafter as the “INSP116 cloned polypeptide”.

Although the Applicant does not wish to be bound by this theory, it is postulated that the first 36 amino acids of the INSP116 cloned polypeptide form the signal peptide. The sequence of the INSP116 cloned polypeptide without the signal sequence is recited in SEQ ID NO: 51. The polypeptide having the sequence recited in SEQ ID NO:51 is referred to hereafter as “the INSP116 cloned mature polypeptide”.

Preferably, the antigenic determinant, fragment or functional equivalent of the eighth embodiment of the third aspect of the invention comprises one or more of the four cysteine residues at amino acid positions 105, 110, 116, 127 of SEQ ID NO:14 or of SEQ ID NO:49. More preferably, one or more of these cysteine residues participate in disulphide bond formation under physiological conditions. Disulphide bond formation is often integral to the correct conformation of a protein and thus, its function. It is therefore important that such cysteine residues be conserved.

In a ninth embodiment of the third aspect of the invention, there is provided a polypeptide which:
(i) comprises the amino acid sequence as recited in SEQ ID NO:18, SEQ ID NO:20, SEQ ID NO:22, SEQ ID NO:24, SEQ ID NO:26, SEQ ID NO:28 and/or SEQ ID NO:30;
(ii) is a fragment thereof which is a member of the EGF domain containing protein family, preferably having biological activity similar to Coagulation Factor X, or having an antigenic determinant in common with the polypeptides of (i); or
(iii) is a functional equivalent of (i) or (ii).

Preferably, the polypeptide according to this third aspect of the invention:
(i) comprises the amino acid sequence as recited in SEQ ID NO:26 and/or SEQ ID NO:30,
(ii) is a fragment thereof which is a member of the EGF domain containing protein family; preferably having biological activity similar to Coagulation Factor X, or having an antigenic determinant in common with the polypeptides of (i); or
(iii) is a functional equivalent of (i) or (ii).

According to a tenth embodiment of the third aspect of the invention, there is provided a polypeptide which:
(i) consists of the amino acid sequence as recited in SEQ ID NO:18, SEQ ID NO:20, SEQ ID NO:22, SEQ ID NO:24, SEQ ID NO:26, SEQ ID NO:28 and/or SEQ ID NO:30;

Preferably, the antigenic determinant, fragment or functional equivalent of the ninth embodiment of the third aspect of the invention comprises one or more of the four cysteine residues at amino acid positions 98, 103, 109 and 120 of SEQ ID NO: 26. More preferably, the cysteine residues at amino acid positions 98 and 109 form a disulphide pair under physiological conditions. It is therefore important that such cysteine residues be conserved. The four cysteine residues of this embodiment of the invention were identified by comparing SEQ ID NO:26 with the similar sequence for 1WHE (FIG. 12).

The polypeptide having the sequence recited in SEQ ID NO:18 is referred to hereafter as “the INSP117 exon 1 polypeptide”. The polypeptide having the sequence recited in SEQ ID NO:20 is referred to hereafter as “the INSP117 exon 2 polypeptide”. The polypeptide having the sequence recited in SEQ ID NO:22 is referred to hereafter as “the INSP117 exon 3 polypeptide”. The polypeptide having the sequence recited in SEQ ID NO:24 is referred to hereafter as “the INSP117 exon 4 polypeptide”.

The polypeptide having the sequence recited in SEQ ID NO:26 is referred to hereafter as “the INSP117 polypeptide”.

Although the Applicant does not wish to be bound by this theory, it is postulated that the first 30 amino acids of INSP117 exon 1 polypeptide forms the signal peptide. The INSP117 exon 1 and full length polypeptide sequences without the signal sequence are recited in SEQ ID NO: 28 and SEQ ID NO:30, respectively. The polypeptide having the sequence recited in SEQ ID NO:28 is referred to hereafter as “the INSP117 exon 1 mature polypeptide”. The polypeptide having the sequence recited in SEQ ID NO:30 is referred to hereafter as “the INSP117 mature polypeptide”.

The term “INSP117 exon polypeptides” as used herein includes polypeptides comprising the INSP117 exon 1 polypeptide, the INSP117 exon 2 polypeptide, the INSP117 exon 1 mature polypeptide, the INSP117 exon 3 polypeptide, the INSP117 exon 4 polypeptide, the INSP117 polypeptide or the INSP117 mature polypeptide, as well as polypeptides consisting of the INSP117 exon 1 polypeptide, the INSP117 exon 2 polypeptide, the INSP117 exon 1 mature polypeptide, the INSP117 exon 3 polypeptide, the INSP117 exon 4 polypeptide, the INSP117 polypeptide or the INSP117 mature polypeptide.

The terms “INSP113, INSP114, INSP115, INSP116 and INSP117 polypeptides” and “INSP113, INSP114, INSP115, INSP116 or INSP117 polypeptides” as used herein include polypeptides comprising the sequences recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53 and/or SEQ ID NO:55.

As already explained in the first aspect of the invention, the identification of novel proteins comprising EGF-like domains is useful since such domains have been found to play an important role in a broad-cross section of diseases including the growth and development of many types of tumours, in particular, human diseases and tumours.

Preferably a polypeptide according to the second or third aspect of the invention is a member of the EGF domain containing protein family, preferably having biological activity similar to Coagulation Factor X. By “having biological activity similar to Coagulation Factor X” is meant that the polypeptide has biological activity similar to Coagulation Factor X which in turn has biological activity similar to the TAFA family which are similar to CC-chemokines (see Tang, Y. T. et al., ‘TAFA: A novel secreted family with homology to CC-chemokines’, Genbank record AAP92046, at http://harvester.embl.de/harvester/Q7Z5/Q7Z5A7.htm; also see Tang et al., ‘TAFA: a novel secreted family with conserved cysteine residues and restricted expression in the brain’, Genomics. 2004 April; 83(4):727-34). Assays for determining whether the poypeptide has biological activity similar to Coagulation Factor X are given in examples 17 and 18.

In a fourth aspect, the invention provides a purified nucleic acid molecule which encodes a polypeptide of the second or third aspect of the invention.

Preferably, the purified nucleic acid molecule comprises the nucleic acid sequence as recited in SEQ ID NO:1 (encoding the INSP113 polypeptide), SEQ ID NO:3 (encoding the INSP113 mature polypeptide), SEQ ID NO:5 (encoding the INSP114 polypeptide), SEQ ID NO:7 (encoding the INSP114 mature polypeptide), SEQ ID NO:9 (encoding the INSP115 polypeptide), SEQ ID NO:11 (encoding the INSP115 mature polypeptide), SEQ ID NO:13 (encoding the INSP116 polypeptide), SEQ ID NO:15 (encoding the INSP116 mature polypeptide), SEQ ID NO:17 (encoding the INSP117 exon 1 polypeptide), SEQ ID NO:19 (encoding the INSP117 exon 2 polypeptide), SEQ ID NO:21 (encoding the INSP117 exon 3 polypeptide), SEQ ID NO:23 (encoding the INSP117 exon 4 polypeptide), SEQ ID NO:25 (encoding the INSP117 polypeptide), SEQ ID NO:27 (encoding the INSP117 mature exon 1 polypeptide), SEQ ID NO:29 (encoding the INSP117 mature polypeptide), SEQ ID NO:36 (encoding the INSP113sv polypeptide), SEQ ID NO:38 (encoding the INSP113sv mature polypeptide), SEQ ID NO:40 (encoding the INSP114-SV2 polypeptide), SEQ ID NO:42 (encoding the INSP114-SV2 mature polypeptide), SEQ ID NO:44 (encoding the INSP115 cloned polypeptide), SEQ ID NO:46 (encoding the INSP115 cloned mature polypeptide), SEQ ID NO:48 (encoding the INSP116 cloned polypeptide), SEQ ID NO:50 (encoding the INSP116 cloned mature polypeptide), SEQ ID NO:52 (encoding the INSP114-SV1 polypeptide) and/or SEQ ID NO:54 (encoding the INSP114-SV1 mature polypeptide) or is a redundant equivalent or fragment of any one of these sequences.

The invention further provides that the purified nucleic acid molecule consists of the nucleic acid sequence as recited in SEQ ID NO:1 (encoding the INSP113 polypeptide), SEQ ID NO:3 (encoding the INSP113 mature polypeptide), SEQ ID NO:5 (encoding the INSP114 polypeptide), SEQ ID NO:7 (encoding the INSP114 mature polypeptide), SEQ ID NO:9 (encoding the INSP115 polypeptide), SEQ ID NO:11 (encoding the INSP115 mature polypeptide), SEQ ID NO:13 (encoding the INSP116 polypeptide), SEQ ID NO:15 (encoding the INSP116 mature polypeptide), SEQ ID NO:17 (encoding the INSP117 exon 1 polypeptide), SEQ ID NO:19 (encoding the INSP117 exon 2 polypeptide), SEQ ID NO:21 (encoding the INSP117 exon 3 polypeptide), SEQ ID NO:23 (encoding the INSP117 exon 4 polypeptide), SEQ ID NO:25 (encoding the INSP117 polypeptide), SEQ ID NO:27 (encoding the INSP117 mature exon 1 polypeptide), SEQ ID NO:29 (encoding the INSP117 mature polypeptide), SEQ ID NO:36 (encoding the INSP113sv polypeptide), SEQ ID NO:38 (encoding the INSP113sv mature polypeptide), SEQ ID NO:40 (encoding the INSP114-SV2 polypeptide), SEQ ID NO:42 (encoding the INSP114-SV2 mature polypeptide), SEQ ID NO:44 (encoding the INSP115 cloned polypeptide), SEQ ID NO:46 (encoding the INSP115 cloned mature polypeptide), SEQ ID NO:48 (encoding the INSP116 cloned polypeptide), SEQ ID NO:50 (encoding the INSP116 cloned mature polypeptide), SEQ ID NO:52 (encoding the INSP114-SV1 polypeptide) and/or SEQ ID NO:54 (encoding the INSP114-SV1 mature polypeptide) or is a redundant equivalent or fragment of any one of these sequences.

Although the sequences recited in this application for SEQ ID NO:52 and SEQ ID NO:54 do not include a stop codon, the recitation of SEQ ID NO:52 and SEQ ID NO:54 as used herein also includes nucleotide sequences having the sequences recited in this application for SEQ ID NO:52 and SEQ ID NO:54 that do also include a stop codon. Similarly, for other SEQ ID NOs recited in this application that do not include a stop codon, recitation of the SEQ ID NO also includes recitation of that sequence including a stop codon.

According to one embodiment of this aspect of the invention, the purified nucleic acid molecule excludes the signal peptide located at the start of INSP117 exon 1 polypeptide (amino acids 1 to 30 of SEQ ID NO:18). According to this embodiment, the purified nucleic acid molecule preferably comprises nucleotides 91 to 115 of SEQ ID NO:17 (shown in SEQ ID NO:27, encoding the INSP117 exon 1 mature polypeptide) or nucleotides 91 to 402 of SEQ ID NO:25 (shown in SEQ ID NO:29, encoding the INSP117 mature polypeptide). The invention further provides a purified nucleic acid molecule consisting of nucleotides 91 to 115 of SEQ ID NO: 17 (shown in SEQ ID NO:27, encoding the INSP117 exon 1 mature polypeptide) or nucleotides 91 to 402 of SEQ ID NO:25 (shown in SEQ ID NO:29, encoding the INSP117 mature polypeptide).

In a fifth aspect, the invention provides a purified nucleic acid molecule which hybridizes under high stringency conditions with a nucleic acid molecule of the fourth aspect of the invention.

In a sixth aspect, the invention provides a vector, such as an expression vector, that contains a nucleic acid molecule of the fourth or fifth aspect of the invention. Preferred vectors include pCR4-TOPO-INSP113 (FIG. 18), pCR4-TOPO-INSP113sv (FIG. 19), pDONR (FIG. 20), pEAK12d (FIG. 21), pDEST12.2 (FIG. 22), pENTR-INSP113-6HIS (FIG. 23), pENTR-INSP113sv-6HIS (FIG. 24), pEAK12d-INSP113-6HIS (FIG. 25), pEAK12d-INSP113sv-6HIS (FIG. 26), pDEST12.2-INSP113-6HIS (FIG. 27), pDEST12.2-INSP113sv-6HIS (FIG. 28), pCR4-TOPO-INSP114 (FIG. 31), pCR4-TOPO-INSP114-GR1 (FIG. 35), pCR4-TOPO-INSP114-SV2 (FIG. 36), pDONR 221 (FIG. 38), pEAK12d (FIG. 39), pDEST12.2 (FIG. 40), pENTR_INSP114-6HIS (FIG. 41), pEAK12d_INSP114-6HIS (FIG. 42), pDEST12.2_INSP114-6HIS (FIG. 43), pENTR_INSP114-SV1-6HIS (FIG. 44), pEAK12d-INSP114-SV1-6HIS (FIG. 45), pDEST12.2_INSP114-SV1-6HIS (FIG. 46), pENTR_INSP114-SV2-6HIS (FIG. 47), pEAK12d_INSP114-SV2-6HIS (FIG. 48), pDEST12.2_INSP114-SV2-6HIS (FIG. 49), pDONR 221 (FIG. 51), pEAK12d (FIG. 52), pDEST12.2 (FIG. 53), pENTR_INSP115-6HIS (FIG. 54), pEAK12d_INSP115-6HIS (FIG. 55), pDEST12.2_INSP115-6HIS (FIG. 56), pDONR 221 (FIG. 58), pEAK12d (FIG. 59), pDEST12.2 (FIG. 60), pENTR_INSP116-6HIS (FIG. 61), pEAK12d_JNSP116-6HIS (FIG. 62), pDEST12.2_INSP116-61HIS (FIG. 63), pCRII-TOPO-INSP117 (FIG. 66), pDONR 221 (FIG. 67), pEAK12d (FIG. 68), pDEST12.2 (FIG. 69), pENTR_INSP117-6HIS (FIG. 70), pEAK12d_INSP17-6HIS (FIG. 71) and pDEST12.2_INSP117-6HIS (FIG. 72).

In a seventh aspect, the invention provides a host cell transformed with a vector of the sixth aspect of the invention.

In an eighth aspect, the invention provides a ligand which binds specifically to a member of the EGF containing protein family of the second or third aspect of the invention. Preferably, the ligand inhibits the function of a polypeptide of the first aspect of the invention which is a member of the EGF domain-containing family of proteins. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a ninth aspect, the invention provides a compound that is effective to alter the expression of a natural gene which encodes a polypeptide of the second or third aspect of the invention or to regulate the activity of a polypeptide of the second or third aspect of the invention.

A compound of the ninth aspect of the invention may either increase (agonise) or decrease (antagonise) the level of expression of the gene or the activity of the polypeptide.

Importantly, the identification of the function of the INSP113, INSP114, INSP115, INSP116 and INSP117 polypeptides allows for the design of screening methods capable of identifying compounds that are effective in the treatment and/or diagnosis of disease. Ligands and compounds according to the eighth and ninth aspects of the invention may be identified using such methods. These methods are included as aspects of the present invention.

In a tenth aspect, the invention provides a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, for use in therapy or diagnosis of diseases in which members of the EGF domain containing protein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions. Preferably, the diseases are those in which EGF domain containing proteins are implicated. These molecules may also be used in the manufacture of a medicament for the treatment of such diseases. These molecules may also be used in contraception or for the treatment of reproductive disorders including infertility.

In a eleventh aspect, the invention provides a method of diagnosing a disease in a patient, comprising assessing the level of expression of a natural gene encoding a polypeptide of the second or third aspect of the invention or the activity of a polypeptide of the second or third aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the second or third aspect of the invention comprises the steps of: (a) contacting a ligand, such as an antibody, of the eighth aspect of the invention with a biological sample under conditions suitable for the formation of a ligand-polypeptide complex; and (b) detecting said complex.

A number of different such methods according to the eleventh aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a twelfth aspect, the invention provides for the use of a polypeptide of the second or third aspect of the invention as an EGF domain containing protein. Suitable uses of the polypeptides of the invention as EGF domain containing proteins include use as a regulator of cellular growth, metabolism or differentiation, use as part of a receptor/ligand pair and use as a diagnostic marker for a physiological or pathological condition.

In an thirteenth aspect, the invention provides a pharmaceutical composition comprising a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, in conjunction with a pharmaceutically-acceptable carrier.

In a fourteenth aspect, the present invention provides a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention, for use in the manufacture of a medicament for the diagnosis or treatment of a disease.

In a fifteenth aspect, the invention provides a method of treating a disease in a patient comprising administering to the patient a polypeptide of the second or third aspect of the invention, or a nucleic acid molecule of the fourth or fifth aspect of the invention, or a vector of the sixth aspect of the invention, or a host cell of the seventh aspect of the invention, or a ligand of the eighth aspect of the invention, or a compound of the ninth aspect of the invention.

For diseases in which the expression of a natural gene encoding a polypeptide of the second or third aspect of the invention, or in which the activity of a polypeptide of the second or third aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

In a sixteenth aspect, the invention provides transgenic or knockout non-human animals that have been transformed to express higher, lower or absent levels of a polypeptide of the second or third aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the second or third aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the third aspect of the invention may be polypeptides that are homologous to the INSP113, INSP114, INSP115, INSP116 and INSP117 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP113, INSP114, INSP115, INSP116 and INSP117 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

Typically, greater than 30% identity between two polypeptides is considered to be an indication of functional equivalence. Preferably, functionally equivalent polypeptides of the second or third aspect of the invention have a degree of sequence identity with the INSP113, INSP114, INSP115, INSP116 or INSP117 polypeptides, or with active fragments thereof, of greater than 80%. More preferred polypeptides have degrees of identity of greater than 85%, 90%, 95%, 98% or 99%, respectively.

The functionally-equivalent polypeptides of the second or third aspect of the invention may also be polypeptides which have been identified using one or more techniques of structural alignment. For example, the Inpharmatica Genome Threader technology that forms one aspect of the search tools used to generate the Biopendium™ search database may be used (see PCT application WO 01/69507) to identify polypeptides of presently-unknown function which, while having low sequence identity as compared to the INSP113, INSP114, INSP115, INSP116 and INSP117 polypeptides, are predicted to be members of the EGF domain containing protein family, by virtue of sharing significant structural homology with the INS 113, INS 114, INS 115, INSP116 and INSP117 polypeptide sequences. By “significant structural homology” is meant that the Inpharmatica Genome Threader predicts two proteins to share structural homology with a certainty of 10% and above.

The polypeptides of the second or third aspect of the invention also include fragments of the INSP113, INSP114, INSP115, INSP116 and INSP117 polypeptides and fragments of the functional equivalents of the INSP113, INSP114, INSP115, INSP116 and INSP117 polypeptides, provided that those fragments are members of the EGF containing protein family or have an antigenic determinant in common with the INSP113, INSP114, INSP115, INSP116 and INSP117 polypeptides.

As used herein, the term “fragment” refers to a polypeptide having an amino acid sequence that is the same as part, but not all, of the amino acid sequence of the INSP113, INSP114, INSP115, INSP116 and INSP117 polypeptides or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant.

Fragments of the full length INSP113, INSP114, INSP115, INSP116 and INSP117 polypeptides may consist of combinations of 2, 3 or 4 of neighbouring exon sequences in the INSP113, INSP114, INSP115, INSP116 and INSP117 polypeptide sequences, respectively.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “protein” means a type of polypeptide including, but not limited to those that function as enzymes. Preferably, the protein or polypeptide of the present invention functions as a ligand. A ligand, in this context means a molecule that binds to another molecule, such as a receptor. A ligand may be a co-factor for an enzyme. The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the second or third aspect of the invention.

By “substantially greater affinity” we mean that there is a measurable increase in the affinity for a polypeptide of the invention as compared with the affinity for known secreted proteins.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known secreted proteins such as members of the EGF domain-containing family of proteins.

If polyclonal antibodies are desired, a selected mammal, such as a mouse, rabbit, goat or horse, may be immunised with a polypeptide of the second or third aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the second or third aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the second or third aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the fourth and fifth aspects of the invention are those which encode a polypeptide sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53 and/or SEQ ID NO:55 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ED NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53 and/or SEQ ID NO:55. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the fourth and fifth aspects of the invention may also encode the fragments or the functional equivalents of the polypeptides and fragments of the second or third aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the second or third aspect of the invention may be ligated to a heterologous sequence so that the combined nucleic acid molecule encodes a fusion protein. Such combined nucleic acid molecules are included within the fourth or fifth aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360(1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (10 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP113, INSP114, INSP115, INSP116 or INSP117 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INSP113, INSP114, INSP115, INSP116 and INSP117 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP113, INSP114, INSP115, INSP116 and INSP117 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP113, INSP114, INSP115, INSP116 and INSP117 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989, 1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52 and SEQ ID NO:54), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaxyotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The vectors pCR4-TOPO-INSP113 (FIG. 18), pCR4-TOPO-INSP113sv (FIG. 19), pDONR (FIG. 20), pEAK12d (FIG. 21), pDEST12.2 (FIG. 22), pENTR-INSP113-6HIS (FIG. 23), pENTR-INSP113sv-6HIS (FIG. 24), pEAK12d-INSP113-6HIS (FIG. 25), pEAK12d-INSP113sv-6HIS (FIG. 26), pDEST12.2-INSP113-6HIS (FIG. 27), pDEST12.2-INSP113sv-6HIS (FIG. 28), pCR4-TOPO-INSP114 (FIG. 31), pCR4-TOPO-INSP114-GR1 (FIG. 35), pCR4-TOPO-INSP114-SV2 (FIG. 36), pDONR 221 (FIG. 38), pEAK12d (FIG. 39), pDEST12.2 (FIG. 40), pENTR_INSP114-6HIS (FIG. 41), pEAK12d_INSP114-6HIS (FIG. 42), pDEST12.2_INSP114-6HIS (FIG. 43), pENTR-INSP114-SV1-6HIS (FIG. 44), pEAK12d_INSP114-SV1-6 mS (FIG. 45), pDEST12.2_INSP114-SV1-6HIS (FIG. 46), pENTR_INSP114-SV2-6HIS (FIG. 47), pEAK12d-INSP114-SV2-6HIS (FIG. 48), pDEST12.2_INSP114-SV2-6HIS (FIG. 49), pDONR 221 (FIG. 51), pEAK12d (FIG. 52), pDEST12.2 (FIG. 53), pENTR_INSP115-6HIS (FIG. 54), pEAK12d_INSP115-6HIS (FIG. 55), pDEST12.2_INSP115-6HIS (FIG. 56), pDONR 221 (FIG. 58), pEAK12d (FIG. 59), pDEST12.2 (FIG. 60), pENTR_INSP116-6HIS (FIG. 61), pEAK12d_INSP116-6HIS (FIG. 62), pDEST12.2_INSP116-6HIS (FIG. 63), pCRII-TOPO-INSP117 (FIG. 66), pDONR 221 (FIG. 67), pEAK12d (FIG. 68), pDEST12.2 (FIG. 69), pENTR_INSP117-6HIS (FIG. 70), pEAK12d_INSP117-6HIS (FIG. 71) and pDEST12.2_INSP117-6HIS (FIG. 72) are preferred examples of suitable vectors for use in accordance with the invention.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)). Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the second or third aspect of the invention or to regulate the activity of a polypeptide of the second or third aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the second or third aspect of the invention, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by measuring the level of a signal generated from the interaction of the compound with the polypeptide.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and

(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises:

determining the inhibition of binding of a ligand such as a receptor to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,

(b) measuring the amount of labelled ligand bound to the whole cell or the cell membrane;

(c) adding a candidate compound to a mixture of labelled ligand and the whole cell or the cell membrane of step (a) and allowing the mixture to attain equilibrium;

(d) measuring the amount of labelled ligand bound to the whole cell or the cell membrane after step (c); and

(e) comparing the difference in the labelled ligand bound in step (b) and (d), such that the compound which causes the reduction in binding in step (d) is considered to be an agonist or antagonist.

The INSP113, INSP114, INSP115, INSP116 and INSP117 polypeptides of the present invention may modulate cellular growth and differentiation. Thus, the biological activity of the INSP113, INSP114, INSP115, INSP116 and INSP117 polypeptides can be examined in systems that allow the study of cellular growth and differentiation such as organ culture assays or in colony assay systems in agarose culture. Stimulation or inhibition of cellular proliferation may be measured by a variety of assays.

For example, for observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture medium turbity or incorporation of labelled thymidine in DNA. Typically, the incorporation of a nucleoside analog into newly synthesised DNA may be employed to measure proliferation (i.e., active cell growth) in a population of cells. For example, bromodeoxyuridine (BrdU) can be employed as a DNA labelling reagent and anti-BrdU mouse monoclonal antibodies can be employed as a detection reagent. This antibody binds only to cells containing DNA which has incorporated bromodeoxyuridine. A number of detection methods may be used in conjunction with this assay including immunofluorescence, immunohistochemical, ELISA, and colorimetric methods. Kits that include bromodeoxyuridine (BrdU) and anti-BrdU mouse monoclonal antibody are commercially available from Boehringer Mannheim (Indianapolis, Ind.).

The effect of the INSP113, INSP114, INSP115, INSP116 and INSP117 polypeptides upon cellular differentiation can be measured by contacting stem cells or embryonic cells with various amounts of the INSP113, INS 114, INSP115, INSP116 and INSP117 polypeptides and observing the effect upon differentiation of the stem cells or embryonic cells. Tissue-specific antibodies and microscopy may be used to identify the resulting cells.

The INSP113, INSP114, INSP115, INSP116 and INSP117 polypeptides may also be found to modulate immune and/or nervous system cell proliferation and differentiation in a dose-dependent manner in the above-described assays. Thus, the “functional equivalents” of the INSP113, INSP114, INSP115, INSP116 and INSP117 polypeptides include polypeptides that exhibit any of the same growth and differentiation regulating activities in the above-described assays in a dose-dependent manner. Although the degree of dose-dependent activity need not be identical to that of the INSP113, INSP114, INSP115, INSP116 and INSP117 polypeptides, preferably the “functional equivalents” will exhibit substantially similar dose-dependence in a given activity assay compared to the INSP113, INSP114, INSP115, INSP116 and INSP117 polypeptides.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [X]is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression-blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-1-0619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;

(b) a polypeptide of the present invention; or

(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in members of the EGF domain containing protein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions. Preferably, the diseases are those in which lymphocyte antigens are implicated. Such kits may also be used for the detection of reproductive disorders including infertility.

Various aspects and embodiments of the present invention will now be described in more detail by way of example, with particular reference to the INSP113, INSP114, INSP115, INSP116 and INSP117 polypeptides.

It will be appreciated that modification of detail may be made without departing from the scope of the invention.

### BRIEF DESCRIPTION OF THE FIGURES

FIG. 1: ClustalW alignment of all 15 polypeptide sequences in the SECFAM1 family, including the INSP113 polypeptide (SEQ ID NO:2), the INSP114 polypeptide (SEQ ID NO:6), the INSP115 polypeptide (SEQ ID NO:10), the INSP116 polypeptide (SEQ ID NO: 14), the INSP117 polypeptide (SEQ ID NO:26) and their orthologues from mouse (AK049880.1, chr6_prediction—SEQ ID NO:31, chr10_prediction—SEQ ID NO:32, AK078681, BAC41130.1 and AAH15306.1), rat (chr2_prediction—SEQ ID NO:33, chr4_prediction —SEQ ID NO:34), macaque (BAB60784.1) and pufferfish (scaffold_{—}3581_prediction —SEQ ID NO:35).

FIG. 2: SignalP signal peptide prediction for INSP113 (SEQ ID NO:2). Signal peptide probability: 0.994. Maximum cleavage site probability: 0.400 between positions 25 and 26.

FIG. 3: SignalP signal peptide prediction for INSP114 (SEQ ID NO:6). Signal peptide probability: 0.969. Maximum cleavage site probability: 0.713 between positions 30 and 31.

FIG. 4: SignalP signal peptide prediction for INSP115 (SEQ ID NO:10). Signal peptide probability: 0.931. Maximum cleavage site probability: 0.274 between positions 42 and 43.

FIG. 5: SignalP signal peptide prediction for INSP116 (SEQ ID NO:14). Signal peptide probability: 0.908. Maximum cleavage site probability: 0.374 between positions 34 and 35.

FIG. 6: SignalP signal peptide prediction for INSP117 (SEQ ID NO:26). Signal peptide probability: 0.989. Maximum cleavage site probability: 0.748 between positions 30 and 31.

FIG. 7: Genome Threader output after querying with CAD28501.1 (INSP113 polypeptide; SEQ ID NO:2).

FIG. 8: Genome Threader output after querying with CAD38865.1 (INS 114 polypeptide; SEQ ID NO:6).

FIG. 9: Genome Threader output after querying with XP_{—}08726.1 (INSP116 polypeptide; SEQ ID NO:14).

FIG. 10: Genome Threader output after querying with INSP117 (SEQ ID NO:26).

FIG. 11: Top fifteen results from BLASTP against the family database using AAY53016 (INSP115; SEQ ID NO:10) and the alignment generated by BLASTP between AAY53016 (INSP115; SEQ ID NO: 10) and INSP114 (SEQ ID NO: 6).

FIG. 12: Genome Threader alignment between INSP113 (SEQ ID NO:2) and the top hit structure 1WHE. The four conserved, disulphide bond forming cysteines can be seen at positions 96, 101, 107 and 118 of the query sequence (lower row). Cysteines 98 and 109 represent a conserved disulphide pair, as defined by the structure 1WHE.

FIG. 13: Profile of the SECFAM1 family of proteins, built around INSP117 (SEQ ID NO:26).

FIG. 14: Family consensus sequence in PROSITE format taken from INSP117 position 44 to 129 amino acids (52-138 amino acids of the alignment (FIG. 1)). Key: -=a spacer between each alignment position; G=100% conserved G residue; [VI]=either a V or an I at that alignment position; P(0,1)=a P residue found once or not at all at this alignment position.

FIG. 15: Nucleotide sequence of INSP113 prediction with translation.

FIG. 16: Nucleotide sequence with translation of INSP113 PCR product cloned using primers INSP113-CP1 and INSP113-CP2.

FIG. 17: Nucleotide sequence with translation of INSP113sv PCR product cloned using primers INSP113-CP1 and INSP113-CP2.

FIG. 18: Map of pCR4-TOPO-INSP113.

FIG. 19: Map of pCR4-TOPO-INSP113sv.

FIG. 20: Map of pDONR 221.

FIG. 21: Map of expression vector pEAK12d.

FIG. 22: Map of Expression vector pDEST12.2.

FIG. 23: Map of pENTR-INSP113-6HIS.

FIG. 24: Map of pENTR-INSP113sv-6HIS.

FIG. 25: Map of pEAK12d-INSP113-6HIS.

FIG. 26: Map of pEAK12d-INSP113sv-6HIS.

FIG. 27: Map of pDEST12.2-INSP113-6HIS.

FIG. 28: Map of pDEST12.2-INSP113sv-6HIS.

FIG. 29: INSP114 sequence with translation of the coding sequence showing the positions of PCR primers.

FIG. 30: Nucleotide sequence with translation of INSP114 PCR product cloned using primers INSP114-CP1 and INSP114-CP2.

FIG. 31: Map of pCR4-TOPO-INSP114.

FIG. 32: Nucleotide alignment of INSP114 cds and corresponding region of IMAGE clone 1616371 (including stop codon).

FIG. 33: Amino acid alignment of INSP114 cds and corresponding region of IMAGE clone 1616371.

FIG. 34: Nucleotide sequence with translation of INSP114-GR1 PCR product cloned showing positions of the primer pair used in the RACE reaction.

FIG. 35: Map of pCR4-TOPO-INSP114-GR1.

FIG. 36: Nucleotide sequence with translation of INSP114-SV2 PCR product cloned using primers INSP114-CP3 and INSP114-CP4.

FIG. 37: Map of pCR4-TOPO-INSP114-SV2.

FIG. 38: Map of pDONR 221.

FIG. 39: Map of Expression vector pEAK12d.

FIG. 40: Map of Expression vector pDEST12.2.

FIG. 41: Map of pENTR_INSP114-6HIS.

FIG. 42: Map of pEAK12d_INSP114-6HIS.

FIG. 43: Map of pDEST12.2_INSP114-6HIS.

FIG. 44: Map of pENTR_INSP114-SV1-6HIS.

FIG. 45: Map of pEAK12d_INSP114-SV1-6HIS.

FIG. 46: Map of pDEST12.2_INSP114-SV1-6HIS.

FIG. 47: Map of pENTR_INSP114-SV2-6HIS.

FIG. 48: Map of pEAK12d_INSP114-SV2-6HIS.

FIG. 49: Map of pDEST12.2_INSP114-SV2-6HIS.

FIG. 50: INSP115 sequence with translation of cds.

FIG. 51: Map of pDONR 221.

FIG. 52: Map of Expression vector pEAK12d.

FIG. 53: Map of Expression vector pDEST12.2.

FIG. 54: Map of pENTR_INSP115-6HIS.

FIG. 55: Map of pEAK12d_INSP115-6HIS.

FIG. 56: Map of pDEST12.2_INSP115-6HIS.

FIG. 57: INSP116 sequence with translation of cds.

FIG. 58: Map of pDONR 221.

FIG. 59: Map of Expression vector pEAK12d.

FIG. 60: Map of Expression vector pDEST12.2.

FIG. 61: Map of pENTR_INSP116-6HIS.

FIG. 62: Map of pEAK12d_INSP116-6HIS.

FIG. 63: Map of pDEST12.2_INSP116-6HIS.

FIG. 64: Nucleotide sequence of INSP117 prediction with translation.

FIG. 65: Nucleotide sequence with translation of INSP117 PCR product cloned using primers INSP117-CP1 and INSP117-CP2.

FIG. 66: Map of pCRII-TOPO-INSP117.

FIG. 67: Map of pDONR 221.

FIG. 68: Map of Expression vector pEAK12d.

FIG. 69: Map of Expression vector pDEST12.2.

FIG. 70: Map of pENTR-INSP117-6HIS.

FIG. 71: Map of pEAK12d-INSP117-6HIS.

FIG. 72: Map of pDEST12.2_INSP117-6HIS.

### EXAMPLES

### Example 1

### Selecting and Aligning the SECFAM1 Family Members

INSP113, INSP114, INSP115, INSP116 and INSP117 have no publicly available annotation, contain a strong secretory protein signature in the form of a signal peptide, and can be clustered with similar proteins, supported by orthologues from other animal species.

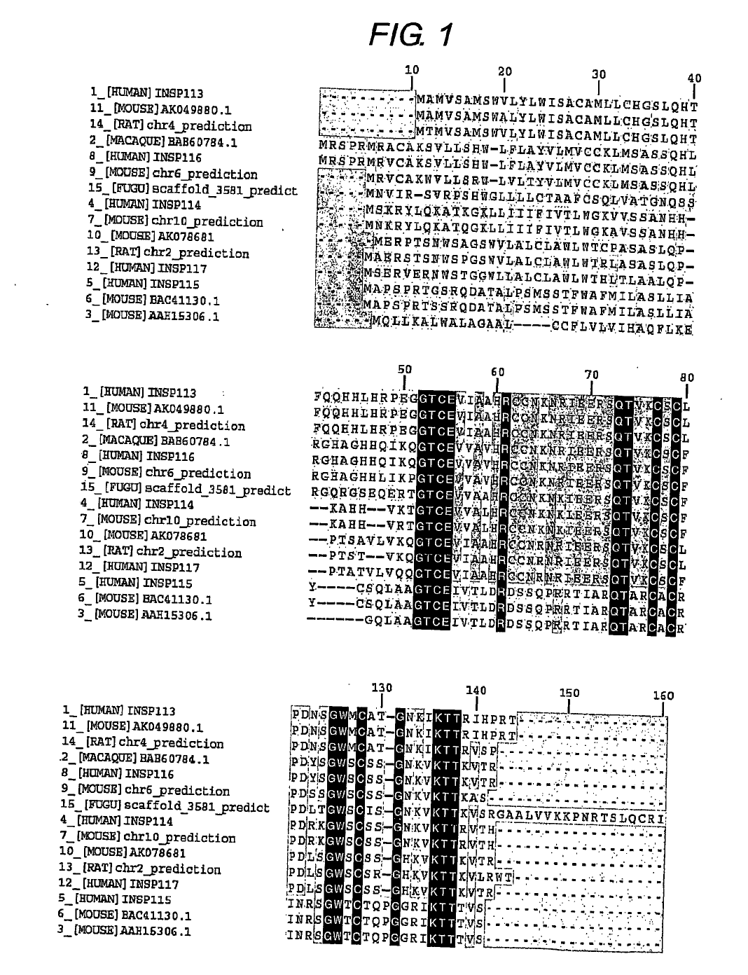

Further examination permitted the construction of an uncharacterised family of proteins consisting of 5 human genes (INSP113-117) and, with mammalian and fish orthologues, 15 sequences in total. These sequences were aligned using the ClustalW tool (Thompson, J. D., Higgins, D. G., Gibson T. J. Nucleic Acids Res 1994 Nov. 11; 22(22):4673-80) (FIG. 1). From this alignment, the similarities and differences in the sequences can be clearly seen.

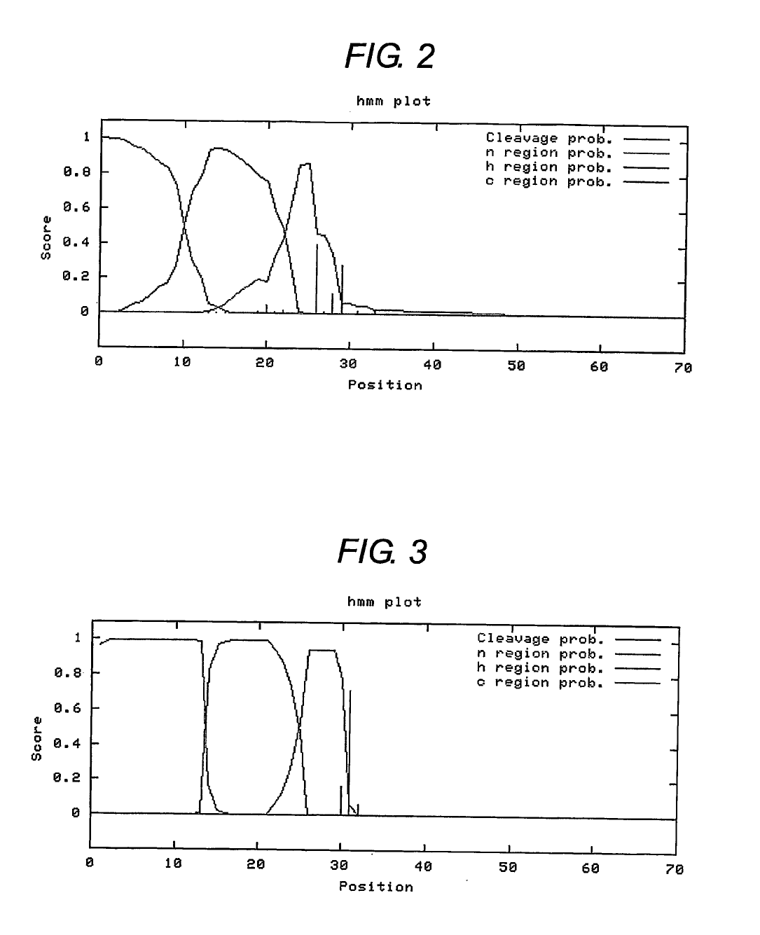

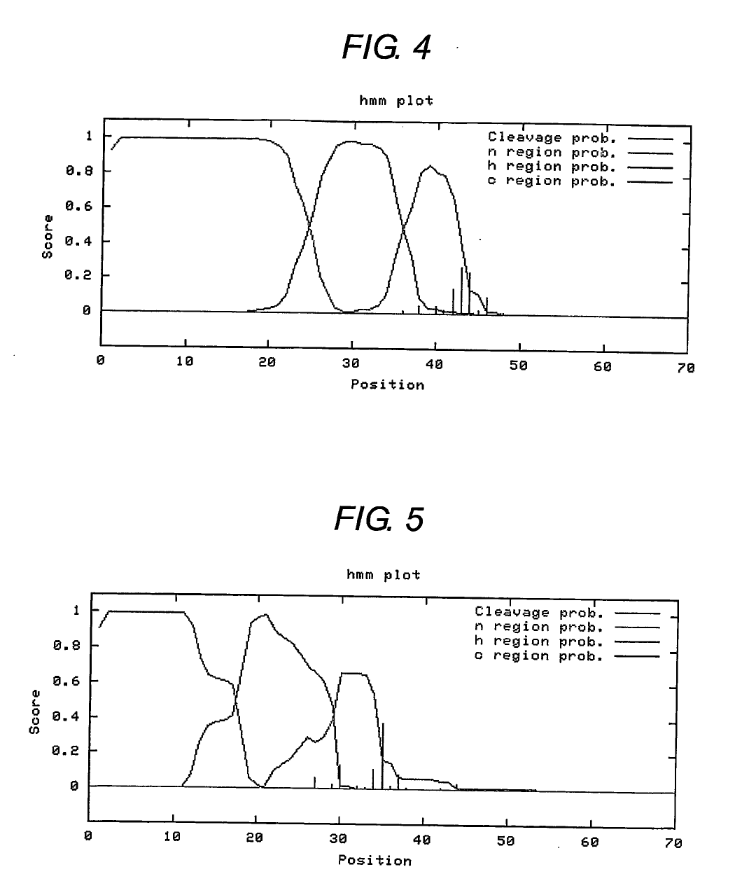

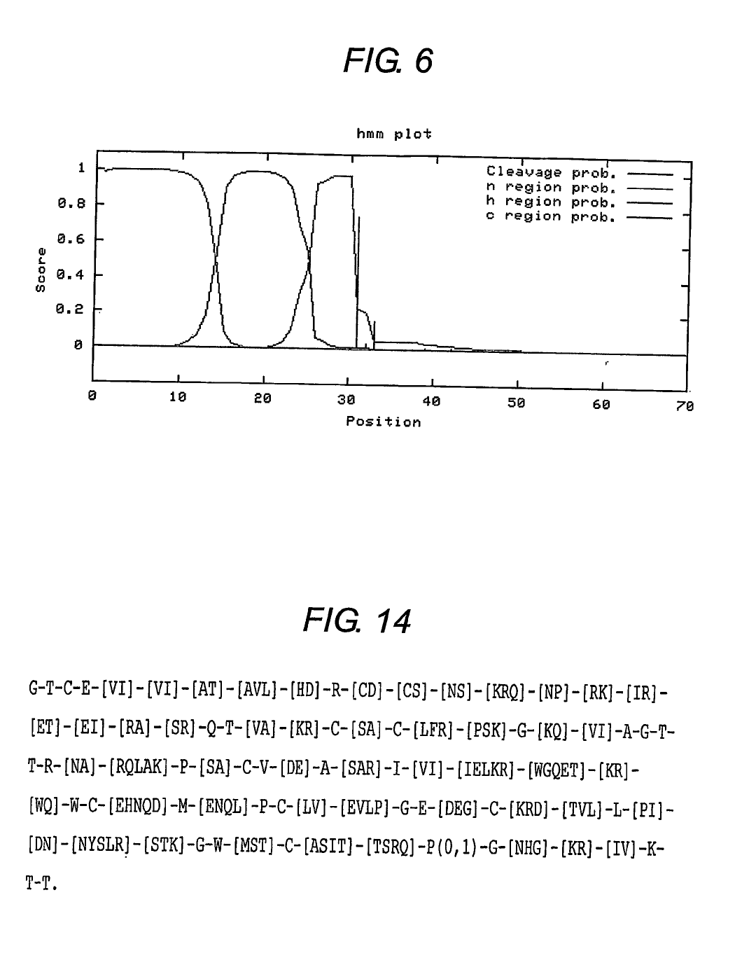

The SignalP program (http://www.cbs.dtu.dk/services/SignalP/), was used to identify the potential signal peptide regions and cleavage sites for the INSP113-117 polypeptides. The SignalP results for INSP113 (SEQ ID NO:2) indicate that the cleavage site is most likely to be between positions 25 and 26 of SEQ ID NO: 2 (FIG. 2). The SignalP results for INSP114 (SEQ ID NO:6) indicate that the cleavage site is most likely to be between positions 30 and 31 of SEQ ID NO:6 (FIG. 3). The SignalP results for INSP115 (SEQ ID NO:10) indicate that the cleavage site is most likely to be between positions 42 and 43 of SEQ ID NO:10 (FIG. 4). The SignalP results for INSP116 indicate that the cleavage site is most likely to be between positions 34 and 35 of SEQ ID NO:14 (FIG. 5). The SignalP results for INSP117 (SEQ ID NO:26) indicate that the cleavage site is most likely to be between positions 30 and 31 of SEQ ID NO:26).

The signal peptide region was found to be more variable compared to the rest of the polypeptide, which displays a high degree of similarity. Overall, within the human sequence, identity was observed to drop to 49% while preserving a strong profile of conserved residues (FIG. 1). This cluster of related sequences is referred to as the “SECFAM1 family”.

### Example 2

### INSP115

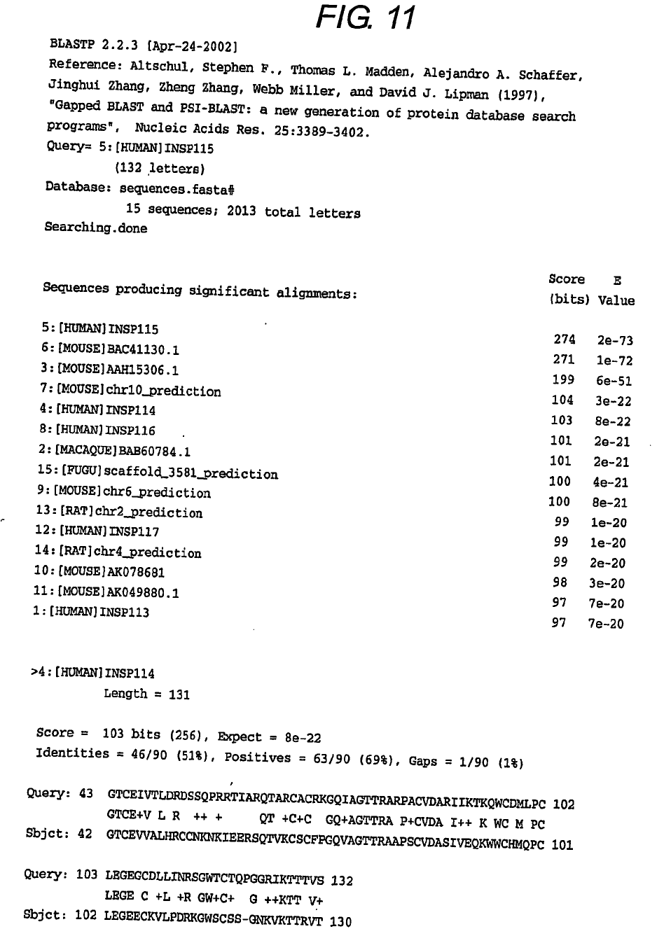

A query using a proprietary bioinformatics program termed “Genome Threader” with AAY53016 (INSP115) did not yield any results. However, a Homo sapiens paralogue of AAY53016 (INSP115) has been identified, and will be referred to herein as CAD38865.1 (INSP114). Residues 42-130 of CAD38865.1 (INSP114) are identified in a BLASTP query of INSP115 against the SECFAM1 family (FIG. 11) as sharing 51% sequence identity with residues 43-132 of AAY53016 (INSP115). Residues 42-130 of CAD38865.1 (INSP114) contain the region (residues 67-121) predicted to adopt the structure of an EGF-like domain. On the basis of the high sequence identity that AAY53016 (INSP115) shares with the region of CAD38865.1 (INSP114) that is predicted to adopt the structure of an EGF-like domain, we predict that AAY53016 (INSP115) also adopts the structure of an EGF-like domain. Chothia and Lesk, 1986 (EMBO Journal vol. 5 pp823) first showed that for proteins with more than 50% sequence identity, 85% of the component residues would adopt the same conformation. Other groups (Sander, C. and Schneider, R. (1991) Proteins vol. 9 pp56; Hubbard, T. J. P. and Blundell, T. L. (1987) Protein Engineering vol. 1 pp 159; Flores, T. P., Orengo, C. A., Moss, D. M. and Thornton, J. M. (1993) Protein Science vol. 2 pp 1811; and Hilbert, M., Bohm, G. and Jaenicke, R. (1993) Proteins vol. 17 pp 138) subsequently extended these studies and have showed that the fold remains the same even if sequence identity falls as low as 30%.

### Example 3

### Supporting Evidence for the INSP117 Gene Model

The INSP117 gene model prediction was verified by an EST (BM94096). This EST spanned three splicing exons that covered residues 7-120 of INSP117 (134 in total) at 100% identity. This EST mapped to chr1:112149567-112151424 (+ strand) of Build31 of the human genome. In addition, the translated gene model aligns very strongly to the other SECFAM1 family members (see FIG. 1).

### Example 4

### Evidence for the Presence of an EGF Domain within the SECFAM1 Proteins

Annotation of the SECFAM1 family of proteins, beyond characterising them as secreted peptides, could not be placed using sequence- or domain profile-based approaches, such as BLAST, CDD, InterPro scan or Inpharmatica Domain Professor. Given that structural folds are maintained between sequences that are quite distantly related at the sequence level, the proprietary Inpharmatica Genome Threader program was used to look for structural relationships to known structures.

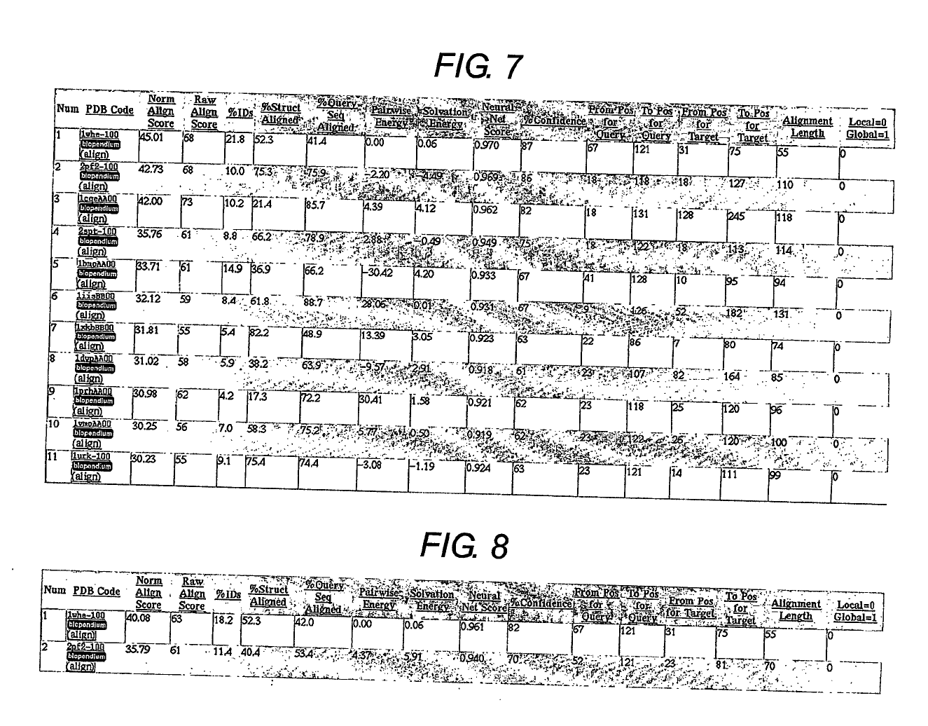

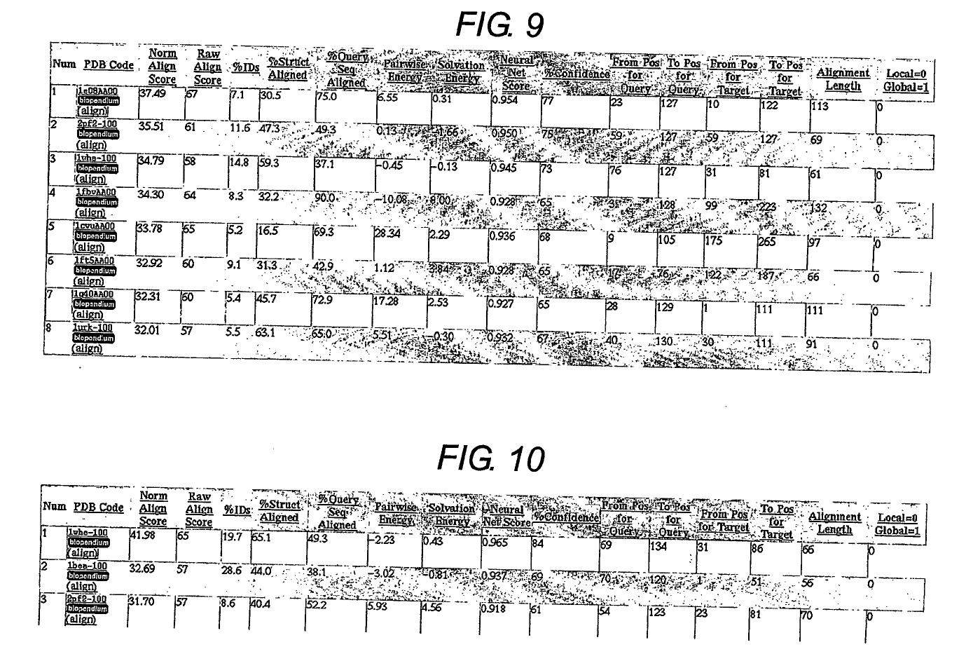

The results from Genome Threader queries with each of the 15 family sequences revealed a trend in the structures being returned. The structures 1WHE, 2 PF2, 2SPT and 1URK were all hit on multiple occasions between the SECFAM1 sequence queries. In addition, they consistently folded over the same regions of the structures with the same regions of the query sequences (as dictated by the family alignment). The percentage confidence of any one fold prediction in the data set was placed in the 70 s or 80 s, and these entries usually constituted the most significant results (FIGS. 7-10 for the human sequence results (INSP115 did not return any results)). Taken alone, the significance of these hits may have been too low to draw any definitive conclusions. However, the fact that these same structures are hit consistently throughout the range of sequences, some of which deviate quite significantly in sequence identity (e.g. 68% ID between INSP113 and INSP114), provides a much stronger relationship. In all cases, the region of the structures over which the family sequences were folded related to EGF-like domains.

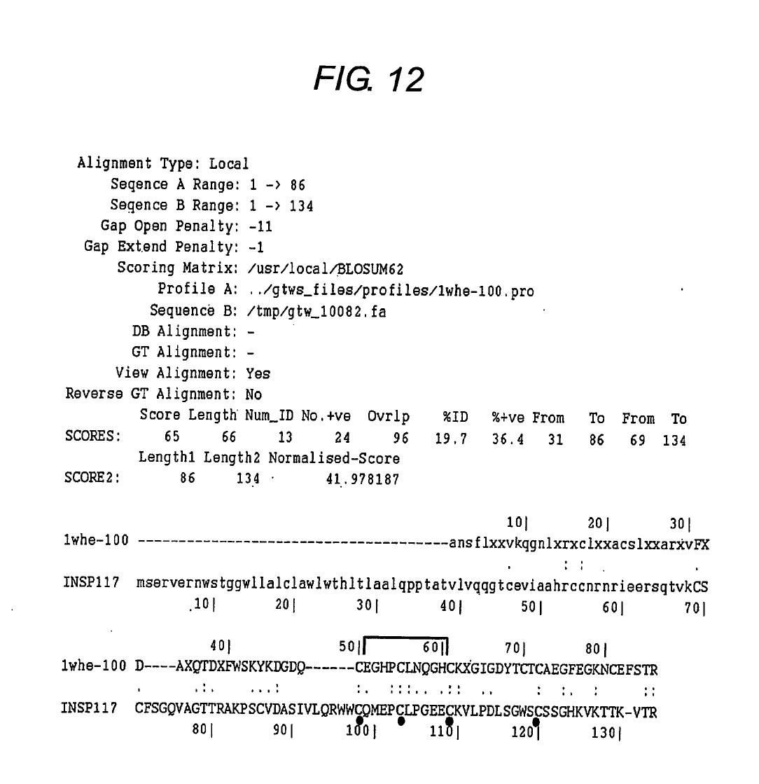

From the resulting alignments, it was seen that there were four consistently conserved cysteines (for example, see FIG. 12). These cysteine residues were equivalent to positions 106, 111, 117 and 128 in the SECFAM1 family alignment (see FIG. 1). Furthermore, these conserved residues are involved in the formation of disulphide bridges within the known structures.

FIG. 8 shows the profile of residue frequency for the SECFAM1 family. This represents the unique signature of the family. This profile was constructed using the novel sequence INSP117 as a template from which the likelihood of finding any one amino acid residue at each of the positions occupied by the individual residues of INSP117 in the alignment was calculated. These calculations were then displayed as a table of probability scores for each of the 20 amino acid residues at each position in the alignment, delimited by the residues INSP117.

### Example 5

### Tissue Distribution

Expressed sequence tags that were seen to splice over more than two exons supported all five sequences. The information for each EST match may be seen in Table 1. This information indicated an up-regulation of INSP113-117 gene expression in the tissues of the central nervous system.

TABLE 1
Expressed Sequence Tag (EST) matches to the five human genes showing
accession numbers and the tissue in which they were seen to be expressed.

| Sequence | EST Accession No. | Tissue |
|---|---|---|
| INSP113 | H23443.1 | Whole brain (infant) |
| | AW955725.1 | Colon cancer |
| INSP114 | AA984082 | Brain: frontal lobes |
| | AA984097 | Brain: frontal lobes |
| | H08484 | Whole brain (infant) |
| INSP115 | BI596893 | Brain: hypothalamus |
| | BI915401 | Brain |
| | BI599742 | Brain: hypothalamus |
| INSP116 | BI599941 | Brain: hypothalamus |
| INSP117 | BM694096 | Eye: retina |
| | BQ638101 | Eye: retina |

### Example 6

### The SECFAM1 Family Profile

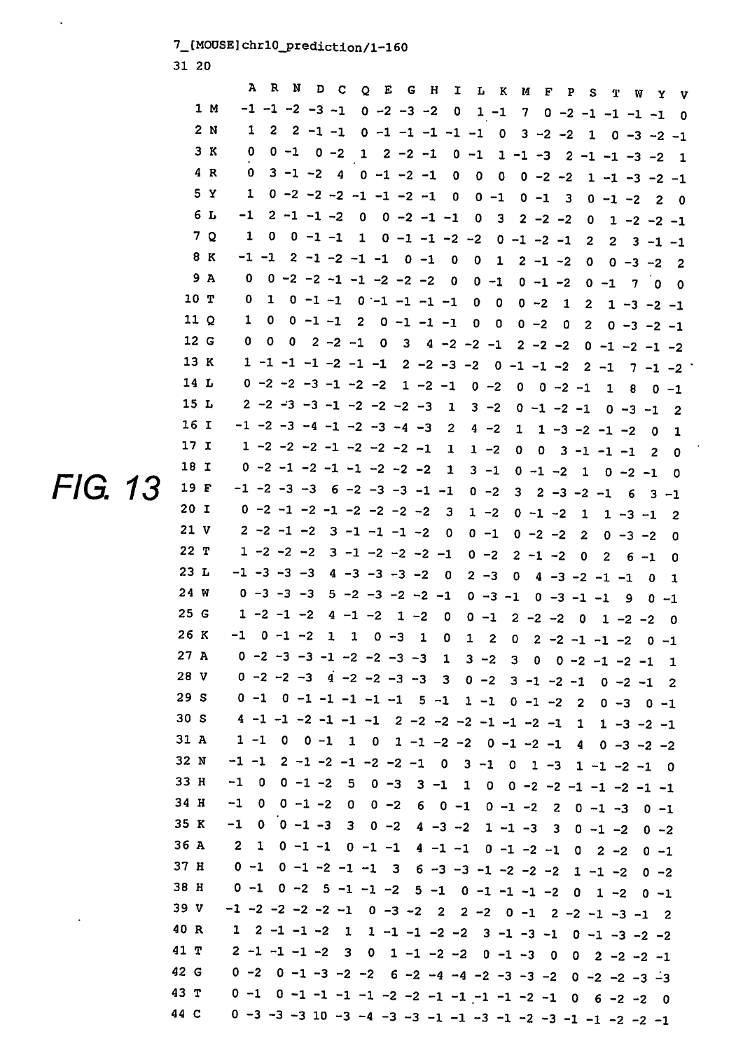

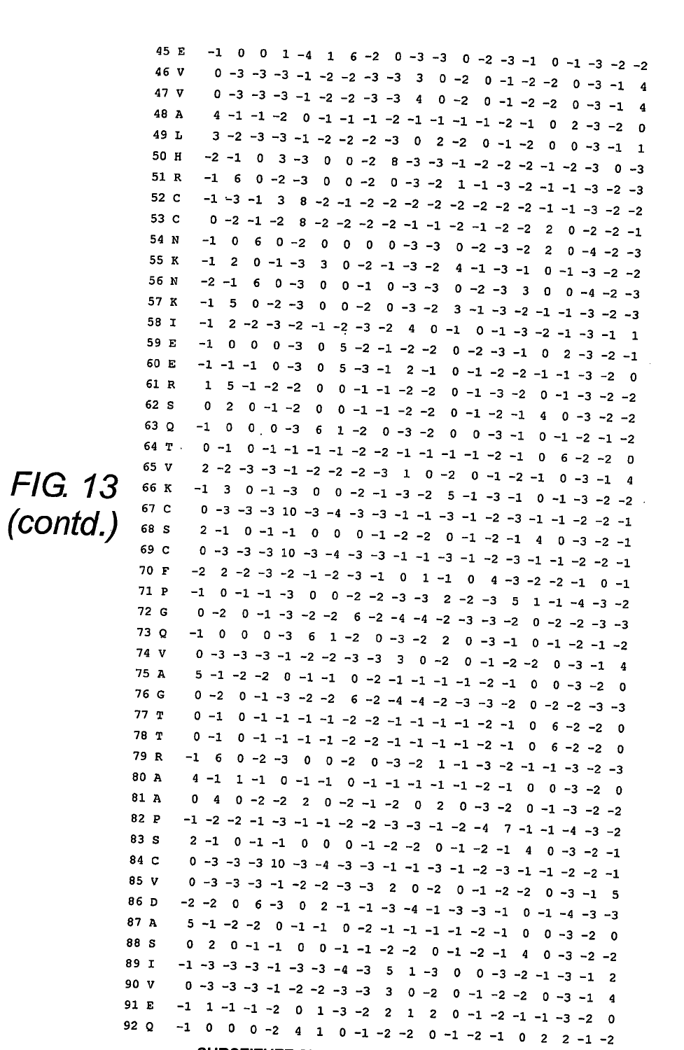

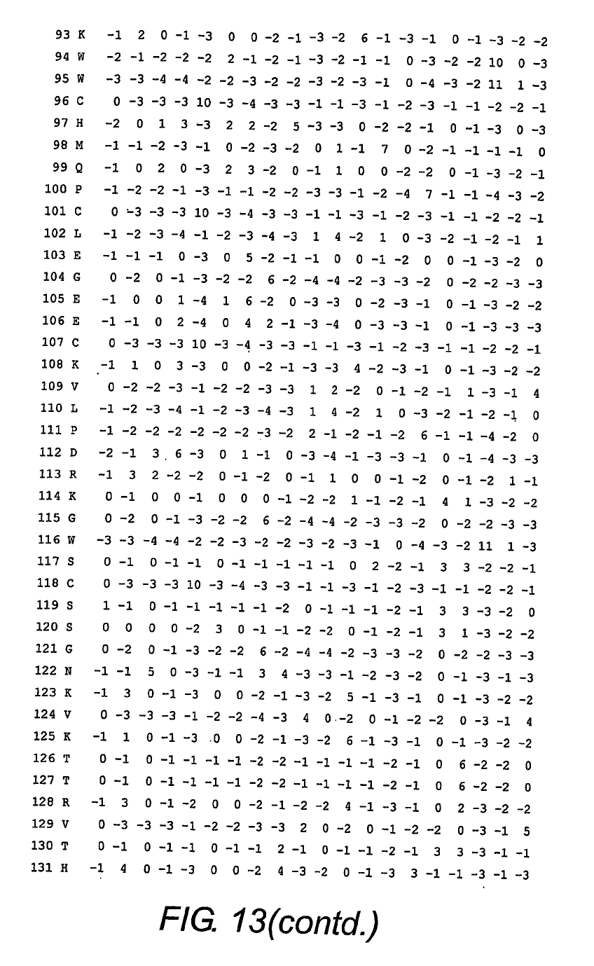

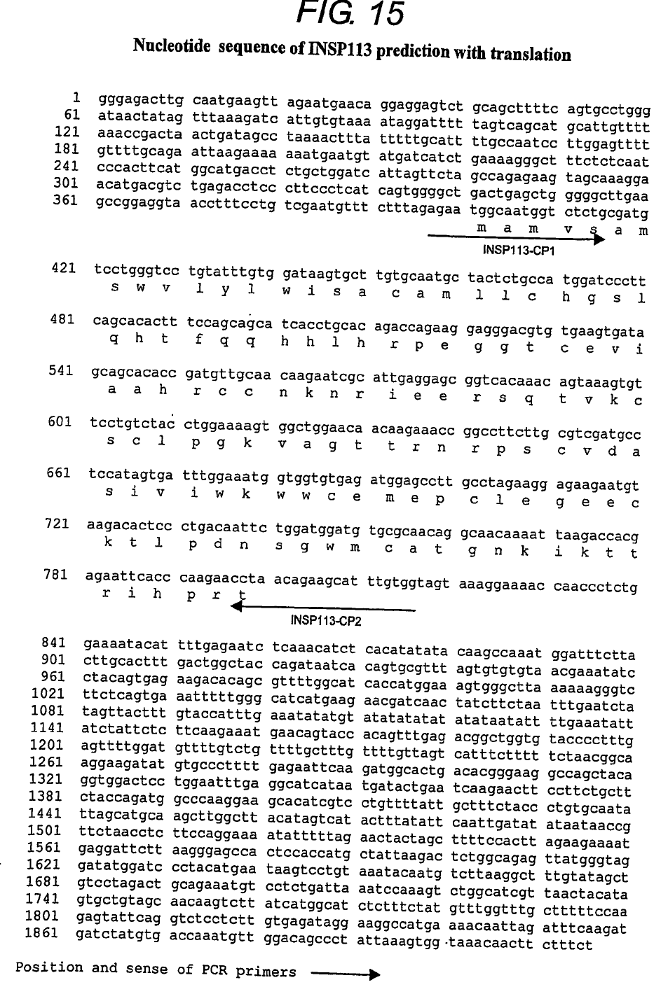

FIG. 13 shows the position-specific score matrix, or profile, for the SECFAM1 family. This represents the unique signature of the family. The profile was generated by first creating a multiple alignment of the sequences. A template sequence was chosen, in this case INSP117, around which to construct a profile. The frequency of each of the possible 20 amino acid types was assessed for each column of the family multiple sequence alignment that was occupied by a residue of the template sequence. The score of each amino acid residue type at each position in the family alignment was calculated based on the frequency scores and the likelihood of seeing a substitution of the dominant residue with this residue type, based on the BLOSUM62 position-independent background matrix (Henikoff & Henikoff, 1992. Proc. Natl. Acad. Sci. USA, 89:10915-9). This matrix is based on a large dataset of family alignment blocks (BLOcks SUbstitution Matrix) where amino acid substitution frequencies were assessed based on alignments clustered at 62% identity or greater. In this case, these factors were pooled to give a logarithm-based score for each amino acid type at each position in the SECFAM1 alignment. The highest positive scores represent those amino acids that are most likely to be found at that position. This profile can be used to find an alignment score of a query sequence. At each position, the corresponding value for that amino acid is extracted and the sum of all such scores for each amino acid of the query sequence constitutes the alignment score for that sequence. If this is above a certain threshold value, the query sequence may be significantly related to the family. The profile, then, forms a sensitive statistical standard for the family.

### Example 7

### Cloning of INSP113

7.1 Preparation of Human cDNA Templates

First strand cDNA was prepared from a variety of normal human tissue total RNA samples (Clontech, Ambion, and in-house samples) using Superscript II RNase H⁻ Reverse Transcriptase (Invitrogen) according to the manufacturer's protocol. Oligo (dT)₁₅ primer (1 μl at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents were collected by brief centrifugation and 4 μl of 5× First-Strand Buffer, 2 μl 0.1 M DTT, and 1 μl RnaseOUT Recombinant Ribonuclease Inhibitor (40 units/μl, Invitrogen) were added. The contents of the tube were mixed gently and incubated at 42° C. for 2 min; then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl. Human cDNA samples used as templates for the amplification of INSP113 were derived from colon, brain and kidney.

7.2 cDNA Libraries

Human cDNA libraries (in bacteriophage lambda (λ) vectors) were purchased from Clontech or made in-house in λ GT10 vectors. Bacteriophage λ DNA was prepared from small scale cultures of infected E. coli host strain using the Wizard Lambda Preps DNA purification system according to the manufacturer's instructions (Promega, Corporation, Madison Wis.). Human cDNA library samples used as templates for the amplification of INSP113 were derived from adult brain cortex, fetal brain and fetal kidney.

7.3 Gene Specific Cloning Primers for PCR

A pair of PCR primers having a length of between 18 and 25 bases were designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP113) with little or no none specific priming.

7.4 PCR Amplification of INSP113 from a Variety of Human cDNA Templates and Phage Library cDNA

Gene-specific cloning primers (INSP113-CP1 and INSP113-CP2, FIG. 15-17 and Table 2) were designed to amplify a cDNA fragment of 420 bp covering the entire 399 bp coding sequence of the INSP113 prediction. Interrogation of public EST sequence databases with the INSP113 prediction suggested that the sequence might be expressed in adult and fetal brain, adult and fetal kidney, and colon cDNA templates. The gene-specific cloning primers INSP113-CP1 and INSP113-CP2 were therefore used with human cDNA samples listed in Section 7.1 and the phage library cDNA samples listed in Section 7.2 as the PCR templates. The PCR was performed in a final volume of 50 μl containing 1× AmpliTaq™ buffer, 200 μM dNTPs, 50 pmoles of each cloning primer, 2.5 units of AmpliTaq™ (Perkin Elmer) and 100 ng of human cDNA template using an MJ Research DNA Engine, programmed as follows: 94° C., 2 min; 40 cycles of 94° C., 1 min, 51° C., 1 min, and 72° C., 1 min; followed by 1 cycle at 72 (C for 7 min and a holding cycle at 4° C. All 50 μl of each amplification product was visualized on a 0.8% agarose gel in 1×TAE buffer (Invitrogen). A single PCR product was seen migrating at approximately the predicted molecular mass in the sample corresponding to the adult brain first strand cDNA template. A second PCR product was seen migrating at approximately 500 bp in the sample corresponding to a fetal brain cDNA library template. These two PCR products were purified from the gel using the Wizard PCR Preps DNA Purification System (Promega). Each PCR product was eluted in 50 μl of sterile water and subcloned directly.

TABLE 2
INSP113 cloning and sequencing primers

| | |
|---|---|
| Primer | Sequence (5′-3′) |

| | |
|---|---|
| INSP113-CP1 | AGA ATG GCA ATG GTC TCT G |
| INSP113-CP2 | CCA CAA ATG CTT CTG TTA GG |
| INSP113-EX1 | AAG CAG GCT TCG CCA CCA TGG CAA TGG |
| | TCT CTG CGA T |
| INSP113-EX2 | GTG ATG GTG ATG GTG GGT TCT TGG GTG |
| | AAT TCT CG |
| INSP113sv-EX1 | AAG CAG GCT TCG CCA CCA TGG CAA TGG |
| | TCT CTG CGA T |
| INSP113sv-EX2 | GTG ATG GTG ATG GTG AGG CCT TGG ATG |
| | ATC TGA AG |
| GCP Forward | G GGG ACA AGT TTG TAC AAA AAA GCA |
| | GGC TTC GCC ACC |
| GCP Reverse | GGG GAC CAC TTT GTA CAA GAA AGC TGG |
| | GTT TCA ATG GTG ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 | TAA TAC GAC TCA CTA TAG G |
| T3 | ATT AAC CCT CAC TAA AGG |
| SP6 | ATT TAG GTG ACA CTA TAG |

Underlined sequence = Kozak sequence
Bold = Stop codon
Italic sequence = His tag

7.5 Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP 10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture (300 μl was then plated on L-broth (LB) plates containing ampicillin (100 μg/ml) and incubated overnight at 37° C.

7.6 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR products were analyzed on 1% agarose gels in 1×TAE buffer. Colonies which gave the expected PCR product size (420 bp or approximately 500 bp cDNA+105 bp due to the multiple cloning site or MCS) were grown up overnight at 37° C. in 5 ml L-Broth (LB) containing ampicillin (100 μg/ml), with shaking at 220 rpm.

7.7 Plasmid DNA Preparation and Sequencing

Miniprep plasmid DNA was prepared from the 5 ml cultures using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or Spectramax 190 photometer (Molecular Devices). Plasmid DNA (200-500 ng) was subjected to DNA sequencing with the T7 primer using the BigDye Terminator system (Applied Biosystems cat. no. 4390246) according to the manufacturer's instructions. The primer sequence is shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

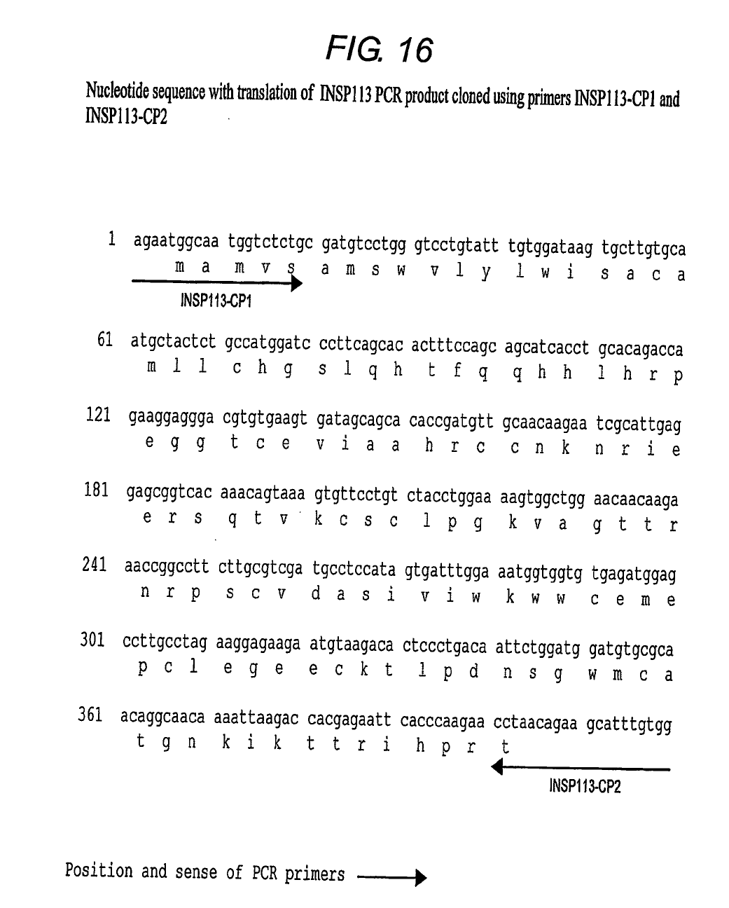

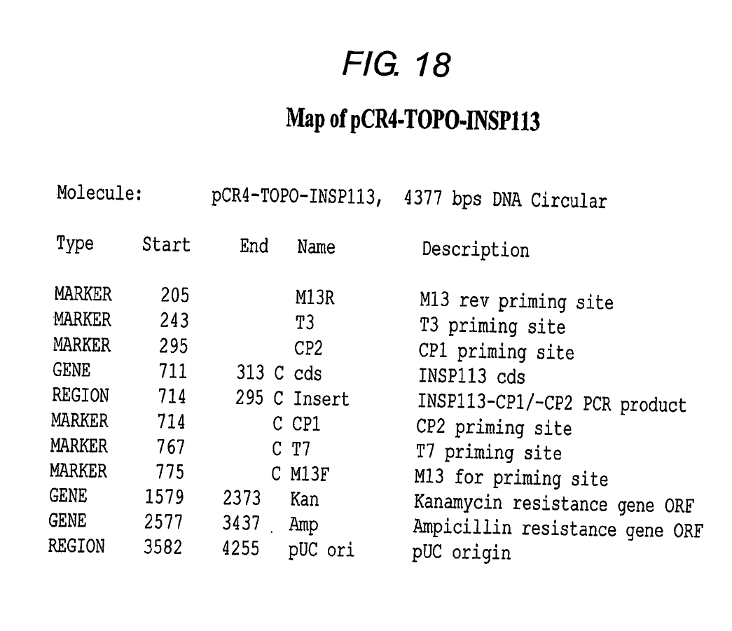

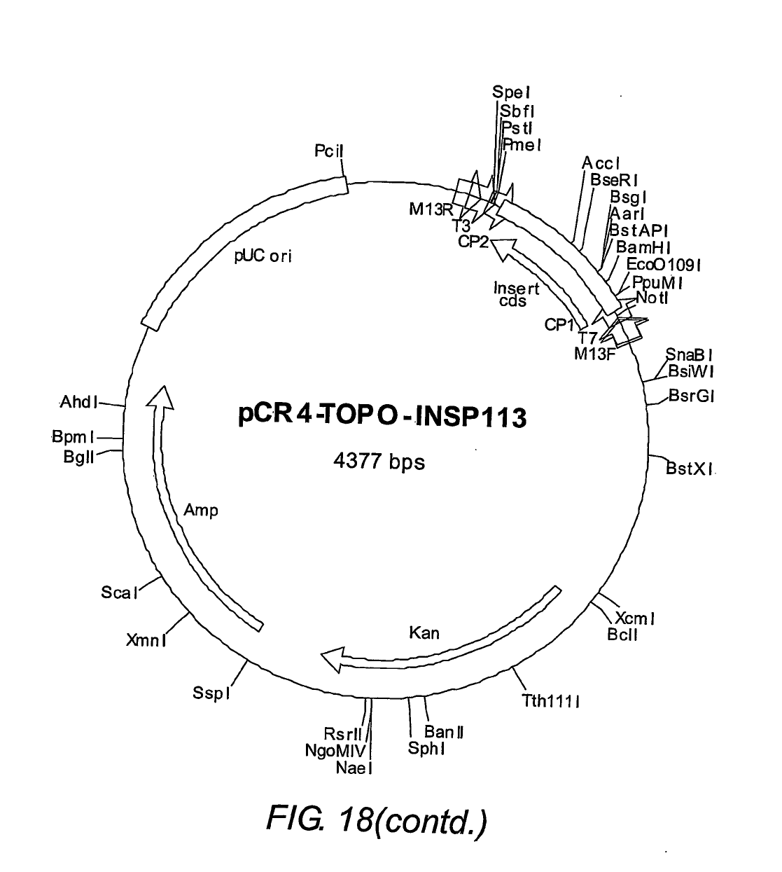

Sequence analysis identified a clone amplified from adult brain cDNA containing a 100% match to the predicted INSP113 sequence. The sequence of the cloned cDNA fragment is shown in FIG. 16. The plasmid map of the cloned PCR product (pCR4-TOPO-INSP113) (plasmid ID13887) is shown in FIG. 18.

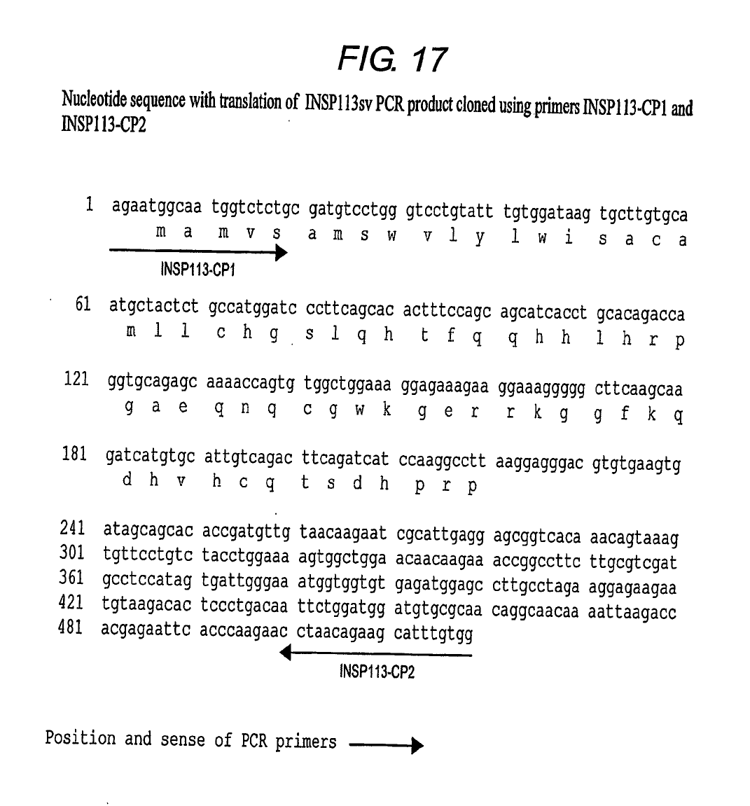

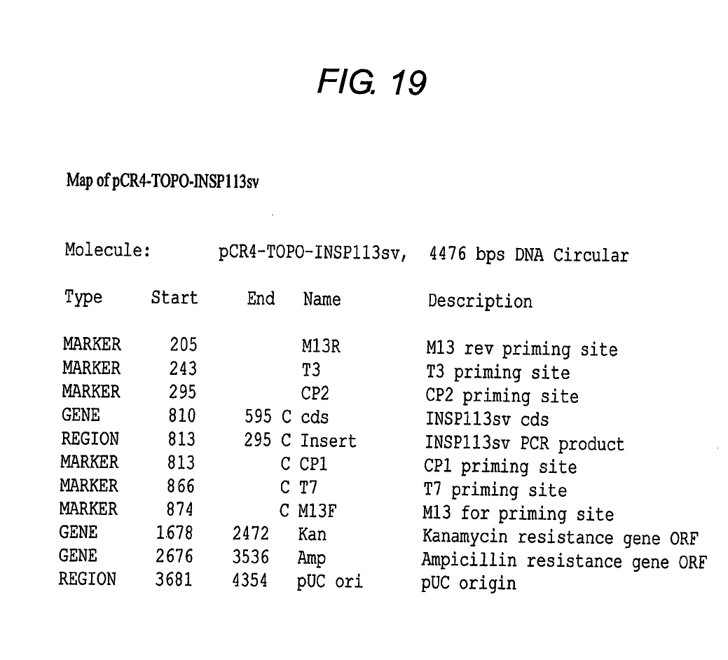

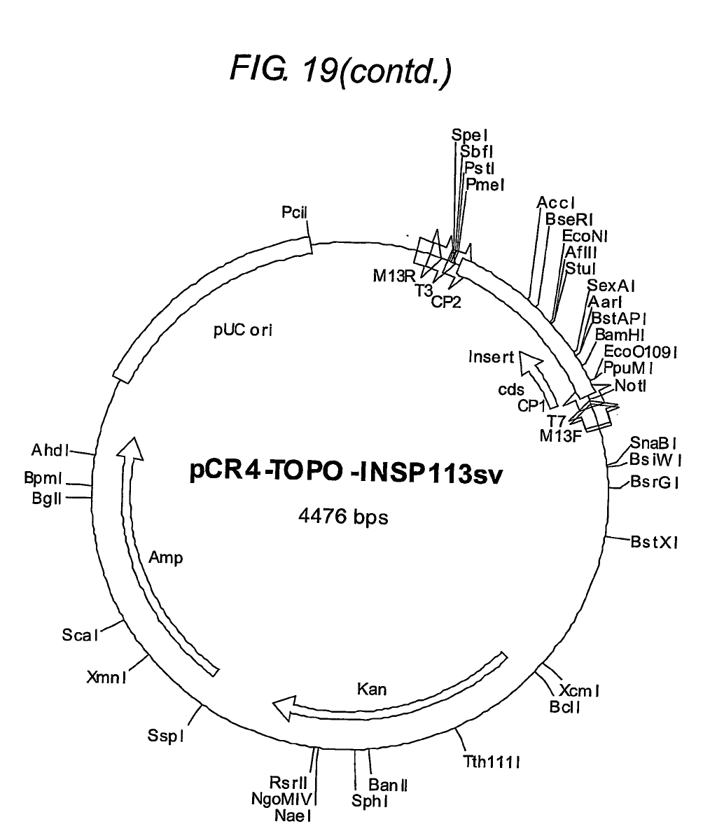

A second clone was identifed which contained a splice variant of the INSP113 prediction. This product bad been amplified from a fetal brain cDNA library. It contained an addition exon between predicted exons 1 and 2, and this additional exon included a stop codon, leading to an ORF of 216 bp. The sequence of the cloned cDNA fragment is shown in FIG. 17. The plasmid map of the cloned PCR product (pCR4-TOPO-INSP113sv) (plasmid ID13888) is shown in FIG. 19.

### Example 8

### Construction of Plasmids for the Expression of INSP113 and INSP113sv in REK293/EBNA Cells

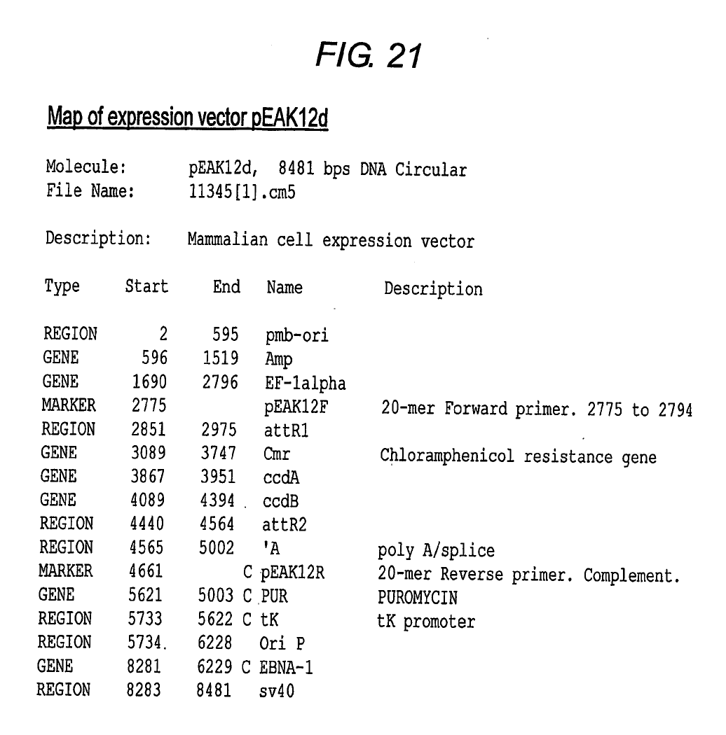

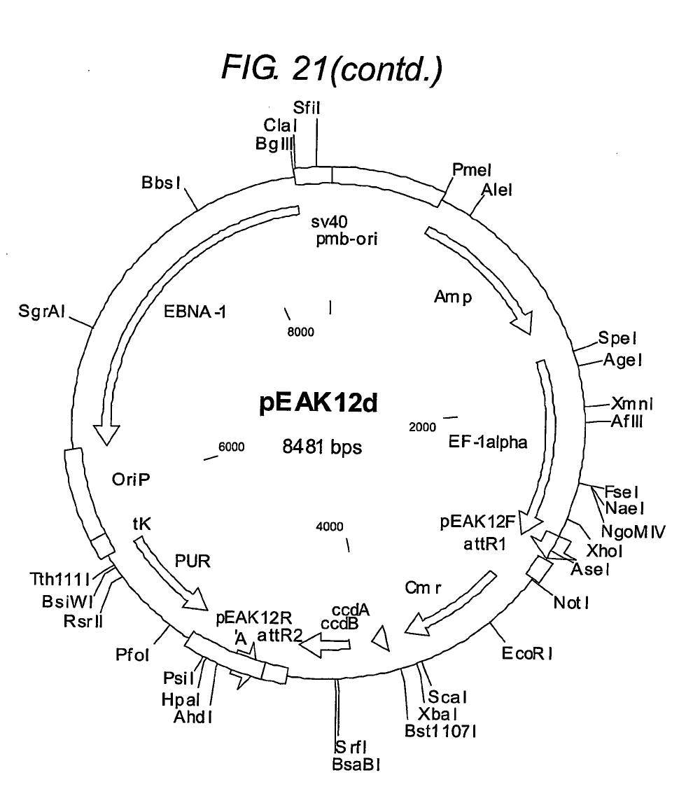

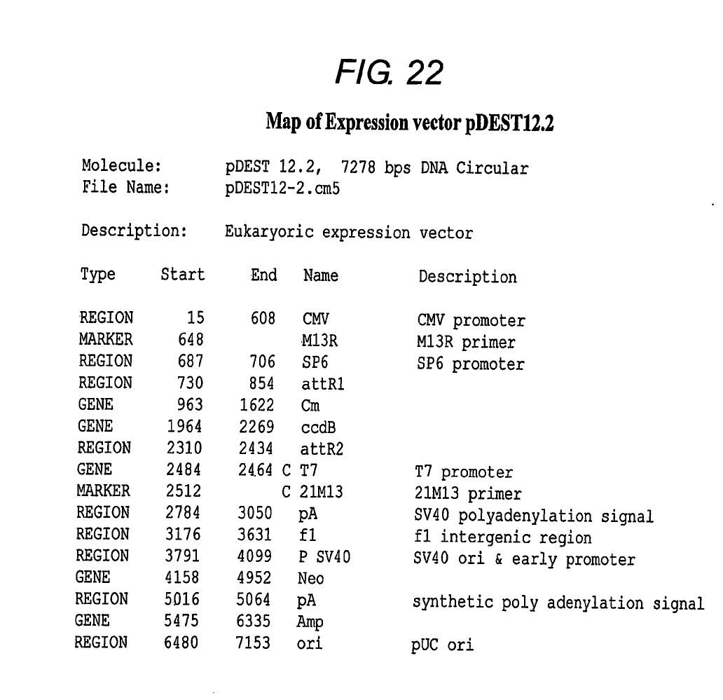

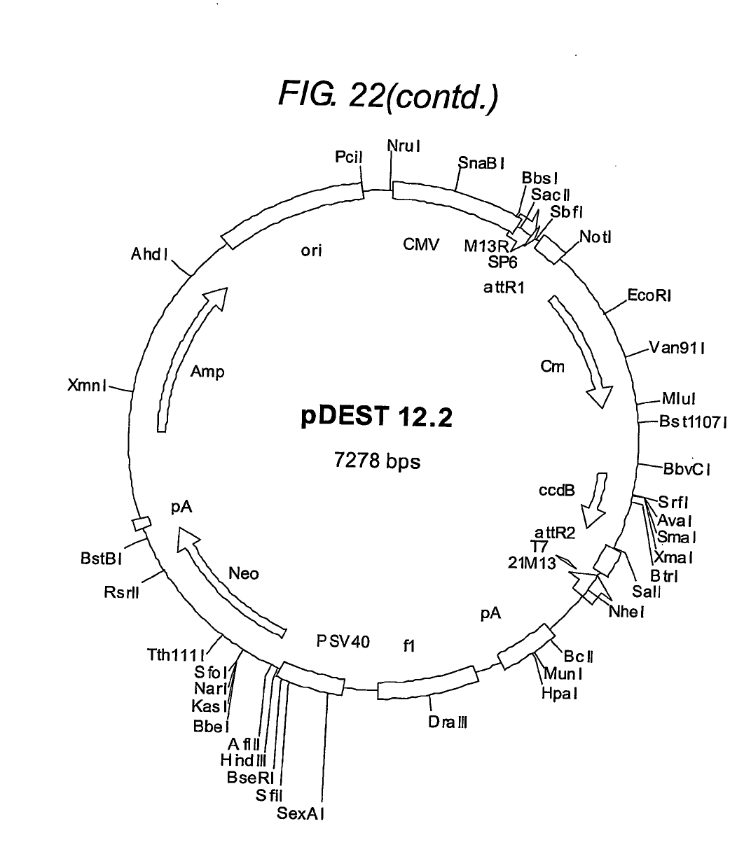

A pCR4-TOPO clone containing the full coding sequence (ORF) of INSP113 or INSP113sv identified by DNA sequencing (pCR4-TOPO-INSP113, plasmid ID 13887 (FIG. 18), or pCR4-TOPO-INSP113 sv, plasmid ID 13888 (FIG. 19)) was then used to subclone each insert into the mammalian cell expression vectors pEAK12d (FIG. 21) and pDEST12.2 (FIG. 22) using the Gateway™ cloning methodology (Invitrogen).

8.1 Generation of Gateway Compatible INSP113 ORF and INSP113sv ORF Fused to an In-Frame 6HIS Tag Sequence

The first stage of the Gateway cloning process involves a two step PCR reaction which generates the ORF of INSP0113 or INSP113sv flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction (in a final volume of 50 μl) contains: 1.5 μl of pCR4-TOPO-INSP113 (plasmid ID 13887) or 1.5 μl of pCR4-TOPO-INSP113sv (plasmid ID 13888), 1.5 μl dNTPs (10 mM), 5 μl of 10× Pfx polymerase buffer, 1 μl MgSO4 (50 mM), 0.5 μl each of gene specific primer (100 μM) (INSP113-EX1 and INSP113-EX2, or INSP113sv-EX1 and INSP113sv-EX2), 2.5 μl 10× Enhancer™ solution (Invitrogen) and 1 μl Platinum Pfx DNA polymerase (Invitrogen). The PCR reaction was performed using an initial denaturing step of 95° C. for 2 min, followed by 15 cycles of 94° C. for 15 s; 55° C. for 30 s and 68° C. for 2 min 30 sec; and a holding cycle of 4° C. The products were purified directly from the amplification reaction using the Wizard PCR prep DNA purification system (Promega) according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 3 min; 19 cycles of 94° C., 15 sec; 55° C., 30 see and 68° C., 3 min; followed by a holding cycle of 4° C. PCR products were gel purified using the Wizard PCR prep DNA purification system (Promega) according to the manufacturer's instructions.

8.2 Subcloning of Gateway Compatible INSP113 ORF and INSP113 sv ORF into Gateway Entry Vector pDONR221 and Expression Vectors pEAK12d and pDEST12.2

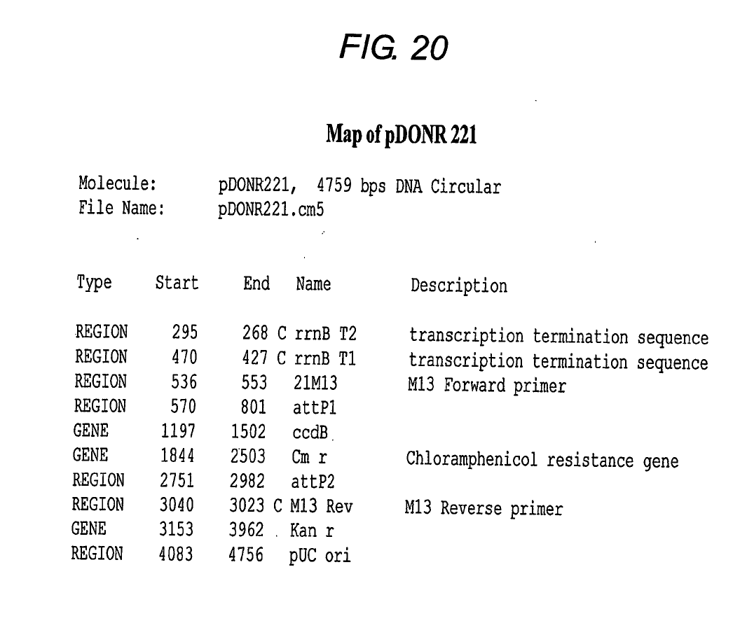

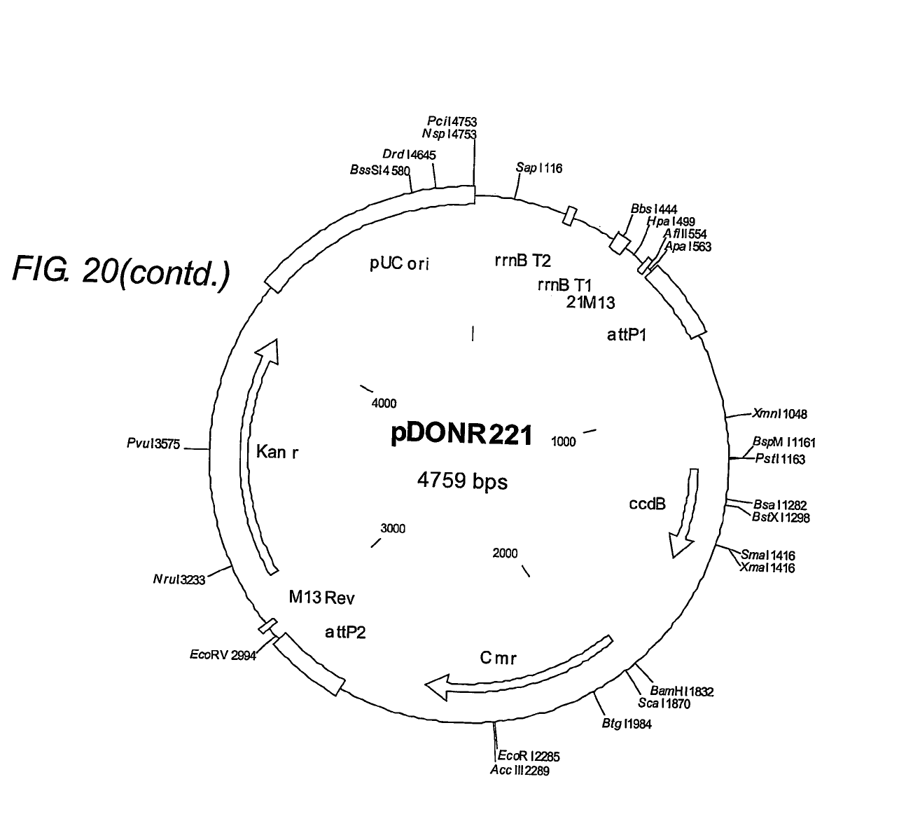

The second stage of the Gateway cloning process involves subcloning of the Gateway modified PCR product into the Gateway entry vector pDONR221 (Invitrogen, FIG. 20) as follows: 5 μl of purified product from PCR2 were incubated with 1 μl pDONR221 vector (0.15 μg/μl), 2 μl BP buffer and 1.5 μl of BP clonase enzyme mix (Invitrogen) in a final volume of 10 μl at RT for 1 h. The reaction was stopped by addition of proteinase K (2 μg) and incubated at 37° C. for a further 10 min. An aliquot of this reaction (2 μl) was used to transform E. coli DH10B cells by electroporation as follows: a 30 μl aliquot of DH10B electrocompetent cells (Invitrogen) was thawed on ice and 2 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (200-500 ng) was subjected to DNA sequencing with 21M13 and M13Rev primers using the BigDyeTerminator system (Applied Biosystems cat. no. 4390246) according to the manufacturer's instructions. The primer sequences are shown in Table 2. Sequencing reactions were purified using Dye-Ex columns (Qiagen) or Montage SEQ 96 cleanup plates (Millipore cat. no. LSKS09624) then analyzed on an Applied Biosystems 3700 sequencer.

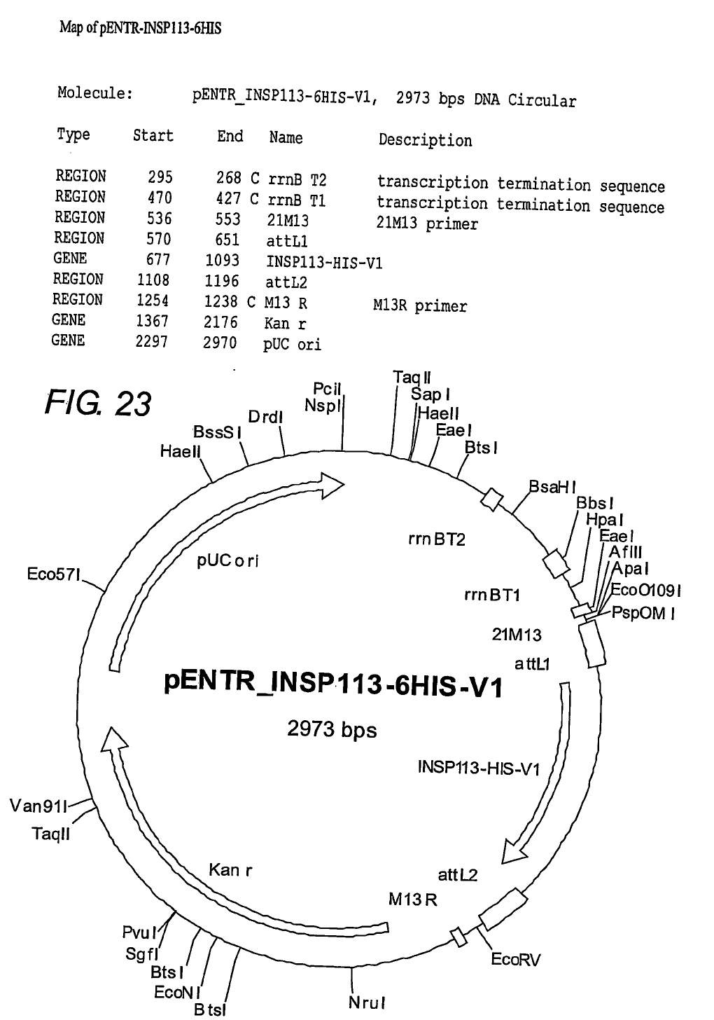

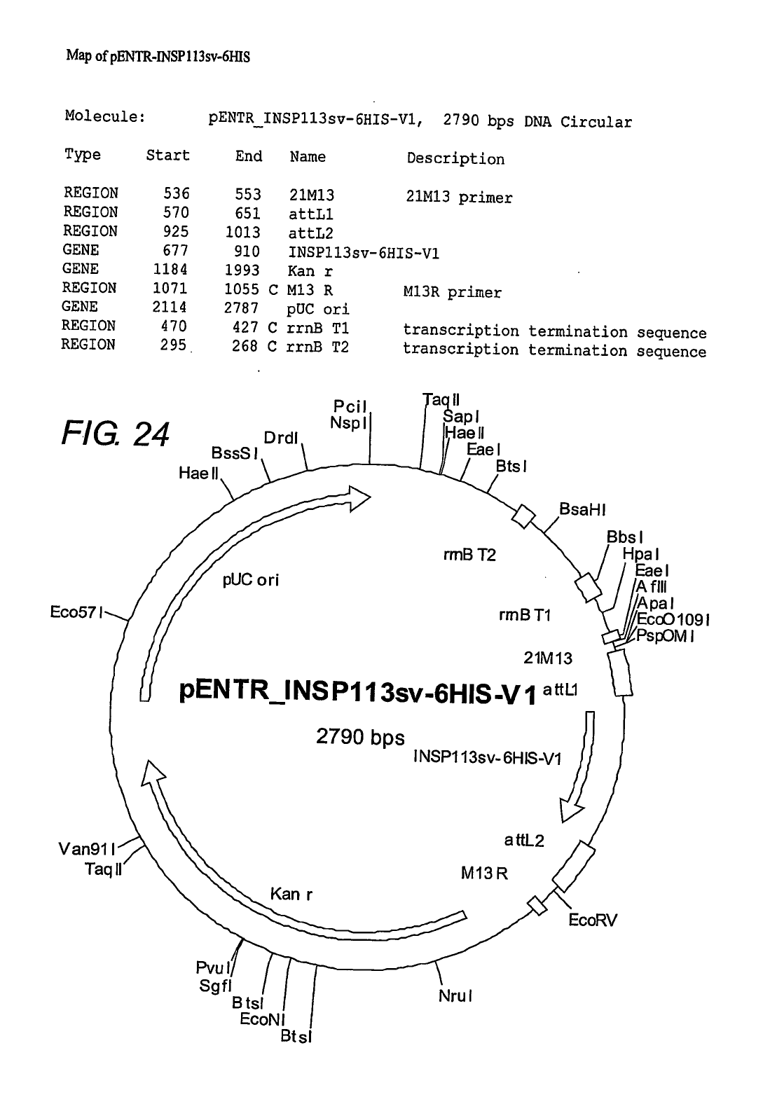

Plasmid eluate (2 μl) from one of the clones which contained the correct sequence (pENTR-INSP113-6HIS, plasmid ID 14216, FIG. 23, and pENTR-INSP113sv-6HIS, plasmid ID 14223, FIG. 24) was then used in a recombination reaction containing 1.5 μl of either pEAK12d vector or pDEST12.2 vector (FIGS. 21 & 22) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 30 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primers sequences are shown in Table 2.

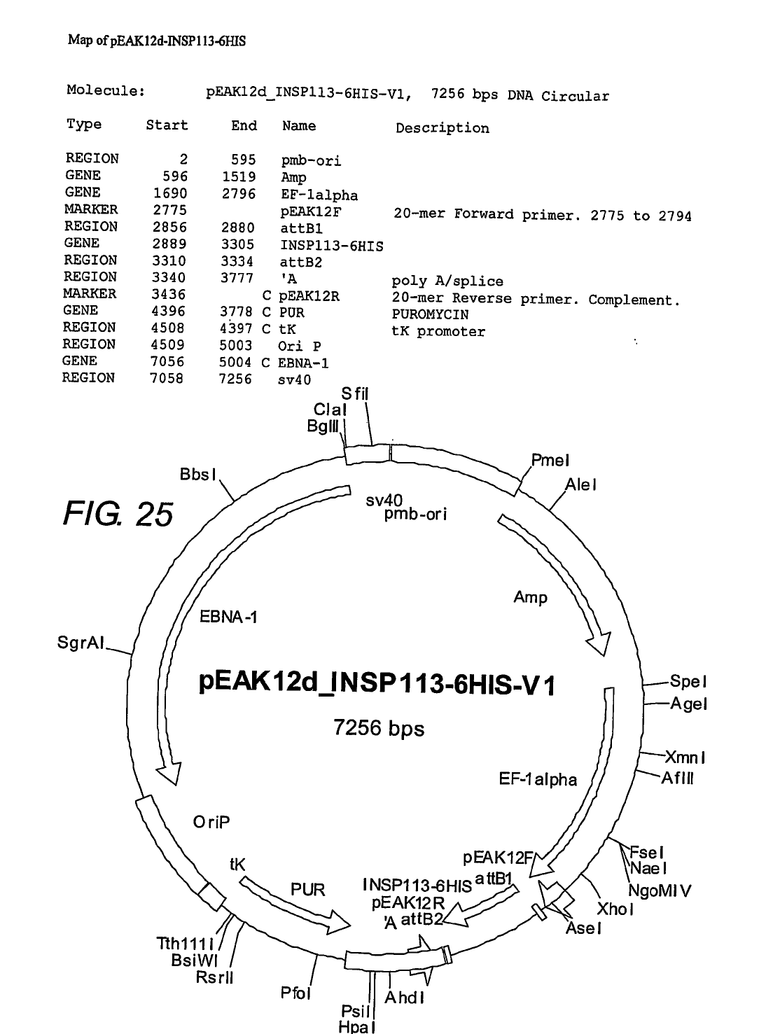

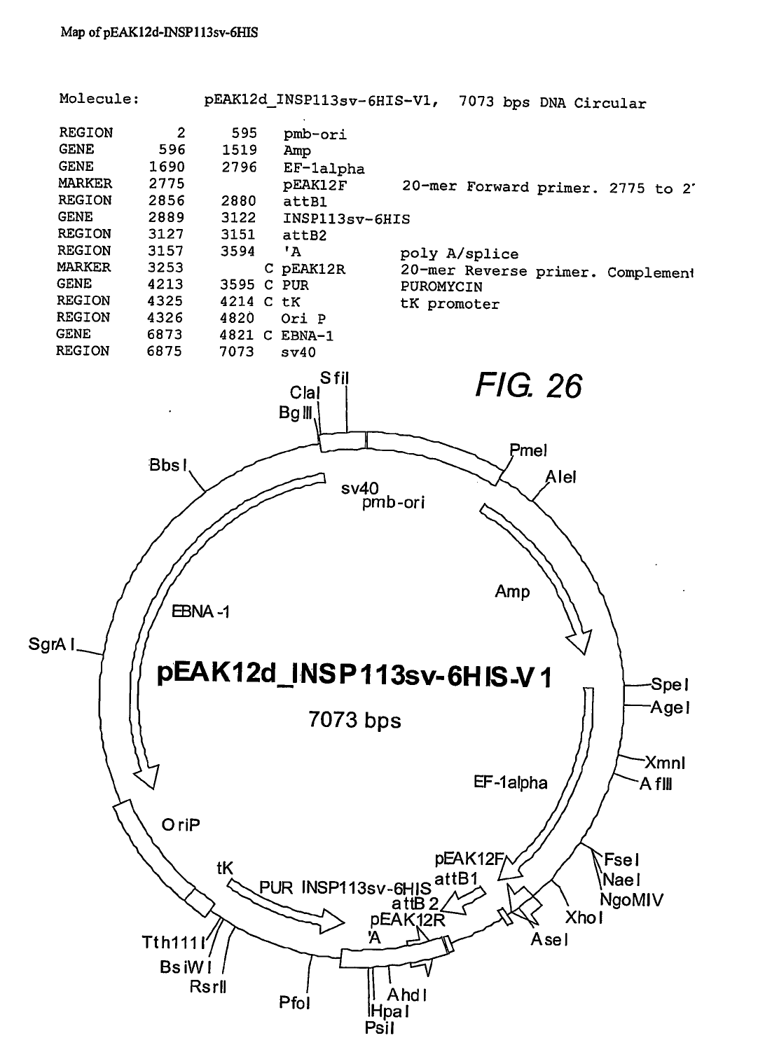

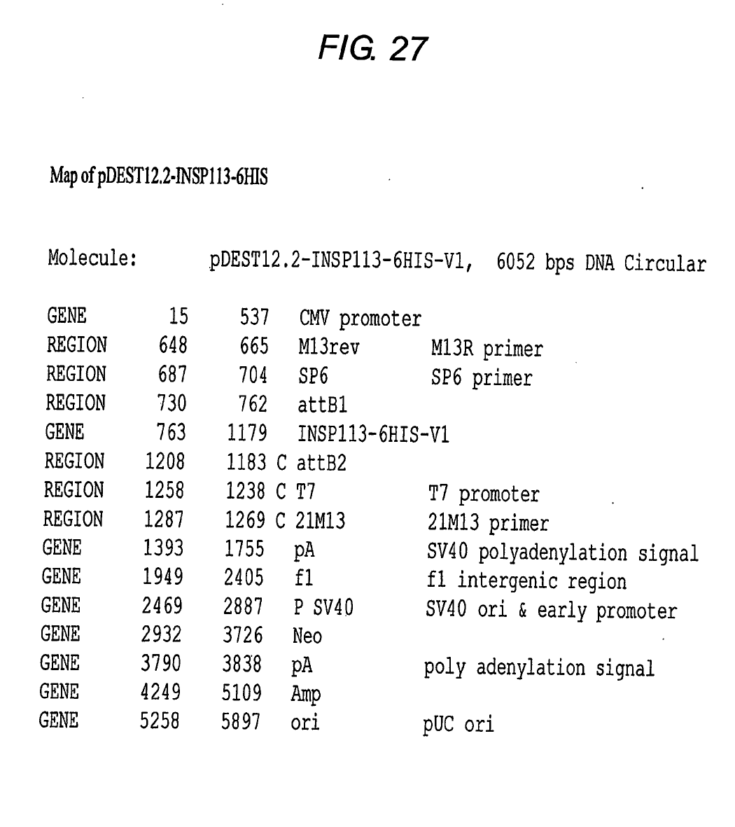

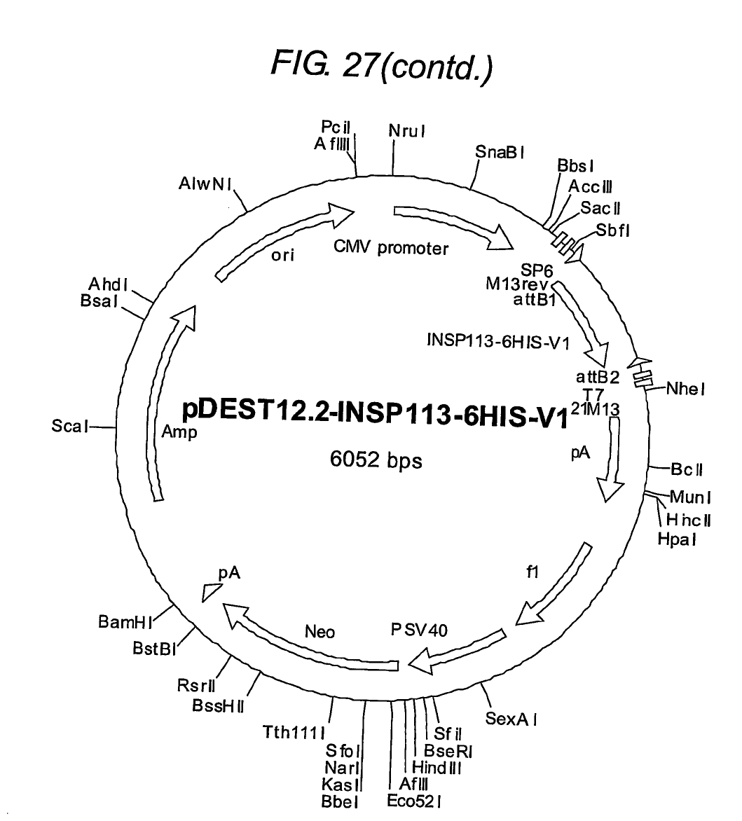

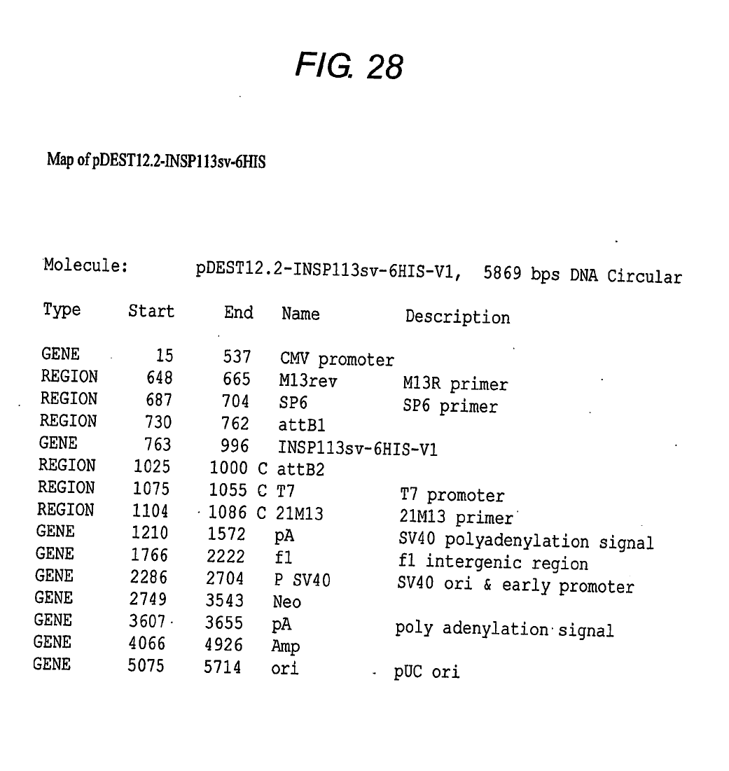

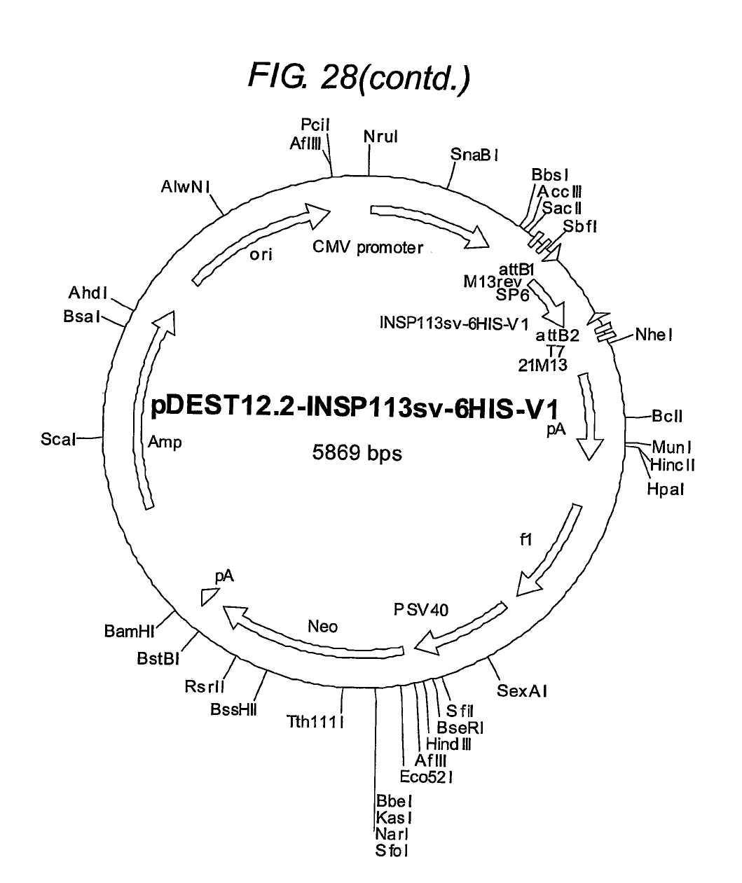

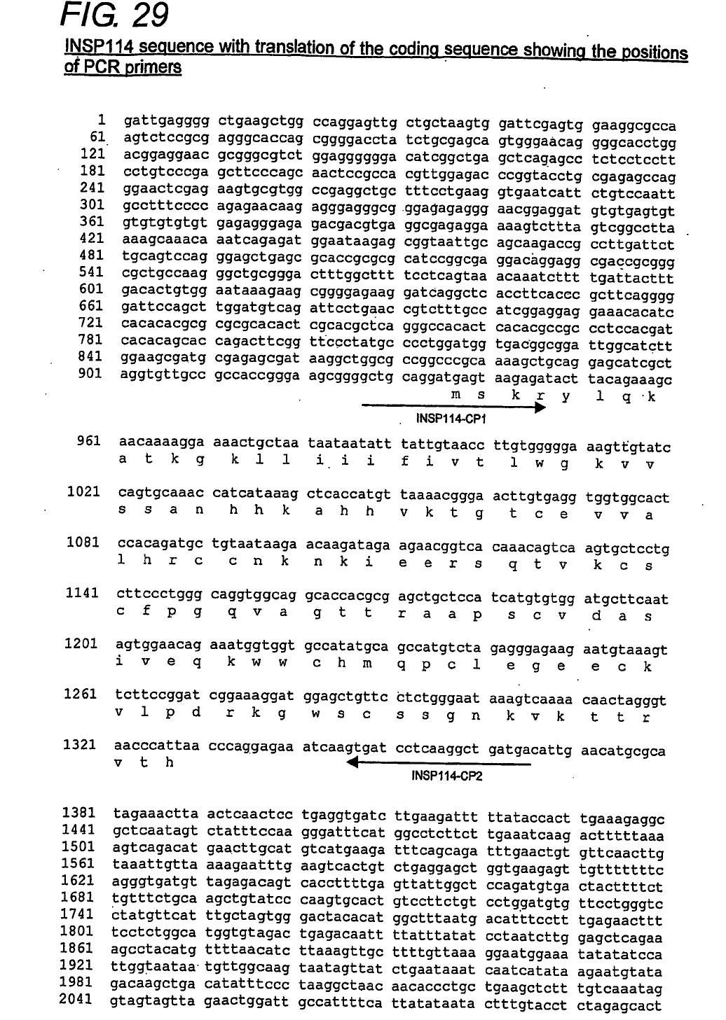

CsCl gradient purified maxi-prep DNA was prepared from a 500 ml culture of one of each of the sequence verified clones (pEAK12d-INSP113-6HIS, plasmid ID number 14225 (FIG. 25), pEAK12d-INSP113sv-6HIS, plasmid ID number 14227 (FIG. 26), pDEST12.2-INSP113-6HIS, plasmid ID 14226 (FIG. 27), pDEST12.2-INSP113sv-6HIS, plasmid ID 14260 (FIG. 28)) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water and stored at −20° C.

### Example 9

### Cloning of INSP114

9.1 Preparation of Human cDNA Templates

First strand cDNA was prepared from a human normal brain total RNA sample (Clontech) using Superscript II RNase H⁻ Reverse Transcriptase (Invitrogen) according to the manufacturer's protocol. Oligo (dT)₁₅ primer (1 μl at 500 μg/ml) (Promega), 2 μg human brain total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents were collected by brief centrifugation and 4 μl of 5× First-Strand Buffer, 2 μl 0.1 M DTT, and 1 μl RnaseOUT Recombinant Ribonuclease Inhibitor (40 units/μl, Invitrogen) were added. The contents of the tube were mixed gently and incubated at 42° C. for 2 min; then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl.

9.2 cDNA Libraries

Human cDNA libraries (in bacteriophage lambda (L) vectors) were purchased from Clontech or made in-house in λ GT10 vectors. Bacteriophage % DNA was prepared from small scale cultures of infected E. coli host strain using the Wizard Lambda Preps DNA purification system according to the manufacturer's instructions (Promega, Corporation, Madison Wis.). Human cDNA library samples used as templates for the amplification of INSP114 were derived from adult and fetal brain.

9.3 Gene Specific Cloning Primers for PCR

A pair of PCR primers having a length of between 18 and 25 bases were designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP114) with little or no none specific priming.

9.4 PCR Amplification of INSP114 from a Variety of Human cDNA Templates and Phage Library cDNA

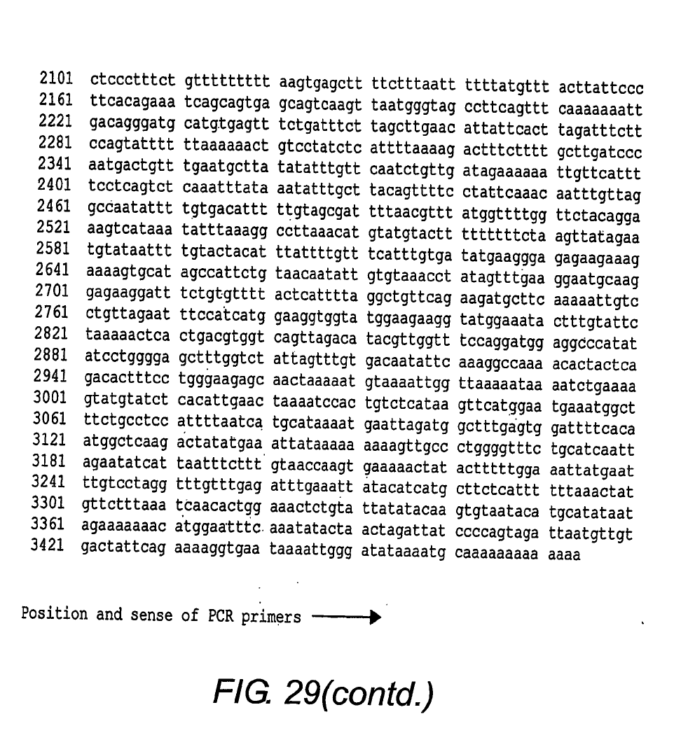

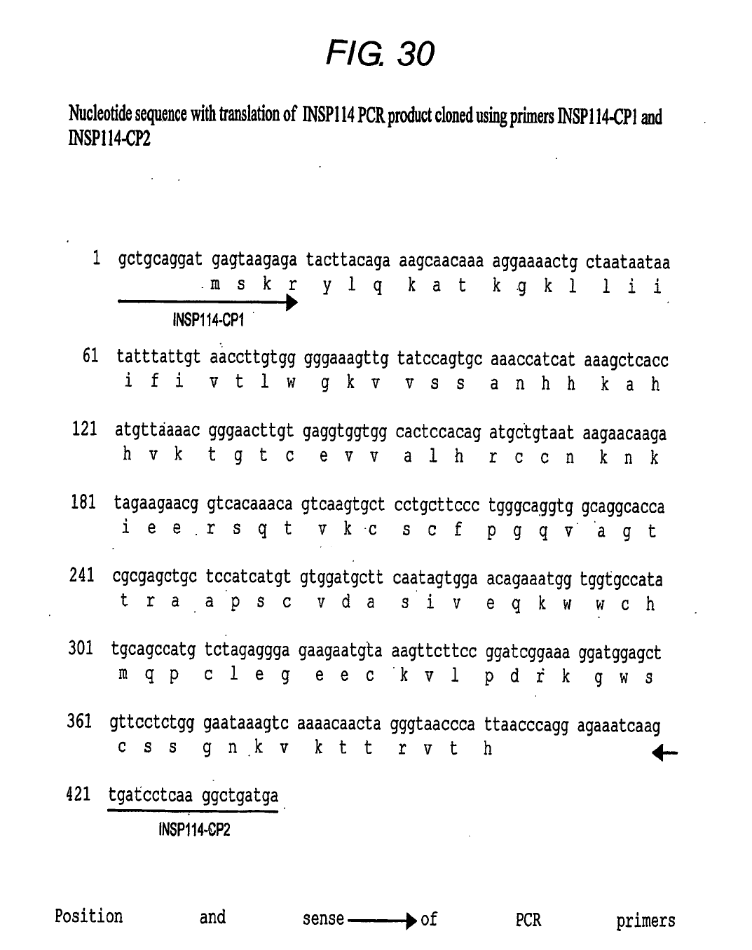

Gene-specific cloning primers (INSP114-CP1 and INSP114-CP2, FIG. 29, FIG. 30 and Table 3) were designed to amplify a cDNA fragment of 439 bp covering the entire 393 bp coding sequence of the INSP114 prediction. Interrogation of public EST sequence databases with the INSP114 prediction suggested that the sequence should be expressed in brain cDNA templates. The gene-specific cloning primers INSP114-CP1 and INSP114-CP2 were therefore used with human cDNA samples listed in Section 8.1 and the phage library cDNA samples listed in Section 8.2 as the PCR templates. The PCR was performed in a final volume of 50 μl containing 1× AmpliTaq™ buffer, 200 μM dNTPs, 50 pmoles of each cloning primer, 2.5 units of AmpliTaq™ (Perkin Elmer) and 100 ng of human cDNA template using an MJ Research DNA Engine, programmed as follows: 94° C., 2 min; 40 cycles of 94° C., 30 sec, 55° C., 30 sec, and 72° C., 1 min; followed by 1 cycle at 72° C. for 7 min and a holding cycle at 4° C.

TABLE 3
INSP114 cloning and sequencing primers

| | |
|---|---|
| Primer | Sequence (5′-3′) |

| | |
|---|---|
| INSP114-Cp1 | GCT GCA GGA TGA GTA AGA GA |
| INSP114-CP2 | TCA TCA GCC TTG AGG ATC AC |
| INSP114-Cp3 | ATG AGT AAG AGA TAC TTA CAG AAA GC |
| INSP114-Cp4 | TCA CCA CCT AGT TGT TTT GAC TTT ATT |
| | C |
| INSP114-GR1-3′ | ACG CGA GCT GCT CCA TCA TGT GT |
| INSP114- | TGG TGC CAT ATG CAG CCA TGT CT |
| GR1nest-3′ | |
| GeneRacer ™ 3′ | GCT GTC AAC GAT ACG CTA CGT AAC G |
| GeneRacer ™ | CGC TAC GTA ACG GCA TGA CAG TG |
| Nested 3′ | |
| GeneRacer ™ | GCT GTC AAC GAT ACG CTA CGT AAC GGC |
| Oligo dT | ATG ACA GTG(T)₁₈ |
| INSP114-EX1 | AA GCA GGC TTC GCC ACC ATG AGT AAG |
| | AGA TAG TTA CA |
| INSP114-EX2 | GTG ATG GTG ATG GTG ATG GGT TAC CCT |
| | AGT TGT TT |
| INSP114-EX3 | GTG ATG GTG ATG GTG CAC GTT TGC CCT |
| | AGT TGT TT |
| INSP114-EX4 | GTG ATG GTG ATG GTG CCA CCT AGT TGT |
| | TTT GAC TT |
| GCP Forward | G GGG ACA AGT TTG TAC AAA AAA GCA |
| | GGC TTC GCC ACC |
| GCP Reverse | GGG GAC CAC TTT GTA CAA GAA AGC TGG |
| | GTT TCA ATG GTG ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 | TAA TAC GAC TCA CTA TAG G |
| T3 | ATT AAC CCT CAC TAA AGG |
| SP6 | ATT TAG GTG ACA CTA TAG |

Underlined sequence = Kozak sequence
Bold = Stop codon
Italic sequence = His tag

All 50 μl of each amplification product was visualized on a 0.8% agarose gel in 1×TAE buffer (Invitrogen) and a single PCR product was seen migrating at approximately the predicted molecular mass in the sample corresponding to the brain first strand cDNA template. This PCR product was purified using the Qiagen MinElute DNA Purification System (Qiagen). The PCR product was eluted in 10 μl of EB buffer (10 mM Tris.Cl, pH 8.5) and subcloned directly.

9.5 Subcloning of PCR Products

The PCR product was subcloned into the topoisomerase I modified cloning vector (pCR4-TOPO) using the TA cloning kit purchased from the Invitrogen Corporation using the conditions specified by the manufacturer. Briefly, 4 μl of gel purified PCR product from the human brain cDNA amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

9.6 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR reaction products were analyzed on 1% agarose gels in 1× TAE buffer. Colonies which gave the expected PCR product size (439 bp cDNA+105 bp due to the multiple cloning site or MCS) were grown up overnight at 37° C. in 5 ml L-Broth (LB) containing ampicillin (100 μg/ml), with shaking at 220 rpm.

9.7 Plasmid DNA Preparation and Sequencing

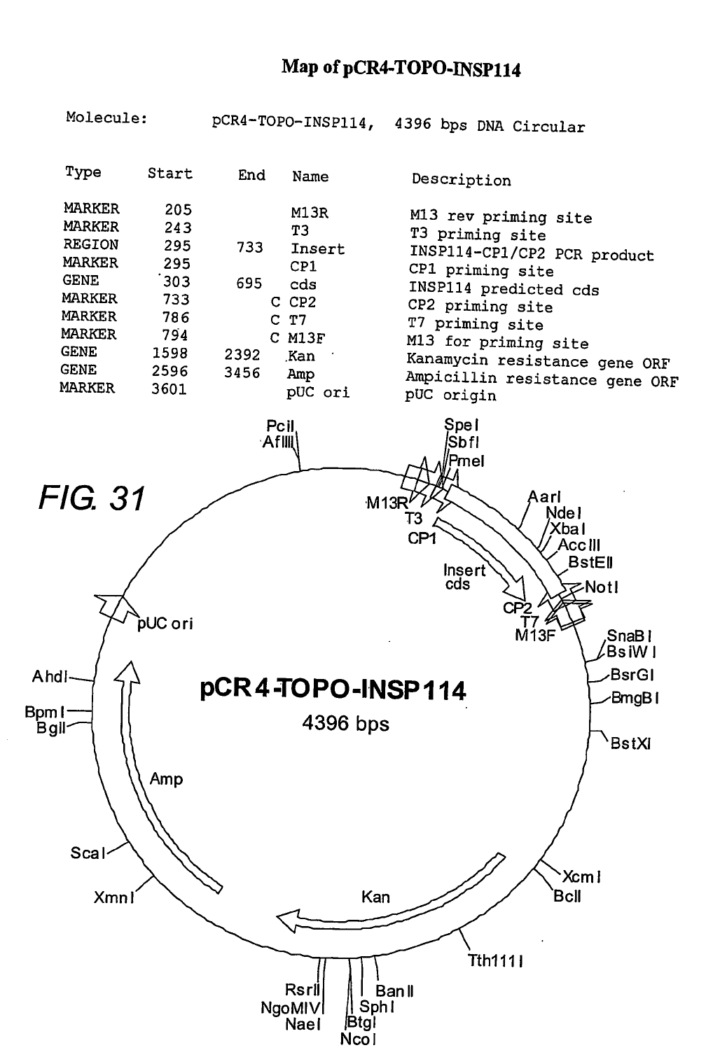

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or Spectramax 190 photometer (Molecular Devices). Plasmid DNA (200-500 ng) was subjected to DNA sequencing with the T7 primer and T3 primer using the BigDye Terminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer. Sequence analysis identified a clone containing a 100% match to the predicted INSP114 sequence. The sequence of the cloned cDNA fragment is shown in FIG. 29. The plasmid map of the cloned PCR product (pCR4-TOPO-INSP114) (plasmid ID.14213) is shown in FIG. 31.

9.8 Identification and Sequencing of IMAGE cDNA Clone

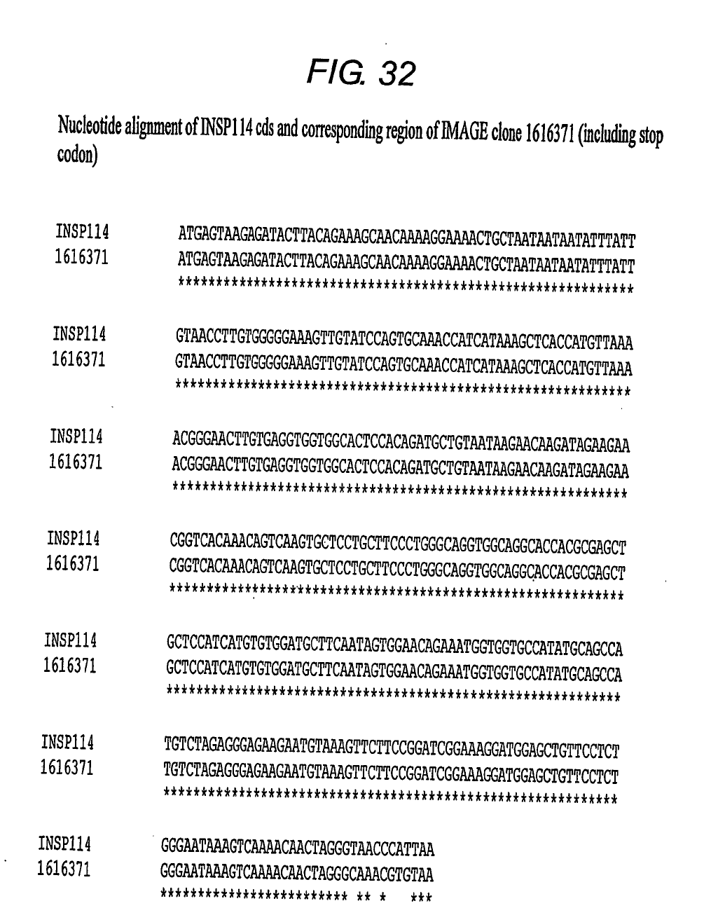

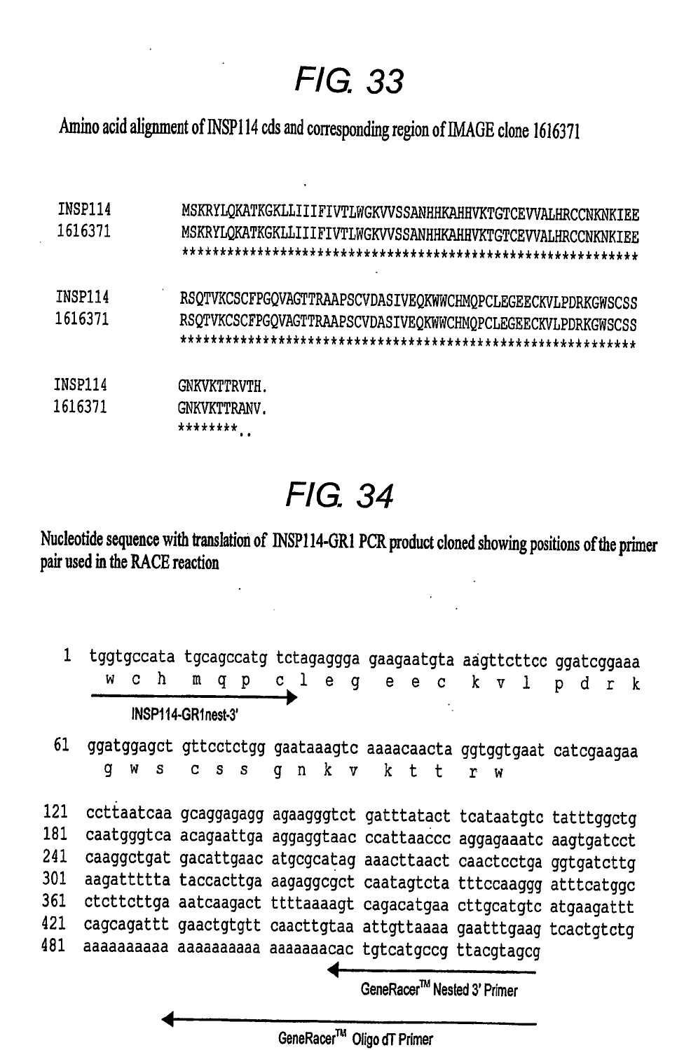

Interrogation of public EST sequence databases with the INSP114 prediction identified a number of human ESTs which corresponded to portions of the INSP114 sequence. These ESTs were derived from brain and testis templates. One EST was identified, GenBank Accession AA984082, which showed 100% match to the first 385 bp of the INSP114 coding sequence. The corresponding clone, IMAGE ID 1616371, was bought from ATCC. The insert of the IMAGE clone was sequenced using the T7 and T3 sequencing primers, and also the amplification primers INSP114-CP1 and INSP114-CP2 (see above). The IMAGE clone insert was found to correspond exactly to the first 3 exons of the INSP114 sequence. The last exon consisted of only 3 amino acids followed by a stop codon. In the INSP114 sequence these amino acids were VTH (Val-Thr-His). In the IMAGE clone these 3 amino acids were found to be ANV (Ala-Asn-Val), followed by a stop codon. The IMAGE clone 1616371 corresponds to INSP114-SV1 and is plasmid ID 14211. The alignment of the INSP114 cds and the corresponding region of IMAGE clone 1616371 is shown in FIGS. 32 and 33.

9.9 3′ RACE (Rapid Amplification of cDNA Ends)

As the IMAGE clone 1616371 was found to contain a different exon 4 from the expected sequence, it was decided to carry out 3′ RACE from the INSP114 prediction to identify whether any further exon 4 sequences would be found. Interrogation of public EST sequence databases with the INSP114 prediction had identified a number of corresponding human ESTs derived from brain and testis templates. It was therefore decided to use brain and testis cDNA samples as the templates for the 3′ RACE reactions.

9.10 3′ RACE Amplification Reactions

3′ RACE was carried out using the GeneRacer™ system (Invitrogen) in accordance with the manufacturer's instructions. All reactions components, except the RNA templates, were supplied with the system. Brain and testis RNA (Clontech, Ambion) was converted to 3′ RACE-ready first strand cDNA using the supplied GeneRacer™ Oligo dT primer (Table 3) and the SuperScript II RNase H⁻ Reverse Transcriptase (Invitrogen) according to the manufacturer's protocol. Briefly, 1 μl GeneRacer™ Oligo dT primer (50 μM), 1 pt dNTP mix (10 mM), 5 μg total RNA sample and DEPC-treated sterile water were mixed in a final volume of 12 μl in a 1.5 ml Eppendorf tube, heated at 65° C. for 5 min and then chilled on ice for 2 min. The contents were collected by brief centrifugation and 4 μl of 5× First-Strand Buffer, 2 μl 0.1 M DTT, 1 μl RnaseOUT Recombinant Ribonuclease Inhibitor (40 units/μl) and 1 μl SuperScript II RT (200 U/μl) were added. The contents were mixed gently and collected by brief centrifugation, incubated at 42° C. for 50 min, then inactivated by heating at 70° C. for 15 min. The mixture was then chilled on ice for 2 min, the contents collected again by brief centrifugation, and 1 μl (2 units) of E. coli RNase H added to remove RNA complementary to the cDNA. The mixture was incubated at 37° C. for 20 min, then chilled on ice. The first strand cDNA was stored at −20° C. before being used in RACE reactions.

A pair of gene specific nested 3′ RACE primers (INSP114-GR1-3′ and INSP114-GR1nest-3′, Table 3) were designed within exon 2 and exon 3, respectively, of the INSP114 sequence. These primers were used in consecutive RACE PCRs in conjunction with the GeneRacer™ 3× primer and the GeneRacer™ 3, Nested Primer, respectively. For the first amplification reaction, 1× Platinum® Taq High Fidelity PCR buffer, 2 mM MgSO₄, 200 μM dNTPs, 0.2 μM of INSP114-GR1-3′ primer, 0.6 μM of GeneRacer™ 3′ Primer, 2.5 units of Platinum® Taq High Fidelity DNA polymerase (Invitrogen), 2 μl of either 3′ GeneRacer™-ready brain first strand cDNA or 3′ GeneRacer™-ready testis first strand cDNA template were combined in a final volume of 50 μl. Thermal cycling was carried out using an MJ Research DNA Engine programmed as follows: 94° C., 2 min; 5 cycles of 94° C., 30 sec, 72° C., 3 min; 5 cycles of 94° C., 30 sec, 70 CC, 3 min; 25 cycles of 94° C., 30 sec, 60° C., 30 sec, 68° C., 3 min; followed by 1 cycle at 68° C. for 10 min and a holding cycle at 4° C.

For the secondary PCR, 1 μl of PCR1 product was combined with 1× Platinum® Taq High Fidelity PCR buffer, 2 mM MgSO₄, 200 μM dNTPs, 0.2 μM of INSP114-GR1nest-3′ primer, 0.2 μM of GeneRacer™ Nested 3′ Primer, and 2.5 units of Platinum® Taq High Fidelity DNA polymerase (Invitrogen) in a final volume of 0.50 μl. Thermal cycling was carried out using an MJ Research DNA Engine programmed as follows: 94° C., 2 min; 25 cycles of 94 CC, 30 sec, 60° C., 30 sec, 68° C., 3 min; followed by 1 cycle at 68° C. for 10 min and a holding cycle at 4° C.

All 50 μl of each amplification product was visualized on a 0.8% agarose gel in 1× TAE buffer (Invitrogen). Several bands were observed in each lane on the gel. One band was excised from the brain cDNA PCR1, one band from the brain cDNA PCR2, and three bands from the testis PCR2. These PCR products were purified using the Qiagen MinElute DNA Purification System (Qiagen). Each product was eluted in 10 μl of EB buffer (10 mM Tris.Cl, pH 8.5) and subcloned directly.

9.11 Subcloning of 3′ RACE PCR Products

Each RACE PCR product was subcloned into the topoisomerase I modified cloning vector (pCR4-TOPO) using the TA cloning kit purchased from the Invitrogen Corporation using the conditions specified by the manufacturer. Briefly, 4 μl of gel purified PCR product from the human brain cDNA amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then beat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

9.12 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min 30 sec or 3 min (depending on the size of the expected insert). Samples were maintained at 4° C. (holding cycle) before further analysis.

PCR reaction products were analyzed on 1% agarose gels in 1×TAE buffer. Colonies which appeared to contain an insert, i.e. gave a PCR product size greater than the 105 bp due to the multiple cloning site, were grown up overnight at 37° C. in 5 ml L-Broth (LB) containing ampicillin (100 μg/ml), with shaking at 220 rpm.

9.13 Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or Spectramax 190 photometer (Molecular Devices). Plasmid DNA (200-500 ng) was subjected to DNA sequencing with the T7 primer and T3 primer using the BigDye Terminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

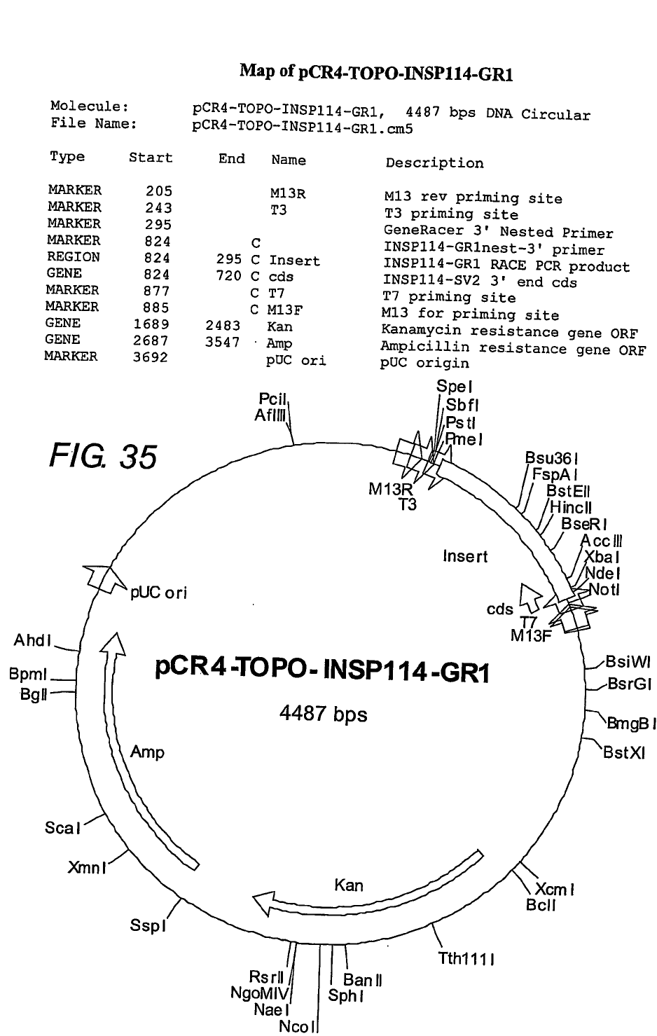

Sequence analysis identified several clones which contained the same exon 4 sequence as in either the pCR4-TOPO-INSP114 clone (plasmid ID 14213) or the IMAGE clone 1616371 (plasmid ID 14211). However, a clone was also identified which contained a third version of exon 4. In this case, exon 4 consisted of just the single amino acid W (Trp) followed by a stop codon. This represented a spliced exon 4 and not a continuation of exon 3 into intronic sequence. The sequence had been amplified from the testis cDNA template. The sequence of the product of the RACE reaction is shown is FIG. 34. The map of the cloned product, pCR4-TOPO-INSP114-GR1, is shown in FIG. 35. This clone is plasmid ID 15939.

9.14 Generation of a Clone Containing the INSP114 Predicted Exons 1-3 and the RACE-Identified Exon 4

The RACE amplification and cloning reactions had identified a novel INSP114 exon 4, but a clone needed to be produced which contained the full length of the INSP114 sequence with this novel exon 4 sequence. It was decided to engineer this version of the sequence, called INSP114-SV2, from the pCR-TOPO-INSP114 clone, plasmid ID 14213, using a pair of PCR primers which would replace the original exon 4 with the new version. These primers were not tested on cDNA templates because of the very short length of the exon 4 sequence, which would have been added by the PCR primer to any cDNA template which contained the exon 1-3 sequence.

A pair of gene-specific cloning primers, INSP114-CP3 and INSP114-CP4 (Table 3), were designed to amplify the INSP114 sequence and at the same time replace the exon 4 sequence. The PCR was carried out in a final volume of 50 μl containing 1× Platinum® Taq High Fidelity PCR buffer, 2 mM MgSO₄, 200 μM dNTPs, 10 pmoles of each cloning primer, 2.5 units of Platinum® Taq High Fidelity DNA polymerase (Invitrogen), and 1 μl of plasmid ID 14213. Thermal cycling was carried out using an MJ Research DNA Engine programmed as follows: 94° C., 2 min; 30 cycles of 94° C., 30 sec, 55° C., 30 sec, 68° C., 30 sec; followed by 1 cycle at 68° C. for 7 min and a holding cycle at 4° C.

All 50 μl of each amplification product was visualized on a 0.8% agarose gel in 1× TAE buffer (Invitrogen) and a single PCR product was seen migrating at approximately the predicted molecular mass. This PCR product was purified using the Wizard PCR Preps DNA Purification System (Promega). The PCR product was eluted in 50 μl of water and subcloned directly.

9.15 Subcloning of PCR Products

The PCR product was subcloned into the topoisomerase I modified cloning vector (pCR4-TOPO) using the TA cloning kit purchased from the Invitrogen Corporation using the conditions specified by the manufacturer. Briefly, 4 if of gel purified PCR product from the human brain cDNA amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

9.16 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of T3 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 30 sec. Samples were maintained at 4° C. (holding cycle) before further analysis.

PCR reaction products were analyzed on 1% agarose gels in 1×TAE buffer. Colonies which gave the expected PCR product size (390 bp cDNA+105 bp due to the multiple cloning site or MCS) were grown up overnight at 37° C. in 5 ml L-Broth (LB) containing ampicillin (100 μg/ml), with shaking at 220 rpm.

9.17 Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer or Spectramax 190 photometer (Molecular Devices). Plasmid DNA (200-500 ng) was subjected to DNA sequencing with the T7 primer and T3 primer using the BigDye Terminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

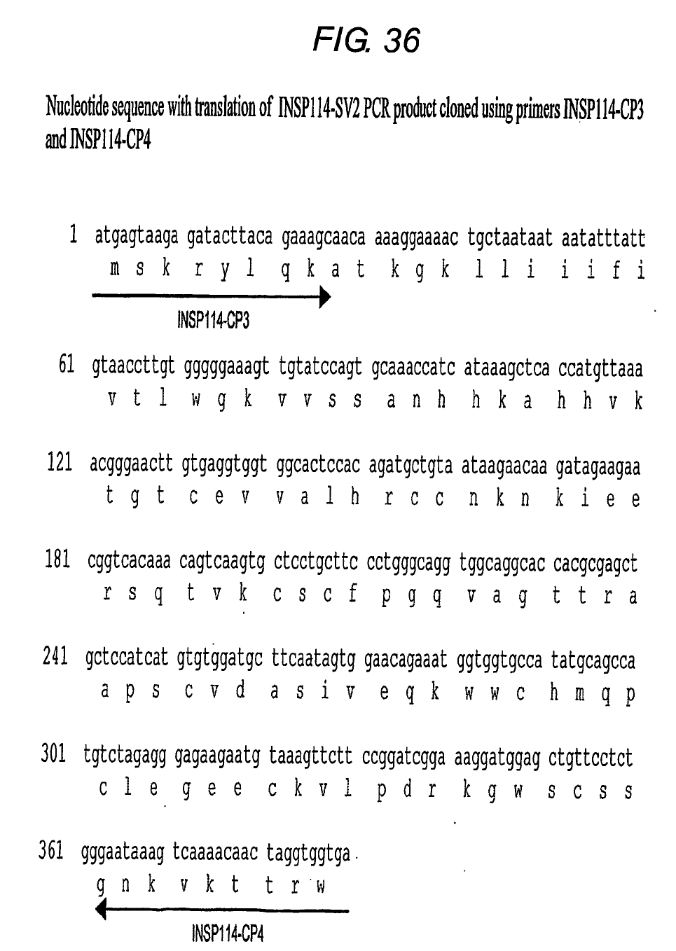

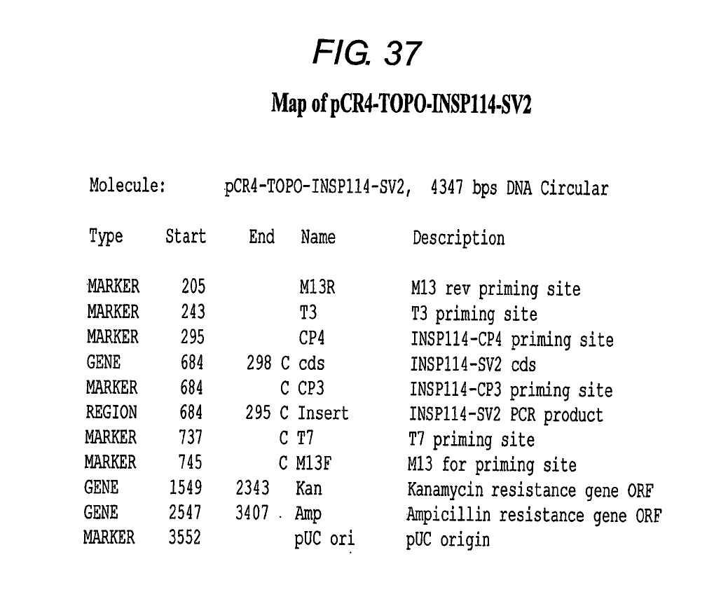

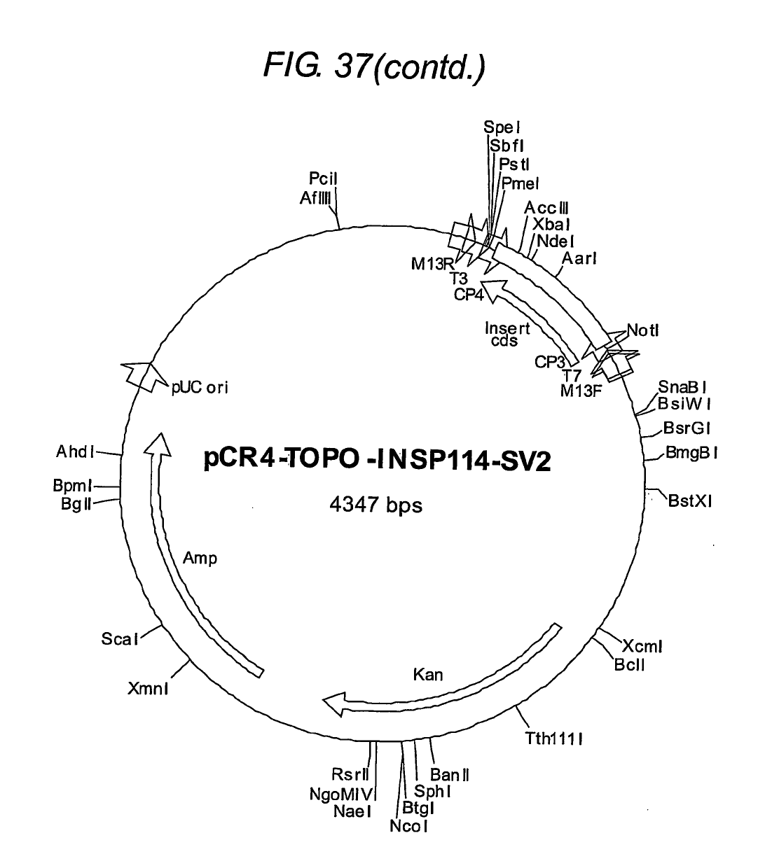

Sequence analysis identified a clone containing a 100% match to the predicted INSP114-SV2 sequence, with the alternative exon 4 sequence. The sequence of the cloned cDNA fragment is shown in FIG. 36. The plasmid map of the cloned PCR product (pCR4-TOPO-INSP114-SV2) (plasmid ID.14426) is shown in FIG. 37.

### Example 10

### Construction of Mammalian Cell Expression Vectors for INSP114, INSP114-SV1, INSP114-SV2

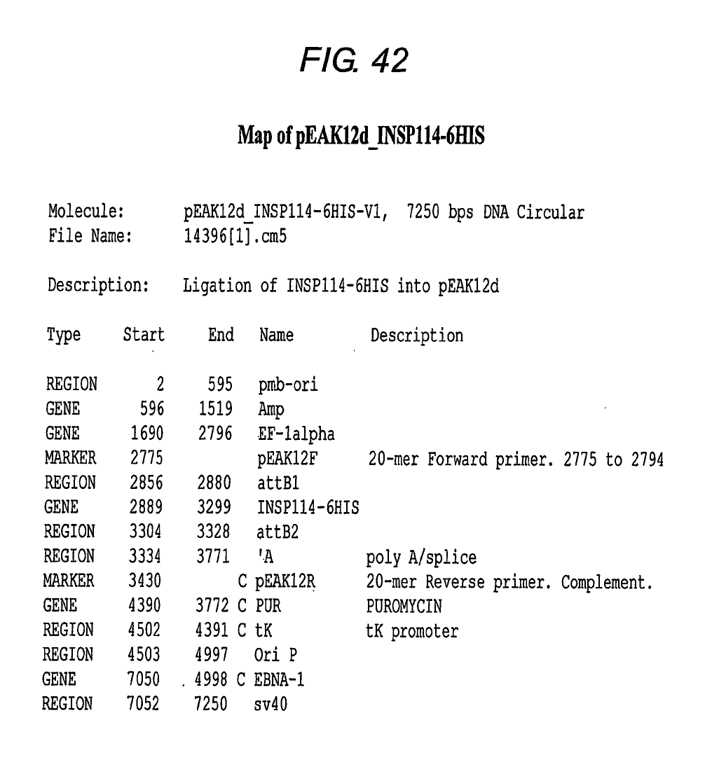

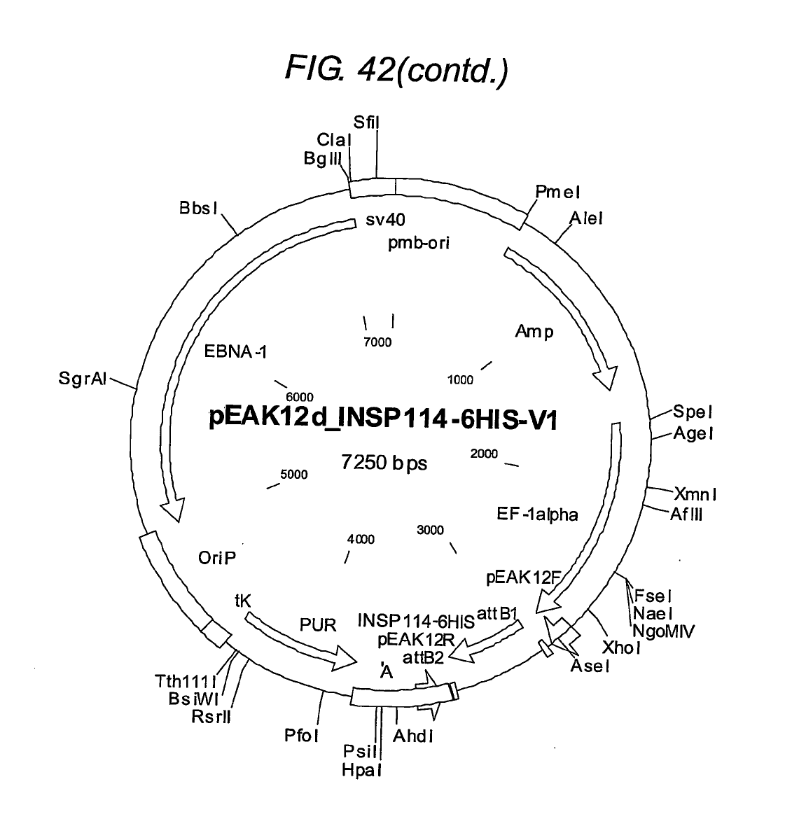

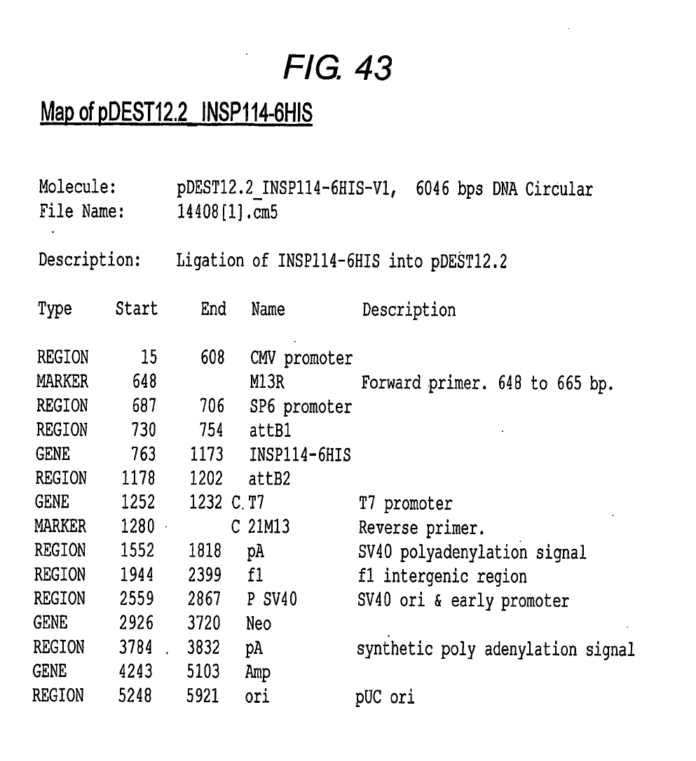

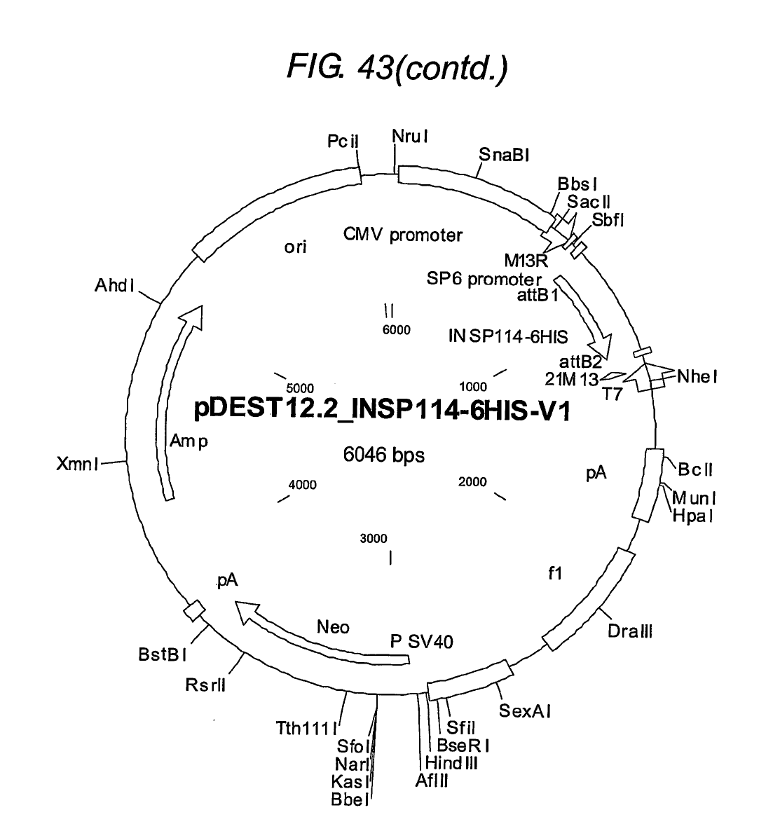

Plasmids 14213, 14211 and 14426 were used as a PCR templates to generate pEAK12d (FIG. 42, 45 & 48) and pDEST12.2 (FIG. 43, 46 & 49) expression clones containing the INSP114, INSP114-SV1 or INSP114-SV2 ORF sequences, respectively, with a 3′ sequence encoding a 6HIS tag using the Gateway™ cloning methodology (Invitrogen).

10.1 Generation of Gateway Compatible INSP114, INSP114-SV1 and INSP114-SV2 ORF Fused to an in Frame 6HIS Tag Sequence.

The first stage of the Gateway cloning process involves a two step PCR reaction which generates the ORFs of INSP114, INSP114-SV1 and INSP114-SV2 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of plasmid 14213, 14211 or 14426, 1.5 μl dNTPs (10 mM), 10 μl of 10× Pfx polymerase buffer, 1 μl MgSO4 (50 mM), 0.5 μl each of gene specific primer (100 μM) (INSP114-EX1 and INSP114-EX2 for INSP114, INSP114-EX1 and INSP114-EX3 for INSP114-SV1, and INSP114-EX1 and INSP114-EX4 for INSP114-SV2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

10.2 Subcloning of Gateway Compatible INSP114, INSP114-SV1 and INSP114-SV2 ORFs into Gateway Entry Vector pDONR221 and Expression Vectors pEAK12d and pDEST12.2

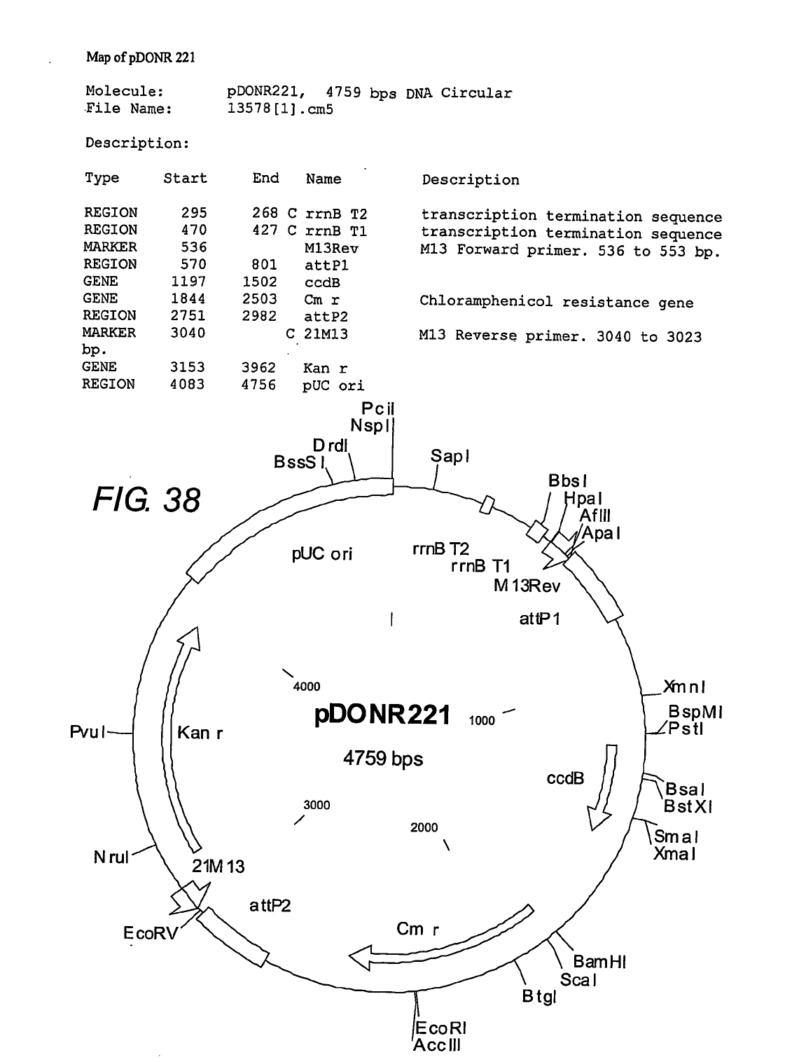

The second stage of the Gateway cloning process involves subcloning of the Gateway modified PCR product into the Gateway entry vector pDONR221 (Invitrogen, FIG. 38) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 3. Sequencing reactions were purified using Dye-Ex columns (Qiagen) or Montage SEQ 96 cleanup plates (Millipore cat. no. LSKS09624) then analyzed on an Applied Biosystems 3700 sequencer.

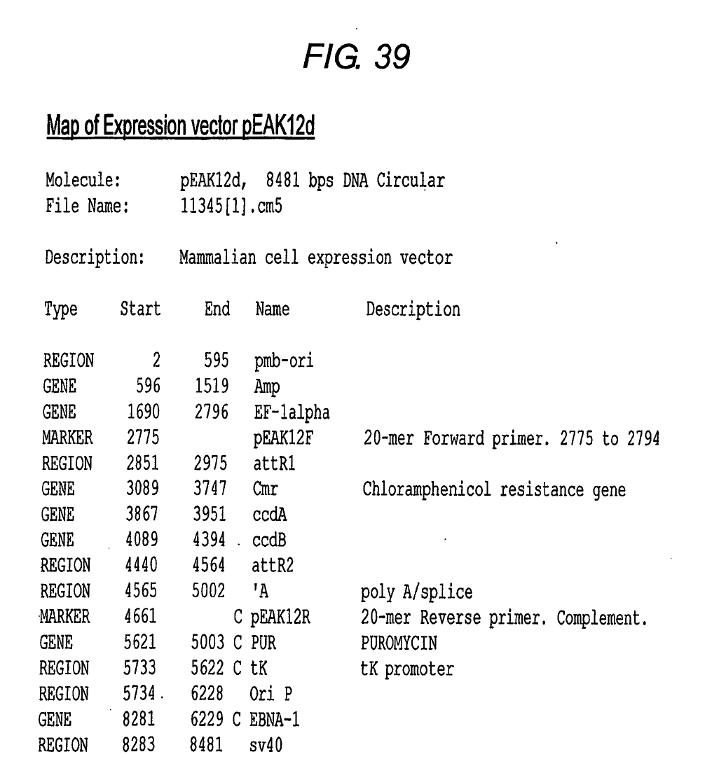

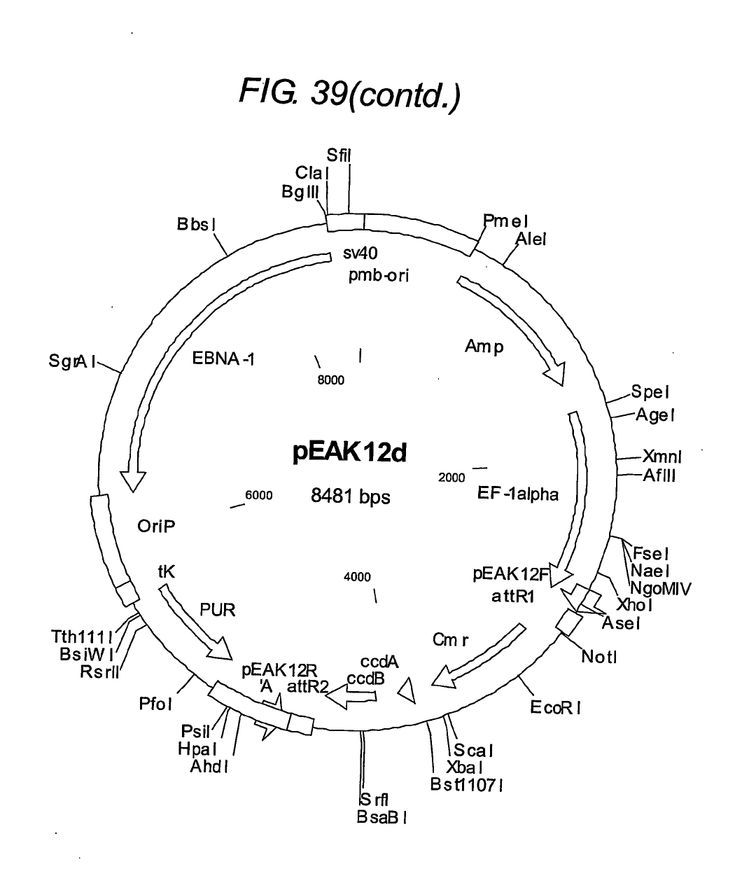

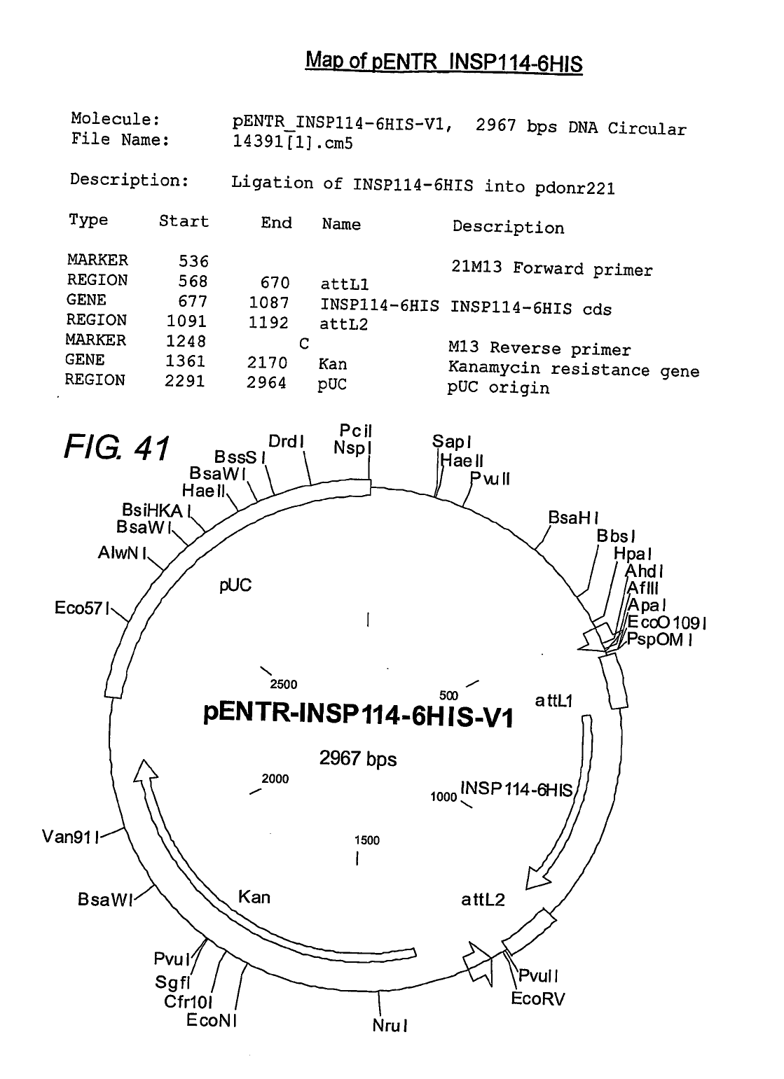

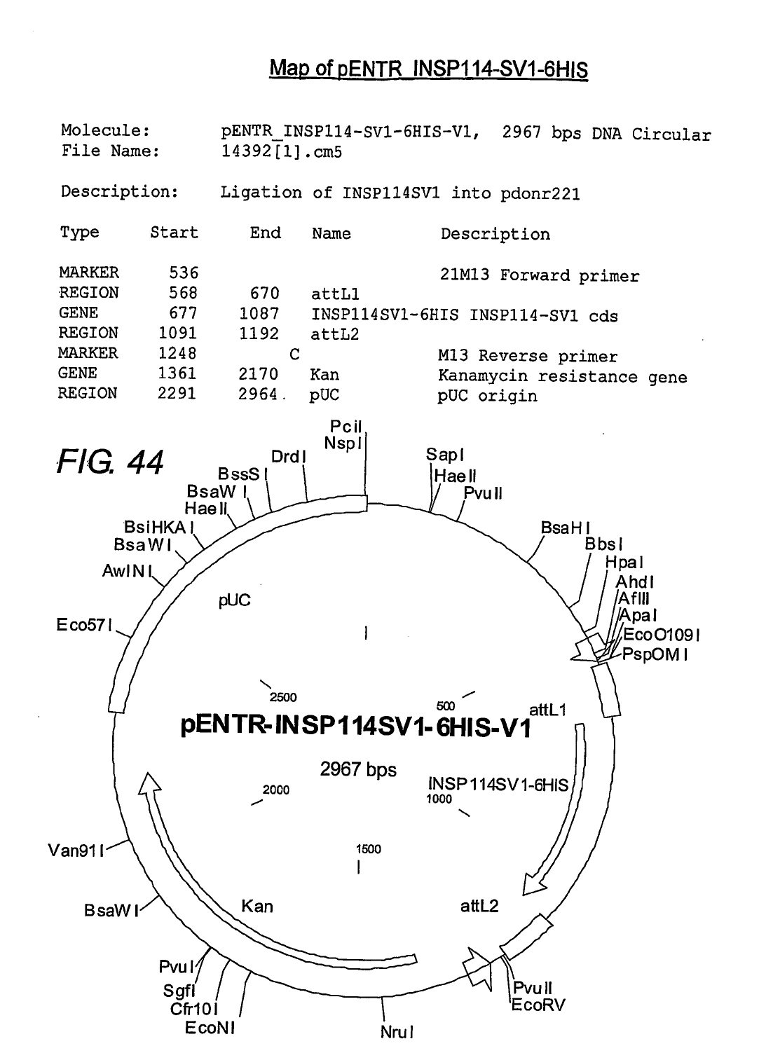

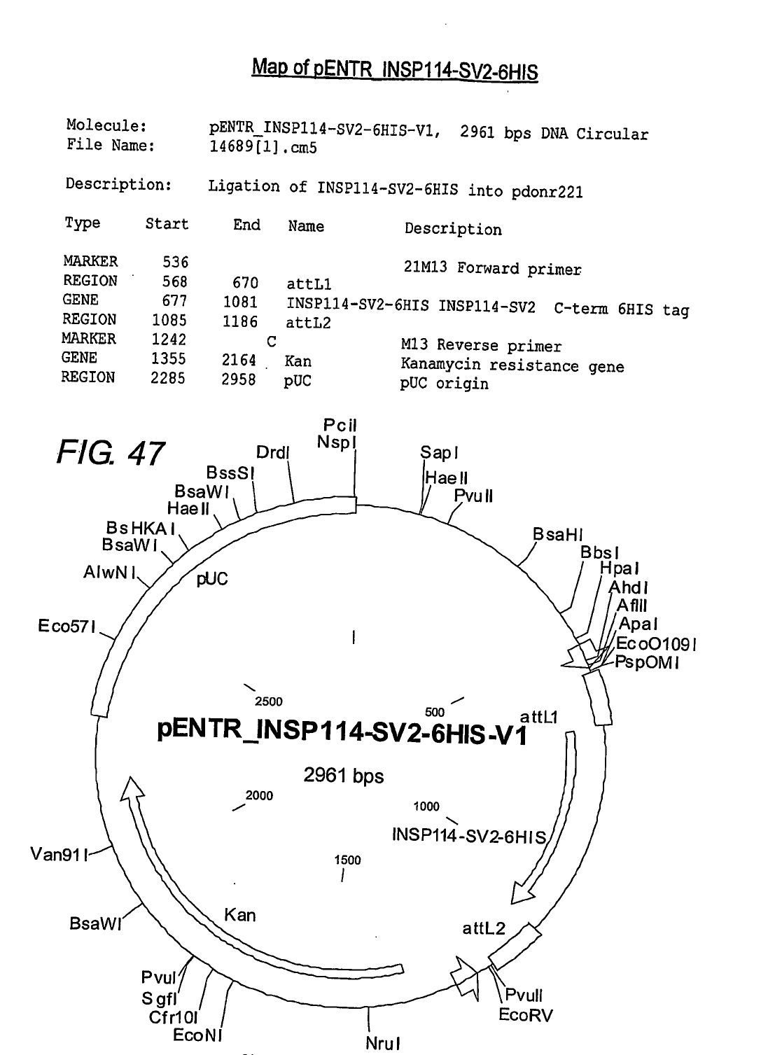

Plasmid eluate (2 μl or approx. 150 ng) from one of the clones which contained the correct sequence (pENTR_INSP114-6HIS, plasmid ID 14391, FIG. 41, pENTR_INSP114-SV1-6HIS, plasmid ID 14392, FIG. 44, or pENTR_INSP114-SV2-6HIS, plasmid ID 14689, FIG. 47) was then used in recombination reactions containing 1.5 μl of either pEAK12d vector or pDEST12.2 vector (FIGS. 39 & 40) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 3.

CsCl gradient purified maxi-prep DNA was prepared from a 500 ml culture of one of each of the sequence verified clones (pEAK12d_INSP114-6HIS, pEAK12d_INSP114-SV1-6HIS and pEAK12d_INSP114-SV2-6HIS, plasmid ID numbers 14396, 14397 and 14695, FIGS. 42, 45 and 48 respectively, and pDEST12.2-INSP114-6HIS, pDEST12.2_INSP114-SV1-6HIS and pDEST12.2_INSP114-SV2-6HIS, plasmid IDs 14408, 14409 and 14696, FIGS. 43, 46 and 49 respectively) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

### Example 11

### Cloning of INSP115

INSP115 was a prediction for a 132 amino acid protein (396 bp) encoded in 4 exons. Using the full length cds to search public EST databases we identified several sequences which matched the INSP115 sequence. One sequence was chosen, GenBank Accession BI599742 corresponding to IMAGE cDNA clone 5299791, and the clone was purchased from ATCC. The insert sequence of the IMAGE clone was sequenced using sequencing primers T7 and T3 (Table 4). The insert sequence was found to contain the INSP115 cds. IMAGE clone 5299791 is plasmid database ID 14210. The full length of the IMAGE clone insert was not sequenced after the region corresponding to INSP115 had been identified.

TABLE 4
INSP115 cloning and sequencing primers:

| | |
|---|---|
| Primer | Sequence (5′-3′) |

| | |
|---|---|
| T7 primer | TAA TAC GAC TCA CTA TAG G |
| T3 primer | ATT AAC CCT CAC TAA AGG |
| INSP115-EX1 | AA GCA GGC TTC GCC ACC ATG GCG CCA TCG |
| | CCC AGG AC |
| INSP115-EX2 | GTG ATG GTG ATG GTG GGA GAC CGT GGT GGT |
| | CTT TA |
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| PEAK12F | GCC AGC TTG GCA CTT GAT GT |
| PEAK12R | GAT GGA GGT GGA CGT GTC AG |
| 21M13 | TGT AAA ACG ACG GCC AGT |
| M13REV | CAG GAA ACA GCT ATG ACC |

Underlined sequence = Kozak sequence
Bold = Stop codon
Italic sequence = His tag

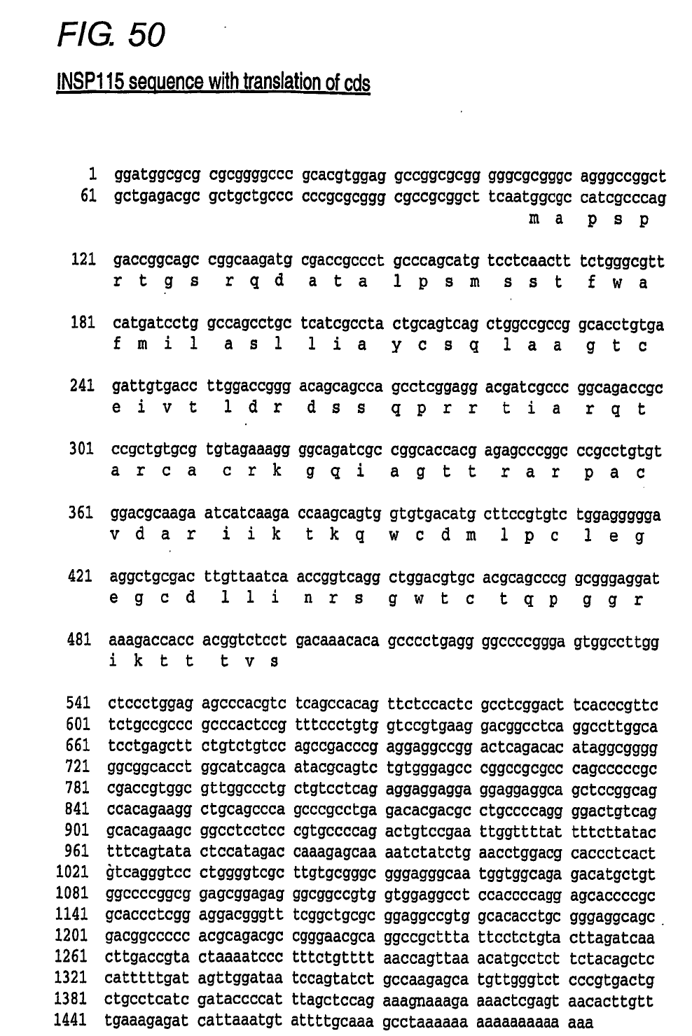

The sequence of the INSP115 prediction is shown in FIG. 50. The corresponding region of the IMAGE clone 5299791 was identical to the INSP115 cds at the nucleotide level.

### Example 12

### Construction of Mammalian Cell Expression Vectors for INSP115

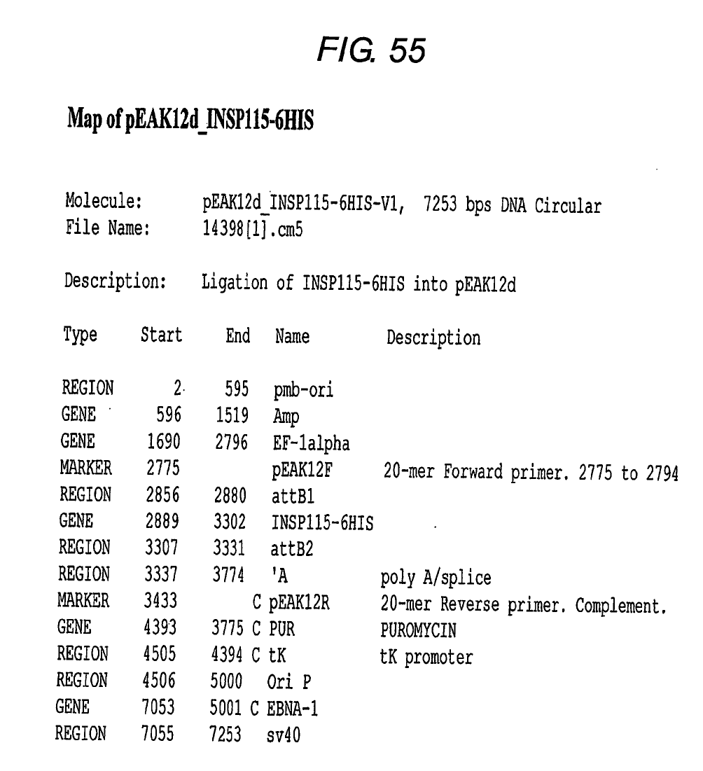

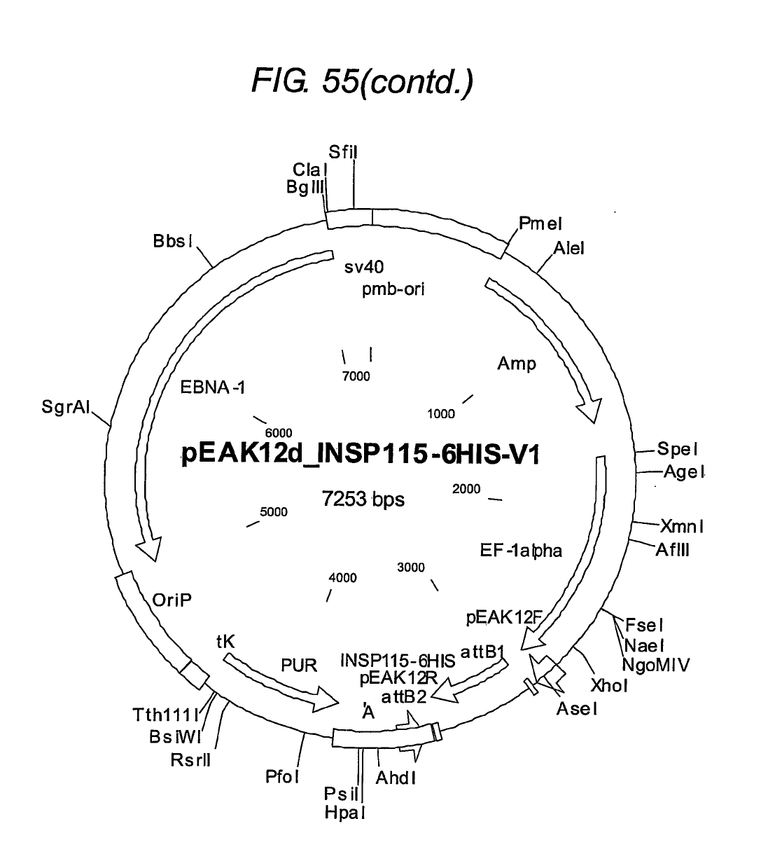

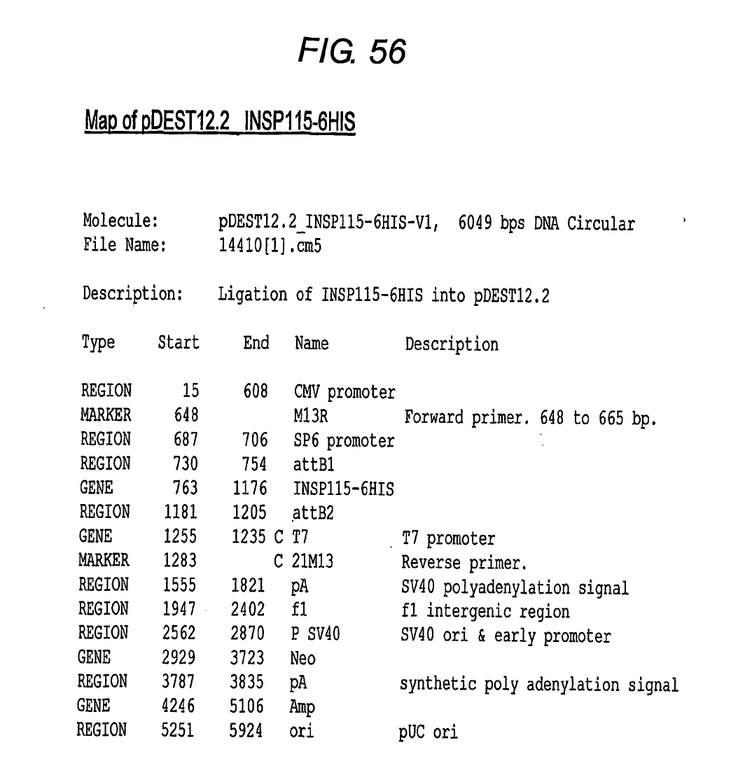

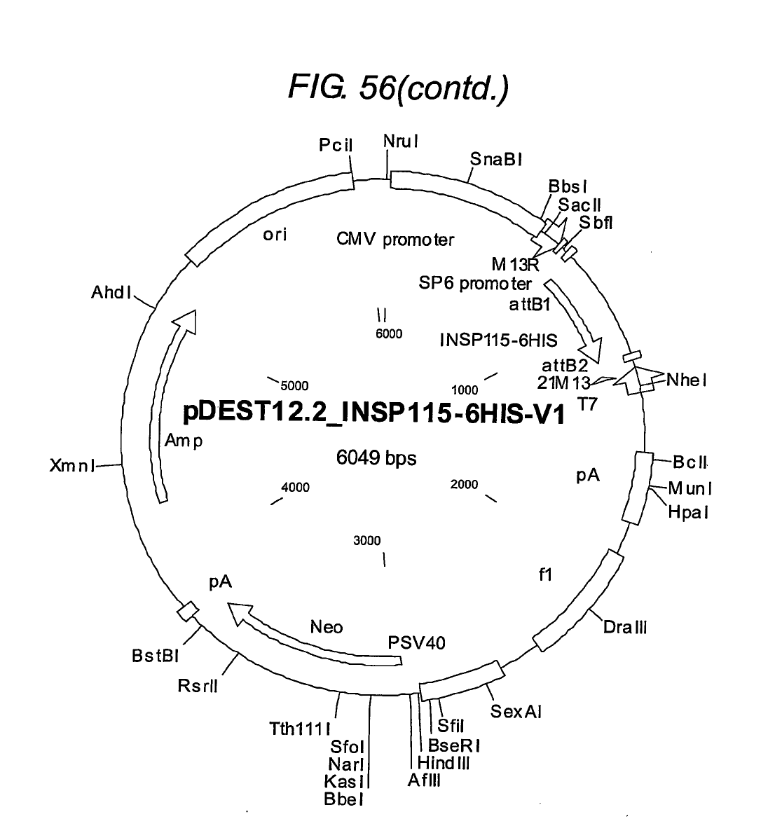

Plasmid 14210 was used as a PCR template to generate pEAK12d (FIG. 55) and pDEST12.2 (FIG. 56) expression clones containing the INSP115 ORF sequence with a 3′ sequence encoding a 6HIS tag using the Gateway cloning methodology (Invitrogen).

12.1 Generation of Gateway Compatible INSP115 ORF Fused to an in Frame 6HIS Tag Sequence.

The first stage of the Gateway cloning process involves a two step PCR reaction which generates the ORF of INSP115 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of plasmid 14210, 1.5 μl dNTPs (10 mM), 10 μl of 10× Pfx polymerase buffer, 1 μl MgSO4 (50 mM), 0.5 μl each of gene specific primer (100 μM) (INSP115-EX1 and INSP115-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

12.2 Subcloning of Gateway Compatible INSP115 ORF into Gateway Entry Vector pDONR221 and Expression Vectors pEAK12d and pDEST12.2

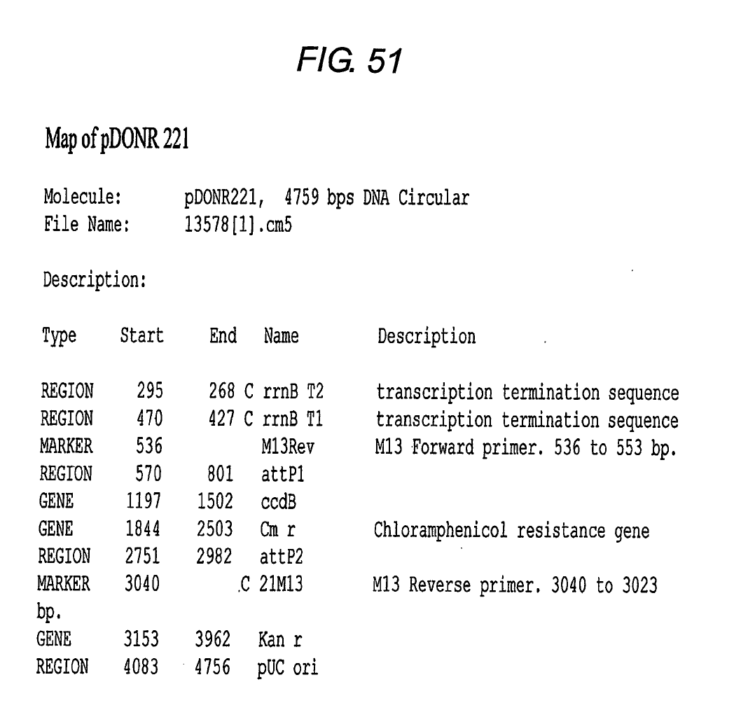

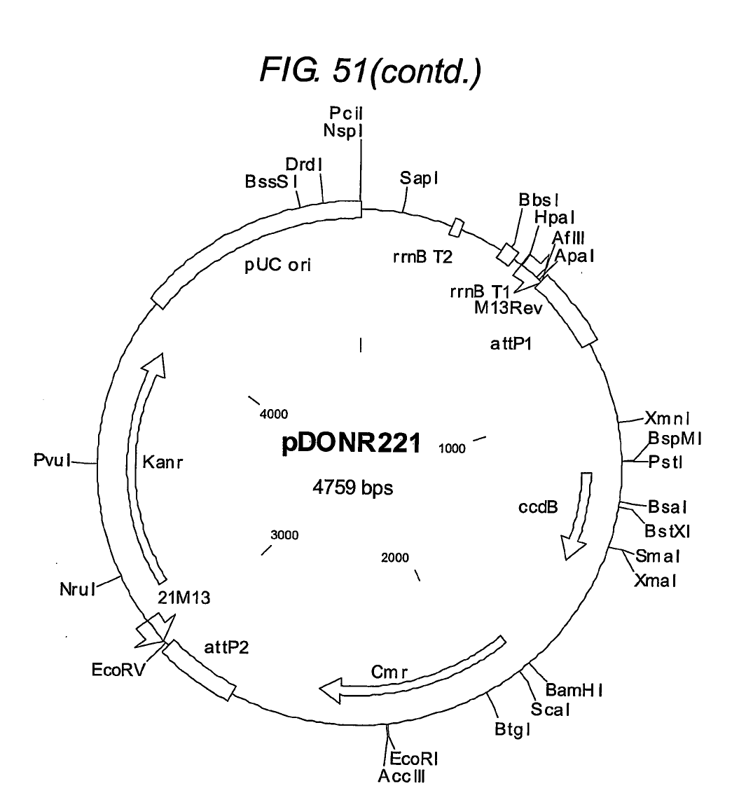

The second stage of the Gateway cloning process involves subcloning of the Gateway modified PCR product into the Gateway entry vector pDONR221 (Invitrogen, FIG. 51) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 4. Sequencing reactions were purified using Dye-Ex columns (Qiagen) or Montage SEQ 96 cleanup plates (Millipore cat. no. LSKS09624) then analyzed on an Applied Biosystems 3700 sequencer.

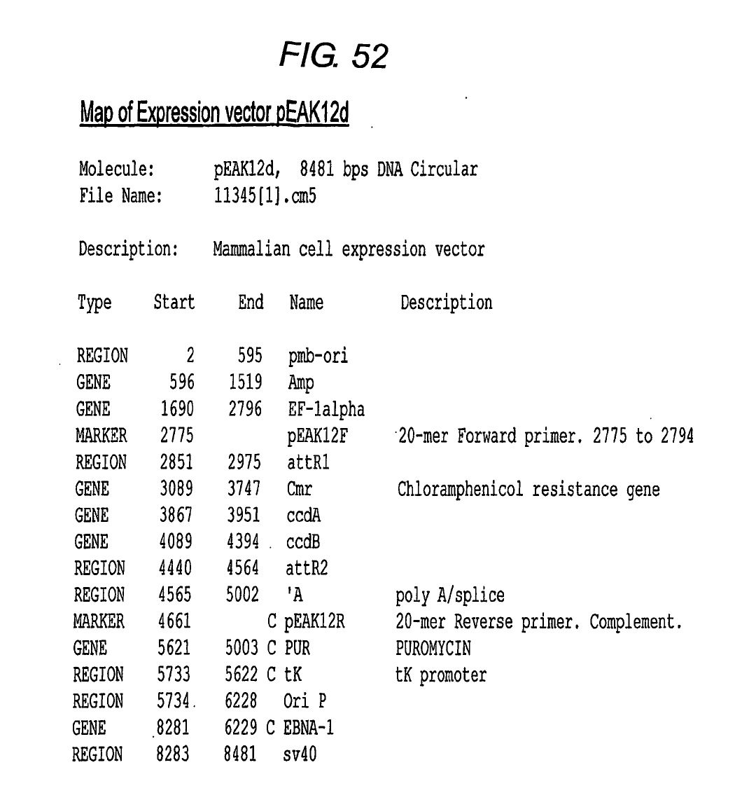

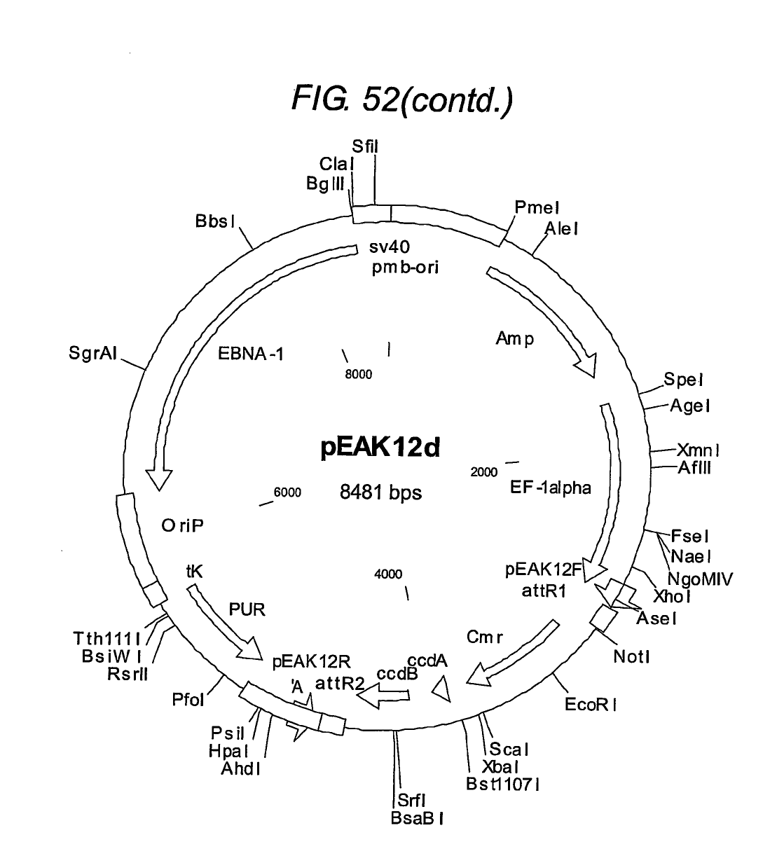

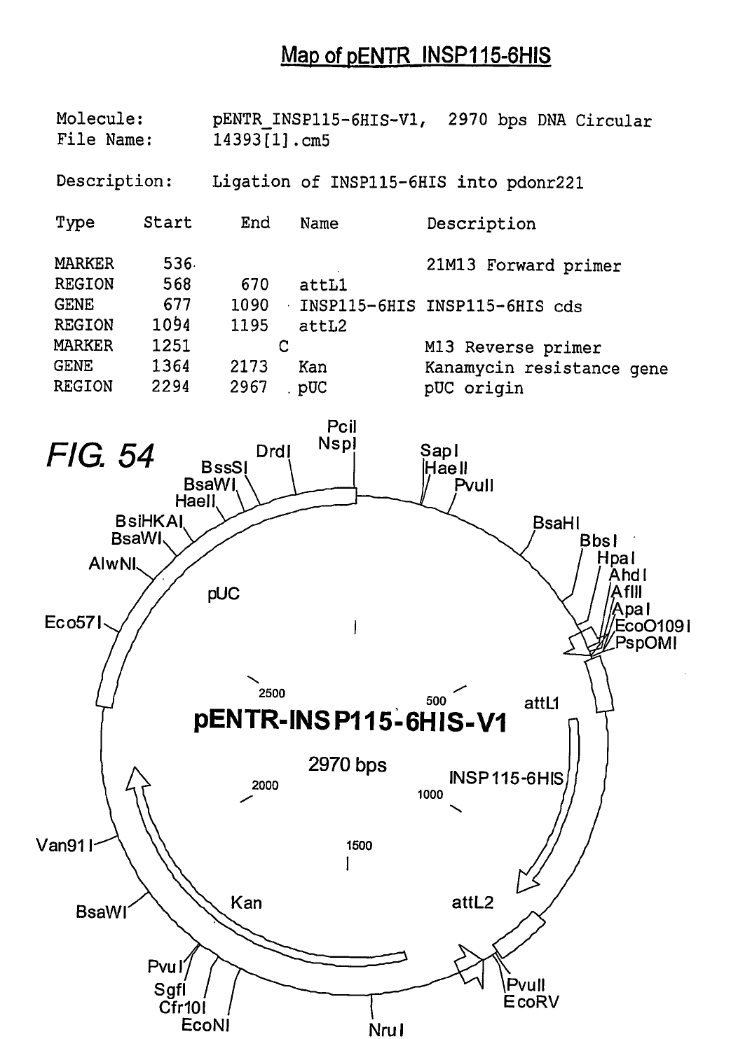

Plasmid eluate (2 μl or approx. 150 ng) from one of the clones which contained the correct sequence (pENTR-INSP115-6HIS, plasmid ID 14393, FIG. 54) was then used in a recombination reaction containing 1.5 μl of either pEAK12d vector or pDEST12.2 vector (FIGS. 52 & 53) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 4.

CsCl gradient purified maxi-prep DNA was prepared from a 500 ml culture of one of each of the sequence verified clones (pEAK12d_INSP115-6HIS, plasmid ID number 14398, FIG. 55, and pDEST12.2_INSP115-6HIS, plasmid ID 14410, FIG. 56) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

### Example 13

### Cloning of INSP116

INSP116 was a prediction for a 140 amino acid protein (420 bp) encoded in 5 coding exons. Using the full length cds to search public EST databases we identified several sequences which matched the INSP116 sequence. One sequence was chosen, GenBank Accession B1599941 corresponding to IMAGE cDNA clone 5302753, and the clone was purchased from ATCC. The insert sequence of the IMAGE clone was sequenced using sequencing primers T7 and T3 (Table 5). The insert sequence was found to contain the INSP116 cds. IMAGE clone 5302753 is plasmid database ID 14206. The full length of the IMAGE clone insert was not sequenced after the region corresponding to INSP116 had been identified.

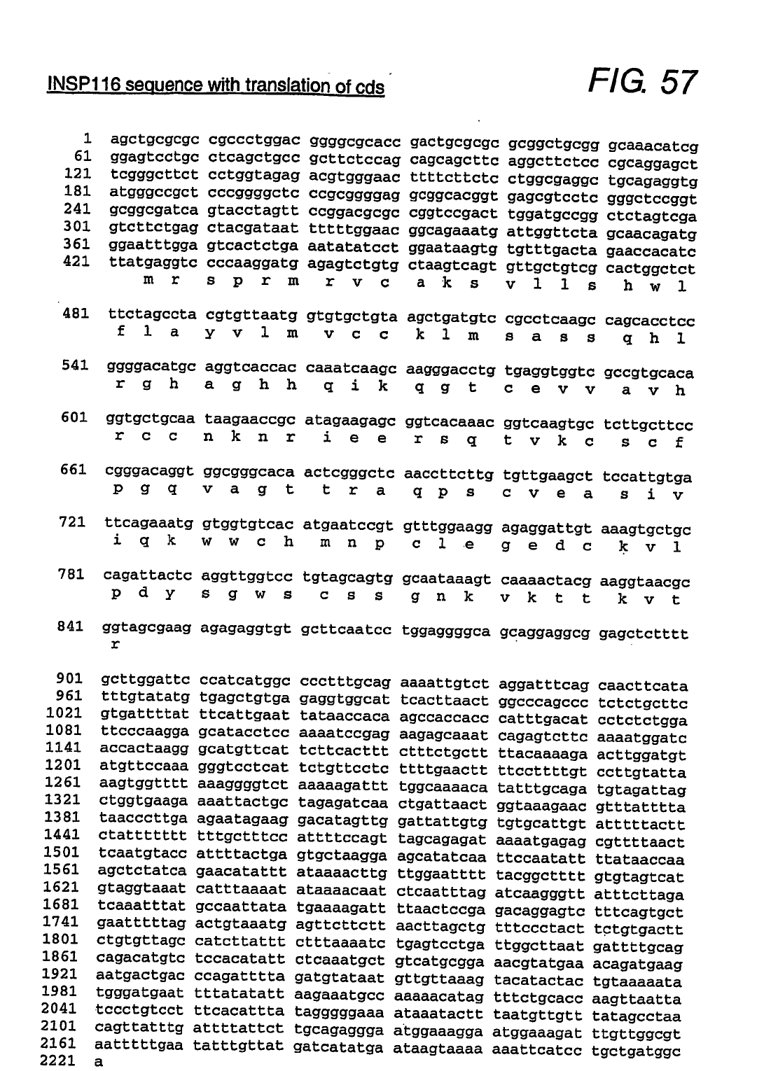

The sequence of the INSP116 prediction is shown in FIG. 57. The corresponding region of the IMAGE clone 5302753 was identical to the INSP116 cds at the nucleotide level.

TABLE 5
INSP116 cloning and sequencing primers

| | |
|---|---|
| Primer | Sequence (5′-3′) |

| | |
|---|---|
| T7 primer | TAA TAC GAC TCA CTA TAG G |
| T3 primer | ATT AAC CCT CAC TAA AGG |
| INSP116-EX1 | AA GCA GGC TTC GCC ACC ATG AGG TCC CCA |
| | AGG ATG AG |
| INSP116-EX2 | GTG ATG GTG ATG GTG CCG CGT TAC CTT CGT |
| | AGT TT |
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |

Underlined sequence = Kozak sequence
Bold = Stop codon
Italic sequence = His tag

### Example 14

### Construction of Mammalian Cell Expression Vectors for INSP116

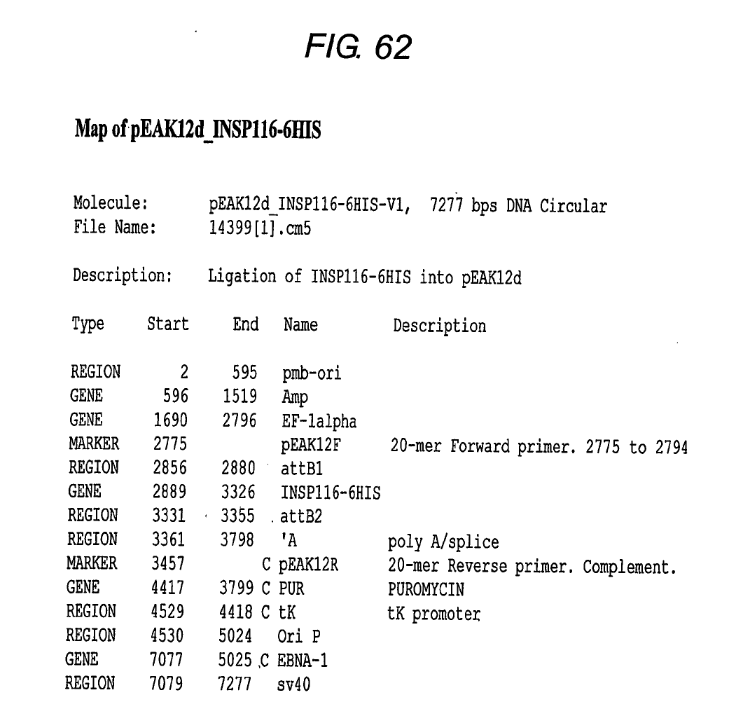

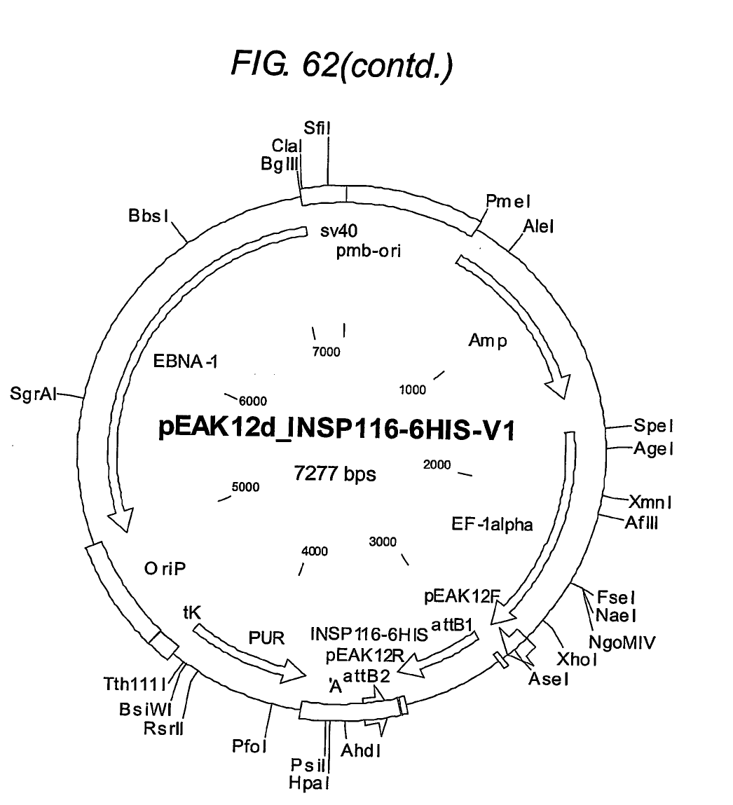

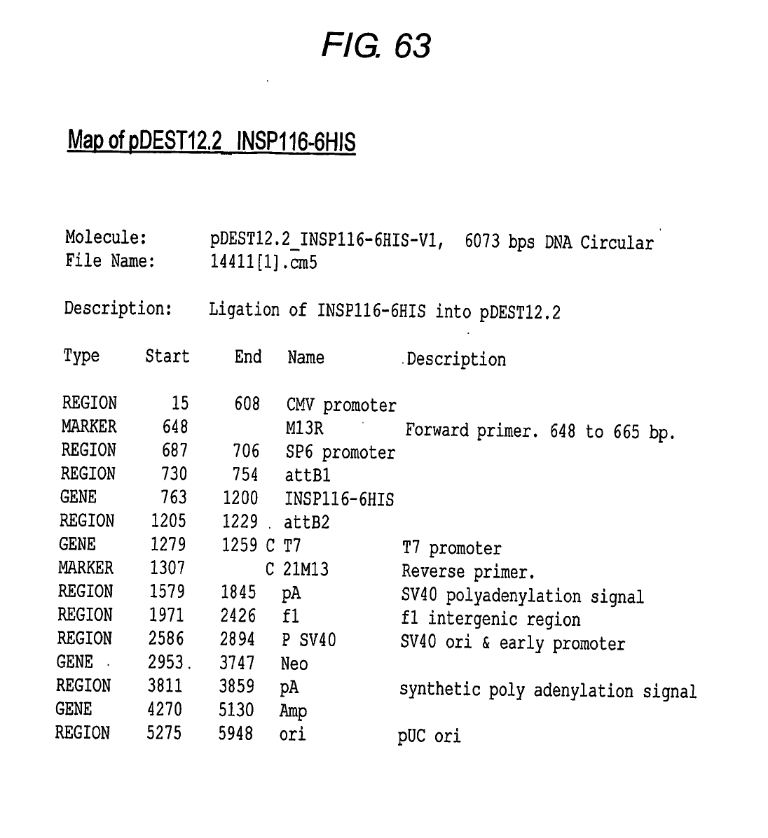

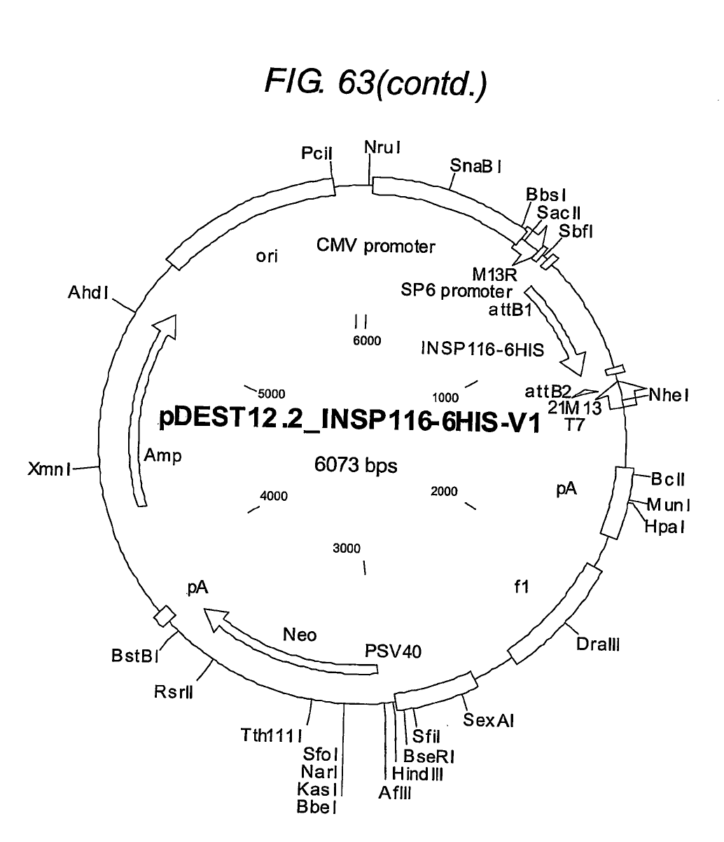

Plasmid 14206 was used as a PCR template to generate pEAK12d (FIG. 62) and pDEST12.2 (FIG. 63) expression clones containing the INSP116 ORF sequence with a 3′ sequence encoding a 6HIS tag using the Gateways cloning methodology (Invitrogen).

14.1 Generation of Gateway Compatible INSP116 ORF Fused to an in Frame 6HIS Tag Sequence.

The first stage of the Gateway cloning process involves a two step PCR reaction which generates the ORF of INSP116 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of plasmid 14206, 1.5 μl dNTPs (10 mM), 10 μl of 10× Pfx polymerase buffer, 1 μl MgSO4 (50 mM), 0.5 μl each of gene specific primer (100 μM) (INSP116-EX1 and INSP116-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), δ 41 of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

14.2 Subcloning of Gateway Compatible INSP116 ORF into Gateway Entry Vector pDONR221 and Expression Vectors pEAK12d and pDEST12.2

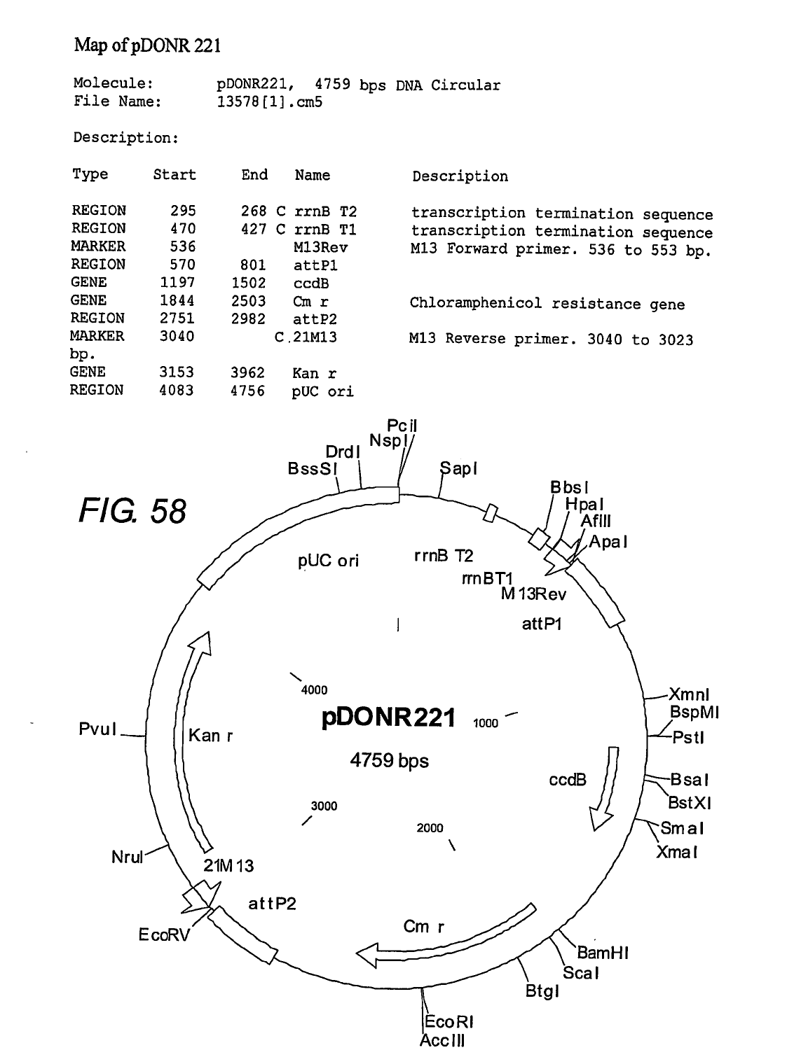

The second stage of the Gateway cloning process involves subcloning of the Gateway modified PCR product into the Gateway entry vector pDONR221 (Invitrogen, FIG. 58) as follows: 5 μl purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight, at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat. no. 4390246) according to the manufacturer's instructions. The primer sequences are shown in Table 5. Sequencing reactions were purified using Dye-Ex columns (Qiagen) or Montage SEQ 96 cleanup plates (Millipore cat. no. LSKS09624) then analyzed on an Applied Biosystems 3700 sequencer.

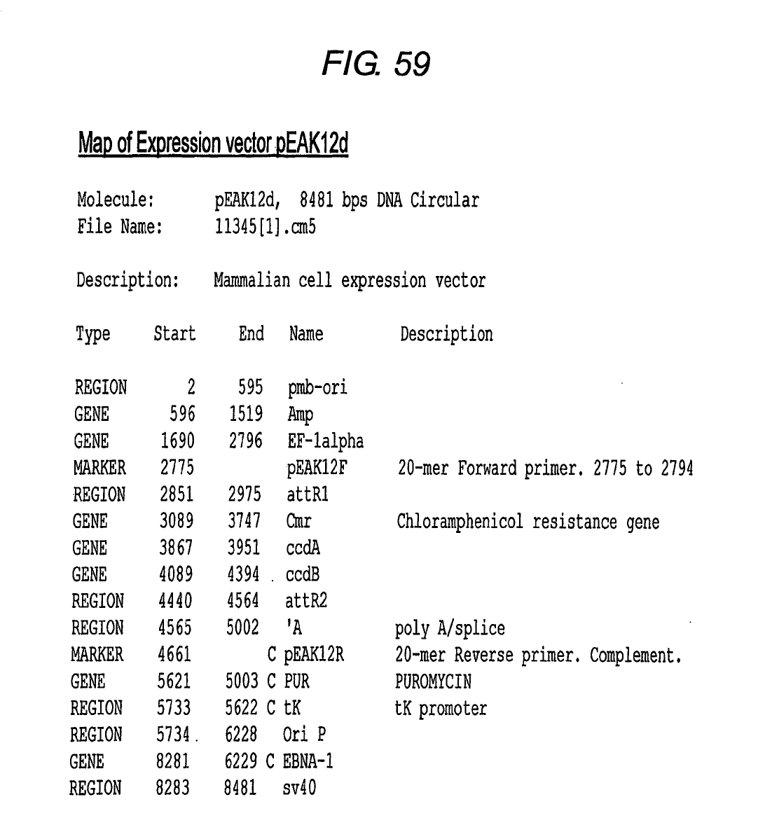

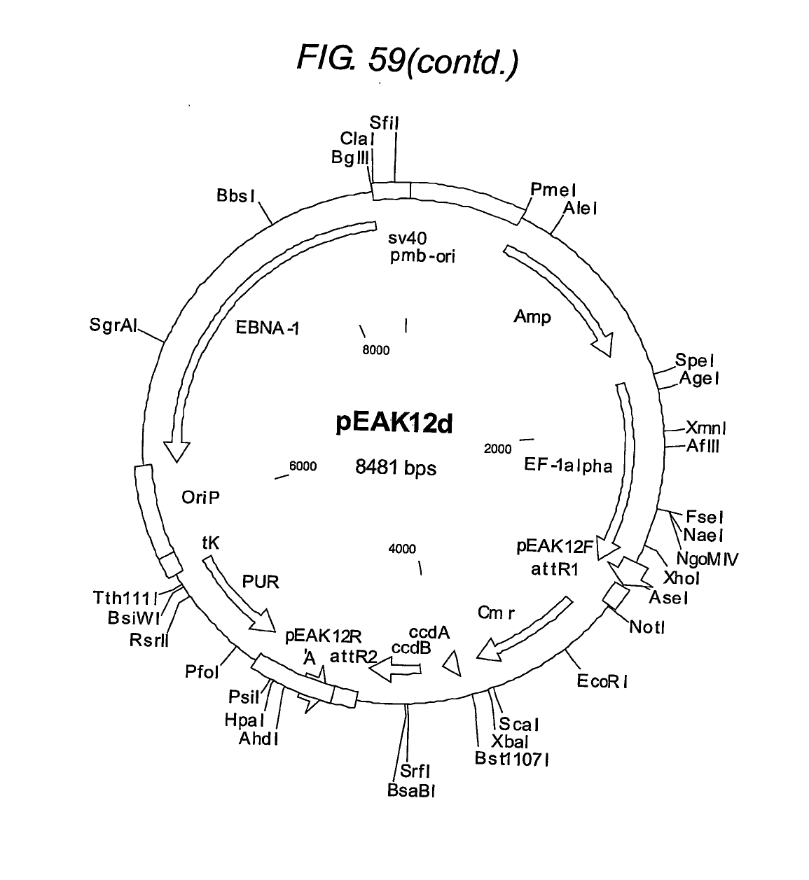

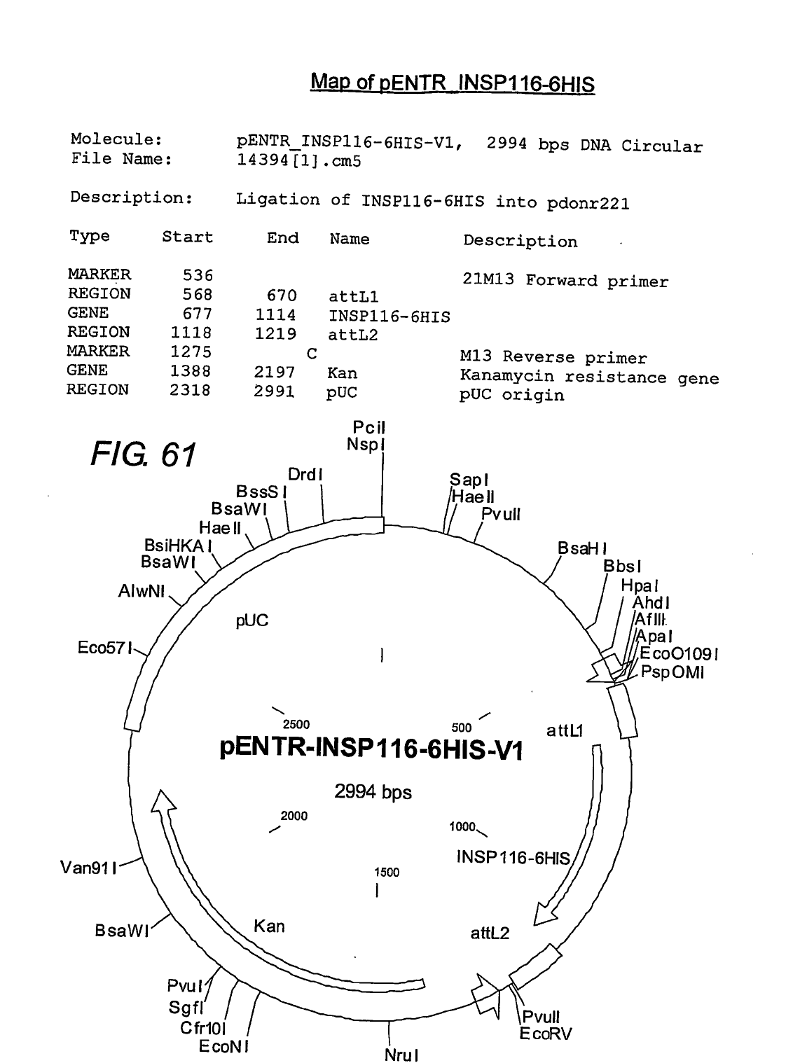

Plasmid eluate (2 μl or approx. 150 ng) from one of the clones which contained the correct sequence (pENTR-INSP116-6HIS, plasmid ID 14394, FIG. 61) was then used in a recombination reaction containing 1.5 μl of either pEAK12d vector or pDEST12.2 vector (FIGS. 59 & 60) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 5.

CsCl gradient purified maxi-prep DNA was prepared from a 500 ml culture of one of each of the sequence verified clones (pEAK12d_INSP116-6HIS, plasmid ID number 14399, FIG. 62, and pDEST12.2_INSP116-6HIS, plasmid ID 14411, FIG. 63) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

### Example 15

### Cloning of INSP117

15.1 Preparation of Human cDNA Templates

First strand cDNA was prepared from a variety of normal human tissue total RNA samples (Clontech, Ambion, and in-house samples) using Superscript II RNase H⁻ Reverse Transcriptase (Invitrogen) according to the manufacturer's protocol. Oligo (dT)₁₅ primer (1 μl at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and then chilled on ice. The contents were collected by brief centrifugation and 4 μl of 5× First-Strand Buffer, 2 μL 0.1 M DTT, and 1 μl RnaseOUT Recombinant Ribonuclease Inhibitor (40 units/μl, Invitrogen) were added. The contents of the tube were mixed gently and incubated at 42° C. for 2 min; then 1 μl (200 units) of SuperScript II enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min. The final 21 μl reaction mix was diluted by adding 179 μl sterile water to give a total volume of 200 μl. Human cDNA samples used as templates for the amplification of INSP117 were derived from placenta, eye and retina. The Universal Reference cell line mix cDNA sample (Stratagene) was also tested.

15.2 cDNA Libraries

Human cDNA libraries (in bacteriophage lambda (λ) vectors) were purchased from Clontech or made in-house in λ GT10 vectors. Bacteriophage λ DNA was prepared from small scale cultures of infected E. coli host strain using the Wizard Lambda Preps DNA purification system according to the manufacturer's instructions (Promega, Corporation, Madison Wis.). Human cDNA library samples used as templates for the amplification of INSP117 were derived from placenta and retina.

15.3 Gene Specific Cloning Primers for PCR

A pair of PCR primers having a length of between 18 and 25 bases were designed for amplifying the complete coding sequence of the virtual cDNA using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP117) with little or no none specific priming.

15.4 PCR Amplification of INSP117 from a Variety of Human cDNA Templates and Phase Library cDNA

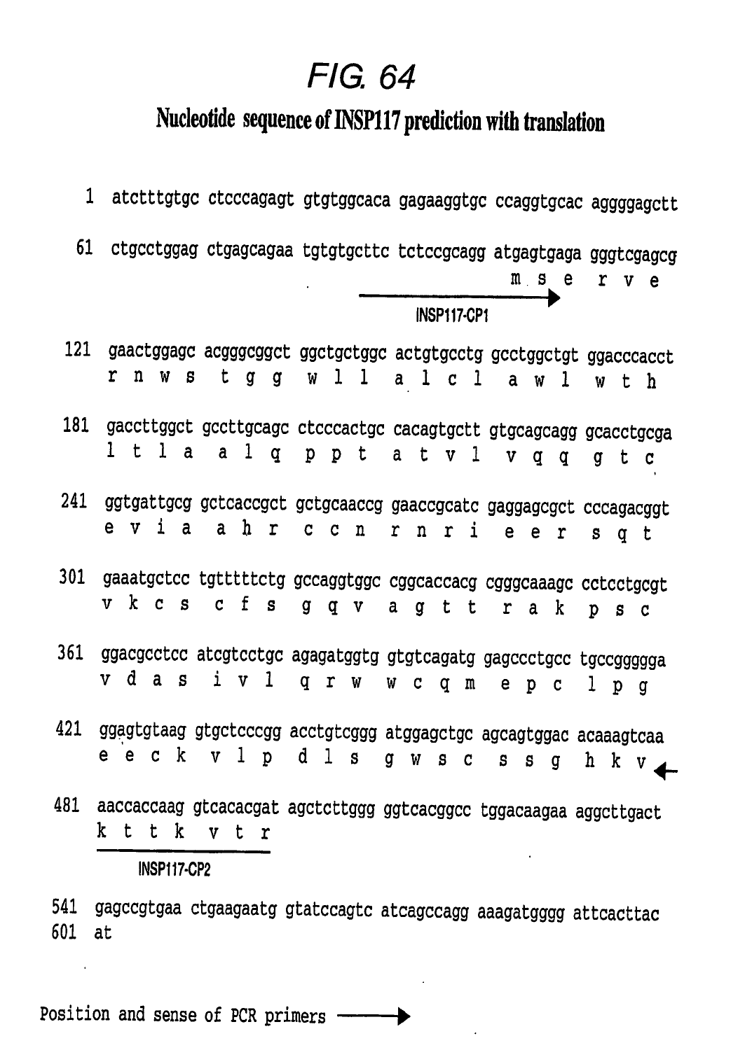

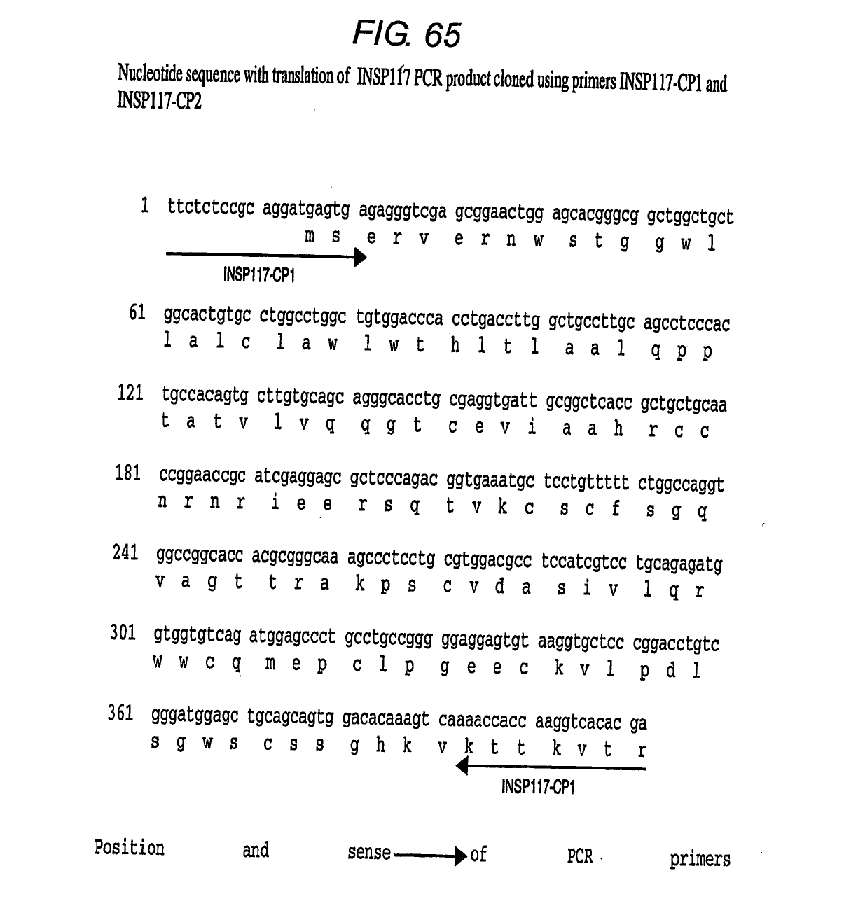

Gene-specific cloning primers (INSP117-CP1 and INSP117-CP2, FIG. 64, FIG. 65 and Table 6) were designed to amplify a cDNA fragment of 412 bp covering the entire 399 bp coding sequence of the INSP117 prediction. Interrogation of public EST sequence databases with the INSP117 prediction suggested that the sequence might be expressed in placenta, eye and retina cDNA templates. The gene-specific cloning primers INSP117-CP1 and INSP117-CP2 were therefore used with human cDNA samples listed in Section 1.1 and the phage library cDNA samples listed in Section 1.2 as the PCR templates. The PCR reactions were performed in a final volume of 50 μl containing 1× Platinum® Taq High Fidelity PCR buffer, 2 mM MgSO₄, 200 μM dNTPs, 0.2 μM of each cloning primer, 2.5 units of Platinum, Taq High Fidelity DNA polymerase (Invitrogen), 100 ng of human cDNA template, and either 0×, 1×, or 2× final concentration of PCRx Enhancer solution (Invitrogen). Thermal cycling was carried out using an MJ Research DNA Engine programmed as follows: 94° C., 2 min; 40 cycles of 94° C., 30 sec, 55° C., 30 sec, 68° C., 1 min, followed by 1 cycle at 68° C. for 7 min and a holding cycle at 4° C.

All 50 μl of each amplification product was visualized on a 0.8% agarose gel in 1× TAE buffer (Invitrogen) and a single PCR product was seen migrating at approximately the predicted molecular mass in the sample corresponding to the retina first strand cDNA template. This PCR product was purified using the Qiagen MinElute DNA Purification System (Qiagen). The PCR product was eluted in 10 μl of EB buffer (10 mM Tris.Cl, pH 8.5) and subcloned directly.

TABLE 6
INSP117 cloning and sequencing primers

| | |
|---|---|
| Primer | Sequence (5′-3′) |

| | |
|---|---|
| INSP117-CP1 | TTC TCT CCG CAG GAT GAG TGA |
| INSP117-CP2 | TCG TGT GAC CTT GGT GGT TT |
| INSP117-EX1 | AAG CAG GCT TCG CCA CCA TGA GTG AGA GGG |
| | TCG AGC G |
| INSP117-EX2 | GTG ATG GTG ATG GTG TCG TGT GAC CTT GGT |
| | GGT TT |
| GCP Forward | G GGG ACA AGT TTG TAC AAA AAA GCA GGC |
| | TTC GCC ACC |
| GCP Reverse | GGG GAC CAC TTT GTA CAA GAA AGC TGG GTT |
| | TCA ATG GTG ATG GTG ATG GTG |
| pEAK12F | GCC AGC TTG GCA CTT GAT GT |
| pEAK12R | GAT GGA GGT GGA CGT GTC AG |
| 21M13 | TGT AAA ACG ACG GCC AGT |
| M13REV | CAG GAA ACA GCT ATG ACC |
| T7 | TAA TAC GAC TCA CTA TAG G |
| SP6 | ATT TAG GTG ACA CTA TAG |

Underlined sequence = Kozak sequence
Bold = Stop codon
Italic sequence = His tag

15.5 Subcloning of PCR Products

The PCR product was subcloned into the topoisomerase I modified cloning vector (pCRII-TOPO) using the TA cloning kit purchased from the Invitrogen Corporation using the conditions specified by the manufacturer. Briefly, 4 μl of gel purified PCR product from the human retina cDNA amplification was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

15.6 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles T7 primer, 20 pmoles of SP6 primer, 1 unit of AmpliTaq™ (Perkin Elmer) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR reaction products were analyzed on 1% agarose gels in 1×TAE buffer. Colonies which gave the expected PCR product size (412 bp cDNA+187 bp due to the multiple cloning site or MCS) were grown up overnight at 37° C. in 5 ml L-Broth (LB) containing ampicillin (100 μg/ml), with shaking at 220 rpm.

15.7 Plasmid DNA Preparation and Sequencing

Miniprep plasmid DNA was prepared from the 5 ml culture using a Qiaprep Turbo 9600 robotic system (Qiagen) or Wizard Plus SV Minipreps kit (Promega cat. no. 1460) according to the manufacturer's instructions. Plasmid DNA was eluted in 100 μl of sterile water. The DNA concentration was measured using an Eppendorf BO photometer. Plasmid DNA (200-500 ng) was subjected to DNA sequencing with the T7 primer and SP6 primer using the BigDye Terminator system (Applied Biosystems cat. no. 4390246) according to the manufacturer's instructions. The primer sequences are shown in Table 6. Sequencing reactions were purified using Dye-Ex columns (Qiagen) or Montage SEQ 96 cleanup plates (Millipore cat. no. LSKS09624) then analyzed on an Applied Biosystems 3700 sequencer.

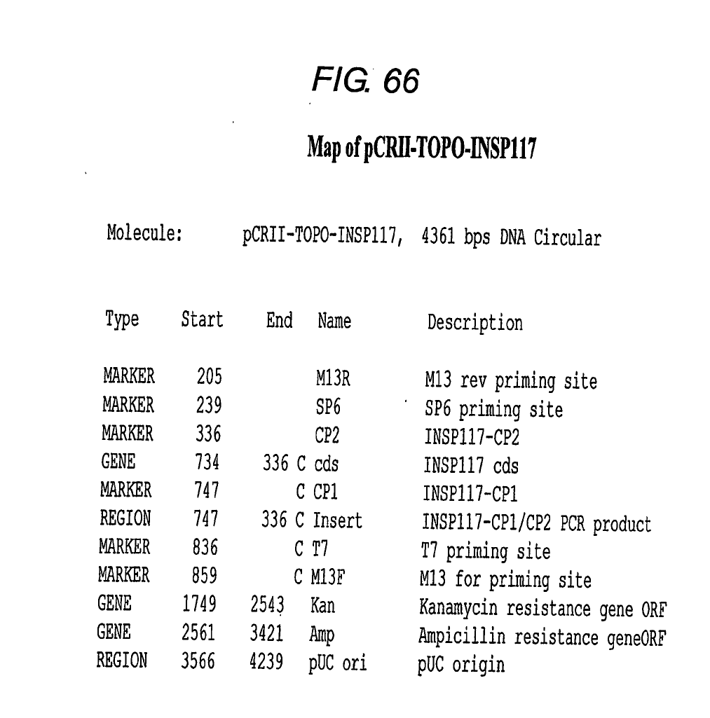

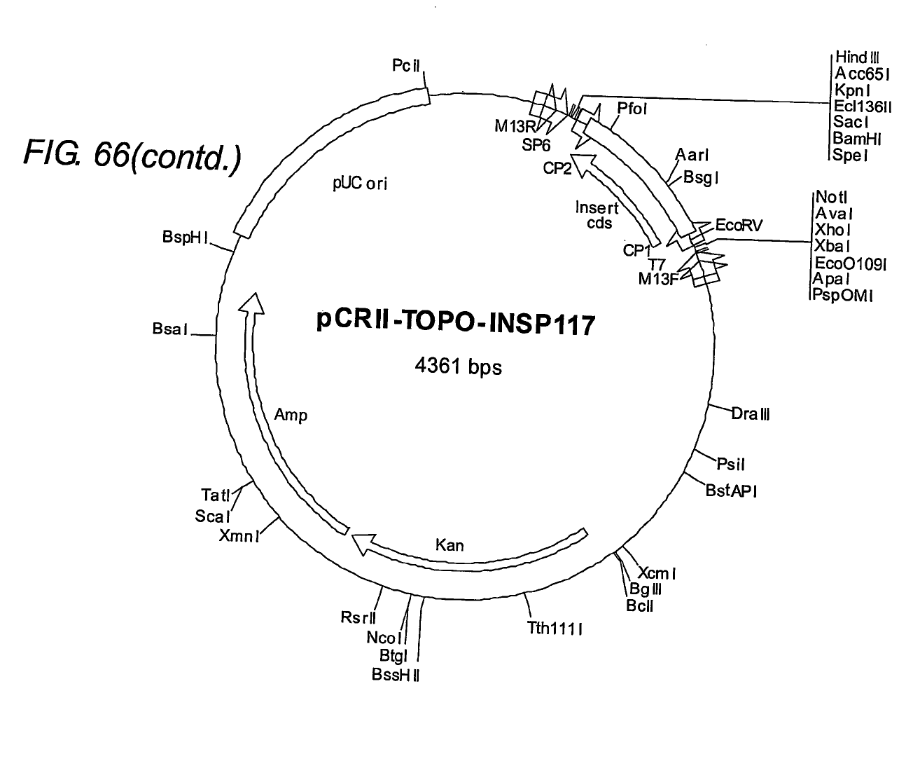

Sequence analysis identified a clone containing a 100% match to the predicted INSP117 sequence. The sequence of the cloned cDNA fragment is shown in FIG. 65. The plasmid map of the cloned PCR product (pCRII-TOPO-INSP117) (plasmid ID.14417) is shown in FIG. 66.

### Example 16

### Construction of Mammalian Cell Expression Vectors for INSP117

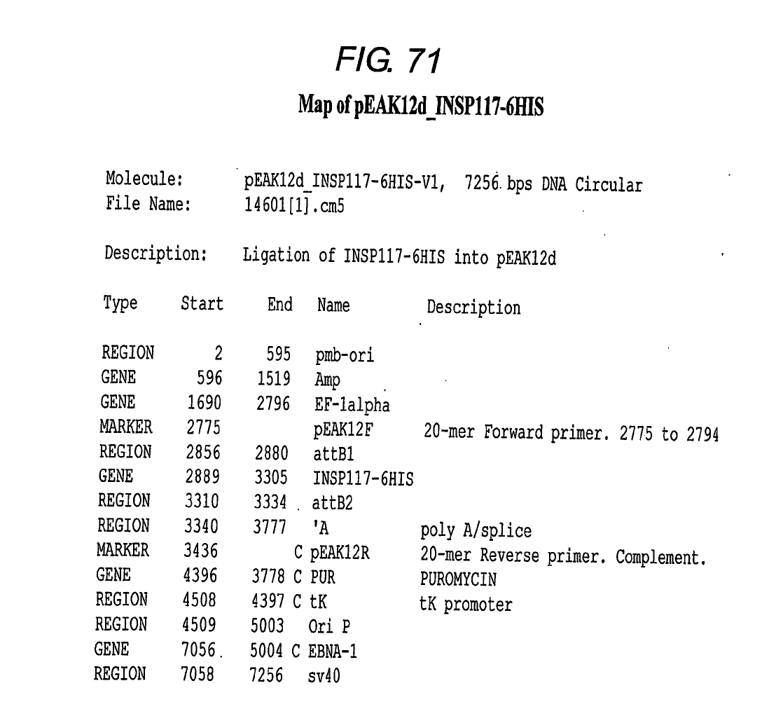

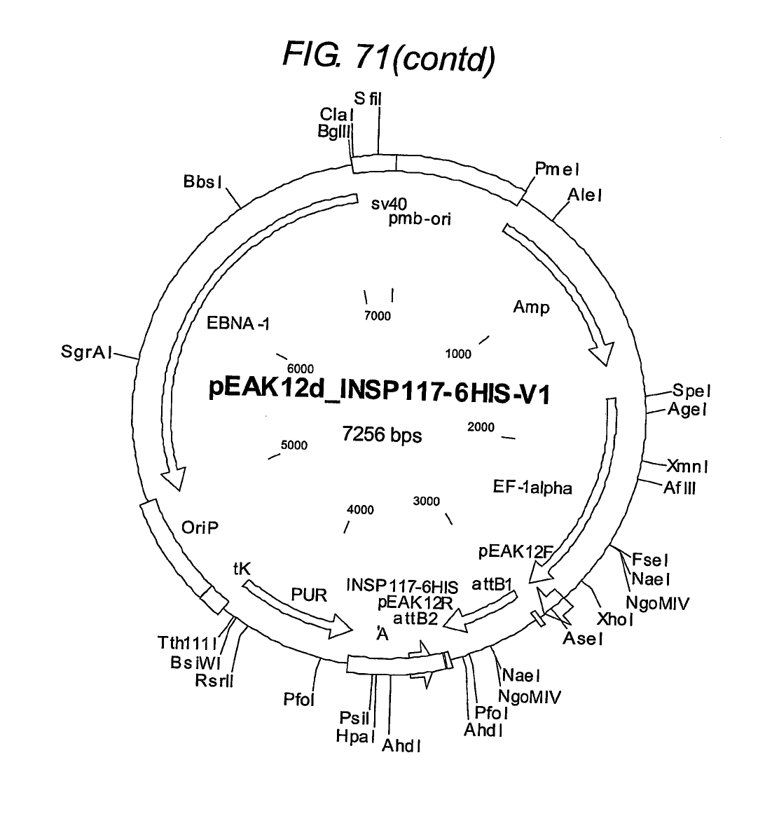

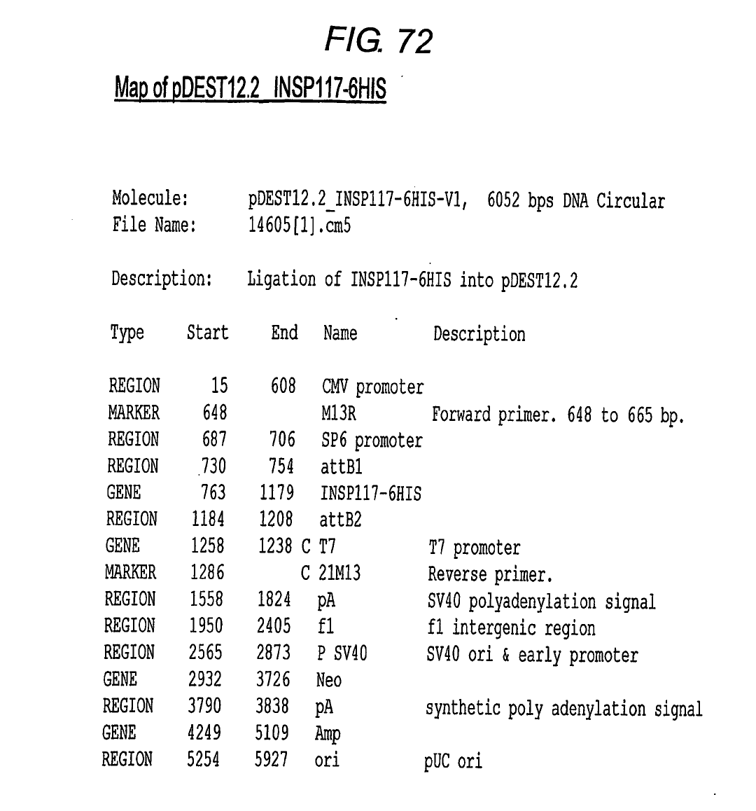

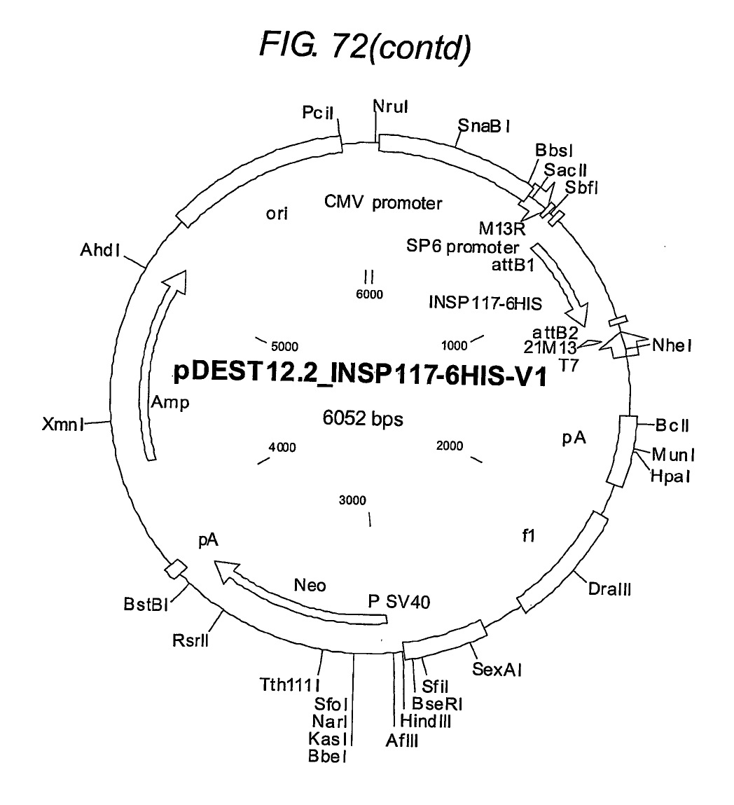

Plasmid 14417 was used as a PCR template to generate pEAK12d (FIG. 71) and pDEST12.2 (FIG. 72) expression clones containing the INSP117 ORF sequence with a 3′ sequence encoding a 6HIS tag using the Gateway™ cloning methodology (Invitrogen).

16.1 Generation of Gateway Compatible INSP117 ORF Fused to an in Frame 6HIS Tag Sequence.

The first stage of the Gateway cloning process involves a two step PCR reaction which generates the ORF of INSP117 flanked at the 5′ end by an attB1 recombination site and Kozak sequence, and flanked at the 3′ end by a sequence encoding an in frame 6 histidine (6HIS) tag, a stop codon and the attB2 recombination site (Gateway compatible cDNA). The first PCR reaction (in a final volume of 50 μl) contains: 1 μl (40 ng) of plasmid 14417, 1.5 μl dNTPs (10 mM), 10 μl of 10× Pfx polymerase buffer, 1 μl MgSO4 (50 mM), 0.5 μl each of gene specific primer (100 μM) (INSP117-EX1 and INSP117-EX2), and 0.5 μl Platinum Pfx DNA polymerase (Invitrogen). The PCR reaction was performed using an initial denaturing step of 95° C. for 2 min, followed by 12 cycles of 94° C. for 15 s; 55° C. for 30 s and 68° C. for 2 min; and a holding cycle of 4° C. The amplification products were visualized on 0.8% agarose gel in 1× TAE buffer (Invitrogen) and a product migrating at the predicted molecular mass was purified from the gel using the Wizard PCR Preps DNA Purification System (Promega) and recovered in 50 μl sterile water according to the manufacturer's instructions.

The second PCR reaction (in a final volume of 50 μl) contained 10 μl purified PCR 1 product, 1.5 μl dNTPs (10 mM), 5 μl of 10× Pfx polymerase buffer, 1 μl MgSO4 (50 mM), 0.5 μl of each Gateway conversion primer (100 μM) (GCP forward and GCP reverse) and 0.5 μl of Platinum Pfx DNA polymerase. The conditions for the 2nd PCR reaction were: 95° C. for 1 min; 4 cycles of 94° C., 15 sec; 50° C., 30 sec and 68° C. for 2 min; 25 cycles of 94° C., 15 sec; 55° C., 30 sec and 68° C., 2 min; followed by a holding cycle of 4° C. PCR products were gel purified using the Wizard PCR prep DNA purification system (Promega) according to the manufacturer's instructions.

16.2 Subcloning of Gateway Compatible INSP117 ORF into Gateway Entry Vector pDONR221 and Expression Vectors pEAK12d and pDEST12.2

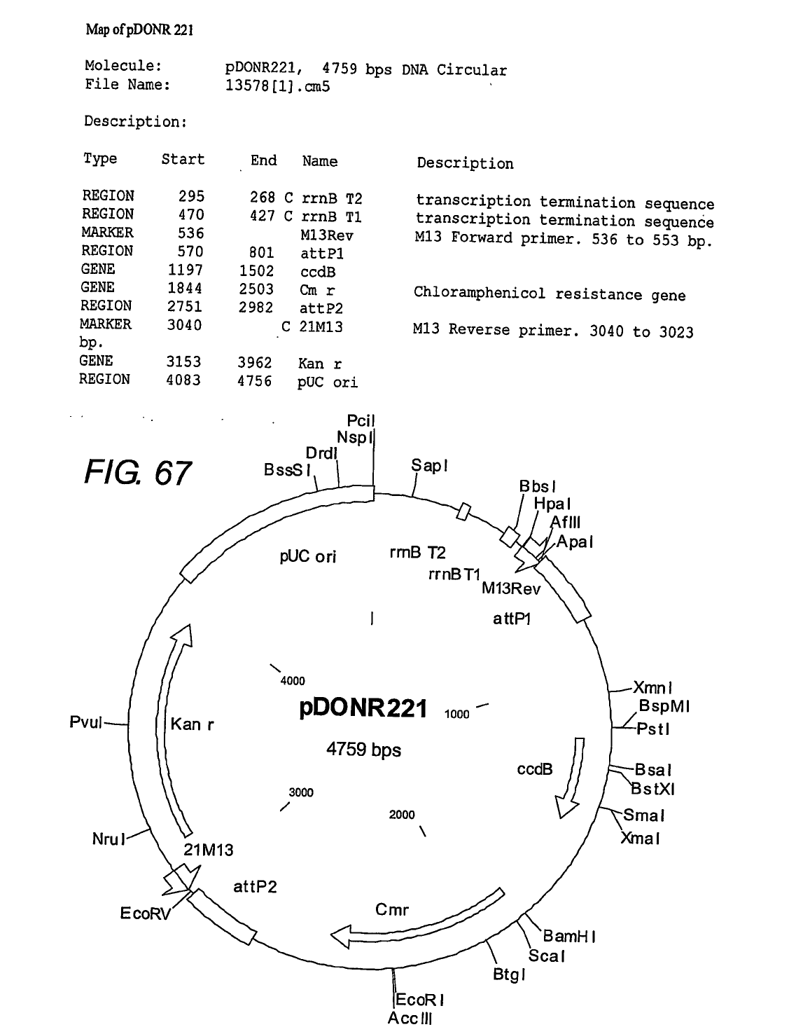

The second stage of the Gateway cloning process involves subcloning of the Gateway modified PCR product into the Gateway entry vector pDONR221 (Invitrogen, FIG. 67) as follows: 5 μl of purified product from PCR2 were incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of proteinase K 1 μl (2 μg/μl) and incubated at 37° C. for a further 10 min. An aliquot of this reaction (1 μl) was used to transform E. coli DH10B cells by electroporation as follows: a 25 μl aliquot of DH10B electrocompetent cells (Invitrogen) was thawed on ice and 1 μl of the BP reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing kanamycin (40 μg/ml) and incubated overnight at 37° C.

Plasmid mini-prep DNA was prepared from 5 ml cultures from 6 of the resultant colonies using a Qiaprep Turbo 9600 robotic system (Qiagen). Plasmid DNA (150-200 ng) was subjected to DNA sequencing with 21M13 and M13Rev primers using the BigDyeTerminator system (Applied Biosystems cat no. 4390246) according to the manufacturer's instructions. The primer sequences are shown in Table 6. Sequencing reactions were purified using Dye-Ex columns (Qiagen) or Montage SEQ 96 cleanup plates (Millipore cat. no. LSKS09624) then analyzed on an Applied Biosystems 3700 sequencer.

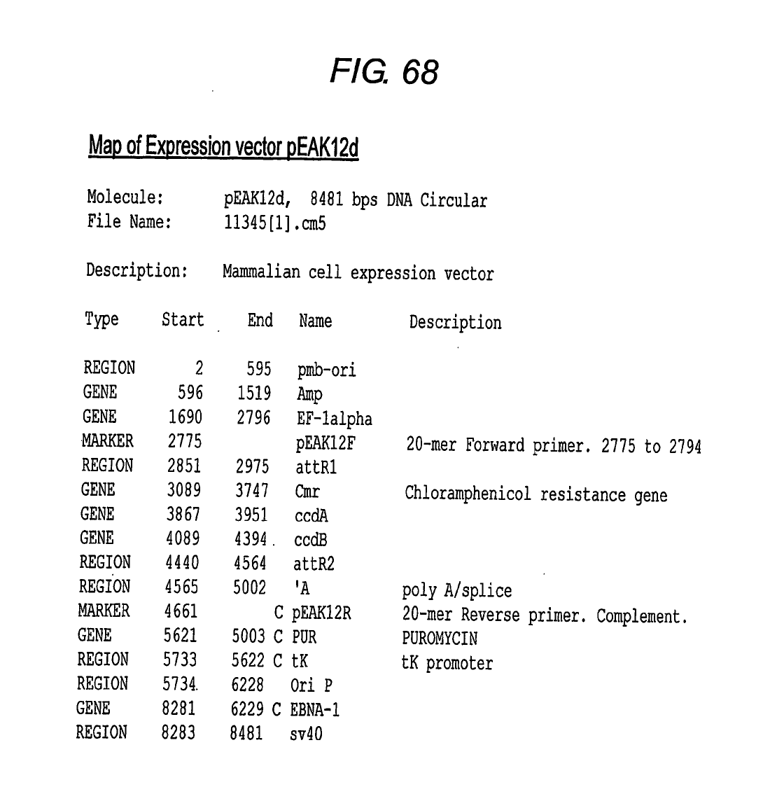

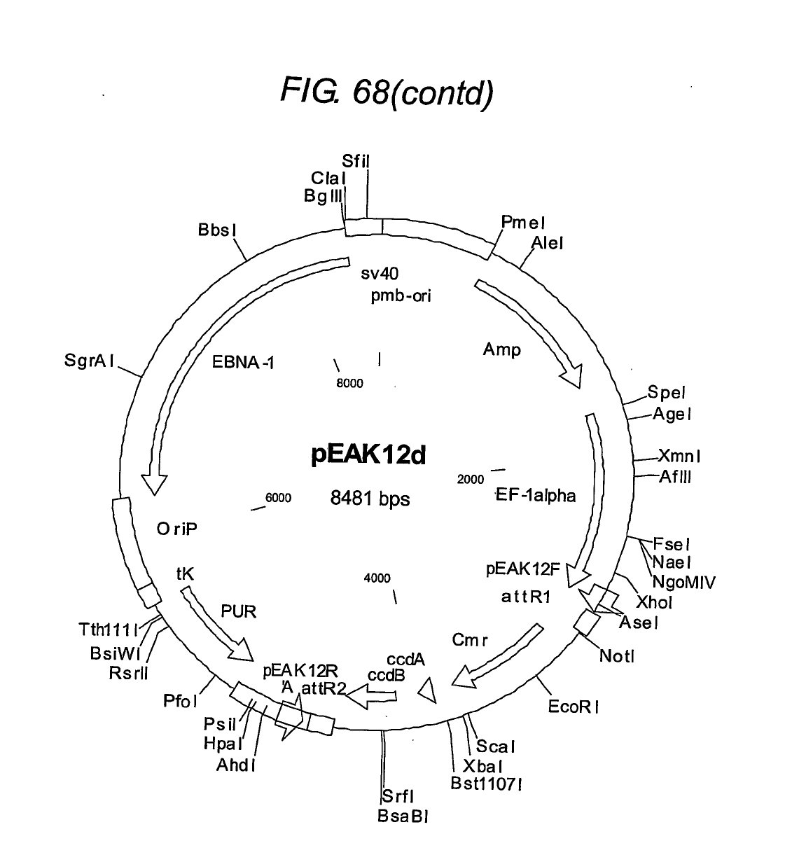

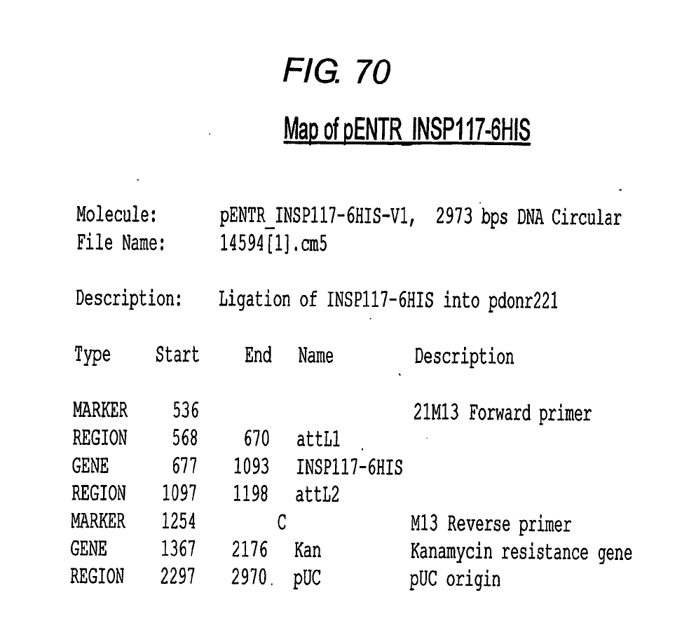

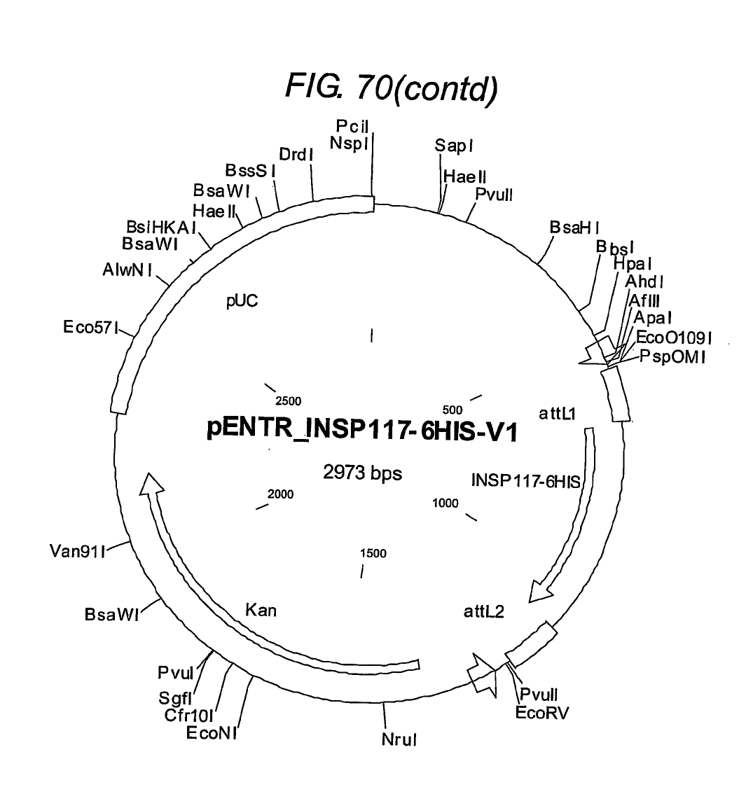

Plasmid eluate (2 μl or approx. 150 ng) from one of the clones which contained the correct sequence (pENTR_INSP117-6HIS, plasmid ID 14594, FIG. 70) was then used in a recombination reaction containing 1.5 μl of either pEAK12d vector or pDEST12.2 vector (FIGS. 68 & 69) (0.1 μg/μl), 2 μl LR buffer and 1.5 μl of LR clonase (Invitrogen) in a final volume of 10 μl. The mixture was incubated at RT for 1 h, stopped by addition of proteinase K (2 μg) and incubated at 37° C. for a further 10 min. An aliquot of this reaction (1 ul) was used to transform E. coli DH10B cells by electroporation as follows: a 25 μl aliquot of DH10B electrocompetent cells (Invitrogen) was thawed on ice and 1 μl of the LR reaction mix was added. The mixture was transferred to a chilled 0.1 cm electroporation cuvette and the cells electroporated using a BioRad Gene-Pulser™ according to the manufacturer's recommended protocol. SOC media (0.5 ml) which had been pre-warmed to room temperature was added immediately after electroporation. The mixture was transferred to a 15 ml snap-cap tube and incubated, with shaking (220 rpm) for 1 h at 37° C. Aliquots of the transformation mixture (10 μl and 50 μl) were then plated on L-broth (LB) plates containing ampicillin (100 μg/ml) and incubated overnight at 37° C.

Plasmid mini-prep DNA was prepared from 5 ml cultures from 6 of the resultant colonies subcloned in each vector using a Qiaprep Turbo 9600 robotic system (Qiagen). Plasmid DNA (200-500 ng) in the pEAK12d vector was subjected to DNA sequencing with pEAK12F and pEAK12R primers as described above. Plasmid DNA (200-500 ng) in the pDEST12.2 vector was subjected to DNA sequencing with 21M13 and M13Rev primers as described above. Primer sequences are shown in Table 6.

CsCl gradient purified maxi-prep DNA was prepared from a 500 ml culture of one of each of the sequence verified clones (pEAK12d_INSP117-6HIS, plasmid ID number 14601, FIG. 71, and pDEST12.2_INSP117-6HIS, plasmid ID 14605, FIG. 72) using the method described by Sambrook J. et al., 1989 (in Molecular Cloning, a Laboratory Manual, 2^{nd} edition, Cold Spring Harbor Laboratory Press), Plasmid DNA was resuspended at a concentration of 1 μg/μl in sterile water (or 10 mM Tris-HCl pH 8.5) and stored at −20° C.

### Example 17

### Expression and Purification of INSP113, INSP114, INSP115, INSP116 and INSP117

Further experiments may now be performed to determine the tissue distribution and expression levels of the INSP113, INSP114, INSP115, INSP116 and INSP117 polypeptides in vivo, on the basis of the nucleotide and amino acid sequences disclosed herein.

The presence of the transcripts for INSP113, INSP114, INSP115, INSP116 and INSP117 may be investigated by PCR of cDNA from different human tissues. The INSP113, INSP114, INSP115, INSP116 and INSP117 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a −ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase (−ve RT control). PCR reactions are set up for each tissue on the reverse transcribed RNA samples and the minus RT controls. INSP113, INSP114, INSP115, INSP116 and INSP117-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP113, INSP114, INSP115, INSP116 and INSP117 transcripts, not only those generated as described above.

The tissue distribution pattern of the INSP113, INSP114, INSP115, INSP116 and INSP117 polypeptides will provide further useful information in relation to the function of those polypeptides.

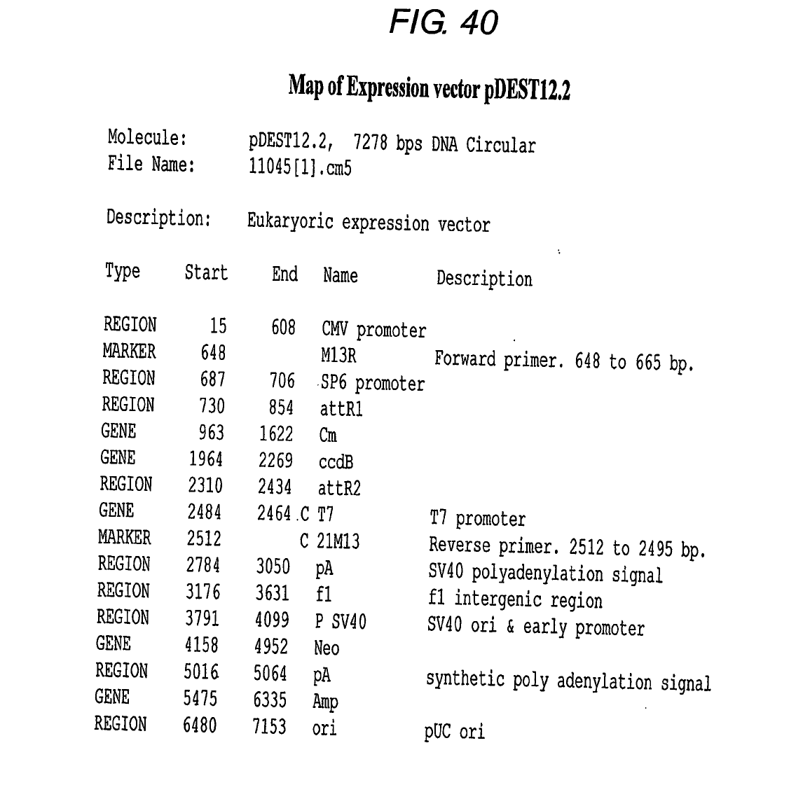

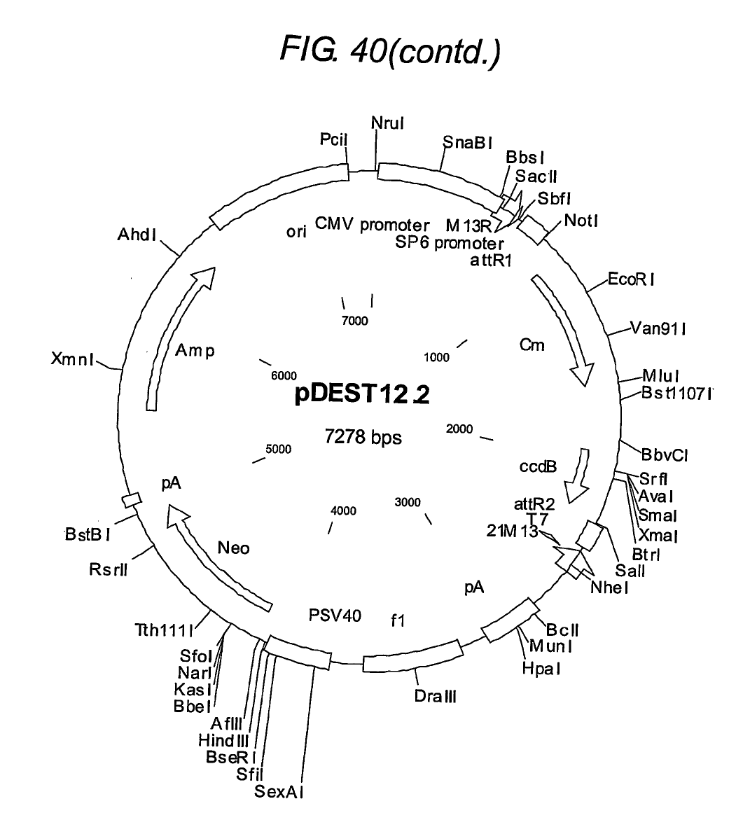

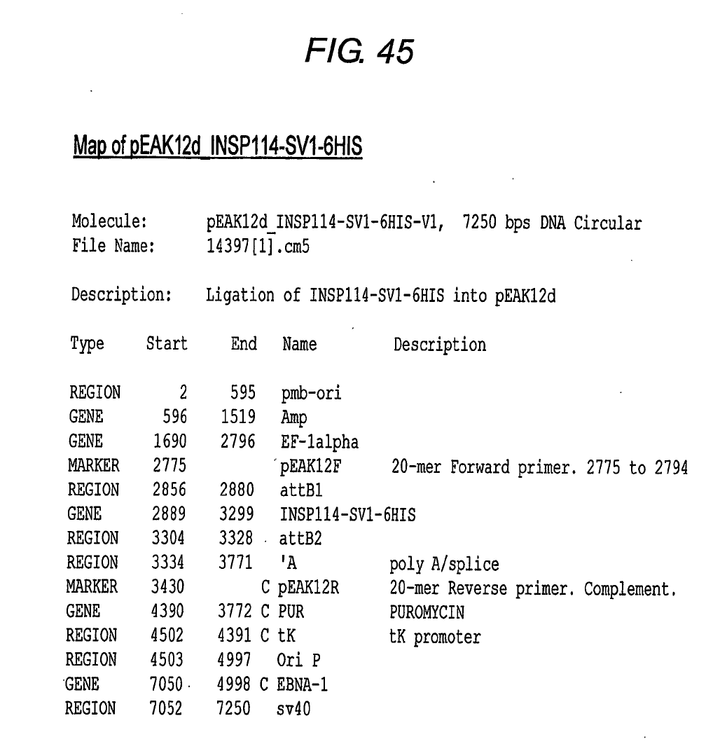

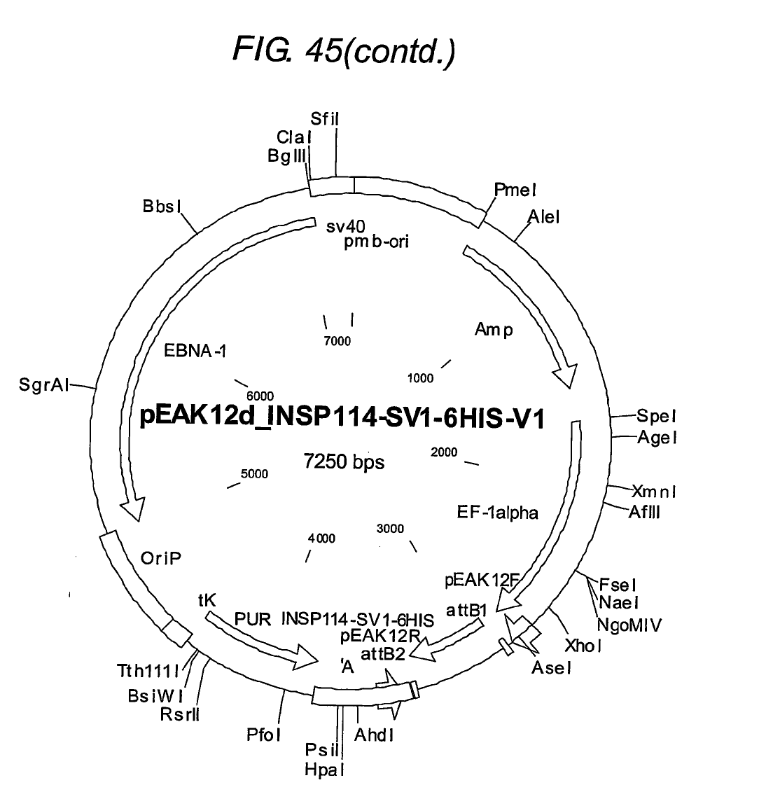

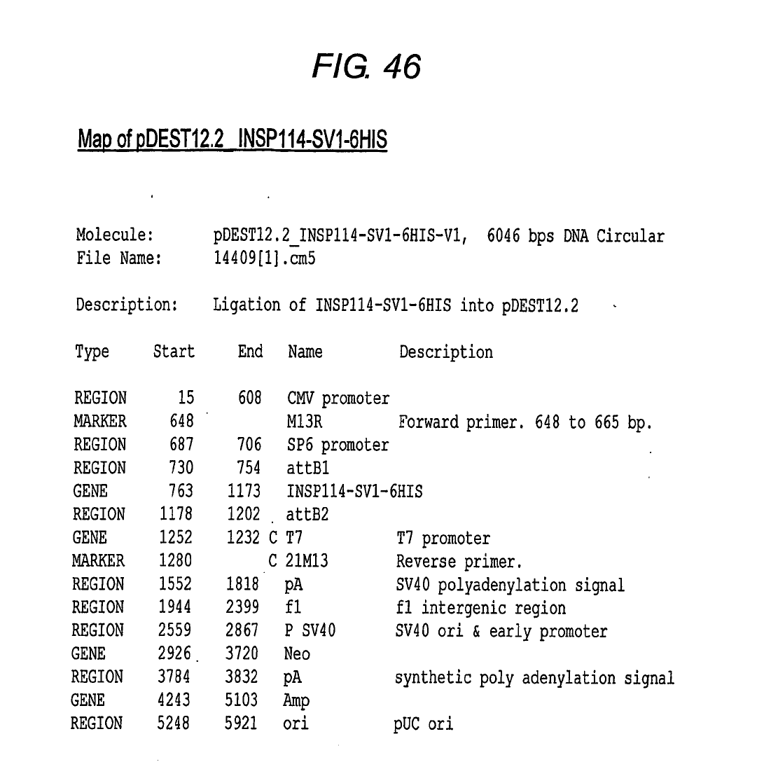

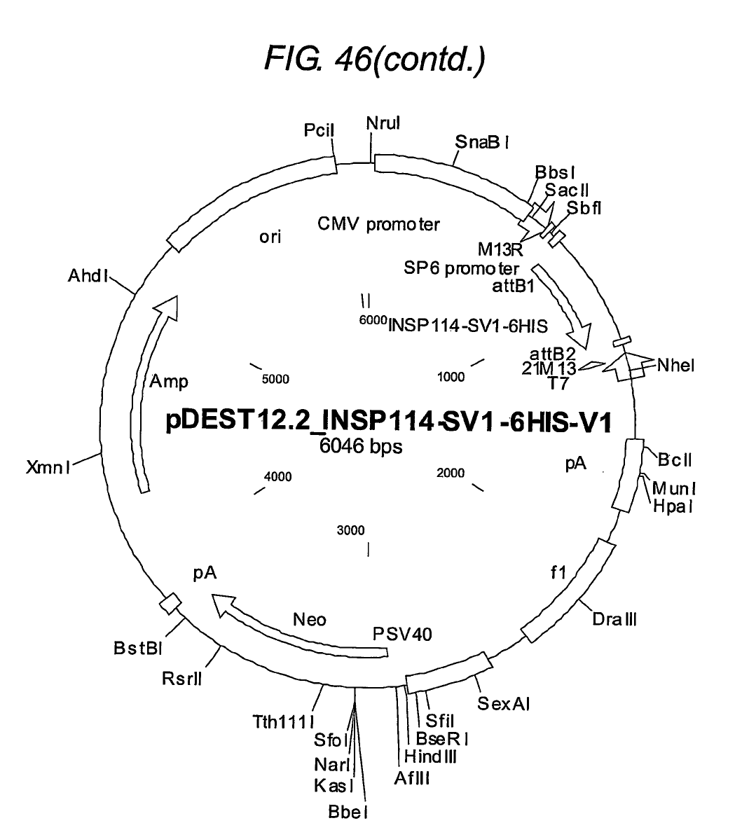

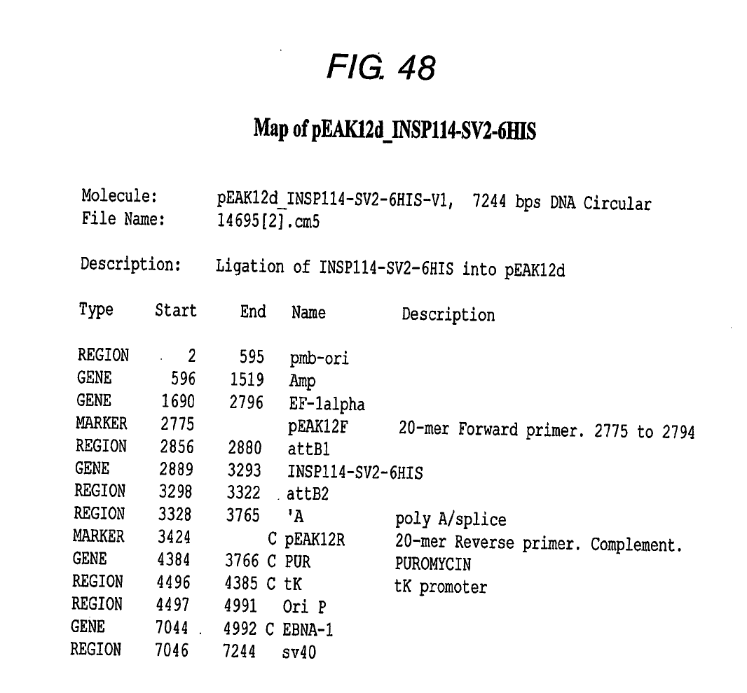

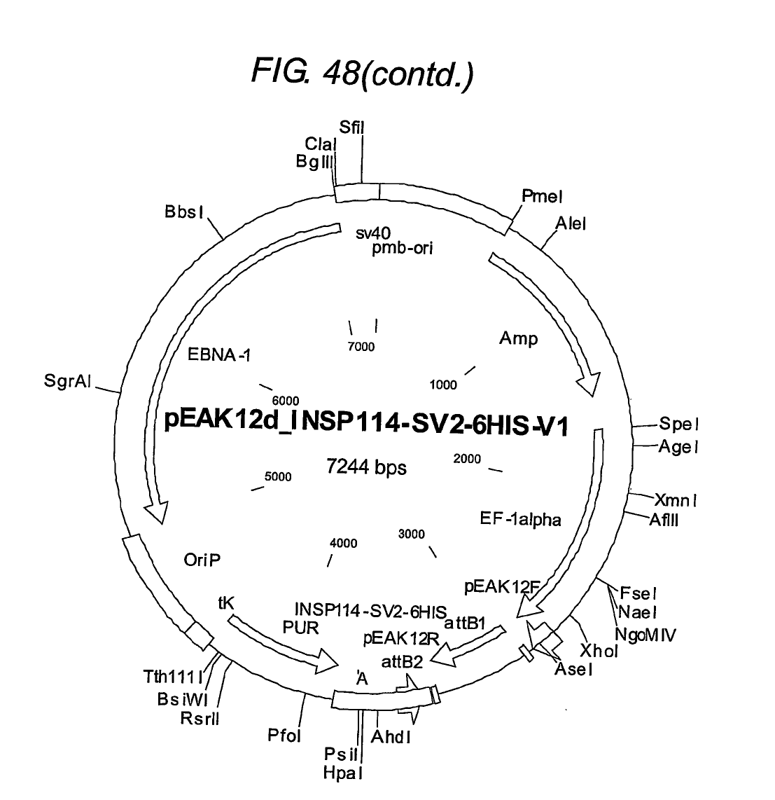

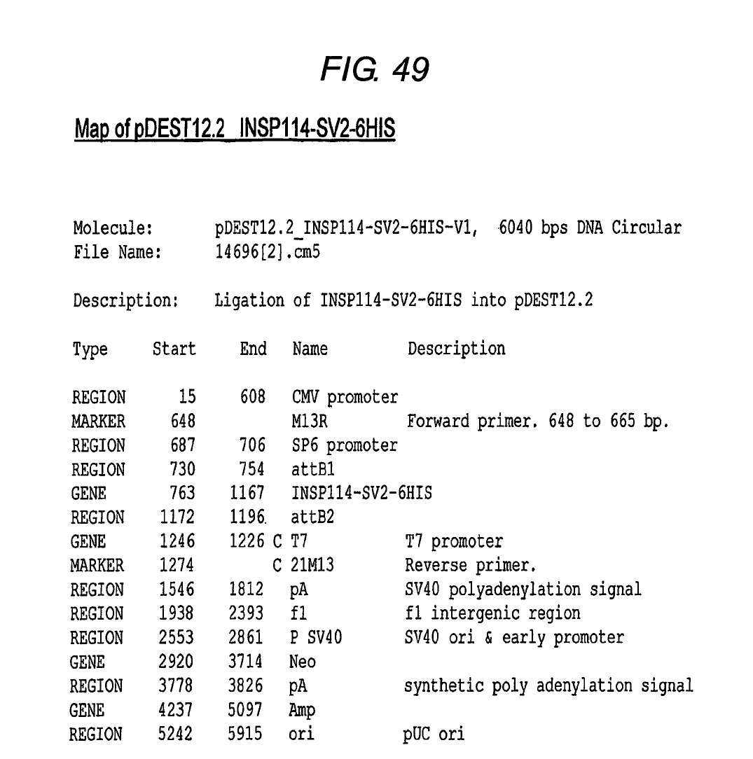

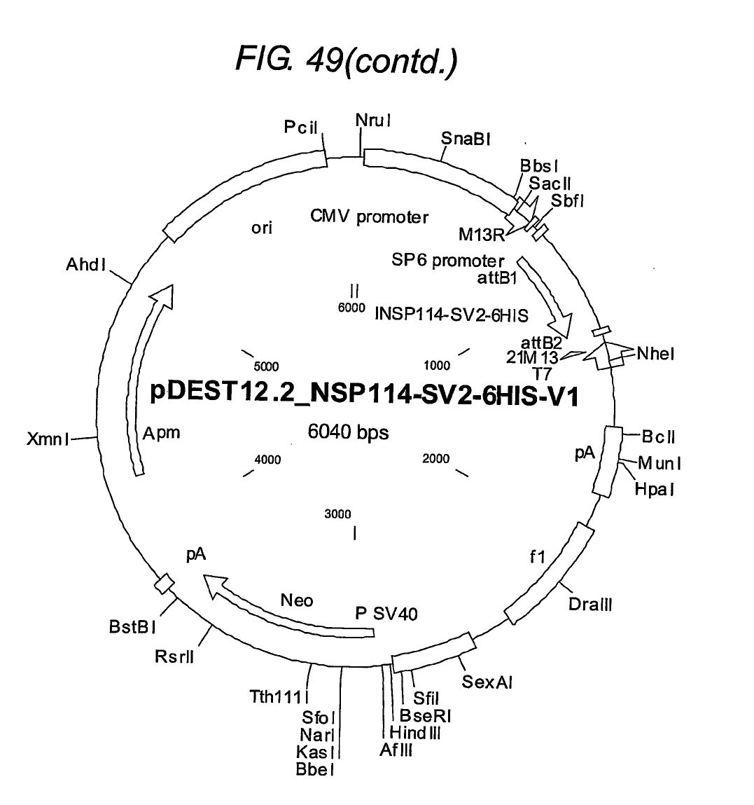

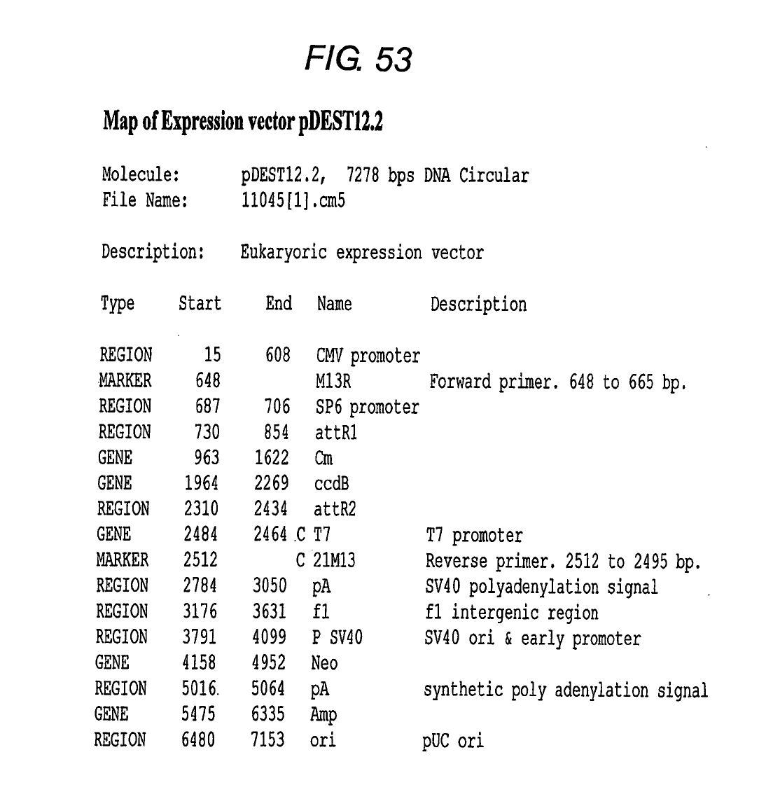

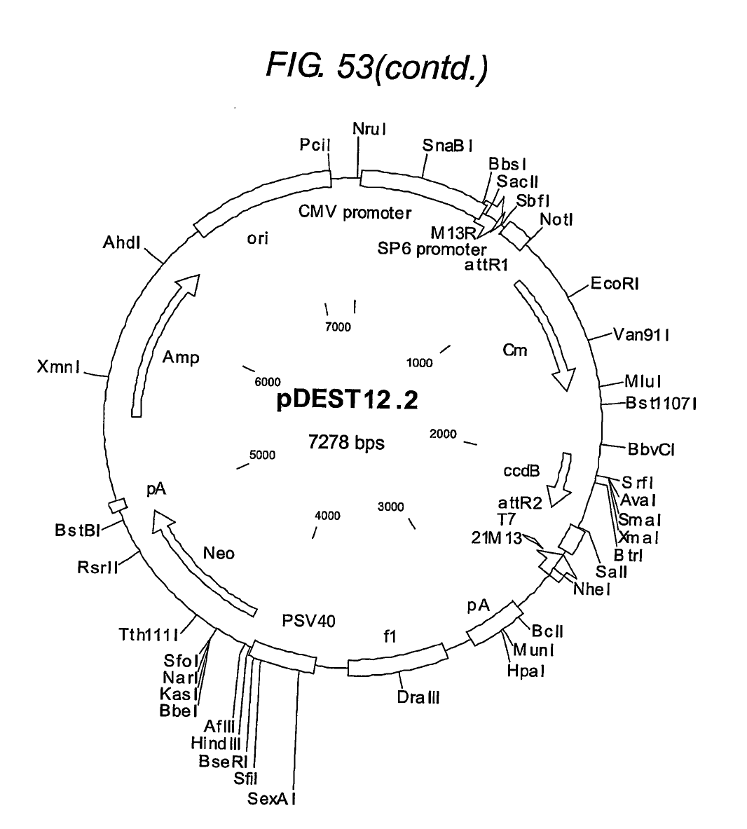

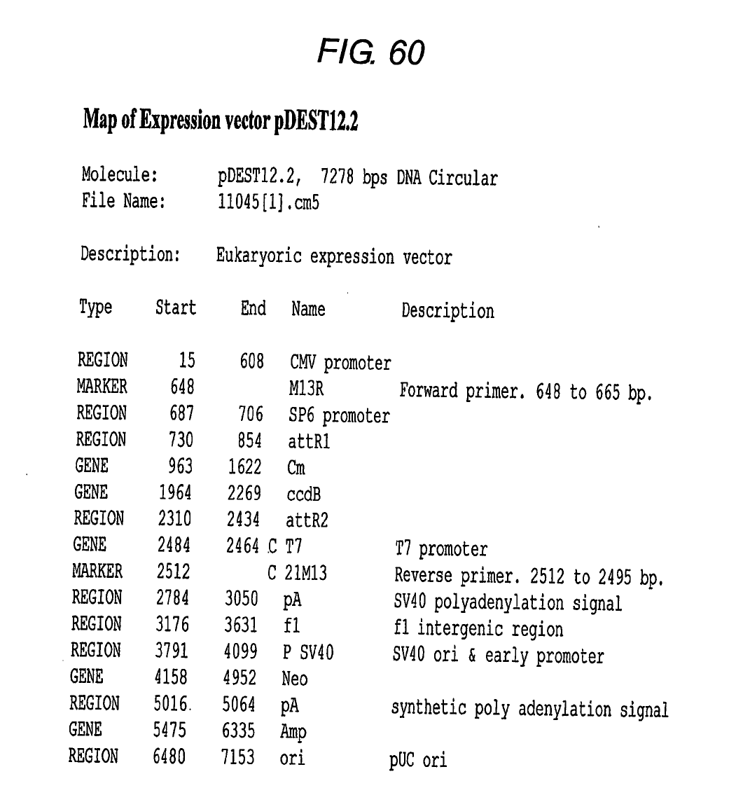

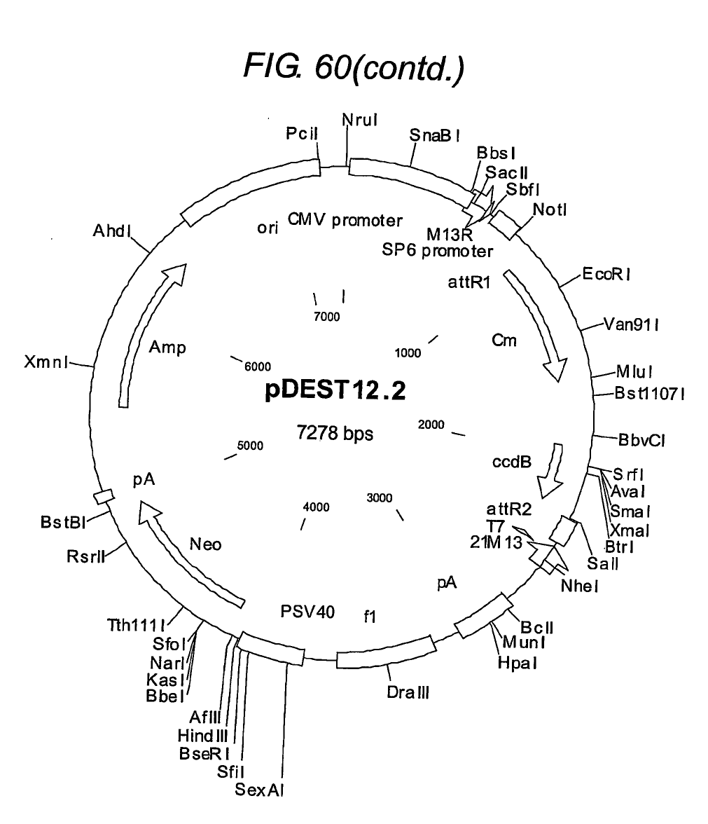

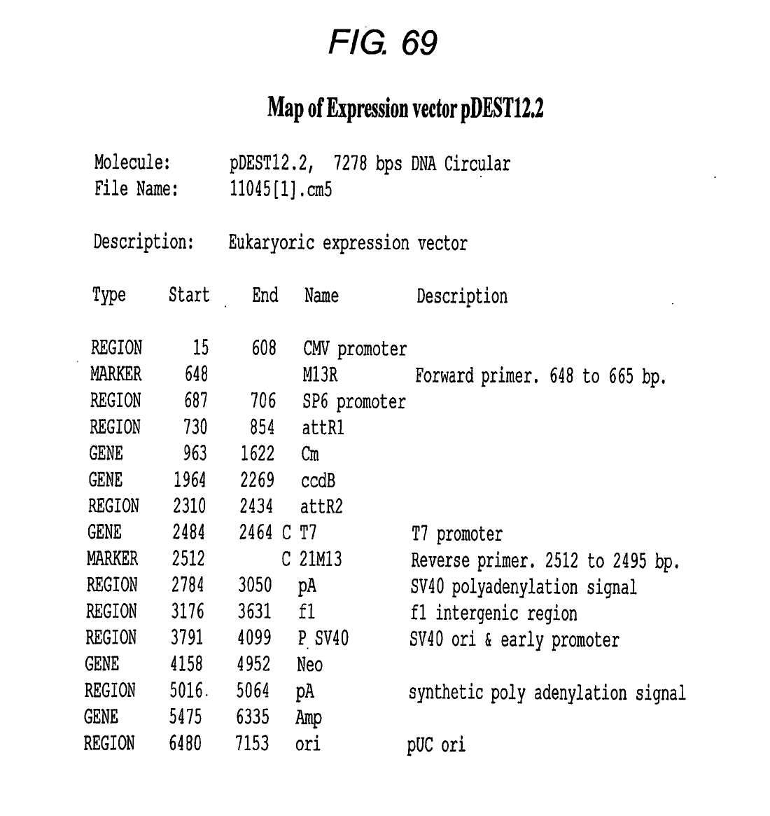

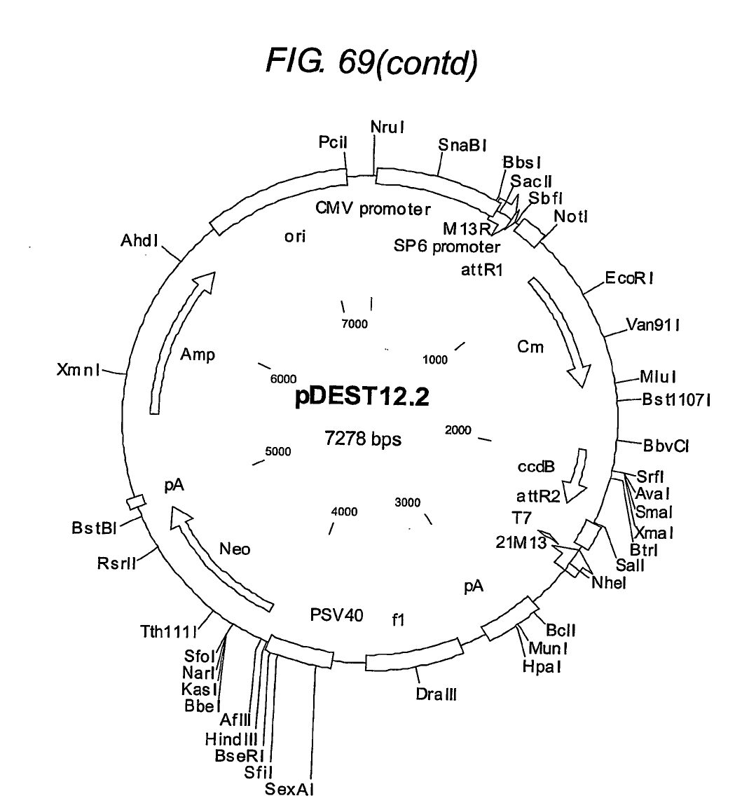

In addition, further experiments may now be performed using the pCR4-TOPO-INSP113 (FIG. 18), pCR4-TOPO-INSP113sv (FIG. 19), pDONR (FIG. 20), pEAK12d (FIG. 21), pDEST12.2 (FIG. 22), pENTR-INSP113-6HIS (FIG. 23), pENTR-INSP113sv-6HIS (FIG. 24), pEAK12d-INSP113-6HIS (FIG. 25), pEAK12d-INSP113sv-6HIS (FIG. 26), pDEST12.2-INSP113-6HIS (FIG. 27), pDEST12.2-INSP113sv-6HIS (FIG. 28), pCR4-TOPO-INSP114 (FIG. 31), pCR4-TOPO-INSP114-GR1 (FIG. 35), pCR4-TOPO-INSP114-SV2 (FIG. 36), pDONR 221 (FIG. 38), pEAK12d (FIG. 39), pDEST12.2 (FIG. 40), pENTR_INSP114-6HIS (FIG. 41), pEAK12d_INSP114-6HIS (FIG. 42), pDEST12.2_INSP114-6HIS (FIG. 43), pENTR-INSP114-SV1-6HIS (FIG. 44), pEAK12d_INSP114-SV1-6HIS (FIG. 45), pDEST12.2_INSP114-SV1-6HIS (FIG. 46), pENTR_INSP114-SV2-6HIS (FIG. 47), pEAK12d_INSP114-SV2-6HIS (FIG. 48), pDEST12.2_INSP114-SV2-6HIS (FIG. 49), pDONR 221 (FIG. 51), pEAK12d (FIG. 52), pDEST12.2 (FIG. 53), pENTR-INSP115-6HIS (FIG. 54), pEAK12d_INSP115-6HIS (FIG. 55), pDEST12.2_INSP115-6HIS (FIG. 56), pDONR 221 (FIG. 58), pEAK12d (FIG. 59), pDEST12.2 (FIG. 60), pENTR_INSP116-6HIS (FIG. 61), pEAK12d_INSP116-6HIS (FIG. 62), pDEST12.2_INSP116-6HIS (FIG. 63), pCRII-TOPO-INSP117 (FIG. 66), pDONR 221 (FIG. 67), pEAK12d (FIG. 68), pDEST12.2 (FIG. 69), pENTR_INSP117-6HIS (FIG. 70), pEAK12d-INSP117-6HIS (FIG. 71) and pDEST12.2_INSP117-6HIS (FIG. 72) expression vectors. Transfection of mammalian cell lines with these vectors may enable the high level expression of the INSP113, INSP114, INSP115, INSP116 and INSP117 proteins and thus enable the continued investigation of the functional characteristics of the INSP113, INSP114, INSP115, INSP116 and INSP117 polypeptides. The following material and methods are an example of those suitable in such experiments:

Cell Culture

Human Embryonic Kidney 293 cells expressing the Epstein-Barr virus Nuclear Antigen (HEK293-EBNA, Invitrogen) are maintained in suspension in Ex-cell VPRO serum-free medium (seed stock, maintenance medium, JRH). Sixteen to 20 hours prior to transfection (Day-1), cells are seeded in 2×T225 flasks (50 ml per flask in DMEM/F12 (1:1) containing 2% FBS seeding medium (JRH) at a density of 2×10⁵ cells/ml). The next day (transfection day 0) transfection takes place using the JetPEI™ reagent (2 μl/μg of plasmid DNA, PolyPlus-transfection). For each flask, plasmid DNA is co-transfected with GFP (fluorescent reporter gene) DNA. The transfection mix is then added to the 2×T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 μl) is kept for QC of the 6His-tagged protein (internal bioprocessing QC).

Scale-up batches may be produced by following the protocol called “PEI transfection of suspension cells”, referenced BP/PEI/HH/02/04, with PolyEthyleneImine from Polysciences as transfection agent.

Purification Process

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amersham Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-His antibodies. The NuPAGE gel may be stained in a 0.1% Coomassie blue R250 staining solution (30% methanol, 10% acetic acid) at room temperature for 1 h and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analysed.

For samples that showed detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP113, INSP114, INSP115, INSP116 and INSP117 polypeptide may be identified by immunoprecipitation combined with Western blotting and immunoprecipitation combined with mass spectroscopy.

### Example 18

### Assays for the Detection of Biological Activity Similar to that of Coagulation Factor X

1. Assays Targeting T Lymphocyte Responses

Fas-Ligand-Induced T Cell Death.

This assay will reveal new modulators of receptor mediated cell death.

In this assay, T cell apoptosis is induced by stimulating Jurkat cells (a human T cell line) with recombinant 6 Histidine-tagged Fas Ligand combined with a monoclonal anti 6-his antibody. Death is quantified by release of LDH, a cytoplasmic enzyme released in the culture medium when cells are dying. The read out is a colorimetric assay read at 490 nm.T cells have been shown to be pathogenic in many autoimmune diseases, being able to control antigen-specific T cell death is a therapeutic strategy (e.g. anti-TNFα treatment in patient with Crohn's disease).

Human-MLR: Proliferation and Cytokine Secretion.

This cell-based assay measures the effects of novel proteins on lymphocyte proliferation and cytokine secretion or inhibition upon stimulation by PBMC from another donor (alloreactivity). These assay address antigen-specific T cell and antigen presenting cell functions, which are crucial cellular responses in any autoimmune diseases. Secreted cytokine (IL-2, 4, 5, 10, TNF-α and IFN-γ) are quantified by CBA.

Note: proliferation and cytokine secretion are independent responses.

Mouse-MLR: Proliferation.

This cell-based assay measures the effects of novel proteins on lymphocyte proliferation or inhibition of mouse spleen cells following stimulation by spleen cells from another donor (mouse strain). This cell-based assay measures the effect of novel proteins on T lymphocyte and antigen presenting cell responses and will be used to confirm activity of positives and hits identify in the h-MLR assays. This assay will be use to select proteins that will be tested in murine model of human diseases.

Human PBMC Stimulated with the Superantigen, TSST.

Superantigens are strong modulators of the immune system affecting T cells. Superantigens influence immunologically mediated disorders such as IBD, inflammatory skin diseases like atopic dermatitis and psoriasis. In this cellular assay, we are specifically targeting T lymphocyte activation via the TCR but with different requirements than the T cell response to classical antigens, in particular in respect to co-stimulatory molecules.

Human PBMC Stimulated with Either ConA or PHA.

These cell-based assays measure the effects of novel proteins on cytokine secretion induced by two different stimuli acting on different cells as measured by a cytokine bead array (CBA) assay (IL-2, IFN-γ, TNF-α, IL-5, IL-4 and IL-10).

Most of cytokines can have dual actions, pro or anti-inflammatory, depending of the injury, milieu and cellular target. Any protein with the capability to modulate cytokine secretion may have a therapeutic potential (e.g. decreasing IFN-γ and TNF-α would be beneficial in Th1-mediated autoimmune disease in contrast decreasing IL-4, IL-5 may be beneficial in Th2-mediated-diseases, inducing IL-10 would interesting in MS and SLE).

2. Assays Targeting Monocyte/Macrophages and Granulocyte Responses

Human PBMC Stimulated with LPS.

This cell-based assay measures the effects of novel proteins on cytokine secretion (IFN-γ, TNF-α) induced by LPS acting on monocytes/macrophages and granulocytes.

Any protein with the capability to modulate IFN-γ and TNF-α secretion would be beneficial in Th1-mediated autoimmune diseases.

3. Assays Targeting Neutrophil Responses

Neutrophils are important in inflammation and autoimmune diseases such as Rheumatoid Arthritis. Leukocyte chemo-attractants such as IL-8 initiate a sequence of adhesive interactions between cells and the micro-vascular endothelium, resulting in activation, adhesion and finally migration of neutrophils. The tissue infiltration of neutrophils depends on a reorganisation of cytoskeleton elements associated with specific changes in cell morphology of these cells.

This cell-based assay measures the effect of novel proteins on cytoskeleton reorganization of human neutrophils.

4. Assays Targeting B Lymphocyte Responses

Autoantibodies as well as infiltrating B cells are thought to be important in the pathogenesis of various autoimmune diseases, such as systemic lupus erithematosus (SLE), rheumatoid arthritis (RA), Sjogren's syndrome and myasthenia gravis. Compelling evidence indicates that a disregulation in B cell homeostasis could affect immune tolerance leading to the inappropriate survival of autoreactive B cells producing pathogenic antibodies and sustained inflammation. The identification of new factors that play critical roles in the regulation of B cell proliferation, survival and differentiation following B cell receptor triggering are of high relevance in the development of novel therapies.

B Cell Proliferation.

This cell-based assay measures the effect of novel proteins on B cell survival.

B Cell Co-Stimulation.

This cell-based assay measures the effect of novel proteins on B cell co-stimulation.

5. Assays Targeting Monocytes and Microglial Responses

THP-1 Calcium Flux.

The Ca⁺-flux in THP1-cell assay measures the effects of novel proteins on their ability to trigger an intracellular calcium release (a generic second messenger events) from the endoplasmic reticulum.

6. Microglia Cell Proliferation (will be Presented to the Next IAC).

During proliferation of microglial progenitors, a number of colony-stimulating factors, including some cytokines, are known to play key roles. Among them, M-CSF is crucial for the final step of maturation of macrophages/microglia and is not replaceable by any other factor. The evaluation of this biological response may represent a way to influence the microglial activity and therefore an opportunity to identify molecules with therapeutic potential fro MS.

A cell-based assay was developed to measure the proliferative response of a microglia cell line to M-CSF. The feasibility and the robustness phases showed optimal results. This assay is in 96 well plates; non-radioactive substrate is required, easily automated.

7. Assays to Detect Chemokine-Like Activity

Studies on structure-activity relationships indicate that chemokines bind and activate receptors by making use of the amino-terminal region. Proteolytic digestion, mutagenesis, or chemical modifications directed to amino acids in this region can generate compounds having antagonistic activity (Loetscher P and Clark-Lewis I, J Leukoc Biol, 69: 881-884, 2001 Lambeir A, et al. J Biol Chem, 276: 29839-29845, 2001, Proost P, et al. Blood, 98 (13):3554-3561, 2001). Thus, antagonistic molecules resulting from specific modifications (deletions, non-conservative substitutions) of one or more residues in the amino-terminal region or in other regions of the corresponding chemokine are considered having therapeutic potential for inflammatory and autoimmune diseases (WO 02/28419; WO 00/27880; WO 99/33989; Schwarz M K and Wells T N, Curr Opin Chem Biol, 3: 407-17, 1999). Therefore, a further object of the present patent application is represented by such kind of antagonists generated by modifying the polypeptides of the invention.

The therapeutic applications of the polypeptides of the invention and of the related reagents can be evaluated (in terms or safety, pharmacokinetics and efficacy) by the means of the in vivo/in vitro assays making use of animal cell, tissues and models (Coleman R A et al., Drug Discov Today, 6: 1116-1126, 2001; Li A P, Drug Discov Today, 6: 357-366, 2001; Methods Mol. Biol. vol. 138, “Chemokines Protocols”, edited by Proudfoot A I et al., Humana Press Inc., 2000; Methods Enzymol, vol. 287 and 288, Academic Press, 1997), or by the means of in silico/computational approaches (Johnson D E and Wolfgang G H, Drug Discov Today, 5: 445-454, 2000), known for the validation of chemokines and other biological products during drug discovery and preclinical development.

The present patent application discloses novel chemokine-like polypeptides and a series of related reagents that may be useful, as active ingredients in pharmaceutical compositions appropriately formulated, in the treatment or prevention of diseases such as cell proliferative disorders, autoimmune/inflammatory disorders, cardiovascular disorders, neurological disorders, developmental disorders, metabolic disorder, infections and other pathological conditions. In particular, given the known properties of chemokines, the disclosed polypeptides and reagents should address conditions involving abnormal or defective cell migration. Non-limitative examples of such conditions are the following: arthritis, rheumatoid arthritis (RA), psoriatic arthritis, osteoarthritis, systemic lupus erythematosus (SLE), systemic sclerosis, scleroderma, polymyositis, glomerulonephritis, fibrosis, lung fibrosis and inflammation, allergic or hypersensitvity diseases, dermatitis, asthma, chronic obstructive pulmonary disease (COPD), inflammatory bowel disease (IBD), Crohn's diseases, ulcerative colitis, multiple sclerosis, septic shock, HIV infection, transplant rejection, wound healing, metastasis, endometriosis, hepatitis, liver fibrosis, cancer, analgesia, and vascular inflammation related to atherosclerosis.

Cell- and Animal-Based Assay for the Validation and Characterization of the Chemokine-Like Polypeptides.

Several assays have been developed for testing specificity, potency, and efficacy of chemokines using cell cultures or animal models, for example in vitro chemotaxis assays (Proudfoot A, et al. J Biol Chem 276: 10620-10626, 2001; Lusti-Narasimhan M et al., J Biol Chem, 270: 2716-21, 1995), or mouse ear swelling (Garrigue J L et al., Contact Dermatitis, 30: 231-7, 1994). Many other assays and technologies for generating useful tools and products (antibodies, transgenic animals, radiolabeled proteins, etc.) have been described in reviews and books dedicated to chemokines (Methods Mol. Biol. vol. 138, “Chemokines Protocols”, edited by Proudfoot A I et al., Humana Press Inc., 2000; Methods Enzymol, vol. 287 and 288, Academic Press, 1997), and can be used to verify, in a more precise manner, the biological activities of the chemokine-like polypeptides of the invention and related reagents in connection with possible therapeutic or diagnostic methods and uses.

Cytokine Expression Modulation Assays

1. Introduction

The following in vitro cell-based tri-replicas assays measure the effects of the protein of the invention on cytokine secretion induced by Concanavalin A (Con A) acting on different human peripheral blood mononuclear cells (hPBMC) cells as measured by a cytokine bead array (CBA) assay for IL-2, IFN-γ, TNF-α, IL-5, IL-4 and IL-10.

The optimal conditions are 100 000 cells/well in 96-well plates and 100 μl final in 2% glycerol.

The optimal concentration of mitogen (ConA) is 5 ng/ml.

The optimal time for the assay is 48 h.

The read-out choice is the CBA.

2 Equipments and Softwares

96 well microtiter plate photometer EX (Labsystem).

Graph Pad Prism Software

Excel software

Flow cytometer Becton-Dickinson

CBA Analysis software

Hood for cell culture

Incubator for cell culture

Centrifuge

Pipettes

3. Materials and Reagents

Buffy coat

DMEM GIBCO

Human serum type AB SIGMA

L-Glutamine GIBCO

Penicillin-Streptomycin GIBCO

Ficoll PHARMACIA

96 well microtiter plate for cell culture COSTAR

Concanavalin A SIGMA

Human Th1/Th2 Cytokine CBA Kit Becton-Dickinson

PBS GIBCO

FALCON 50 ml sterile Becton-Dickinson

BSA SIGMA

Glycerol MERCK

DMSO SIGMA

96 well microtiter plate conical bottom NUNC

4 Method

4.1 Purification of Human PBMC from a Buffy Coat

The buffy coat 1 to 2 was diluted with DMEM. 25 ml of diluted blood was thereafter slowly added onto a 15 ml layer of Ficoll in a 50 ml Falcon tube, and tubes were centrifuged (2000 rpm, 20 min, at RT without brake). The interphase (ring) was then collected and the cells were washed with 25 ml of DMEM followed by a centrifuge step (1200 rpm, 5 min). This procedure was repeated three times. A buffy coat gave approximately 600×10⁶ total cells.

4.2 Screening

80 μl of 1.25×10⁶ cells/ml were diluted in DMEM+2.5% Human Serum+1% L-Glutamine+1% Penicillin-Streptomycin and thereafter added to a 96 well microtiter plate.

10 μl were added per well (one condition per well): Proteins were diluted in PBS+20% Glycerol (the final dilution of the proteins is 1/10).

10 μl of the ConA Stimuli were then added per well (one condition per well):

ConA 50 μg/ml (the final concentration of ConA is 5 μg/ml)

After 48 h, cell supernatants were collected and human cytokines were measured by Human Th1/Th2 Cytokine CBA Kit Becton-Dickinson.

4.3 CBA Analysis

(For More Details, Refer to the Booklet in the CBA Kit)

i) Preparation of Mixed Human Th1/Th2 Capture Beads

The number of assay tubes that were required for the experiment was determined.

Each capture bead suspension was vigorously vortexed for a few seconds before mixing. For each assay to be analysed, 10 μl aliquot of each capture bead were added into a single tube labelled “mixed capture beads”. The Bead mixture was thoroughly vortexed.

ii) Preparation of Test Samples

Supernatants were diluted (1:4) using the Assay Diluent (20 μl of supernatants+60 μl of Assay Diluent). The sample dilution was then mixed before transferring samples into a 96 wells microtiter plate conical bottom (Nunc).

iii) Human Th1/Th2 Cytokine CBA Assay Procedure

50 μl of the diluted supernatants were added into a 96 wells microtiter plate conical bottom (Nunc). ═μl of the mixed capture beads were added followed by 50 μl addition of the Human Th1/Th2 PE Detection Reagent. The plate was then incubated for 3 hours at RT and protected from direct exposure to light followed by centrifugation at 1500 rpm for 5 minutes. The supernatant was then carefully discarded. In a subsequent step, 2001 μl of wash buffer were twice added to each well, centrifuged at 1500 rpm for 5 minutes and supernatant carefully discarded. 130 μl of wash buffer were thereafter added to each well to resuspend the bead pellet. The samples were finally analysed on a flow cytometer. The data were analysed using the CBA Application Software, Activity Base and Microsoft Excel software.

From the read-out of the assay it can be evaluated whether in-vitro, the protein of the invention has a consistent inhibitory effect on all cytokines tested (IFN-γ, TNF-α, IL-2, IL-4, IL-5, IL-10). Moreover, based on the EC50 value, it can be easily evaluated which is the best inhibited cytokine and then arrive at the specific auto-immune/inflammatory disease, which is known to be linked to such cytokine particularly.

We claim:

1. A method of identifying a member of the SECFAM1 family, comprising:
searching a database of translated nucleic acid sequences or amino acid sequences to identify an amino acid sequence that matches the following sequence profile:

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −1 | −1 | −2 | −3 | −1 | 0 | −2 | −3 | −2 | 0 | 1 | −1 | 7 | 0 | −2 | −1 | −1 | −1 | −1 | 0 |
| 2 | N | 1 | 2 | 2 | −1 | −1 | 0 | −1 | −1 | −1 | −1 | −1 | 0 | 3 | −2 | −2 | 1 | 0 | −3 | −2 | −1 |
| 3 | K | 0 | 0 | −1 | 0 | −2 | 1 | 2 | −2 | −1 | 0 | −1 | 1 | −1 | −3 | 2 | −1 | −1 | −3 | −2 | 1 |
| 4 | R | 0 | 3 | −1 | −2 | 4 | 0 | −1 | −2 | −1 | 0 | 0 | 0 | 0 | −2 | −2 | 1 | −1 | −3 | −2 | −1 |
| 5 | Y | 1 | 0 | −2 | −2 | −2 | −1 | −1 | −2 | −1 | 0 | 0 | −1 | 0 | −1 | 3 | 0 | −1 | −2 | 2 | 0 |
| 6 | L | −1 | 2 | −1 | −1 | −2 | 0 | 0 | −2 | −1 | −1 | 0 | 3 | 2 | −2 | −2 | 0 | 1 | −2 | −2 | −1 |
| 7 | Q | 1 | 0 | 0 | −1 | −1 | 1 | 0 | −1 | −1 | −2 | −2 | 0 | −1 | −2 | −1 | 2 | 2 | 3 | −1 | −1 |
| 8 | K | −1 | −1 | 2 | −1 | −2 | −1 | −1 | 0 | −1 | 0 | 0 | 1 | 2 | −1 | −2 | 0 | 0 | −3 | −2 | 2 |
| 9 | A | 0 | 0 | −2 | −2 | −1 | −1 | −2 | −2 | −2 | 0 | 0 | −1 | 0 | −1 | −2 | 0 | −1 | 7 | 0 | 0 |
| 10 | T | 0 | 1 | 0 | −1 | −1 | 0 | −1 | −1 | −1 | −1 | 0 | 0 | 0 | −2 | 1 | 2 | 1 | −3 | −2 | −1 |
| 11 | Q | 1 | 0 | 0 | −1 | −1 | 2 | 0 | −1 | −1 | −1 | 0 | 0 | 0 | −2 | 0 | 2 | 0 | −3 | −2 | −1 |
| 12 | G | 0 | 0 | 0 | 2 | −2 | −1 | 0 | 3 | 4 | −2 | −2 | −1 | 2 | −2 | −2 | 0 | −1 | −2 | −1 | −2 |
| 13 | K | 1 | −1 | −1 | −1 | −2 | −1 | −1 | 2 | −2 | −3 | −2 | 0 | −1 | −1 | −2 | 2 | −1 | 7 | −1 | −2 |
| 14 | L | 0 | −2 | −2 | −3 | −1 | −2 | −2 | 1 | −2 | −1 | 0 | −2 | 0 | 0 | −2 | −1 | 1 | 8 | 0 | −1 |
| 15 | L | 2 | −2 | −3 | −3 | −1 | −2 | −2 | −2 | −3 | 1 | 3 | −2 | 0 | −1 | −2 | −1 | 0 | −3 | −1 | 2 |
| 16 | I | −1 | −2 | −3 | −4 | −1 | −2 | −3 | −4 | −3 | 2 | 4 | −2 | 1 | 1 | −3 | −2 | −1 | −2 | 0 | 1 |
| 17 | I | 1 | −2 | −2 | −2 | −1 | −2 | −2 | −2 | −1 | 1 | 1 | −2 | 0 | 0 | 3 | −1 | −1 | −1 | 2 | 0 |
| 18 | I | 0 | −2 | −1 | −2 | −1 | −1 | −2 | −2 | −2 | 1 | 3 | −1 | 0 | −1 | −2 | 1 | 0 | −2 | −1 | 0 |
| 19 | F | −1 | −2 | −3 | −3 | 6 | −2 | −3 | −3 | −1 | −1 | 0 | −2 | 3 | 2 | −3 | −2 | −1 | 6 | 3 | −1 |
| 20 | I | 0 | −2 | −1 | −2 | −1 | −2 | −2 | −2 | −2 | 3 | 1 | −2 | 0 | −1 | −2 | 1 | 1 | −3 | −1 | 2 |
| 21 | V | 2 | −2 | −1 | −2 | 3 | −1 | −1 | −1 | −2 | 0 | 0 | −1 | 0 | −2 | −2 | 2 | 0 | −3 | −2 | 0 |
| 22 | T | 1 | −2 | −2 | −2 | 3 | −1 | −2 | −2 | −2 | −1 | 0 | −2 | 2 | −1 | −2 | 0 | 2 | 6 | −1 | 0 |
| 23 | L | −1 | −3 | −3 | −3 | 4 | −3 | −3 | −3 | −2 | 0 | 2 | −3 | 0 | 4 | −3 | −2 | −1 | −1 | 0 | 1 |
| 24 | W | 0 | −3 | −3 | −3 | 5 | −2 | −3 | −2 | −2 | −1 | 0 | −3 | −1 | 0 | −3 | −1 | −1 | 9 | 0 | −1 |
| 25 | G | 1 | −2 | −1 | −2 | 4 | −1 | −2 | 1 | −2 | 0 | 0 | −1 | 2 | −2 | −2 | 0 | 1 | −2 | −2 | 0 |
| 26 | K | −1 | 0 | −1 | −2 | 1 | 1 | 0 | −3 | 1 | 0 | 1 | 2 | 0 | 2 | −2 | −1 | −1 | −2 | 0 | −1 |
| 27 | A | 0 | −2 | −3 | −3 | −1 | −2 | −2 | −3 | −3 | 1 | 3 | −2 | 3 | 0 | 0 | −2 | −1 | −2 | −1 | 1 |
| 28 | V | 0 | −2 | −2 | −3 | 4 | −2 | −2 | −3 | −3 | 3 | 0 | −2 | 3 | −1 | −2 | −1 | 0 | −2 | −1 | 2 |
| 29 | S | 0 | −1 | 0 | −1 | −1 | −1 | −1 | −1 | 5 | −1 | 1 | −1 | 0 | −1 | −2 | 2 | 0 | −3 | 0 | −1 |
| 30 | S | 4 | −1 | −1 | −2 | −1 | −1 | −1 | 2 | −2 | −2 | −2 | −1 | −1 | −2 | −1 | 1 | 1 | −3 | −2 | −1 |
| 31 | A | 1 | −1 | 0 | 0 | −1 | 1 | 0 | 1 | −1 | −2 | −2 | 0 | −1 | −2 | −1 | 4 | 0 | −3 | −2 | −2 |
| 32 | N | −1 | −1 | 2 | −1 | −2 | −1 | −2 | −2 | −1 | 0 | 3 | −1 | 0 | 1 | −3 | 1 | −1 | −2 | −1 | 0 |
| 33 | H | −1 | 0 | 0 | −1 | −2 | 5 | 0 | −3 | 3 | −1 | 1 | 0 | 0 | −2 | −2 | −1 | −1 | −2 | −1 | −1 |
| 34 | H | −1 | 0 | 0 | −1 | −2 | 0 | 0 | −2 | 6 | 0 | −1 | 0 | −1 | −2 | 2 | 0 | −1 | −3 | 0 | −1 |
| 35 | K | −1 | 0 | 0 | −1 | −3 | 3 | 0 | −2 | 4 | −3 | −2 | 1 | −1 | −3 | 3 | 0 | −1 | −2 | 0 | −2 |
| 36 | A | 2 | 1 | 0 | −1 | −1 | 0 | −1 | −1 | 4 | −1 | −1 | 0 | −1 | −2 | −1 | 0 | 2 | −2 | 0 | −1 |
| 37 | H | 0 | −1 | 0 | −1 | −2 | −1 | −1 | 3 | 6 | −3 | −3 | −1 | −2 | −2 | −2 | 1 | −1 | −2 | 0 | −2 |
| 38 | H | 0 | −1 | 0 | −2 | 5 | −1 | −1 | −2 | 5 | −1 | 0 | −1 | −1 | −1 | −2 | 0 | 1 | −2 | 0 | −1 |
| 39 | V | −1 | −2 | −2 | −2 | −2 | −1 | 0 | −3 | −2 | 2 | 2 | −2 | 0 | −1 | 2 | −2 | −1 | −3 | −1 | 2 |
| 40 | R | 1 | 2 | −1 | −1 | −2 | 1 | 1 | −1 | −1 | −2 | −2 | 3 | −1 | −3 | −1 | 0 | −1 | −3 | −2 | −2 |
| 41 | T | 2 | −1 | −1 | −1 | −2 | 3 | 0 | 1 | −1 | −2 | −2 | 0 | −1 | −3 | 0 | 0 | 2 | −2 | −2 | −1 |
| 42 | G | 0 | −2 | 0 | −1 | −3 | −2 | −2 | 6 | −2 | −4 | −4 | −2 | −3 | −3 | −2 | 0 | −2 | −2 | −3 | −3 |
| 43 | T | 0 | −1 | 0 | −1 | −1 | −1 | −1 | −2 | −2 | −1 | −1 | −1 | −1 | −2 | −1 | 0 | 6 | −2 | −2 | 0 |
| 44 | C | 0 | −3 | −3 | −3 | 10 | −3 | −4 | −3 | −3 | −1 | −1 | −3 | −1 | −2 | −3 | −1 | −1 | −2 | −2 | −1 |
| 45 | E | −1 | 0 | 0 | 1 | −4 | 1 | 6 | −2 | 0 | −3 | −3 | 0 | −2 | −3 | −1 | 0 | −1 | −3 | −2 | −2 |
| 46 | V | 0 | −3 | −3 | −3 | −1 | −2 | −2 | −3 | −3 | 3 | 0 | −2 | 0 | −1 | −2 | −2 | 0 | −3 | −1 | 4 |
| 47 | V | 0 | −3 | −3 | −3 | −1 | −2 | −2 | −3 | −3 | 4 | 0 | −2 | 0 | −1 | −2 | −2 | 0 | −3 | −1 | 4 |
| 48 | A | 4 | −1 | −1 | −2 | 0 | −1 | −1 | −1 | −2 | −1 | −1 | −1 | −1 | −2 | −1 | 0 | 2 | −3 | −2 | 0 |
| 49 | L | 3 | −2 | −3 | −3 | −1 | −2 | −2 | −2 | −3 | 0 | 2 | −2 | 0 | −1 | −2 | 0 | 0 | −3 | −1 | 1 |
| 50 | H | −2 | −1 | 0 | 3 | −3 | 0 | 0 | −2 | 8 | −3 | −3 | −1 | −2 | −2 | −2 | −1 | −2 | −3 | 0 | −3 |
| 51 | R | −1 | 6 | 0 | −2 | −3 | 0 | 0 | −2 | 0 | −3 | −2 | 1 | −1 | −3 | −2 | −1 | −1 | −3 | −2 | −3 |
| 52 | C | −1 | −3 | −1 | 3 | 8 | −2 | −1 | −2 | −2 | −2 | −2 | −2 | −2 | −2 | −2 | −1 | −1 | −3 | −2 | −2 |
| 53 | C | 0 | −2 | −1 | −2 | 8 | −2 | −2 | −2 | −2 | −1 | −1 | −2 | −1 | −2 | −2 | 2 | 0 | −2 | −2 | −1 |
| 54 | N | −1 | 0 | 6 | 0 | −2 | 0 | 0 | 0 | 0 | −3 | −3 | 0 | −2 | −3 | −2 | 2 | 0 | −4 | −2 | −3 |
| 55 | K | −1 | 2 | 0 | −1 | −3 | 3 | 0 | −2 | −1 | −3 | −2 | 4 | −1 | −3 | −1 | 0 | −1 | −3 | −2 | −2 |
| 56 | N | −2 | −1 | 6 | 0 | −3 | 0 | 0 | −1 | 0 | −3 | −3 | 0 | −2 | −3 | 3 | 0 | 0 | −4 | −2 | −3 |
| 57 | K | −1 | 5 | 0 | −2 | −3 | 0 | 0 | −2 | 0 | −3 | −2 | 3 | −1 | −3 | −2 | −1 | −1 | −3 | −2 | −3 |
| 58 | I | −1 | 2 | −2 | −3 | −2 | −1 | −2 | −3 | −2 | 4 | 0 | −1 | 0 | −1 | −3 | −2 | −1 | −3 | −1 | 1 |
| 59 | E | −1 | 0 | 0 | 0 | −3 | 0 | 5 | −2 | −1 | −2 | −2 | 0 | −2 | −3 | −1 | 0 | 2 | −3 | −2 | −1 |
| 60 | E | −1 | −1 | −1 | 0 | −3 | 0 | 5 | −3 | −1 | 2 | −1 | 0 | −1 | −2 | −2 | −1 | −1 | −3 | −2 | 0 |
| 61 | R | 1 | 5 | −1 | −2 | −2 | 0 | 0 | −1 | −1 | −2 | −2 | 0 | −1 | −3 | −2 | 0 | −1 | −3 | −2 | −2 |
| 62 | S | 0 | 2 | 0 | −1 | −2 | 0 | 0 | −1 | −1 | −2 | −2 | 0 | −1 | −2 | −1 | 4 | 0 | −3 | −2 | −2 |
| 63 | Q | −1 | 0 | 0 | 0 | −3 | 6 | 1 | −2 | 0 | −3 | −2 | 0 | 0 | −3 | −1 | 0 | −1 | −2 | −1 | −2 |
| 64 | T | 0 | −1 | 0 | −1 | −1 | −1 | −1 | −2 | −2 | −1 | −1 | −1 | −1 | −2 | −1 | 0 | 6 | −2 | −2 | 0 |
| 65 | V | 2 | −2 | −3 | −3 | −1 | −2 | −2 | −2 | −3 | 1 | 0 | −2 | 0 | −1 | −2 | −1 | 0 | −3 | −1 | 4 |
| 66 | K | −1 | 3 | 0 | −1 | −3 | 0 | 0 | −2 | −1 | −3 | −2 | 5 | −1 | −3 | −1 | 0 | −1 | −3 | −2 | −2 |
| 67 | C | 0 | −3 | −3 | −3 | 10 | −3 | −4 | −3 | −3 | −1 | −1 | −3 | −1 | −2 | −3 | −1 | −1 | −2 | −2 | −1 |
| 68 | S | 2 | −1 | 0 | −1 | −1 | 0 | 0 | 0 | −1 | −2 | −2 | 0 | −1 | −2 | −1 | 4 | 0 | −3 | −2 | −1 |
| 69 | C | 0 | −3 | −3 | −3 | 10 | −3 | −4 | −3 | −3 | −1 | −1 | −3 | −1 | −2 | −3 | −1 | −1 | −2 | −2 | −1 |
| 70 | F | −2 | 2 | −2 | −3 | −2 | −1 | −2 | −3 | −1 | 0 | 1 | −1 | 0 | 4 | −3 | −2 | −2 | −1 | 0 | −1 |
| 71 | P | −1 | 0 | −1 | −1 | −3 | 0 | 0 | −2 | −2 | −3 | −3 | 2 | −2 | −3 | 5 | 1 | −1 | −4 | −3 | −2 |
| 72 | G | 0 | −2 | 0 | −1 | −3 | −2 | −2 | 6 | −2 | −4 | −4 | −2 | −3 | −3 | −2 | 0 | −2 | −2 | −3 | −3 |
| 73 | Q | −1 | 0 | 0 | 0 | −3 | 6 | 1 | −2 | 0 | −3 | −2 | 2 | 0 | −3 | −1 | 0 | −1 | −2 | −1 | −2 |
| 74 | V | 0 | −3 | −3 | −3 | −1 | −2 | −2 | −3 | −3 | 3 | 0 | −2 | 0 | −1 | −2 | −2 | 0 | −3 | −1 | 4 |
| 75 | A | 5 | −1 | −2 | −2 | 0 | −1 | −1 | 0 | −2 | −1 | −1 | −1 | −1 | −2 | −1 | 0 | 0 | −3 | −2 | 0 |
| 76 | G | 0 | −2 | 0 | −1 | −3 | −2 | −2 | 6 | −2 | −4 | −4 | −2 | −3 | −3 | −2 | 0 | −2 | −2 | −3 | −3 |
| 77 | T | 0 | −1 | 0 | −1 | −1 | −1 | −1 | −2 | −2 | −1 | −1 | −1 | −1 | −2 | −1 | 0 | 6 | −2 | −2 | 0 |
| 78 | T | 0 | −1 | 0 | −1 | −1 | −1 | −1 | −2 | −2 | −1 | −1 | −1 | −1 | −2 | −1 | 0 | 6 | −2 | −2 | 0 |
| 79 | R | −1 | 6 | 0 | −2 | −3 | 0 | 0 | −2 | 0 | −3 | −2 | 1 | −1 | −3 | −2 | −1 | −1 | −3 | −2 | −3 |
| 80 | A | 4 | −1 | 1 | −1 | 0 | −1 | −1 | 0 | −1 | −1 | −1 | −1 | −1 | −2 | −1 | 0 | 0 | −3 | −2 | 0 |
| 81 | A | 0 | 4 | 0 | −2 | −2 | 2 | 0 | −2 | −1 | −2 | 0 | 2 | 0 | −3 | −2 | 0 | −1 | −3 | −2 | −2 |
| 82 | P | −1 | −2 | −2 | −1 | −3 | −1 | −1 | −2 | −2 | −3 | −3 | −1 | −2 | −4 | 7 | −1 | −1 | −4 | −3 | −2 |
| 83 | S | 2 | −1 | 0 | −1 | −1 | 0 | 0 | 0 | −1 | −2 | −2 | 0 | −1 | −2 | −1 | 4 | 0 | −3 | −2 | −1 |
| 84 | C | 0 | −3 | −3 | −3 | 10 | −3 | −4 | −3 | −3 | −1 | −1 | −3 | −1 | −2 | −3 | −1 | −1 | −2 | −2 | −1 |
| 85 | V | 0 | −3 | −3 | −3 | −1 | −2 | −2 | −3 | −3 | 2 | 0 | −2 | 0 | −1 | −2 | −2 | 0 | −3 | −1 | 5 |
| 86 | D | −2 | −2 | 0 | 6 | −3 | 0 | 2 | −1 | −1 | −3 | −4 | −1 | −3 | −3 | −1 | 0 | −1 | −4 | −3 | −3 |
| 87 | A | 5 | −1 | −2 | −2 | 0 | −1 | −1 | 0 | −2 | −1 | −1 | −1 | −1 | −2 | −1 | 0 | 0 | −3 | −2 | 0 |
| 88 | S | 0 | 2 | 0 | −1 | −1 | 0 | 0 | −1 | −1 | −2 | −2 | 0 | −1 | −2 | −1 | 4 | 0 | −3 | −2 | −2 |
| 89 | I | −1 | −3 | −3 | −3 | −1 | −3 | −3 | −4 | −3 | 5 | 1 | −3 | 0 | 0 | −3 | −2 | −1 | −3 | −1 | 2 |
| 90 | V | 0 | −3 | −3 | −3 | −1 | −2 | −2 | −3 | −3 | 3 | 0 | −2 | 0 | −1 | −2 | −2 | 0 | −3 | −1 | 4 |
| 91 | E | −1 | 1 | −1 | −1 | −2 | 0 | 1 | −3 | −2 | 2 | 1 | 2 | 0 | −1 | −2 | −1 | −1 | −3 | −2 | 0 |
| 92 | Q | −1 | 0 | 0 | 0 | −2 | 4 | 1 | 0 | −1 | −2 | −2 | 0 | −1 | −2 | −1 | 0 | 2 | 2 | −1 | −2 |
| 93 | K | −1 | 2 | 0 | −1 | −3 | 0 | 0 | −2 | −1 | −3 | −2 | 6 | −1 | −3 | −1 | 0 | −1 | −3 | −2 | −2 |
| 94 | W | −2 | −1 | −2 | −2 | −2 | 2 | −1 | −2 | −1 | −3 | −2 | −1 | −1 | 0 | −3 | −2 | −2 | 10 | 0 | −3 |
| 95 | W | −3 | −3 | −4 | −4 | −2 | −2 | −3 | −2 | −2 | −3 | −2 | −3 | −1 | 0 | −4 | −3 | −2 | 11 | 1 | −3 |
| 96 | C | 0 | −3 | −3 | −3 | 10 | −3 | −4 | −3 | −3 | −1 | −1 | −3 | −1 | −2 | −3 | −1 | −1 | −2 | −2 | −1 |
| 97 | H | −2 | 0 | 1 | 3 | −3 | 2 | 2 | −2 | 5 | −3 | −3 | 0 | −2 | −2 | −1 | 0 | −1 | −3 | 0 | −3 |
| 98 | M | −1 | −1 | −2 | −3 | −1 | 0 | −2 | −3 | −2 | 0 | 1 | −1 | 7 | 0 | −2 | −1 | −1 | −1 | −1 | 0 |
| 99 | Q | −1 | 0 | 2 | 0 | −3 | 2 | 3 | −2 | 0 | −1 | 1 | 0 | 0 | −2 | −2 | 0 | −1 | −3 | −2 | −1 |
| 100 | P | −1 | −2 | −2 | −1 | −3 | −1 | −1 | −2 | −2 | −3 | −3 | −1 | −2 | −4 | 7 | −1 | −1 | −4 | −3 | −2 |
| 101 | C | 0 | −3 | −3 | −3 | 10 | −3 | −4 | −3 | −3 | −1 | −1 | −3 | −1 | −2 | −3 | −1 | −1 | −2 | −2 | −1 |
| 102 | L | −1 | −2 | −3 | −4 | −1 | −2 | −3 | −4 | −3 | 1 | 4 | −2 | 1 | 0 | −3 | −2 | −1 | −2 | −1 | 1 |
| 103 | E | −1 | −1 | −1 | 0 | −3 | 0 | 5 | −2 | −1 | −1 | 0 | 0 | −1 | −2 | 0 | 0 | −1 | −3 | −2 | 0 |
| 104 | G | 0 | −2 | 0 | −1 | −3 | −2 | −2 | 6 | −2 | −4 | −4 | −2 | −3 | −3 | −2 | 0 | −2 | −2 | −3 | −3 |
| 105 | E | −1 | 0 | 0 | 1 | −4 | 1 | 6 | −2 | 0 | −3 | −3 | 0 | −2 | −3 | −1 | 0 | −1 | −3 | −2 | −2 |
| 106 | E | −1 | −1 | 0 | 2 | −4 | 0 | 4 | 2 | −1 | −3 | −4 | 0 | −3 | −3 | −1 | 0 | −1 | −3 | −3 | −3 |
| 107 | C | 0 | −3 | −3 | −3 | 10 | −3 | −4 | −3 | −3 | −1 | −1 | −3 | −1 | −2 | −3 | −1 | −1 | −2 | −2 | −1 |
| 108 | K | −1 | 1 | 0 | 3 | −3 | 0 | 0 | −2 | −1 | −3 | −3 | 4 | −2 | −3 | −1 | 0 | −1 | −3 | −2 | −2 |
| 109 | V | 0 | −2 | −2 | −3 | −1 | −2 | −2 | −3 | −3 | 1 | 2 | −2 | 0 | −1 | −2 | −1 | 1 | −3 | −1 | 4 |
| 110 | L | −1 | −2 | −3 | −4 | −1 | −2 | −3 | −4 | −3 | 1 | 4 | −2 | 1 | 0 | −3 | −2 | −1 | −2 | −1 | 0 |
| 111 | P | −1 | −2 | −2 | −2 | −2 | −2 | −2 | −3 | −2 | 2 | −1 | −2 | −1 | −2 | 6 | −1 | −1 | −4 | −2 | 0 |
| 112 | D | −2 | −1 | 3 | 6 | −3 | 0 | 1 | −1 | 0 | −3 | −4 | −1 | −3 | −3 | −1 | 0 | −1 | −4 | −3 | −3 |
| 113 | R | −1 | 3 | 2 | −2 | −2 | 0 | −1 | −2 | 0 | −1 | 1 | 0 | 0 | −1 | −2 | 0 | −1 | −2 | 1 | −1 |
| 114 | K | 0 | −1 | 0 | 0 | −1 | 0 | 0 | 0 | −1 | −2 | −2 | 1 | −1 | −2 | −1 | 4 | 1 | −3 | −2 | −2 |
| 115 | G | 0 | −2 | 0 | −1 | −3 | −2 | −2 | 6 | −2 | −4 | −4 | −2 | −3 | −3 | −2 | 0 | −2 | −2 | −3 | −3 |
| 116 | W | −3 | −3 | −4 | −4 | −2 | −2 | −3 | −2 | −2 | −3 | −2 | −3 | −1 | 0 | −4 | −3 | −2 | 11 | 1 | −3 |
| 117 | S | 0 | −1 | 0 | −1 | −1 | 0 | −1 | −1 | −1 | −1 | −1 | 0 | 2 | −2 | −1 | 3 | 3 | −2 | −2 | −1 |
| 118 | C | 0 | −3 | −3 | −3 | 10 | −3 | −4 | −3 | −3 | −1 | −1 | −3 | −1 | −2 | −3 | −1 | −1 | −2 | −2 | −1 |
| 119 | S | 1 | −1 | 0 | −1 | −1 | −1 | −1 | −1 | −2 | 0 | −1 | −1 | −1 | −2 | −1 | 3 | 3 | −3 | −2 | 0 |
| 120 | S | 0 | 0 | 0 | 0 | −2 | 3 | 0 | −1 | −1 | −2 | −2 | 0 | −1 | −2 | −1 | 3 | 1 | −3 | −2 | −2 |
| 121 | G | 0 | −2 | 0 | −1 | −3 | −2 | −2 | 6 | −2 | −4 | −4 | −2 | −3 | −3 | −2 | 0 | −2 | −2 | −3 | −3 |
| 122 | N | −1 | −1 | 5 | 0 | −3 | −1 | −1 | 3 | 4 | −3 | −3 | −1 | −2 | −3 | −2 | 0 | −1 | −3 | −1 | −3 |
| 123 | K | −1 | 3 | 0 | −1 | −3 | 0 | 0 | −2 | −1 | −3 | −2 | 5 | −1 | −3 | −1 | 0 | −1 | −3 | −2 | −2 |
| 124 | V | 0 | −3 | −3 | −3 | −1 | −2 | −2 | −4 | −3 | 4 | 0 | −2 | 0 | −1 | −2 | −2 | 0 | −3 | −1 | 4 |
| 125 | K | −1 | 1 | 0 | −1 | −3 | 0 | 0 | −2 | −1 | −3 | −2 | 6 | −1 | −3 | −1 | 0 | −1 | −3 | −2 | −2 |
| 126 | T | 0 | −1 | 0 | −1 | −1 | −1 | −1 | −2 | −2 | −1 | −1 | −1 | −1 | −2 | −1 | 0 | 6 | −2 | −2 | 0 |
| 127 | T | 0 | −1 | 0 | −1 | −1 | −1 | −1 | −2 | −2 | −1 | −1 | −1 | −1 | −2 | −1 | 0 | 6 | −2 | −2 | 0 |
| 128 | R | −1 | 3 | 0 | −1 | −2 | 0 | 0 | −2 | −1 | −2 | −2 | 4 | −1 | −3 | −1 | 0 | 2 | −3 | −2 | −2 |
| 129 | V | 0 | −3 | −3 | −3 | −1 | −2 | −2 | −3 | −3 | 2 | 0 | −2 | 0 | −1 | −2 | −2 | 0 | −3 | −1 | 5 |
| 130 | T | 0 | −1 | 0 | −1 | −1 | 0 | −1 | −1 | 2 | −1 | 0 | −1 | −1 | −2 | −1 | 3 | 3 | −3 | −1 | −1 |
| 131 | H | −1 | 4 | 0 | −1 | −3 | 0 | 0 | −2 | 4 | −3 | −2 | 0 | −1 | −3 | 3 | −1 | −1 | −3 | −1 | −3 |

wherein, when said profile is input as a query sequence into the search program BLAST, using the default parameters specified by the NCBI (the National Center for Biotechnology Information) [Blosum 62 matrix; gap open penalty=11 and gap extension penalty=1], members of the SECFAM1 family are those which have an E value of 10⁻² or less.

2. The method of claim 1 wherein said E value is 10⁻⁵ or less.

3. The method of claim 1 wherein the database of translated nucleic acid sequences is derived from cDNA, EST, mRNA, whole or partial genome databases.

4. The method of claim 1 wherein the database is an EST database.

5. The method of claim 1 wherein the database is a human sequence database.

6-67. (canceled)

68. A composition of matter comprising:
a) an isolated polypeptide:
1) comprising an amino acid sequence that has an E value of 10⁻² or less when the profile below is input as a query sequence into the search program BLAST, using the default parameters specified by the NCBI (the National Center for Biotechnology Information) and has Blosum 62 matrix; gap open penalty=11 and gap extension penalty=1

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −1 | −1 | −2 | −3 | −1 | 0 | −2 | −3 | −2 | 0 | 1 | −1 | 7 | 0 | −2 | −1 | −1 | −1 | −1 | 0 |
| 2 | N | 1 | 2 | 2 | −1 | −1 | 0 | −1 | −1 | −1 | −1 | −1 | 0 | 3 | −2 | −2 | 1 | 0 | −3 | −2 | −1 |
| 3 | K | 0 | 0 | −1 | 0 | −2 | 1 | 2 | −2 | −1 | 0 | −1 | 1 | −1 | −3 | 2 | −1 | −1 | −3 | −2 | 1 |
| 4 | R | 0 | 3 | −1 | −2 | 4 | 0 | −1 | −2 | −1 | 0 | 0 | 0 | 0 | −2 | −2 | 1 | −1 | −3 | −2 | −1 |
| 5 | Y | 1 | 0 | −2 | −2 | −2 | −1 | −1 | −2 | −1 | 0 | 0 | −1 | 0 | −1 | 3 | 0 | −1 | −2 | 2 | 0 |
| 6 | L | −1 | 2 | −1 | −1 | −2 | 0 | 0 | −2 | −1 | −1 | 0 | 3 | 2 | −2 | −2 | 0 | 1 | −2 | −2 | −1 |
| 7 | Q | 1 | 0 | 0 | −1 | −1 | 1 | 0 | −1 | −1 | −2 | −2 | 0 | −1 | −2 | −1 | 2 | 2 | 3 | −1 | −1 |
| 8 | K | −1 | −1 | 2 | −1 | −2 | −1 | −1 | 0 | −1 | 0 | 0 | 1 | 2 | −1 | −2 | 0 | 0 | −3 | −2 | 2 |
| 9 | A | 0 | 0 | −2 | −2 | −1 | −1 | −2 | −2 | −2 | 0 | 0 | −1 | 0 | −1 | −2 | 0 | −1 | 7 | 0 | 0 |
| 10 | T | 0 | 1 | 0 | −1 | −1 | 0 | −1 | −1 | −1 | −1 | 0 | 0 | 0 | −2 | 1 | 2 | 1 | −3 | −2 | −1 |
| 11 | Q | 1 | 0 | 0 | −1 | −1 | 2 | 0 | −1 | −1 | −1 | 0 | 0 | 0 | −2 | 0 | 2 | 0 | −3 | −2 | −1 |
| 12 | G | 0 | 0 | 0 | 2 | −2 | −1 | 0 | 3 | 4 | −2 | −2 | −1 | 2 | −2 | −2 | 0 | −1 | −2 | −1 | −2 |
| 13 | K | 1 | −1 | −1 | −1 | −2 | −1 | −1 | 2 | −2 | −3 | −2 | 0 | −1 | −1 | −2 | 2 | −1 | 7 | −1 | −2 |
| 14 | L | 0 | −2 | −2 | −3 | −1 | −2 | −2 | 1 | −2 | −1 | 0 | −2 | 0 | 0 | −2 | −1 | 1 | 8 | 0 | −1 |
| 15 | L | 2 | −2 | −3 | −3 | −1 | −2 | −2 | −2 | −3 | 1 | 3 | −2 | 0 | −1 | −2 | −1 | 0 | −3 | −1 | 2 |
| 16 | I | −1 | −2 | −3 | −4 | −1 | −2 | −3 | −4 | −3 | 2 | 4 | −2 | 1 | 1 | −3 | −2 | −1 | −2 | 0 | 1 |
| 17 | I | 1 | −2 | −2 | −2 | −1 | −2 | −2 | −2 | −1 | 1 | 1 | −2 | 0 | 0 | 3 | −1 | −1 | −1 | 2 | 0 |
| 18 | I | 0 | −2 | −1 | −2 | −1 | −1 | −2 | −2 | −2 | 1 | 3 | −1 | 0 | −1 | −2 | 1 | 0 | −2 | −1 | 0 |
| 19 | F | −1 | −2 | −3 | −3 | 6 | −2 | −3 | −3 | −1 | −1 | 0 | −2 | 3 | 2 | −3 | −2 | −1 | 6 | 3 | −1 |
| 20 | I | 0 | −2 | −1 | −2 | −1 | −2 | −2 | −2 | −2 | 3 | 1 | −2 | 0 | −1 | −2 | 1 | 1 | −3 | −1 | 2 |
| 21 | V | 2 | −2 | −1 | −2 | 3 | −1 | −1 | −1 | −2 | 0 | 0 | −1 | 0 | −2 | −2 | 2 | 0 | −3 | −2 | 0 |
| 22 | T | 1 | −2 | −2 | −2 | 3 | −1 | −2 | −2 | −2 | −1 | 0 | −2 | 2 | −1 | −2 | 0 | 2 | 6 | −1 | 0 |
| 23 | L | −1 | −3 | −3 | −3 | 4 | −3 | −3 | −3 | −2 | 0 | 2 | −3 | 0 | 4 | −3 | −2 | −1 | −1 | 0 | 1 |
| 24 | W | 0 | −3 | −3 | −3 | 5 | −2 | −3 | −2 | −2 | −1 | 0 | −3 | −1 | 0 | −3 | −1 | −1 | 9 | 0 | −1 |
| 25 | G | 1 | −2 | −1 | −2 | 4 | −1 | −2 | 1 | −2 | 0 | 0 | −1 | 2 | −2 | −2 | 0 | 1 | −2 | −2 | 0 |
| 26 | K | −1 | 0 | −1 | −2 | 1 | 1 | 0 | −3 | 1 | 0 | 1 | 2 | 0 | 2 | −2 | −1 | −1 | −2 | 0 | −1 |
| 27 | A | 0 | −2 | −3 | −3 | −1 | −2 | −2 | −3 | −3 | 1 | 3 | −2 | 3 | 0 | 0 | −2 | −1 | −2 | −1 | 1 |
| 28 | V | 0 | −2 | −2 | −3 | 4 | −2 | −2 | −3 | −3 | 3 | 0 | −2 | 3 | −1 | −2 | −1 | 0 | −2 | −1 | 2 |
| 29 | S | 0 | −1 | 0 | −1 | −1 | −1 | −1 | −1 | 5 | −1 | 1 | −1 | 0 | −1 | −2 | 2 | 0 | −3 | 0 | −1 |
| 30 | S | 4 | −1 | −1 | −2 | −1 | −1 | −1 | 2 | −2 | −2 | −2 | −1 | −1 | −2 | −1 | 1 | 1 | −3 | −2 | −1 |
| 31 | A | 1 | −1 | 0 | 0 | −1 | 1 | 0 | 1 | −1 | −2 | −2 | 0 | −1 | −2 | −1 | 4 | 0 | −3 | −2 | −2 |
| 32 | N | −1 | −1 | 2 | −1 | −2 | −1 | −2 | −2 | −1 | 0 | 3 | −1 | 0 | 1 | −3 | 1 | −1 | −2 | −1 | 0 |
| 33 | H | −1 | 0 | 0 | −1 | −2 | 5 | 0 | −3 | 3 | −1 | 1 | 0 | 0 | −2 | −2 | −1 | −1 | −2 | −1 | −1 |
| 34 | H | −1 | 0 | 0 | −1 | −2 | 0 | 0 | −2 | 6 | 0 | −1 | 0 | −1 | −2 | 2 | 0 | −1 | −3 | 0 | −1 |
| 35 | K | −1 | 0 | 0 | −1 | −3 | 3 | 0 | −2 | 4 | −3 | −2 | 1 | −1 | −3 | 3 | 0 | −1 | −2 | 0 | −2 |
| 36 | A | 2 | 1 | 0 | −1 | −1 | 0 | −1 | −1 | 4 | −1 | −1 | 0 | −1 | −2 | −1 | 0 | 2 | −2 | 0 | −1 |
| 37 | H | 0 | −1 | 0 | −1 | −2 | −1 | −1 | 3 | 6 | −3 | −3 | −1 | −2 | −2 | −2 | 1 | −1 | −2 | 0 | −2 |
| 38 | H | 0 | −1 | 0 | −2 | 5 | −1 | −1 | −2 | 5 | −1 | 0 | −1 | −1 | −1 | −2 | 0 | 1 | −2 | 0 | −1 |
| 39 | V | −1 | −2 | −2 | −2 | −2 | −1 | 0 | −3 | −2 | 2 | 2 | −2 | 0 | −1 | 2 | −2 | −1 | −3 | −1 | 2 |
| 40 | R | 1 | 2 | −1 | −1 | −2 | 1 | 1 | −1 | −1 | −2 | −2 | 3 | −1 | −3 | −1 | 0 | −1 | −3 | −2 | −2 |
| 41 | T | 2 | −1 | −1 | −1 | −2 | 3 | 0 | 1 | −1 | −2 | −2 | 0 | −1 | −3 | 0 | 0 | 2 | −2 | −2 | −1 |
| 42 | G | 0 | −2 | 0 | −1 | −3 | −2 | −2 | 6 | −2 | −4 | −4 | −2 | −3 | −3 | −2 | 0 | −2 | −2 | −3 | −3 |
| 43 | T | 0 | −1 | 0 | −1 | −1 | −1 | −1 | −2 | −2 | −1 | −1 | −1 | −1 | −2 | −1 | 0 | 6 | −2 | −2 | 0 |
| 44 | C | 0 | −3 | −3 | −3 | 10 | −3 | −4 | −3 | −3 | −1 | −1 | −3 | −1 | −2 | −3 | −1 | −1 | −2 | −2 | −1 |
| 45 | E | −1 | 0 | 0 | 1 | −4 | 1 | 6 | −2 | 0 | −3 | −3 | 0 | −2 | −3 | −1 | 0 | −1 | −3 | −2 | −2 |
| 46 | V | 0 | −3 | −3 | −3 | −1 | −2 | −2 | −3 | −3 | 3 | 0 | −2 | 0 | −1 | −2 | −2 | 0 | −3 | −1 | 4 |
| 47 | V | 0 | −3 | −3 | −3 | −1 | −2 | −2 | −3 | −3 | 4 | 0 | −2 | 0 | −1 | −2 | −2 | 0 | −3 | −1 | 4 |
| 48 | A | 4 | −1 | −1 | −2 | 0 | −1 | −1 | −1 | −2 | −1 | −1 | −1 | −1 | −2 | −1 | 0 | 2 | −3 | −2 | 0 |
| 49 | L | 3 | −2 | −3 | −3 | −1 | −2 | −2 | −2 | −3 | 0 | 2 | −2 | 0 | −1 | −2 | 0 | 0 | −3 | −1 | 1 |
| 50 | H | −2 | −1 | 0 | 3 | −3 | 0 | 0 | −2 | 8 | −3 | −3 | −1 | −2 | −2 | −2 | −1 | −2 | −3 | 0 | −3 |
| 51 | R | −1 | 6 | 0 | −2 | −3 | 0 | 0 | −2 | 0 | −3 | −2 | 1 | −1 | −3 | −2 | −1 | −1 | −3 | −2 | −3 |
| 52 | C | −1 | −3 | −1 | 3 | 8 | −2 | −1 | −2 | −2 | −2 | −2 | −2 | −2 | −2 | −2 | −1 | −1 | −3 | −2 | −2 |
| 53 | C | 0 | −2 | −1 | −2 | 8 | −2 | −2 | −2 | −2 | −1 | −1 | −2 | −1 | −2 | −2 | 2 | 0 | −2 | −2 | −1 |
| 54 | N | −1 | 0 | 6 | 0 | −2 | 0 | 0 | 0 | 0 | −3 | −3 | 0 | −2 | −3 | −2 | 2 | 0 | −4 | −2 | −3 |
| 55 | K | −1 | 2 | 0 | −1 | −3 | 3 | 0 | −2 | −1 | −3 | −2 | 4 | −1 | −3 | −1 | 0 | −1 | −3 | −2 | −2 |
| 56 | N | −2 | −1 | 6 | 0 | −3 | 0 | 0 | −1 | 0 | −3 | −3 | 0 | −2 | −3 | 3 | 0 | 0 | −4 | −2 | −3 |
| 57 | K | −1 | 5 | 0 | −2 | −3 | 0 | 0 | −2 | 0 | −3 | −2 | 3 | −1 | −3 | −2 | −1 | −1 | −3 | −2 | −3 |
| 58 | I | −1 | 2 | −2 | −3 | −2 | −1 | −2 | −3 | −2 | 4 | 0 | −1 | 0 | −1 | −3 | −2 | −1 | −3 | −1 | 1 |
| 59 | E | −1 | 0 | 0 | 0 | −3 | 0 | 5 | −2 | −1 | −2 | −2 | 0 | −2 | −3 | −1 | 0 | 2 | −3 | −2 | −1 |
| 60 | E | −1 | −1 | −1 | 0 | −3 | 0 | 5 | −3 | −1 | 2 | −1 | 0 | −1 | −2 | −2 | −1 | −1 | −3 | −2 | 0 |
| 61 | R | 1 | 5 | −1 | −2 | −2 | 0 | 0 | −1 | −1 | −2 | −2 | 0 | −1 | −3 | −2 | 0 | −1 | −3 | −2 | −2 |
| 62 | S | 0 | 2 | 0 | −1 | −2 | 0 | 0 | −1 | −1 | −2 | −2 | 0 | −1 | −2 | −1 | 4 | 0 | −3 | −2 | −2 |
| 63 | Q | −1 | 0 | 0 | 0 | −3 | 6 | 1 | −2 | 0 | −3 | −2 | 0 | 0 | −3 | −1 | 0 | −1 | −2 | −1 | −2 |
| 64 | T | 0 | −1 | 0 | −1 | −1 | −1 | −1 | −2 | −2 | −1 | −1 | −1 | −1 | −2 | −1 | 0 | 6 | −2 | −2 | 0 |
| 65 | V | 2 | −2 | −3 | −3 | −1 | −2 | −2 | −2 | −3 | 1 | 0 | −2 | 0 | −1 | −2 | −1 | 0 | −3 | −1 | 4 |
| 66 | K | −1 | 3 | 0 | −1 | −3 | 0 | 0 | −2 | −1 | −3 | −2 | 5 | −1 | −3 | −1 | 0 | −1 | −3 | −2 | −2 |
| 67 | C | 0 | −3 | −3 | −3 | 10 | −3 | −4 | −3 | −3 | −1 | −1 | −3 | −1 | −2 | −3 | −1 | −1 | −2 | −2 | −1 |
| 68 | S | 2 | −1 | 0 | −1 | −1 | 0 | 0 | 0 | −1 | −2 | −2 | 0 | −1 | −2 | −1 | 4 | 0 | −3 | −2 | −1 |
| 69 | C | 0 | −3 | −3 | −3 | 10 | −3 | −4 | −3 | −3 | −1 | −1 | −3 | −1 | −2 | −3 | −1 | −1 | −2 | −2 | −1 |
| 70 | F | −2 | 2 | −2 | −3 | −2 | −1 | −2 | −3 | −1 | 0 | 1 | −1 | 0 | 4 | −3 | −2 | −2 | −1 | 0 | −1 |
| 71 | P | −1 | 0 | −1 | −1 | −3 | 0 | 0 | −2 | −2 | −3 | −3 | 2 | −2 | −3 | 5 | 1 | −1 | −4 | −3 | −2 |
| 72 | G | 0 | −2 | 0 | −1 | −3 | −2 | −2 | 6 | −2 | −4 | −4 | −2 | −3 | −3 | −2 | 0 | −2 | −2 | −3 | −3 |
| 73 | Q | −1 | 0 | 0 | 0 | −3 | 6 | 1 | −2 | 0 | −3 | −2 | 2 | 0 | −3 | −1 | 0 | −1 | −2 | −1 | −2 |
| 74 | V | 0 | −3 | −3 | −3 | −1 | −2 | −2 | −3 | −3 | 3 | 0 | −2 | 0 | −1 | −2 | −2 | 0 | −3 | −1 | 4 |
| 75 | A | 5 | −1 | −2 | −2 | 0 | −1 | −1 | 0 | −2 | −1 | −1 | −1 | −1 | −2 | −1 | 0 | 0 | −3 | −2 | 0 |
| 76 | G | 0 | −2 | 0 | −1 | −3 | −2 | −2 | 6 | −2 | −4 | −4 | −2 | −3 | −3 | −2 | 0 | −2 | −2 | −3 | −3 |
| 77 | T | 0 | −1 | 0 | −1 | −1 | −1 | −1 | −2 | −2 | −1 | −1 | −1 | −1 | −2 | −1 | 0 | 6 | −2 | −2 | 0 |
| 78 | T | 0 | −1 | 0 | −1 | −1 | −1 | −1 | −2 | −2 | −1 | −1 | −1 | −1 | −2 | −1 | 0 | 6 | −2 | −2 | 0 |
| 79 | R | −1 | 6 | 0 | −2 | −3 | 0 | 0 | −2 | 0 | −3 | −2 | 1 | −1 | −3 | −2 | −1 | −1 | −3 | −2 | −3 |
| 80 | A | 4 | −1 | 1 | −1 | 0 | −1 | −1 | 0 | −1 | −1 | −1 | −1 | −1 | −2 | −1 | 0 | 0 | −3 | −2 | 0 |
| 81 | A | 0 | 4 | 0 | −2 | −2 | 2 | 0 | −2 | −1 | −2 | 0 | 2 | 0 | −3 | −2 | 0 | −1 | −3 | −2 | −2 |
| 82 | P | −1 | −2 | −2 | −1 | −3 | −1 | −1 | −2 | −2 | −3 | −3 | −1 | −2 | −4 | 7 | −1 | −1 | −4 | −3 | −2 |
| 83 | S | 2 | −1 | 0 | −1 | −1 | 0 | 0 | 0 | −1 | −2 | −2 | 0 | −1 | −2 | −1 | 4 | 0 | −3 | −2 | −1 |
| 84 | C | 0 | −3 | −3 | −3 | 10 | −3 | −4 | −3 | −3 | −1 | −1 | −3 | −1 | −2 | −3 | −1 | −1 | −2 | −2 | −1 |
| 85 | V | 0 | −3 | −3 | −3 | −1 | −2 | −2 | −3 | −3 | 2 | 0 | −2 | 0 | −1 | −2 | −2 | 0 | −3 | −1 | 5 |
| 86 | D | −2 | −2 | 0 | 6 | −3 | 0 | 2 | −1 | −1 | −3 | −4 | −1 | −3 | −3 | −1 | 0 | −1 | −4 | −3 | −3 |
| 87 | A | 5 | −1 | −2 | −2 | 0 | −1 | −1 | 0 | −2 | −1 | −1 | −1 | −1 | −2 | −1 | 0 | 0 | −3 | −2 | 0 |
| 88 | S | 0 | 2 | 0 | −1 | −1 | 0 | 0 | −1 | −1 | −2 | −2 | 0 | −1 | −2 | −1 | 4 | 0 | −3 | −2 | −2 |
| 89 | I | −1 | −3 | −3 | −3 | −1 | −3 | −3 | −4 | −3 | 5 | 1 | −3 | 0 | 0 | −3 | −2 | −1 | −3 | −1 | 2 |
| 90 | V | 0 | −3 | −3 | −3 | −1 | −2 | −2 | −3 | −3 | 3 | 0 | −2 | 0 | −1 | −2 | −2 | 0 | −3 | −1 | 4 |
| 91 | E | −1 | 1 | −1 | −1 | −2 | 0 | 1 | −3 | −2 | 2 | 1 | 2 | 0 | −1 | −2 | −1 | −1 | −3 | −2 | 0 |
| 92 | Q | −1 | 0 | 0 | 0 | −2 | 4 | 1 | 0 | −1 | −2 | −2 | 0 | −1 | −2 | −1 | 0 | 2 | 2 | −1 | −2 |
| 93 | K | −1 | 2 | 0 | −1 | −3 | 0 | 0 | −2 | −1 | −3 | −2 | 6 | −1 | −3 | −1 | 0 | −1 | −3 | −2 | −2 |
| 94 | W | −2 | −1 | −2 | −2 | −2 | 2 | −1 | −2 | −1 | −3 | −2 | −1 | −1 | 0 | −3 | −2 | −2 | 10 | 0 | −3 |
| 95 | W | −3 | −3 | −4 | −4 | −2 | −2 | −3 | −2 | −2 | −3 | −2 | −3 | −1 | 0 | −4 | −3 | −2 | 11 | 1 | −3 |
| 96 | C | 0 | −3 | −3 | −3 | 10 | −3 | −4 | −3 | −3 | −1 | −1 | −3 | −1 | −2 | −3 | −1 | −1 | −2 | −2 | −1 |
| 97 | H | −2 | 0 | 1 | 3 | −3 | 2 | 2 | −2 | 5 | −3 | −3 | 0 | −2 | −2 | −1 | 0 | −1 | −3 | 0 | −3 |
| 98 | M | −1 | −1 | −2 | −3 | −1 | 0 | −2 | −3 | −2 | 0 | 1 | −1 | 7 | 0 | −2 | −1 | −1 | −1 | −1 | 0 |
| 99 | Q | −1 | 0 | 2 | 0 | −3 | 2 | 3 | −2 | 0 | −1 | 1 | 0 | 0 | −2 | −2 | 0 | −1 | −3 | −2 | −1 |
| 100 | P | −1 | −2 | −2 | −1 | −3 | −1 | −1 | −2 | −2 | −3 | −3 | −1 | −2 | −4 | 7 | −1 | −1 | −4 | −3 | −2 |
| 101 | C | 0 | −3 | −3 | −3 | 10 | −3 | −4 | −3 | −3 | −1 | −1 | −3 | −1 | −2 | −3 | −1 | −1 | −2 | −2 | −1 |
| 102 | L | −1 | −2 | −3 | −4 | −1 | −2 | −3 | −4 | −3 | 1 | 4 | −2 | 1 | 0 | −3 | −2 | −1 | −2 | −1 | 1 |
| 103 | E | −1 | −1 | −1 | 0 | −3 | 0 | 5 | −2 | −1 | −1 | 0 | 0 | −1 | −2 | 0 | 0 | −1 | −3 | −2 | 0 |
| 104 | G | 0 | −2 | 0 | −1 | −3 | −2 | −2 | 6 | −2 | −4 | −4 | −2 | −3 | −3 | −2 | 0 | −2 | −2 | −3 | −3 |
| 105 | E | −1 | 0 | 0 | 1 | −4 | 1 | 6 | −2 | 0 | −3 | −3 | 0 | −2 | −3 | −1 | 0 | −1 | −3 | −2 | −2 |
| 106 | E | −1 | −1 | 0 | 2 | −4 | 0 | 4 | 2 | −1 | −3 | −4 | 0 | −3 | −3 | −1 | 0 | −1 | −3 | −3 | −3 |
| 107 | C | 0 | −3 | −3 | −3 | 10 | −3 | −4 | −3 | −3 | −1 | −1 | −3 | −1 | −2 | −3 | −1 | −1 | −2 | −2 | −1 |
| 108 | K | −1 | 1 | 0 | 3 | −3 | 0 | 0 | −2 | −1 | −3 | −3 | 4 | −2 | −3 | −1 | 0 | −1 | −3 | −2 | −2 |
| 109 | V | 0 | −2 | −2 | −3 | −1 | −2 | −2 | −3 | −3 | 1 | 2 | −2 | 0 | −1 | −2 | −1 | 1 | −3 | −1 | 4 |
| 110 | L | −1 | −2 | −3 | −4 | −1 | −2 | −3 | −4 | −3 | 1 | 4 | −2 | 1 | 0 | −3 | −2 | −1 | −2 | −1 | 0 |
| 111 | P | −1 | −2 | −2 | −2 | −2 | −2 | −2 | −3 | −2 | 2 | −1 | −2 | −1 | −2 | 6 | −1 | −1 | −4 | −2 | 0 |
| 112 | D | −2 | −1 | 3 | 6 | −3 | 0 | 1 | −1 | 0 | −3 | −4 | −1 | −3 | −3 | −1 | 0 | −1 | −4 | −3 | −3 |
| 113 | R | −1 | 3 | 2 | −2 | −2 | 0 | −1 | −2 | 0 | −1 | 1 | 0 | 0 | −1 | −2 | 0 | −1 | −2 | 1 | −1 |
| 114 | K | 0 | −1 | 0 | 0 | −1 | 0 | 0 | 0 | −1 | −2 | −2 | 1 | −1 | −2 | −1 | 4 | 1 | −3 | −2 | −2 |
| 115 | G | 0 | −2 | 0 | −1 | −3 | −2 | −2 | 6 | −2 | −4 | −4 | −2 | −3 | −3 | −2 | 0 | −2 | −2 | −3 | −3 |
| 116 | W | −3 | −3 | −4 | −4 | −2 | −2 | −3 | −2 | −2 | −3 | −2 | −3 | −1 | 0 | −4 | −3 | −2 | 11 | 1 | −3 |
| 117 | S | 0 | −1 | 0 | −1 | −1 | 0 | −1 | −1 | −1 | −1 | −1 | 0 | 2 | −2 | −1 | 3 | 3 | −2 | −2 | −1 |
| 118 | C | 0 | −3 | −3 | −3 | 10 | −3 | −4 | −3 | −3 | −1 | −1 | −3 | −1 | −2 | −3 | −1 | −1 | −2 | −2 | −1 |
| 119 | S | 1 | −1 | 0 | −1 | −1 | −1 | −1 | −1 | −2 | 0 | −1 | −1 | −1 | −2 | −1 | 3 | 3 | −3 | −2 | 0 |
| 120 | S | 0 | 0 | 0 | 0 | −2 | 3 | 0 | −1 | −1 | −2 | −2 | 0 | −1 | −2 | −1 | 3 | 1 | −3 | −2 | −2 |
| 121 | G | 0 | −2 | 0 | −1 | −3 | −2 | −2 | 6 | −2 | −4 | −4 | −2 | −3 | −3 | −2 | 0 | −2 | −2 | −3 | −3 |
| 122 | N | −1 | −1 | 5 | 0 | −3 | −1 | −1 | 3 | 4 | −3 | −3 | −1 | −2 | −3 | −2 | 0 | −1 | −3 | −1 | −3 |
| 123 | K | −1 | 3 | 0 | −1 | −3 | 0 | 0 | −2 | −1 | −3 | −2 | 5 | −1 | −3 | −1 | 0 | −1 | −3 | −2 | −2 |
| 124 | V | 0 | −3 | −3 | −3 | −1 | −2 | −2 | −4 | −3 | 4 | 0 | −2 | 0 | −1 | −2 | −2 | 0 | −3 | −1 | 4 |
| 125 | K | −1 | 1 | 0 | −1 | −3 | 0 | 0 | −2 | −1 | −3 | −2 | 6 | −1 | −3 | −1 | 0 | −1 | −3 | −2 | −2 |
| 126 | T | 0 | −1 | 0 | −1 | −1 | −1 | −1 | −2 | −2 | −1 | −1 | −1 | −1 | −2 | −1 | 0 | 6 | −2 | −2 | 0 |
| 127 | T | 0 | −1 | 0 | −1 | −1 | −1 | −1 | −2 | −2 | −1 | −1 | −1 | −1 | −2 | −1 | 0 | 6 | −2 | −2 | 0 |
| 128 | R | −1 | 3 | 0 | −1 | −2 | 0 | 0 | −2 | −1 | −2 | −2 | 4 | −1 | −3 | −1 | 0 | 2 | −3 | −2 | −2 |
| 129 | V | 0 | −3 | −3 | −3 | −1 | −2 | −2 | −3 | −3 | 2 | 0 | −2 | 0 | −1 | −2 | −2 | 0 | −3 | −1 | 5 |
| 130 | T | 0 | −1 | 0 | −1 | −1 | 0 | −1 | −1 | 2 | −1 | 0 | −1 | −1 | −2 | −1 | 3 | 3 | −3 | −1 | −1 |
| 131 | H | −1 | 4 | 0 | −1 | −3 | 0 | 0 | −2 | 4 | −3 | −2 | 0 | −1 | −3 | 3 | −1 | −1 | −3 | −1 | −3; |

or
2) comprising a fragment of the amino acid sequence of a1), wherein said fragment is a member of the EGF domain containing protein family, or has an antigenic determinant in common with the amino acid sequence of a1); or
3) comprising a functional equivalent of the amino acid sequence of a1) or the fragment of a2); or
4) consisting of the amino acid sequence of a1); or
5) of any of a1)-a4), wherein the polypeptide has a maximum threshold E value of 10⁻²; or
6) comprising an amino acid sequence satisfying the consensus amino acid sequence G-T-C-E-[VI]-[VI]-[AT]-[AVL]-[HD]-R-[CD]-[CS]-[NS]-[KRQ]-[NP]-[RK]-[IR]-[ET]-[EI]-[RA]-[SR]-Q-T-[VA]-[KR]-C-[SA]-C-[LFR]-[PSK]-G-[KQ]-[VI]-A-G-T-T-R-[NA]-[RQLAK]-P-[SA]-C-V-[DE]-A-[SAR]-I-[VI]-[IELKR]-[WGQET]-[KR]-[WQ]-W-C-[EHNQD]-M-[ENQL]-P-C-[LV]-[EVLP]-G-E-[DEG]-C-[KRD]-[TVL]-L-[PI]-[DN]-[NYSLR]-[STK]-G-W-[MST]-C-[ASIT]-[TSRQ]-P(0,1)-G-[NHG]-[KR]-[IV]-K-T-T; or
7) consisting of an amino acid sequence satisfying the consensus amino acid sequence G-T-C-E-[VI]-[VI]-[AT]-[AVL]-[HD]-R-[CD]-[CS]-[NS]-[KRQ]-[NP]-[RK]-[IR]-[ET]-[EI]-[RA]-[SR]-Q-T-[VA]-[KR]-C-[SA]-C-[LFR]-[PSK]-G-[KQ]-[VI]-A-G-T-T-R-[NA]-[RQLAK]-P-[SA]-C-V-[DE]-A-[SAR]-I-[VI]-[IELKR]-[WGQET]-[KR]-[WQ]-W-C-[EHNQD]-M-[ENQL]-P-C-[LV]-[EVLP]-G-E-[DEG]-C-[KRD]-[TVL]-L-[PI]-[DN]-[NYSLR]-[STK]-G-W-[MST]-C-[ASIT]-[TSRQ]-P(0,1)-G-[NHG]-[KR]-[IV]-K-T-T; or
8) comprising an amino acid sequence selected from the group consisting of SEQ ID NO:18, SEQ ID NO:20, SEQ ID NO:22, SEQ ID NO:24, SEQ ID NO:26, SEQ ID NO:28 and SEQ ID NO:30; or
9) consisting of an amino acid sequence selected from the group consisting of SEQ ID NO:18, SEQ ID NO:20, SEQ ID NO:22, SEQ ID NO:24, SEQ ID NO:26, SEQ ID NO:28 and SEQ ID NO:30; or
10) comprising an amino acid sequence selected from the group consisting of SEQ ID NO:2, SEQ ID NO:4, SEQ ID NO:37 and SEQ ID NO:39; or
11) consisting of an amino acid sequence selected from the group consisting of SEQ ID NO:2, SEQ ID NO:4, SEQ ID NO:37 and SEQ ID NO:39; or
12) comprising an amino acid sequence selected from the group consisting of SEQ ID NO:6, SEQ ID NO:8, SEQ ID NO:41, SEQ ID NO:43, SEQ ID NO:53 and SEQ ID NO:55; or
13) consisting of an amino acid sequence selected from the group consisting of SEQ ID NO:6, SEQ ID NO:8, SEQ ID NO:41, SEQ ID NO:43, SEQ ID NO:53 and SEQ ID NO:55; or
14) comprising an amino acid sequence selected from the group consisting of SEQ ID NO:10, SEQ ID NO:12, SEQ ID NO:45 and SEQ ID NO:47; or
15) consisting of an amino acid sequence selected from the group consisting of SEQ ID NO:10, SEQ ID NO:12, SEQ ID NO:45 and SEQ ID NO:47; or
16) comprising an amino acid sequence selected from the group consisting of SEQ ID NO:14, SEQ ID NO:16, SEQ ID NO:49 and SEQ ID NO:51; or
17) consisting of an amino acid sequence selected from the group consisting of SEQ ID NO:14, SEQ ID NO:16, SEQ ID NO:49 and SEQ ID NO:51; or
18) comprising a fragment of any of the amino acid sequences of a6)-a17), wherein said fragment is a member of the EGF domain containing protein family, or has an antigenic determinant in common with any of the amino acid sequences of a6)-a17); or
19) comprising a functional equivalent of any of the polypeptides of a6)-a18); or
20) comprising the functional equivalent of a3) or a19), wherein the polypeptide is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55, and wherein the polypeptide is a member of the EGF domain containing protein family; or
21) comprising the fragment or functional equivalent of any of a2), a3), a18), a19), or a20), wherein the fragment or functional equivalent has greater than 80% sequence identity with an amino acid sequence selected from the group consisting of SEQ ID NO:2, SEQ ID NO:4, SEQ ID NO:6, SEQ ID NO: 8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55, or with an active fragment of any of the foregoing; or
22) comprising the functional equivalent of any of a3), a19), or a20), wherein the functional equivalent exhibits significant structural homology with a polypeptide having an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55; or
23) comprising the fragment of any of a2), a18), or a21), wherein the fragment has an antigenic determinant in common with the polypeptide of a1) or any one of a6)-a17), wherein the antigenic determinant consists of 7 or more amino acid residues from an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55; or
24) according to any of a1)-a23), wherein the polypeptide has biological activity similar to Coagulation Factor X; or
b) a purified nucleic acid molecule:
1) comprising a nucleic acid sequence encoding a polypeptide according to any one of a1)-a24); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, and SEQ ID NO:54, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, and SEQ ID NO:54, or a redundant equivalent or fragment of any of the foregoing; or
4) that hybridizes under high stringency conditions with a nucleic acid molecule of any one of b1) to b3); or
c) a vector comprising a nucleic acid molecule according to any one of b1) to b4); or
d) a host cell transformed with a vector according to c); or
e) a ligand:
1) that binds specifically to the polypeptide of any of a1) to a24); or
2) which is an antibody that binds specifically to the polypeptide of any of a1) to a24); or
f) a compound:
1) that increases the level of expression or activity of a polypeptide according to any of a1) to a24); or
2) that decreases the level of expression or activity of a polypeptide according to any of a1) to a24); or
g) the compound of f1) or f2), wherein the compound binds to a polypeptide according to any of a1) to a24) without inducing any of the biological effects of the polypeptide; or
h) the compound of g), wherein the compound is a natural or modified substrate, ligand, enzyme, receptor or structural or functional mimetic; or
i) a pharmaceutical composition comprising any one of a) to h), and a pharmaceutically acceptable carrier; or
j) a vaccine composition comprising any one of a1) to a24) or b1) to b4); or
k) a kit for diagnosing disease, comprising a first container containing a nucleic acid probe that hybridizes under stringent conditions with a nucleic acid molecule of any one of b1) to b4), a second container containing primers useful for amplifying the nucleic acid molecule, and instructions for using the probe and primers for facilitating the diagnosis of disease; or
l) a kit for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
m) a kit comprising an array of nucleic acid molecules, at least one of which is a nucleic acid molecule according to any one of b1) to b4); or
n) a kit comprising one or more antibodies that bind to a polypeptide as recited in any one of a1) to a24); and a reagent useful for the detection of a binding reaction between the one or more antibodies and the polypeptide; or
o) a transgenic or knockout non-human animal that has been transformed to express higher, lower, or absent levels of a polypeptide according to any one of a1) to a24).

69. A method of using a composition of matter, comprising obtaining a composition of matter according to claim 68 and using said composition of matter in a method selected from: diagnosing a disease in a patient; treatment of a disease in a patient; monitoring the therapeutic treatment of a disease; identification of a compound that is effective in the treatment and/or diagnosis of a disease; and screening candidate compounds.

70. The method of claim 69, wherein said method of using a composition of matter comprises the method for treatment of a disease, comprising administering to the patient:
a) an isolated polypeptide:
1) comprising an amino acid sequence that has an E value of 10⁻² or less when the profile below is input as a query sequence into the search program BLAST, using the default parameters specified by the NCBI (the National Center for Biotechnology Information) and has Blosum 62 matrix; gap open penalty=11 and gap extension penalty=1

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | M | −1 | −1 | −2 | −3 | −1 | 0 | −2 | −3 | −2 | 0 | 1 | −1 | 7 | 0 | −2 | −1 | −1 | −1 | −1 | 0 |
| 2 | N | 1 | 2 | 2 | −1 | −1 | 0 | −1 | −1 | −1 | −1 | −1 | 0 | 3 | −2 | −2 | 1 | 0 | −3 | −2 | −1 |
| 3 | K | 0 | 0 | −1 | 0 | −2 | 1 | 2 | −2 | −1 | 0 | −1 | 1 | −1 | −3 | 2 | −1 | −1 | −3 | −2 | 1 |
| 4 | R | 0 | 3 | −1 | −2 | 4 | 0 | −1 | −2 | −1 | 0 | 0 | 0 | 0 | −2 | −2 | 1 | −1 | −3 | −2 | −1 |
| 5 | Y | 1 | 0 | −2 | −2 | −2 | −1 | −1 | −2 | −1 | 0 | 0 | −1 | 0 | −1 | 3 | 0 | −1 | −2 | 2 | 0 |
| 6 | L | −1 | 2 | −1 | −1 | −2 | 0 | 0 | −2 | −1 | −1 | 0 | 3 | 2 | −2 | −2 | 0 | 1 | −2 | −2 | −1 |
| 7 | Q | 1 | 0 | 0 | −1 | −1 | 1 | 0 | −1 | −1 | −2 | −2 | 0 | −1 | −2 | −1 | 2 | 2 | 3 | −1 | −1 |
| 8 | K | −1 | −1 | 2 | −1 | −2 | −1 | −1 | 0 | −1 | 0 | 0 | 1 | 2 | −1 | −2 | 0 | 0 | −3 | −2 | 2 |
| 9 | A | 0 | 0 | −2 | −2 | −1 | −1 | −2 | −2 | −2 | 0 | 0 | −1 | 0 | −1 | −2 | 0 | −1 | 7 | 0 | 0 |
| 10 | T | 0 | 1 | 0 | −1 | −1 | 0 | −1 | −1 | −1 | −1 | 0 | 0 | 0 | −2 | 1 | 2 | 1 | −3 | −2 | −1 |
| 11 | Q | 1 | 0 | 0 | −1 | −1 | 2 | 0 | −1 | −1 | −1 | 0 | 0 | 0 | −2 | 0 | 2 | 0 | −3 | −2 | −1 |
| 12 | G | 0 | 0 | 0 | 2 | −2 | −1 | 0 | 3 | 4 | −2 | −2 | −1 | 2 | −2 | −2 | 0 | −1 | −2 | −1 | −2 |
| 13 | K | 1 | −1 | −1 | −1 | −2 | −1 | −1 | 2 | −2 | −3 | −2 | 0 | −1 | −1 | −2 | 2 | −1 | 7 | −1 | −2 |
| 14 | L | 0 | −2 | −2 | −3 | −1 | −2 | −2 | 1 | −2 | −1 | 0 | −2 | 0 | 0 | −2 | −1 | 1 | 8 | 0 | −1 |
| 15 | L | 2 | −2 | −3 | −3 | −1 | −2 | −2 | −2 | −3 | 1 | 3 | −2 | 0 | −1 | −2 | −1 | 0 | −3 | −1 | 2 |
| 16 | I | −1 | −2 | −3 | −4 | −1 | −2 | −3 | −4 | −3 | 2 | 4 | −2 | 1 | 1 | −3 | −2 | −1 | −2 | 0 | 1 |
| 17 | I | 1 | −2 | −2 | −2 | −1 | −2 | −2 | −2 | −1 | 1 | 1 | −2 | 0 | 0 | 3 | −1 | −1 | −1 | 2 | 0 |
| 18 | I | 0 | −2 | −1 | −2 | −1 | −1 | −2 | −2 | −2 | 1 | 3 | −1 | 0 | −1 | −2 | 1 | 0 | −2 | −1 | 0 |
| 19 | F | −1 | −2 | −3 | −3 | 6 | −2 | −3 | −3 | −1 | −1 | 0 | −2 | 3 | 2 | −3 | −2 | −1 | 6 | 3 | −1 |
| 20 | I | 0 | −2 | −1 | −2 | −1 | −2 | −2 | −2 | −2 | 3 | 1 | −2 | 0 | −1 | −2 | 1 | 1 | −3 | −1 | 2 |
| 21 | V | 2 | −2 | −1 | −2 | 3 | −1 | −1 | −1 | −2 | 0 | 0 | −1 | 0 | −2 | −2 | 2 | 0 | −3 | −2 | 0 |
| 22 | T | 1 | −2 | −2 | −2 | 3 | −1 | −2 | −2 | −2 | −1 | 0 | −2 | 2 | −1 | −2 | 0 | 2 | 6 | −1 | 0 |
| 23 | L | −1 | −3 | −3 | −3 | 4 | −3 | −3 | −3 | −2 | 0 | 2 | −3 | 0 | 4 | −3 | −2 | −1 | −1 | 0 | 1 |
| 24 | W | 0 | −3 | −3 | −3 | 5 | −2 | −3 | −2 | −2 | −1 | 0 | −3 | −1 | 0 | −3 | −1 | −1 | 9 | 0 | −1 |
| 25 | G | 1 | −2 | −1 | −2 | 4 | −1 | −2 | 1 | −2 | 0 | 0 | −1 | 2 | −2 | −2 | 0 | 1 | −2 | −2 | 0 |
| 26 | K | −1 | 0 | −1 | −2 | 1 | 1 | 0 | −3 | 1 | 0 | 1 | 2 | 0 | 2 | −2 | −1 | −1 | −2 | 0 | −1 |
| 27 | A | 0 | −2 | −3 | −3 | −1 | −2 | −2 | −3 | −3 | 1 | 3 | −2 | 3 | 0 | 0 | −2 | −1 | −2 | −1 | 1 |
| 28 | V | 0 | −2 | −2 | −3 | 4 | −2 | −2 | −3 | −3 | 3 | 0 | −2 | 3 | −1 | −2 | −1 | 0 | −2 | −1 | 2 |
| 29 | S | 0 | −1 | 0 | −1 | −1 | −1 | −1 | −1 | 5 | −1 | 1 | −1 | 0 | −1 | −2 | 2 | 0 | −3 | 0 | −1 |
| 30 | S | 4 | −1 | −1 | −2 | −1 | −1 | −1 | 2 | −2 | −2 | −2 | −1 | −1 | −2 | −1 | 1 | 1 | −3 | −2 | −1 |
| 31 | A | 1 | −1 | 0 | 0 | −1 | 1 | 0 | 1 | −1 | −2 | −2 | 0 | −1 | −2 | −1 | 4 | 0 | −3 | −2 | −2 |
| 32 | N | −1 | −1 | 2 | −1 | −2 | −1 | −2 | −2 | −1 | 0 | 3 | −1 | 0 | 1 | −3 | 1 | −1 | −2 | −1 | 0 |
| 33 | H | −1 | 0 | 0 | −1 | −2 | 5 | 0 | −3 | 3 | −1 | 1 | 0 | 0 | −2 | −2 | −1 | −1 | −2 | −1 | −1 |
| 34 | H | −1 | 0 | 0 | −1 | −2 | 0 | 0 | −2 | 6 | 0 | −1 | 0 | −1 | −2 | 2 | 0 | −1 | −3 | 0 | −1 |
| 35 | K | −1 | 0 | 0 | −1 | −3 | 3 | 0 | −2 | 4 | −3 | −2 | 1 | −1 | −3 | 3 | 0 | −1 | −2 | 0 | −2 |
| 36 | A | 2 | 1 | 0 | −1 | −1 | 0 | −1 | −1 | 4 | −1 | −1 | 0 | −1 | −2 | −1 | 0 | 2 | −2 | 0 | −1 |
| 37 | H | 0 | −1 | 0 | −1 | −2 | −1 | −1 | 3 | 6 | −3 | −3 | −1 | −2 | −2 | −2 | 1 | −1 | −2 | 0 | −2 |
| 38 | H | 0 | −1 | 0 | −2 | 5 | −1 | −1 | −2 | 5 | −1 | 0 | −1 | −1 | −1 | −2 | 0 | 1 | −2 | 0 | −1 |
| 39 | V | −1 | −2 | −2 | −2 | −2 | −1 | 0 | −3 | −2 | 2 | 2 | −2 | 0 | −1 | 2 | −2 | −1 | −3 | −1 | 2 |
| 40 | R | 1 | 2 | −1 | −1 | −2 | 1 | 1 | −1 | −1 | −2 | −2 | 3 | −1 | −3 | −1 | 0 | −1 | −3 | −2 | −2 |
| 41 | T | 2 | −1 | −1 | −1 | −2 | 3 | 0 | 1 | −1 | −2 | −2 | 0 | −1 | −3 | 0 | 0 | 2 | −2 | −2 | −1 |
| 42 | G | 0 | −2 | 0 | −1 | −3 | −2 | −2 | 6 | −2 | −4 | −4 | −2 | −3 | −3 | −2 | 0 | −2 | −2 | −3 | −3 |
| 43 | T | 0 | −1 | 0 | −1 | −1 | −1 | −1 | −2 | −2 | −1 | −1 | −1 | −1 | −2 | −1 | 0 | 6 | −2 | −2 | 0 |
| 44 | C | 0 | −3 | −3 | −3 | 10 | −3 | −4 | −3 | −3 | −1 | −1 | −3 | −1 | −2 | −3 | −1 | −1 | −2 | −2 | −1 |
| 45 | E | −1 | 0 | 0 | 1 | −4 | 1 | 6 | −2 | 0 | −3 | −3 | 0 | −2 | −3 | −1 | 0 | −1 | −3 | −2 | −2 |
| 46 | V | 0 | −3 | −3 | −3 | −1 | −2 | −2 | −3 | −3 | 3 | 0 | −2 | 0 | −1 | −2 | −2 | 0 | −3 | −1 | 4 |
| 47 | V | 0 | −3 | −3 | −3 | −1 | −2 | −2 | −3 | −3 | 4 | 0 | −2 | 0 | −1 | −2 | −2 | 0 | −3 | −1 | 4 |
| 48 | A | 4 | −1 | −1 | −2 | 0 | −1 | −1 | −1 | −2 | −1 | −1 | −1 | −1 | −2 | −1 | 0 | 2 | −3 | −2 | 0 |
| 49 | L | 3 | −2 | −3 | −3 | −1 | −2 | −2 | −2 | −3 | 0 | 2 | −2 | 0 | −1 | −2 | 0 | 0 | −3 | −1 | 1 |
| 50 | H | −2 | −1 | 0 | 3 | −3 | 0 | 0 | −2 | 8 | −3 | −3 | −1 | −2 | −2 | −2 | −1 | −2 | −3 | 0 | −3 |
| 51 | R | −1 | 6 | 0 | −2 | −3 | 0 | 0 | −2 | 0 | −3 | −2 | 1 | −1 | −3 | −2 | −1 | −1 | −3 | −2 | −3 |
| 52 | C | −1 | −3 | −1 | 3 | 8 | −2 | −1 | −2 | −2 | −2 | −2 | −2 | −2 | −2 | −2 | −1 | −1 | −3 | −2 | −2 |
| 53 | C | 0 | −2 | −1 | −2 | 8 | −2 | −2 | −2 | −2 | −1 | −1 | −2 | −1 | −2 | −2 | 2 | 0 | −2 | −2 | −1 |
| 54 | N | −1 | 0 | 6 | 0 | −2 | 0 | 0 | 0 | 0 | −3 | −3 | 0 | −2 | −3 | −2 | 2 | 0 | −4 | −2 | −3 |
| 55 | K | −1 | 2 | 0 | −1 | −3 | 3 | 0 | −2 | −1 | −3 | −2 | 4 | −1 | −3 | −1 | 0 | −1 | −3 | −2 | −2 |
| 56 | N | −2 | −1 | 6 | 0 | −3 | 0 | 0 | −1 | 0 | −3 | −3 | 0 | −2 | −3 | 3 | 0 | 0 | −4 | −2 | −3 |
| 57 | K | −1 | 5 | 0 | −2 | −3 | 0 | 0 | −2 | 0 | −3 | −2 | 3 | −1 | −3 | −2 | −1 | −1 | −3 | −2 | −3 |
| 58 | I | −1 | 2 | −2 | −3 | −2 | −1 | −2 | −3 | −2 | 4 | 0 | −1 | 0 | −1 | −3 | −2 | −1 | −3 | −1 | 1 |
| 59 | E | −1 | 0 | 0 | 0 | −3 | 0 | 5 | −2 | −1 | −2 | −2 | 0 | −2 | −3 | −1 | 0 | 2 | −3 | −2 | −1 |
| 60 | E | −1 | −1 | −1 | 0 | −3 | 0 | 5 | −3 | −1 | 2 | −1 | 0 | −1 | −2 | −2 | −1 | −1 | −3 | −2 | 0 |
| 61 | R | 1 | 5 | −1 | −2 | −2 | 0 | 0 | −1 | −1 | −2 | −2 | 0 | −1 | −3 | −2 | 0 | −1 | −3 | −2 | −2 |
| 62 | S | 0 | 2 | 0 | −1 | −2 | 0 | 0 | −1 | −1 | −2 | −2 | 0 | −1 | −2 | −1 | 4 | 0 | −3 | −2 | −2 |
| 63 | Q | −1 | 0 | 0 | 0 | −3 | 6 | 1 | −2 | 0 | −3 | −2 | 0 | 0 | −3 | −1 | 0 | −1 | −2 | −1 | −2 |
| 64 | T | 0 | −1 | 0 | −1 | −1 | −1 | −1 | −2 | −2 | −1 | −1 | −1 | −1 | −2 | −1 | 0 | 6 | −2 | −2 | 0 |
| 65 | V | 2 | −2 | −3 | −3 | −1 | −2 | −2 | −2 | −3 | 1 | 0 | −2 | 0 | −1 | −2 | −1 | 0 | −3 | −1 | 4 |
| 66 | K | −1 | 3 | 0 | −1 | −3 | 0 | 0 | −2 | −1 | −3 | −2 | 5 | −1 | −3 | −1 | 0 | −1 | −3 | −2 | −2 |
| 67 | C | 0 | −3 | −3 | −3 | 10 | −3 | −4 | −3 | −3 | −1 | −1 | −3 | −1 | −2 | −3 | −1 | −1 | −2 | −2 | −1 |
| 68 | S | 2 | −1 | 0 | −1 | −1 | 0 | 0 | 0 | −1 | −2 | −2 | 0 | −1 | −2 | −1 | 4 | 0 | −3 | −2 | −1 |
| 69 | C | 0 | −3 | −3 | −3 | 10 | −3 | −4 | −3 | −3 | −1 | −1 | −3 | −1 | −2 | −3 | −1 | −1 | −2 | −2 | −1 |
| 70 | F | −2 | 2 | −2 | −3 | −2 | −1 | −2 | −3 | −1 | 0 | 1 | −1 | 0 | 4 | −3 | −2 | −2 | −1 | 0 | −1 |
| 71 | P | −1 | 0 | −1 | −1 | −3 | 0 | 0 | −2 | −2 | −3 | −3 | 2 | −2 | −3 | 5 | 1 | −1 | −4 | −3 | −2 |
| 72 | G | 0 | −2 | 0 | −1 | −3 | −2 | −2 | 6 | −2 | −4 | −4 | −2 | −3 | −3 | −2 | 0 | −2 | −2 | −3 | −3 |
| 73 | Q | −1 | 0 | 0 | 0 | −3 | 6 | 1 | −2 | 0 | −3 | −2 | 2 | 0 | −3 | −1 | 0 | −1 | −2 | −1 | −2 |
| 74 | V | 0 | −3 | −3 | −3 | −1 | −2 | −2 | −3 | −3 | 3 | 0 | −2 | 0 | −1 | −2 | −2 | 0 | −3 | −1 | 4 |
| 75 | A | 5 | −1 | −2 | −2 | 0 | −1 | −1 | 0 | −2 | −1 | −1 | −1 | −1 | −2 | −1 | 0 | 0 | −3 | −2 | 0 |
| 76 | G | 0 | −2 | 0 | −1 | −3 | −2 | −2 | 6 | −2 | −4 | −4 | −2 | −3 | −3 | −2 | 0 | −2 | −2 | −3 | −3 |
| 77 | T | 0 | −1 | 0 | −1 | −1 | −1 | −1 | −2 | −2 | −1 | −1 | −1 | −1 | −2 | −1 | 0 | 6 | −2 | −2 | 0 |
| 78 | T | 0 | −1 | 0 | −1 | −1 | −1 | −1 | −2 | −2 | −1 | −1 | −1 | −1 | −2 | −1 | 0 | 6 | −2 | −2 | 0 |
| 79 | R | −1 | 6 | 0 | −2 | −3 | 0 | 0 | −2 | 0 | −3 | −2 | 1 | −1 | −3 | −2 | −1 | −1 | −3 | −2 | −3 |
| 80 | A | 4 | −1 | 1 | −1 | 0 | −1 | −1 | 0 | −1 | −1 | −1 | −1 | −1 | −2 | −1 | 0 | 0 | −3 | −2 | 0 |
| 81 | A | 0 | 4 | 0 | −2 | −2 | 2 | 0 | −2 | −1 | −2 | 0 | 2 | 0 | −3 | −2 | 0 | −1 | −3 | −2 | −2 |
| 82 | P | −1 | −2 | −2 | −1 | −3 | −1 | −1 | −2 | −2 | −3 | −3 | −1 | −2 | −4 | 7 | −1 | −1 | −4 | −3 | −2 |
| 83 | S | 2 | −1 | 0 | −1 | −1 | 0 | 0 | 0 | −1 | −2 | −2 | 0 | −1 | −2 | −1 | 4 | 0 | −3 | −2 | −1 |
| 84 | C | 0 | −3 | −3 | −3 | 10 | −3 | −4 | −3 | −3 | −1 | −1 | −3 | −1 | −2 | −3 | −1 | −1 | −2 | −2 | −1 |
| 85 | V | 0 | −3 | −3 | −3 | −1 | −2 | −2 | −3 | −3 | 2 | 0 | −2 | 0 | −1 | −2 | −2 | 0 | −3 | −1 | 5 |
| 86 | D | −2 | −2 | 0 | 6 | −3 | 0 | 2 | −1 | −1 | −3 | −4 | −1 | −3 | −3 | −1 | 0 | −1 | −4 | −3 | −3 |
| 87 | A | 5 | −1 | −2 | −2 | 0 | −1 | −1 | 0 | −2 | −1 | −1 | −1 | −1 | −2 | −1 | 0 | 0 | −3 | −2 | 0 |
| 88 | S | 0 | 2 | 0 | −1 | −1 | 0 | 0 | −1 | −1 | −2 | −2 | 0 | −1 | −2 | −1 | 4 | 0 | −3 | −2 | −2 |
| 89 | I | −1 | −3 | −3 | −3 | −1 | −3 | −3 | −4 | −3 | 5 | 1 | −3 | 0 | 0 | −3 | −2 | −1 | −3 | −1 | 2 |
| 90 | V | 0 | −3 | −3 | −3 | −1 | −2 | −2 | −3 | −3 | 3 | 0 | −2 | 0 | −1 | −2 | −2 | 0 | −3 | −1 | 4 |
| 91 | E | −1 | 1 | −1 | −1 | −2 | 0 | 1 | −3 | −2 | 2 | 1 | 2 | 0 | −1 | −2 | −1 | −1 | −3 | −2 | 0 |
| 92 | Q | −1 | 0 | 0 | 0 | −2 | 4 | 1 | 0 | −1 | −2 | −2 | 0 | −1 | −2 | −1 | 0 | 2 | 2 | −1 | −2 |
| 93 | K | −1 | 2 | 0 | −1 | −3 | 0 | 0 | −2 | −1 | −3 | −2 | 6 | −1 | −3 | −1 | 0 | −1 | −3 | −2 | −2 |
| 94 | W | −2 | −1 | −2 | −2 | −2 | 2 | −1 | −2 | −1 | −3 | −2 | −1 | −1 | 0 | −3 | −2 | −2 | 10 | 0 | −3 |
| 95 | W | −3 | −3 | −4 | −4 | −2 | −2 | −3 | −2 | −2 | −3 | −2 | −3 | −1 | 0 | −4 | −3 | −2 | 11 | 1 | −3 |
| 96 | C | 0 | −3 | −3 | −3 | 10 | −3 | −4 | −3 | −3 | −1 | −1 | −3 | −1 | −2 | −3 | −1 | −1 | −2 | −2 | −1 |
| 97 | H | −2 | 0 | 1 | 3 | −3 | 2 | 2 | −2 | 5 | −3 | −3 | 0 | −2 | −2 | −1 | 0 | −1 | −3 | 0 | −3 |
| 98 | M | −1 | −1 | −2 | −3 | −1 | 0 | −2 | −3 | −2 | 0 | 1 | −1 | 7 | 0 | −2 | −1 | −1 | −1 | −1 | 0 |
| 99 | Q | −1 | 0 | 2 | 0 | −3 | 2 | 3 | −2 | 0 | −1 | 1 | 0 | 0 | −2 | −2 | 0 | −1 | −3 | −2 | −1 |
| 100 | P | −1 | −2 | −2 | −1 | −3 | −1 | −1 | −2 | −2 | −3 | −3 | −1 | −2 | −4 | 7 | −1 | −1 | −4 | −3 | −2 |
| 101 | C | 0 | −3 | −3 | −3 | 10 | −3 | −4 | −3 | −3 | −1 | −1 | −3 | −1 | −2 | −3 | −1 | −1 | −2 | −2 | −1 |
| 102 | L | −1 | −2 | −3 | −4 | −1 | −2 | −3 | −4 | −3 | 1 | 4 | −2 | 1 | 0 | −3 | −2 | −1 | −2 | −1 | 1 |
| 103 | E | −1 | −1 | −1 | 0 | −3 | 0 | 5 | −2 | −1 | −1 | 0 | 0 | −1 | −2 | 0 | 0 | −1 | −3 | −2 | 0 |
| 104 | G | 0 | −2 | 0 | −1 | −3 | −2 | −2 | 6 | −2 | −4 | −4 | −2 | −3 | −3 | −2 | 0 | −2 | −2 | −3 | −3 |
| 105 | E | −1 | 0 | 0 | 1 | −4 | 1 | 6 | −2 | 0 | −3 | −3 | 0 | −2 | −3 | −1 | 0 | −1 | −3 | −2 | −2 |
| 106 | E | −1 | −1 | 0 | 2 | −4 | 0 | 4 | 2 | −1 | −3 | −4 | 0 | −3 | −3 | −1 | 0 | −1 | −3 | −3 | −3 |
| 107 | C | 0 | −3 | −3 | −3 | 10 | −3 | −4 | −3 | −3 | −1 | −1 | −3 | −1 | −2 | −3 | −1 | −1 | −2 | −2 | −1 |
| 108 | K | −1 | 1 | 0 | 3 | −3 | 0 | 0 | −2 | −1 | −3 | −3 | 4 | −2 | −3 | −1 | 0 | −1 | −3 | −2 | −2 |
| 109 | V | 0 | −2 | −2 | −3 | −1 | −2 | −2 | −3 | −3 | 1 | 2 | −2 | 0 | −1 | −2 | −1 | 1 | −3 | −1 | 4 |
| 110 | L | −1 | −2 | −3 | −4 | −1 | −2 | −3 | −4 | −3 | 1 | 4 | −2 | 1 | 0 | −3 | −2 | −1 | −2 | −1 | 0 |
| 111 | P | −1 | −2 | −2 | −2 | −2 | −2 | −2 | −3 | −2 | 2 | −1 | −2 | −1 | −2 | 6 | −1 | −1 | −4 | −2 | 0 |
| 112 | D | −2 | −1 | 3 | 6 | −3 | 0 | 1 | −1 | 0 | −3 | −4 | −1 | −3 | −3 | −1 | 0 | −1 | −4 | −3 | −3 |
| 113 | R | −1 | 3 | 2 | −2 | −2 | 0 | −1 | −2 | 0 | −1 | 1 | 0 | 0 | −1 | −2 | 0 | −1 | −2 | 1 | −1 |
| 114 | K | 0 | −1 | 0 | 0 | −1 | 0 | 0 | 0 | −1 | −2 | −2 | 1 | −1 | −2 | −1 | 4 | 1 | −3 | −2 | −2 |
| 115 | G | 0 | −2 | 0 | −1 | −3 | −2 | −2 | 6 | −2 | −4 | −4 | −2 | −3 | −3 | −2 | 0 | −2 | −2 | −3 | −3 |
| 116 | W | −3 | −3 | −4 | −4 | −2 | −2 | −3 | −2 | −2 | −3 | −2 | −3 | −1 | 0 | −4 | −3 | −2 | 11 | 1 | −3 |
| 117 | S | 0 | −1 | 0 | −1 | −1 | 0 | −1 | −1 | −1 | −1 | −1 | 0 | 2 | −2 | −1 | 3 | 3 | −2 | −2 | −1 |
| 118 | C | 0 | −3 | −3 | −3 | 10 | −3 | −4 | −3 | −3 | −1 | −1 | −3 | −1 | −2 | −3 | −1 | −1 | −2 | −2 | −1 |
| 119 | S | 1 | −1 | 0 | −1 | −1 | −1 | −1 | −1 | −2 | 0 | −1 | −1 | −1 | −2 | −1 | 3 | 3 | −3 | −2 | 0 |
| 120 | S | 0 | 0 | 0 | 0 | −2 | 3 | 0 | −1 | −1 | −2 | −2 | 0 | −1 | −2 | −1 | 3 | 1 | −3 | −2 | −2 |
| 121 | G | 0 | −2 | 0 | −1 | −3 | −2 | −2 | 6 | −2 | −4 | −4 | −2 | −3 | −3 | −2 | 0 | −2 | −2 | −3 | −3 |
| 122 | N | −1 | −1 | 5 | 0 | −3 | −1 | −1 | 3 | 4 | −3 | −3 | −1 | −2 | −3 | −2 | 0 | −1 | −3 | −1 | −3 |
| 123 | K | −1 | 3 | 0 | −1 | −3 | 0 | 0 | −2 | −1 | −3 | −2 | 5 | −1 | −3 | −1 | 0 | −1 | −3 | −2 | −2 |
| 124 | V | 0 | −3 | −3 | −3 | −1 | −2 | −2 | −4 | −3 | 4 | 0 | −2 | 0 | −1 | −2 | −2 | 0 | −3 | −1 | 4 |
| 125 | K | −1 | 1 | 0 | −1 | −3 | 0 | 0 | −2 | −1 | −3 | −2 | 6 | −1 | −3 | −1 | 0 | −1 | −3 | −2 | −2 |
| 126 | T | 0 | −1 | 0 | −1 | −1 | −1 | −1 | −2 | −2 | −1 | −1 | −1 | −1 | −2 | −1 | 0 | 6 | −2 | −2 | 0 |
| 127 | T | 0 | −1 | 0 | −1 | −1 | −1 | −1 | −2 | −2 | −1 | −1 | −1 | −1 | −2 | −1 | 0 | 6 | −2 | −2 | 0 |
| 128 | R | −1 | 3 | 0 | −1 | −2 | 0 | 0 | −2 | −1 | −2 | −2 | 4 | −1 | −3 | −1 | 0 | 2 | −3 | −2 | −2 |
| 129 | V | 0 | −3 | −3 | −3 | −1 | −2 | −2 | −3 | −3 | 2 | 0 | −2 | 0 | −1 | −2 | −2 | 0 | −3 | −1 | 5 |
| 130 | T | 0 | −1 | 0 | −1 | −1 | 0 | −1 | −1 | 2 | −1 | 0 | −1 | −1 | −2 | −1 | 3 | 3 | −3 | −1 | −1 |
| 131 | H | −1 | 4 | 0 | −1 | −3 | 0 | 0 | −2 | 4 | −3 | −2 | 0 | −1 | −3 | 3 | −1 | −1 | −3 | −1 | −3; |

or
2) comprising a fragment of the amino acid sequence of a1), wherein said fragment is a member of the EGF domain containing protein family, or has an antigenic determinant in common with the amino acid sequence of a1); or
3) comprising a functional equivalent of the amino acid sequence of a1) or the fragment of a2); or
4) consisting of the amino acid sequence of a1); or
5) of any of a1)-a4), wherein the polypeptide has a maximum threshold E value of 10⁻²; or
6) comprising an amino acid sequence satisfying the consensus amino acid sequence G-T-C-E-[VI]-[VI]-[AT]-[AVL]-[HD]-R-[CD]-[CS]-[NS]-[KRQ]-[NP]-[RK]-[IR]-[ET]-[EI]-[RA]-[SR]-Q-T-[VA]-[KR]-C-[SA]-C-[LFR]-[PSK]-G-[KQ]-[VI]-A-G-T-T-R-[NA]-[RQLAK]-P-[SA]-C-V-[DE]-A-[SAR]-I-[VI]-[IELKR]-[WGQET]-[KR]-[WQ]-W-C-[EHNQD]-M-[ENQL]-P-C-[LV]-[EVLP]-G-E-[DEG]-C-[KRD]-[TVL]-L-[PI]-[DN]-[NYSLR]-[STK]-G-W-[MST]-C-[ASIT]-[TSRQ]-P(0,1)-G-[NHG]-[KR]-[IV]-K-T-T; or
7) consisting of an amino acid sequence satisfying the consensus amino acid sequence G-T-C-E-[VI]-[VI]-[AT]-[AVL]-[HD]-R-[CD]-[CS]-[NS]-[KRQ]-[NP]-[RK]-[IR]-[ET]-[EI]-[RA]-[SR]-Q-T-[VA]-[KR]-C-[SA]-C-[LFR]-[PSK]-G-[KQ]-[VI]-A-G-T-T-R-[NA]-[RQLAK]-P-[SA]-C-V-[DE]-A-[SAR]-I-[VI]-[IELKR]-[WGQET]-[KR]-[WQ]-W-C-[EHNQD]-M-[ENQL]-P-C-[LV]-[EVLP]-G-E-[DEG]-C-[KRD]-[TVL]-L-[PI]-[DN]-[NYSLR]-[STK]-G-W-[MST]-C-[ASIT]-[TSRQ]-P(0,1)-G-[NHG]-[KR]-[IV]-K-T-T; or
8) comprising an amino acid sequence selected from the group consisting of SEQ ID NO:18, SEQ ID NO:20, SEQ ID NO:22, SEQ ID NO:24, SEQ ID NO:26, SEQ ID NO:28 and SEQ ID NO:30; or
9) consisting of an amino acid sequence selected from the group consisting of SEQ ID NO:18, SEQ ID NO:20, SEQ ID NO:22, SEQ ID NO:24, SEQ ID NO:26, SEQ ID NO:28 and SEQ ID NO:30; or
10) comprising an amino acid sequence selected from the group consisting of SEQ ID NO:2, SEQ ID NO:4, SEQ ID NO:37 and SEQ ID NO:39; or
11) consisting of an amino acid sequence selected from the group consisting of SEQ ID NO:2, SEQ ID NO:4, SEQ ID NO:37 and SEQ ID NO:39; or
12) comprising an amino acid sequence selected from the group consisting of SEQ ID NO:6, SEQ ID NO:8, SEQ ID NO:41, SEQ ID NO:43, SEQ ID NO:53 and SEQ ID NO:55; or
13) consisting of an amino acid sequence selected from the group consisting of SEQ ID NO:6, SEQ ID NO:8, SEQ ID NO:41, SEQ ID NO:43, SEQ ID NO:53 and SEQ ID NO:55; or
14) comprising an amino acid sequence selected from the group consisting of SEQ ID NO:10, SEQ ID NO:12, SEQ ID NO:45 and SEQ ID NO:47; or
15) consisting of an amino acid sequence selected from the group consisting of SEQ ID NO:10, SEQ ID NO:12, SEQ ID NO:45 and SEQ ID NO:47; or
16) comprising an amino acid sequence selected from the group consisting of SEQ ID NO:14, SEQ ID NO:16, SEQ ID NO:49 and SEQ ID NO:51; or
17) consisting of an amino acid sequence selected from the group consisting of SEQ ID NO:14, SEQ ID NO:16, SEQ ID NO:49 and SEQ ID NO:51; or
18) comprising a fragment of any of the amino acid sequences of a6)-a17), wherein said fragment is a member of the EGF domain containing protein family, or has an antigenic determinant in common with any of the amino acid sequences of a6)-a17); or
19) comprising a functional equivalent of any of the polypeptides of a6)-a18); or
20) comprising the functional equivalent of a3) or a19), wherein the polypeptide is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55, and wherein the polypeptide is a member of the EGF domain containing protein family; or
21) comprising the fragment or functional equivalent of any of a2), a3), a18), a19), or a20), wherein the fragment or functional equivalent has greater than 80% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55, or with an active fragment of any of the foregoing; or
22) comprising the functional equivalent of any of a3), a19), or a20), wherein the functional equivalent exhibits significant structural homology with a polypeptide having an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55; or
23) comprising the fragment of any of a2), a18), or a21), wherein the fragment has an antigenic determinant in common with the polypeptide of a1) or any one of a6)-a17), wherein the antigenic determinant consists of 7 or more amino acid residues from an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55; or
24) according to any of a1)-a23), wherein the polypeptide has biological activity similar to Coagulation Factor X; or
b) a purified nucleic acid molecule:
1) comprising a nucleic acid sequence encoding a polypeptide according to any one of a1)-a24); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, and SEQ ID NO:54, or a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, and SEQ ID NO:54, or a redundant equivalent or fragment of any of the foregoing; or
4) that hybridizes under high stringency conditions with a nucleic acid molecule of any one of b1) to b3); or
c) a vector comprising a nucleic acid molecule according to any one of b1) to b4); or
d) a host cell transformed with a vector according to c); or
e) a ligand:
1) that binds specifically to the polypeptide of any of a1) to a24); or
2) which is an antibody that binds specifically to the polypeptide of any of a1) to a24); or
f) a compound:
1) that increases the level of expression or activity of a polypeptide according to any of a1) to a24); or
2) that decreases the level of expression or activity of a polypeptide according to any of a1) to a24); or
g) the compound of f1) or f2), wherein the compound binds to a polypeptide according to any of a1) to a24) without inducing any of the biological effects of the polypeptide; or
h) the compound of g), wherein the compound is a natural or modified substrate, ligand, enzyme, receptor or structural or functional mimetic; or
i) a pharmaceutical composition comprising any one of a) to h), and a pharmaceutically acceptable carrier.

71. The method of claim 70, wherein the disease includes one or more of among fertility disorders, including infertility, cell proliferative disorders, including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection.

72. The method of claim 70, wherein the disease includes one or more of among arthritis, rheumatoid arthritis (RA), psoriatic arthritis, osteoarthritis, systemic lupus erythematosus (SLE), systemic sclerosis, scleroderma, polymyositis, glomerulonephritis, fibrosis, lung fibrosis and inflammation, allergic or hypersensitivity diseases, dermatitis, asthma, chronic obstructive pulmonary disease (COPD), inflammatory bowel disease (IBD), Crohn's diseases, ulcerative colitis, multiple sclerosis, septic shock, HIV infection, transplant rejection, wound healing, metastasis, endometriosis, hepatitis, liver fibrosis, cancer, analgesia, and vascular inflammation related to atherosclerosis, Crohn's disease, inflammatory skin diseases such as atopic dermatitis and psoriasis, Sjogren's syndrome and/or myasthenia gravis.

73. The method of claim 70, wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

74. The method of claim 70, wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

75. The method of claim 69, wherein said method of using a composition of matter comprises the method for diagnosing a disease in a patient, comprising assessing the level of expression of a natural gene encoding a polypeptide of claim 68, or assessing the activity of a polypeptide of claim 68, in tissue from said patient; and comparing said level of expression or activity to a control level, wherein a level that is different to said control level is indicative of disease.

76. The method of claim 75, which is carried out in vitro.

77. The method of claim 75, comprising:
a) contacting a ligand of claim 68 with a biological sample under conditions suitable for the formation of a ligand-polypeptide complex; and
b) detecting said complex.

78. The method of claim 75, comprising:
a) contacting a sample of tissue from the patient with a nucleic acid probe under stringent conditions that allow the formation of a hybrid complex between a nucleic acid molecule of claim 68 and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

79. The method of claim 75, comprising:
a) contacting a sample of nucleic acid from tissue of the patient with a nucleic acid primer under stringent conditions that allow the formation of a hybrid complex between a nucleic acid molecule of claim 68 and the primer;
b) contacting a control sample with said primer under the same conditions used in step a);
c) amplifying the sampled nucleic acid; and
d) detecting the level of amplified nucleic acid from both patient and control samples;
wherein detection of levels of the amplified nucleic acid in the patient sample that differ significantly from levels of the amplified nucleic acid in the control sample is indicative of disease.

80. The method of claim 75, comprising:
a) obtaining a tissue sample from a patient being tested for disease;
b) isolating a nucleic acid molecule of claim 68 from said tissue sample; and
c) diagnosing the patient for disease by detecting the presence of a mutation which is associated with disease in the nucleic acid molecule as an indication of the disease.

81. The method of claim 80, further comprising amplifying the nucleic acid molecule to form an amplified product and detecting the presence or absence of a mutation in the amplified product.

82. The method of claim 80, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

83. The method of claim 75, wherein said disease includes one or more of among fertility disorders, including infertility, cell proliferative disorders, including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection.

84. The method of claim 75, wherein said disease is a disease in which lymph antigens are implicated.

85. The method of claim 69, wherein said method of using a composition of matter comprises the method of monitoring the therapeutic treatment of a disease, comprising monitoring over a period of time the level of expression or activity of a polypeptide of claim 68, or the level of expression of a nucleic acid molecule of claim 68, in tissue from said patient, wherein altering said level of expression or activity over the period of time towards a control level is indicative of regression of said disease.

86. The method of claim 69, wherein said method of using a composition of matter comprises the method for identification of a compound that is effective in the treatment and/or diagnosis of a disease, comprising contacting a polypeptide of claim 68 or a nucleic acid molecule of claim 68 with one or more compounds suspected of possessing binding affinity for said polypeptide or nucleic acid molecule, and selecting a compound that binds specifically to said nucleic acid molecule or polypeptide.

87. The method of claim 69, wherein said method of using a composition of matter comprises the method for screening candidate compounds, comprising contacting a non-human transgenic animal of claim 68 with a candidate compound and determining the effect of the compound on the disease of the animal.

88. An isolated polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55.

89. The isolated polypeptide of claim 88, wherein said polypeptide consists of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, and SEQ ID NO:55.
